(19) United States
(12) Patent Application Publication
(10) Pub. No.: US 2007/0128606 A1
(43) Pub. Date: Jun. 7, 2007

(54) Prkcs as modifiers of the beta catenin pathway and methods of use

(75) Inventors: Helen Francis-Lang, San Francisco, CA (US); Christopher G. Winter, Needham, MA (US); Richard Benn Abegania Ventura, Daly City, CA (US); Joanne I. Adamkewicz, Novato, CA (US); Timothy S. Heuer, El Granada, CA (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: EXELIXIS INC, SOUTH SAN FRANSICO CALIFORNIA, CA (US)

(21) Appl. No.: 10/567,950

(22) PCT Filed: Aug. 13, 2004
(86) PCT No.: PCT/US04/26361

§ 371 Date: Sep. 8, 2006

Related U.S. Application Data
(60) Provisional application No. 60/495,172, filed on Aug. 14, 2003.

(51) Int. Cl. C12Q 1/68; G01N 33/574

(52) U.S. Cl. 435/6; 435/7.23

(57) ABSTRACT

Human PRKC genes are identified as modulators of the beta catenin pathway, and thus are therapeutic targets for disorders associated with defective beta catenin function. Methods for identifying modulators of beta catenin, comprising screening for agents that modulate the activity of PRKC are provided.

### REFERENCE TO RELATED APPLICATIONS

This application claims priority to U.S. provisional patent application 60/495,172 filed Aug. 14, 2003. The contents of the prior application are hereby incorporated in their entirety.

### BACKGROUND OF THE INVENTION

The Drosophila Melanogaster Armadillo/beta-catenin protein is implicated in multiple cellular functions. The protein functions in cell signaling via the Wingless (Wg)/Wnt signaling pathway. It also functions as a cell adhesion protein at the cell membrane in a complex with E-cadherin and alpha-catenin (Cox et al. (1996) J. Cell Biol. 134: 133-148; Godt and Tepass (1998) Nature 395: 387-391; White et al. (1998) J Cell biol. 140:183-195). These two roles of beta-catenin can be separated from each other (Orsulic and Peifer (1996) J. Cell Biol. 134: 1283-1300; Sanson et al. (1996) Nature 383: 627-630).

In Wingless cell signaling, beta-catenin levels are tightly regulated by a complex containing APC, Axin, and GSK3 beta/SGG/ZW3 (Peifer et al. (1994) Development 120: 369-380).

The Wingless/beta-catenin signaling pathway is frequently mutated in human cancers, particularly those of the colon. Mutations in the tumor suppressor gene APC, as well as point mutations in beta-catenin itself lead to the stabilization of the beta-catenin protein and inappropriate activation of this pathway.

The protein kinase C (PKC) family of serine/threonine protein kinases has at least eight members, which are differentially expressed and are involved in a wide variety of cellular processes such as proliferation, differentiation and secretion. Protein kinase C iota (PRKCI) belongs to the PKC family, and is calcium-independent and phospholipid-dependent. PRKCI can be recruited to vesicle tubular clusters (VTCs) by direct interaction with the small GTPase RAB2, where this kinase phosphorylates glyceraldehydes-3-phosphate dehydrogenase (GAPD/GAPDH) and plays a role in microtubule dynamics in the early secretory pathway. PRKCI is necessary for BCL-ABL-mediated resistance to drug-induced apoptosis and therefore protects leukemia cells against drug-induced apoptosis.

Protein kinase C (PKC) zeta is another member of the PKC family of serine/threonine kinases. Unlike the classical PKC isoenzymes which are calcium-dependent, PKC zeta exhibits a constitutive kinase activity which is independent of calcium. Mice with targeted disruption of PRKCZ gene are known in the art (Leitges M et al (2001) Molec Cell 8:771-780).

The ability to manipulate the genomes of model organisms such as Drosophila provides a powerful means to analyze biochemical processes that, due to significant evolutionary conservation, have direct relevance to more complex vertebrate organisms. Due to a high level of gene and pathway conservation, the strong similarity of cellular processes, and the functional conservation of genes between these model organisms and mammals, identification of the involvement of novel genes in particular pathways and their functions in such model organisms can directly contribute to the understanding of the correlative pathways and methods of modulating them in mammals (see, for example, Mechler B M et al., 1985 EMBO J 4:1551-1557; Gateff E. 1982 Adv. Cancer Res. 37: 33-74; Watson K L., et al., 1994 J Cell Sci. 18: 19-33; Miklos G L, and Rubin G M. 1996 Cell 86:521-529; Wassarman D A, et al., 1995 Curr Opin Gen Dev 5: 44-50; and Booth D R. 1999 Cancer Metastasis Rev. 18: 261-284). For example, a genetic screen can be carried out in an invertebrate model organism having underexpression (e.g. knockout) or overexpression of a gene (referred to as a “genetic entry point”) that yields a visible phenotype. Additional genes are mutated in a random or targeted manner. When a gene mutation changes the original phenotype caused by the mutation in the genetic entry point, the gene is identified as a “modifier” involved in the same or overlapping pathway as the genetic entry point. When the genetic entry point is an ortholog of a human gene implicated in a disease pathway, such as beta catenin, modifier genes can be identified that may be attractive candidate targets for novel therapeutics.

All references cited herein, including patents, patent applications, publications, and sequence information in referenced Genbank identifier numbers, are incorporated herein in their entireties.

### SUMMARY OF THE INVENTION

We have discovered genes that modify the beta catenin pathway in Drosophila, and identified their human orthologs, hereinafter referred to as Protein Kinase C (PRKC). The invention provides methods for utilizing these beta catenin modifier genes and polypeptides to identify PRKC-modulating agents that are candidate therapeutic agents that can be used in the treatment of disorders associated with defective or impaired beta catenin function and/or PRKC function. Preferred PRKC-modulating agents specifically bind to PRKC polypeptides and restore beta catenin function. Other preferred PRKC-modulating agents are nucleic acid modulators such as antisense oligomers and RNAi that repress PRKC gene expression or product activity by, for example, binding to and inhibiting the respective nucleic acid (i.e. DNA or mRNA).

PRKC modulating agents may be evaluated by any convenient in vitro or in vivo assay for molecular interaction with a PRKC polypeptide or nucleic acid. In one embodiment, candidate PRKC modulating agents are tested with an assay system comprising a PRKC polypeptide or nucleic acid. Agents that produce a change in the activity of the assay system relative to controls are identified as candidate beta catenin modulating agents. The assay system may be cell-based or cell-free. PRKC-modulating agents include PRKC related proteins (e.g. dominant negative mutants, and biotherapeutics); PRKC-specific antibodies; PRKC-specific antisense oligomers and other nucleic acid modulators; and chemical agents that specifically bind to or interact with PRKC or compete with PRKC binding partner (e.g. by binding to a PRKC binding partner). In one specific embodiment, a small molecule modulator is identified using a kinase assay. In specific embodiments, the screening assay system is selected from a binding assay, an apoptosis assay, a cell proliferation assay, an angiogenesis assay, and a hypoxic induction assay.

In another embodiment, candidate beta catenin pathway modulating agents are further tested using a second assay system that detects changes in the beta catenin pathway, such as angiogenic, apoptotic, or cell proliferation changes produced by the originally identified candidate agent or an agent derived from the original agent. The second assay system may use cultured cells or non-human animals. In specific embodiments, the secondary assay system uses non-human animals, including animals predetermined to have a disease or disorder implicating the beta catenin pathway, such as an angiogenic, apoptotic, or cell proliferation disorder (e.g. cancer).

The invention further provides methods for modulating the PRKC function and/or the beta catenin pathway in a mammalian cell by contacting the mammalian cell with an agent that specifically binds a PRKC polypeptide or nucleic acid. The agent may be a small molecule modulator, a nucleic acid modulator, or an antibody and may be administered to a mammalian animal predetermined to have a pathology associated with the beta catenin pathway.

### DETAILED DESCRIPTION OF THE INVENTION

In a screen to identify enhancers and suppressors of the Wg signaling pathway, we generated activated beta-catenin models in Drosophila based on human tumor data (Polakis (2000) Genes and Development 14: 1837-1851). We identified modifiers of the Wg pathway and identified their orthologs. The APKC gene was identified as a modifier of the beta catenin pathway. Accordingly, vertebrate orthologs of these modifiers, and preferably the human orthologs, PRKC genes (i.e., nucleic acids and polypeptides) are attractive drug targets for the treatment of pathologies associated with a defective beta catenin signaling pathway, such as cancer.

In vitro and in vivo methods of assessing PRKC function are provided herein. Modulation of the PRKC or their respective binding partners is useful for understanding the association of the beta catenin pathway and its members in normal and disease conditions and for developing diagnostics and therapeutic modalities for beta catenin related pathologies. PRKC-modulating agents that act by inhibiting or enhancing PRKC expression, directly or indirectly, for example, by affecting a PRKC function such as enzymatic (e.g., catalytic) or binding activity, can be identified using methods provided herein. PRKC modulating agents are useful in diagnosis, therapy and pharmaceutical development.

Nucleic Acids and Polypeptides of the Invention

Sequences related to PRKC nucleic acids and polypeptides that can be used in the invention are disclosed in Genbank (referenced by Genbank identifier (GI) number) as GI#s 4506070 (SEQ ID NO:1), 18314568 (SEQ ID NO:2), 34222297 (SEQ ID NO:4), 34191041 (SEQ ID NO:5), 10864649 (SEQ ID NO:6), 14165514 (SEQ ID NO:7), 307355 (SEQ ID NO:8), 33873791 (SEQ ID NO:9), and 33878518 (SEQ ID NO: 10) for nucleic acid, and GI#s 4506071 (SEQ ID NO:11) and 10864650 (SEQ ID NO:12) for polypeptides. Additionally, nucleic acid of SEQ ID NO:3 can also be used in the invention.

The term “PRKC polypeptide” refers to a full-length PRKC protein or a functionally active fragment or derivative thereof. A “functionally active” PRKC fragment or derivative exhibits one or more functional activities associated with a full-length, wild-type PRKC protein, such as antigenic or immunogenic activity, enzymatic activity, ability to bind natural cellular substrates, etc. The functional activity of PRKC proteins, derivatives and fragments can be assayed by various methods known to one skilled in the art (Current Protocols in Protein Science (1998) Coligan et al., eds., John Wiley & Sons, Inc., Somerset, N.J.) and as further discussed below. In one embodiment, a functionally active PRKC polypeptide is a PRKC derivative capable of rescuing defective endogenous PRKC activity, such as in cell based or animal assays; the rescuing derivative may be from the same or a different species. For purposes herein, functionally active fragments also include those fragments that comprise one or more structural domains of a PRKC, such as a kinase domain or a binding domain. Protein domains can be identified using the PFAM program (Bateman A., et al., Nucleic Acids Res, 1999, 27:260-2). For example, the kinase domain (PFAM 00069) of PRKC from GI#s 4506071 and 10864650 (SEQ ID NOs:11 and 12, respectively) is located respectively at approximately amino acid residues 245 to 513 and 252 to 518. Further, the Kinase C terminal domain (PFAM 00433) of the same proteins is located respectively at approximately amino acid residues 514 to 580 and 519 to 585. Methods for obtaining PRKC polypeptides are also further described below. In some embodiments, preferred fragments are functionally active, domain-containing fragments comprising at least 25 contiguous amino acids, preferably at least 50, more preferably 75, and most preferably at least 100 contiguous amino acids of a PRKC. In further preferred embodiments, the fragment comprises the entire functionally active domain.

The term “PRKC nucleic acid” refers to a DNA or RNA molecule that encodes a PRKC polypeptide. Preferably, the PRKC polypeptide or nucleic acid or fragment thereof is from a human, but can also be an ortholog, or derivative thereof with at least 70% sequence identity, preferably at least 80%, more preferably 85%, still more preferably 90%, and most preferably at least 95% sequence identity with human PRKC. Methods of identifying orthlogs are known in the art. Normally, orthologs in different species retain the same function, due to presence of one or more protein motifs and/or 3-dimensional structures. Orthologs are generally identified by sequence homology analysis, such as BLAST analysis, usually using protein bait sequences. Sequences are assigned as a potential ortholog if the best hit sequence from the forward BLAST result retrieves the original query sequence in the reverse BLAST (Huynen M A and Bork P, Proc Natl Acad Sci (1998) 95:5849-5856; Huynen M A et al., Genome Research (2000) 10:1204-1210). Programs for multiple sequence alignment, such as CLUSTAL (Thompson J D et al, 1994, Nucleic Acids Res 22:4673-4680) may be used to highlight conserved regions and/or residues of orthologous proteins and to generate phylogenetic trees. In a phylogenetic tree representing multiple homologous sequences from diverse species (e.g., retrieved through BLAST analysis), orthologous sequences from two species generally appear closest on the tree with respect to all other sequences from these two species. Structural threading or other analysis of protein folding (e.g., using software by ProCeryon, Biosciences, Salzburg, Austria) may also identify potential orthologs. In evolution, when a gene duplication event follows speciation, a single gene in one species, such as Drosophzila, may correspond to multiple genes (paralogs) in another, such as human. As used herein, the term “orthologs” encompasses paralogs. As used herein, “percent (%) sequence identity” with respect to a subject sequence, or a specified portion of a subject sequence, is defined as the percentage of nucleotides or amino acids in the candidate derivative sequence identical with the nucleotides or amino acids in the subject sequence (or specified portion thereof), after aligning the sequences and introducing gaps, if necessary to achieve the maximum percent sequence identity, as generated by the program WU-BLAST-2.0a19 (Altschul et al., J. Mol. Biol. (1997) 215:403-410) with all the search parameters set to default values. The HSP S and HSP S2 parameters are dynamic values and are established by the program itself depending upon the composition of the particular sequence and composition of the particular database against which the sequence of interest is being searched. A % identity value is determined by the number of matching identical nucleotides or amino acids divided by the sequence length for which the percent identity is being reported. “Percent (%) amino acid sequence similarity” is determined by doing the same calculation as for determining % amino acid sequence identity, but including conservative amino acid substitutions in addition to identical amino acids in the computation.

A conservative amino acid substitution is one in which an amino acid is substituted for another amino acid having similar properties such that the folding or activity of the protein is not significantly affected. Aromatic amino acids that can be substituted for each other are phenylalanine, tryptophan, and tyrosine; interchangeable hydrophobic amino acids are leucine, isoleucine, methionine, and valine; interchangeable polar amino acids are glutamine and asparagine; interchangeable basic amino acids are arginine, lysine and histidine; interchangeable acidic amino acids are aspartic acid and glutamic acid; and interchangeable small amino acids are alanine, serine, threonine, cysteine and glycine.

Alternatively, an alignment for nucleic acid sequences is provided by the local homology algorithm of Smith and Waterman (Smith and Waterman, 1981, Advances in Applied Mathematics 2:482-489; database: European Bioinformatics Institute; Smith and Waterman, 1981, J. of Molec. Biol., 147:195-197; Nicholas et al., 1998, “A Tutorial on Searching Sequence Databases and Sequence Scoring Methods” (www.psc.edu) and references cited therein.; W. R. Pearson, 1991, Genomics 11:635-650). This algorithm can be applied to amino acid sequences by using the scoring matrix developed by Dayhoff (Dayhoff: Atlas of Protein Sequences and Structure, M. O. Dayhoff ed., 5 suppl. 3:353-358, National Biomedical Research Foundation, Washington, D.C., USA), and normalized by Gribskov (Gribskov 1986 Nucl. Acids Res. 14(6):6745-6763). The Smith-Waterman algorithm may be employed where default parameters are used for scoring (for example, gap open penalty of 12, gap extension penalty of two). From the data generated, the “Match” value reflects “sequence identity.”

Derivative nucleic acid molecules of the subject nucleic acid molecules include sequences that hybridize to the nucleic acid sequence of a PRKC. The stringency of hybridization can be controlled by temperature, ionic strength, pH, and the presence of denaturing agents such as formamide during hybridization and washing. Conditions routinely used are set out in readily available procedure texts (e.g., Current Protocol in Molecular Biology, Vol. 1, Chap. 2.10, John Wiley & Sons, Publishers (1994); Sambrook et al., Molecular Cloning, Cold Spring Harbor (1989)). In some embodiments, a nucleic acid molecule of the invention is capable of hybridizing to a nucleic acid molecule containing the nucleotide sequence of a PRKC under high stringency hybridization conditions that are: prehybridization of filters containing nucleic acid for 8 hours to overnight at 65° C. in a solution comprising 6× single strength citrate (SSC) (1×SSC is 0.15 M NaCl, 0.015 M Na citrate; pH 7.0), 5× Denhardt's solution, 0.05% sodium pyrophosphate and 100 μg/ml herring sperm DNA; hybridization for 18-20 hours at 65° C. in a solution containing 6×SSC, 1× Denhardt's solution, 100 μg/ml yeast tRNA and 0.05% sodium pyrophosphate; and washing of filters at 65° C. for 1 h in a solution containing 0.1×SSC and 0.1% SDS (sodium dodecyl sulfate).

In other embodiments, moderately stringent hybridization conditions are used that are: pretreatment of filters containing nucleic acid for 6 h at 40° C. in a solution containing 35% formamide, 5×SSC, 50 mM Tris-HCl (pH7.5), 5 mM EDTA, 0.1% PVP, 0.1% Ficoll, 1% BSA, and 500 μg/ml denatured salmon sperm DNA; hybridization for 18-20 h at 40° C. in a solution containing 35% formamide, 5×SSC, 50 mM Tris-HCl (pH7.5), 5 mM EDTA, 0.02% PVP, 0.02% Ficoll, 0.2% BSA, 100 μg/ml salmon sperm DNA, and 10% (wt/vol) dextran sulfate; followed by washing twice for 1 hour at 55° C. in a solution containing 2×SSC and 0.1% SDS.

Alternatively, low stringency conditions can be used that are: incubation for 8 hours to overnight at 37° C. in a solution comprising 20% formamide, 5×SSC, 50 mM sodium phosphate (pH 7.6), 5× Denhardt's solution, 10% dextran sulfate, and 20 μg/ml denatured sheared salmon sperm DNA; hybridization in the same buffer for 18 to 20 hours; and washing of filters in 1×SSC at about 37° C. for 1 hour.

Isolation, Production, Expression, and Mis-Expression of PRKC Nucleic Acids and Polypeptides

PRKC nucleic acids and polypeptides are useful for identifying and testing agents that modulate PRKC function and for other applications related to the involvement of PRKC in the beta catenin pathway. PRKC nucleic acids and derivatives and orthologs thereof may be obtained using any available method. For instance, techniques for isolating cDNA or genomic DNA sequences of interest by screening DNA libraries or by using polymerase chain reaction (PCR) are well known in the art. In general, the particular use for the protein will dictate the particulars of expression, production, and purification methods. For instance, production of proteins for use in screening for modulating agents may require methods that preserve specific biological activities of these proteins, whereas production of proteins for antibody generation may require structural integrity of particular epitopes. Expression of proteins to be purified for screening or antibody production may require the addition of specific tags (e.g., generation of fusion proteins). Overexpression of a PRKC protein for assays used to assess PRKC function, such as involvement in cell cycle regulation or hypoxic response, may require expression in eukaryotic cell lines capable of these cellular activities. Techniques for the expression, production, and purification of proteins are well known in the art; any suitable means therefore may be used (e.g., Higgins S J and Hames B D (eds.) Protein Expression: A Practical Approach, Oxford University Press Inc., New York 1999; Stanbury P F et al., Principles of Fermentation Technology, 2^{nd} edition, Elsevier Science, New York, 1995; Doonan S (ed.) Protein Purification Protocols, Humana Press, New Jersey, 1996; Coligan J E et al, Current Protocols in Protein Science (eds.), 1999, John Wiley & Sons, New York). In particular embodiments, recombinant PRKC is expressed in a cell line known to have defective beta catenin function. The recombinant cells are used in cell-based screening assay systems of the invention, as described further below.

The nucleotide sequence encoding a PRKC polypeptide can be inserted into any appropriate expression vector. The necessary transcriptional and translational signals, including promoter/enhancer element, can derive from the native PRKC gene and/or its flanking regions or can be heterologous. A variety of host-vector expression systems may be utilized, such as mammalian cell systems infected with virus (e.g. vaccinia virus, adenovirus, etc.); insect cell systems infected with virus (e.g. baculovirus); microorganisms such as yeast containing yeast vectors, or bacteria transformed with bacteriophage, plasmid, or cosmid DNA. An isolated host cell strain that modulates the expression of, modifies, and/or specifically processes the gene product may be used.

To detect expression of the PRKC gene product, the expression vector can comprise a promoter operably linked to a PRKC gene nucleic acid, one or more origins of replication, and, one or more selectable markers (e.g. thymidine kinase activity, resistance to antibiotics, etc.). Alternatively, recombinant expression vectors can be identified by assaying for the expression of the PRKC gene product based on the physical or functional properties of the PRKC protein in in vitro assay systems (e.g. immunoassays).

The PRKC protein, fragment, or derivative may be optionally expressed as a fusion, or chimeric protein product (i.e. it is joined via a peptide bond to a heterologous protein sequence of a different protein), for example to facilitate purification or detection. A chimeric product can be made by ligating the appropriate nucleic acid sequences encoding the desired amino acid sequences to each other using standard methods and expressing the chimeric product. A chimeric product may also be made by protein synthetic techniques, e.g. by use of a peptide synthesizer (Hunkapiller et al., Nature (1984) 310:105-111).

Once a recombinant cell that expresses the PRKC gene sequence is identified, the gene product can be isolated and purified using standard methods (e.g. ion exchange, affinity, and gel exclusion chromatography; centrifugation; differential solubility; electrophoresis). Alternatively, native PRKC proteins can be purified from natural sources, by standard methods (e.g. immunoaffinity purification). Once a protein is obtained, it may be quantified and its activity measured by appropriate methods, such as immunoassay, bioassay, or other measurements of physical properties, such as crystallography.

The methods of this invention may also use cells that have been engineered for altered expression (mis-expression) of PRKC or other genes associated with the beta catenin pathway. As used herein, mis-expression encompasses ectopic expression, over-expression, under-expression, and non-expression (e.g. by gene knock-out or blocking expression that would otherwise normally occur).

Genetically Modified Animals

Animal models that have been genetically modified to alter PRKC expression may be used in in vivo assays to test for activity of a candidate beta catenin modulating agent, or to further assess the role of PRKC in a beta catenin pathway process such as apoptosis or cell proliferation. Preferably, the altered PRKC expression results in a detectable phenotype, such as decreased or increased levels of cell proliferation, angiogenesis, or apoptosis compared to control animals having normal PRKC expression. The genetically modified animal may additionally have altered beta catenin expression (e.g. beta catenin knockout). Preferred genetically modified animals are mammals such as primates, rodents (preferably mice or rats), among others. Preferred non-mammalian species include zebrafish, C. elegans, and Drosophila. Preferred genetically modified animals are transgenic animals having a heterologous nucleic acid sequence present as an extrachromosomal element in a portion of its cells, i.e. mosaic animals (see, for example, techniques described by Jakobovits, 1994, Curr. Biol. 4:761-763.) or stably integrated into its germ line DNA (i.e., in the genomic sequence of most or all of its cells). Heterologous nucleic acid is introduced into the germ line of such transgenic animals by genetic manipulation of, for example, embryos or embryonic stem cells of the host animal.

Methods of making transgenic animals are well-known in the art (for transgenic mice see Brinster et al., Proc. Nat. Acad. Sci. USA 82: 4438-4442 (1985), U.S. Pat. Nos. 4,736,866 and 4,870,009, both by Leder et al., U.S. Pat. No. 4,873,191 by Wagner et al., and Hogan, B., Manipulating the Mouse Embryo, Cold Spring Harbor Laboratory Press, Cold Spring Harbor, N.Y., (1986); for particle bombardment see U.S. Pat. No., 4,945,050, by Sandford et al.; for transgenic Drosophila see Rubin and Spradling, Science (1982) 218:348-53 and U.S. Pat. No. 4,670,388; for transgenic insects see Berghammer A. J. et al., A Universal Marker for Transgenic Insects (1999) Nature 402:370-371; for transgenic Zebrafish see Lin S., Transgenic Zebrafish, Methods Mol Biol. (2000); 136:375-3830); for microinjection procedures for fish, amphibian eggs and birds see Houdebine and Chourrout, Experientia (1991) 47:897-905; for transgenic rats see Hammer et al., Cell (1990) 63:1099-1112; and for culturing of embryonic stem (ES) cells and the subsequent production of transgenic animals by the introduction of DNA into ES cells using methods such as electroporation, calcium phosphate/DNA precipitation and direct injection see, e.g., Teratocarcinomas and Embryonic Stem Cells, A Practical Approach, E. J. Robertson, ed., IRL Press (1987)). Clones of the nonhuman transgenic animals can be produced according to available methods (see Wilmut, I. et al. (1997) Nature 385:810-813; and PCT International Publication Nos. WO 97/07668 and WO 97/07669).

In one embodiment, the transgenic animal is a “knock-out” animal having a heterozygous or homozygous alteration in the sequence of an endogenous PRKC gene that results in a decrease of PRKC function, preferably such that PRKC expression is undetectable or insignificant. Knockout animals are typically generated by homologous recombination with a vector comprising a transgene having at least a portion of the gene to be knocked out. Typically a deletion, addition or substitution has been introduced into the transgene to functionally disrupt it. The transgene can be a human gene (e.g., from a human genomic clone) but more preferably is an ortholog of the human gene derived from the transgenic host species. For example, a mouse PRKC gene is used to construct a homologous recombination vector suitable for altering an endogenous PRKC gene in the mouse genome. Detailed methodologies for homologous recombination in mice are available (see Capecchi, Science (1989) 244:1288-1292; Joyner et al., Nature (1989) 338:153-156). Procedures for the production of non-rodent transgenic mammals and other animals are also available (Houdebine and Chourrout, supra; Pursel et al., Science (1989) 244:1281-1288; Simms et al., Bio/Technology (1988) 6:179-183). In a preferred embodiment, knock-out animals, such as mice harboring a knockout of a specific gene, may be used to produce antibodies against the human counterpart of the gene that has been knocked out (Claesson M H et al., (1994) Scan J Immunol 40:257-264; Declerck P J et al., (1995) J Biol Chem. 270:8397-400).

In another embodiment, the transgenic animal is a “knock-in” animal having an alteration in its genome that results in altered expression (e.g., increased (including ectopic) or decreased expression) of the PRKC gene, e.g., by introduction of additional copies of PRKC, or by operatively inserting a regulatory sequence that provides for altered expression of an endogenous copy of the PRKC gene. Such regulatory sequences include inducible, tissue-specific, and constitutive promoters and enhancer elements. The knock-in can be homozygous or heterozygous.

Transgenic nonhuman animals can also be produced that contain selected systems allowing for regulated expression of the transgene. One example of such a system that may be produced is the cre/loxP recombinase system of bacteriophage P1 (Lakso et al., PNAS (1992) 89:6232-6236; U.S. Pat. No. 4,959,317). If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase. Another example of a recombinase system is the FLP recombinase system of Saccharomyces cerevisiae (O'Gorman et al. (1991) Science 251:1351-1355; U.S. Pat. No. 5,654,182). In a preferred embodiment, both Cre-LoxP and Flp-Frt are used in the same system to regulate expression of the transgene, and for sequential deletion of vector sequences in the same cell (Sun X et al (2000) Nat Genet 25:83-6).

The genetically modified animals can be used in genetic studies to further elucidate the beta catenin pathway, as animal models of disease and disorders implicating defective beta catenin function, and for in vivo testing of candidate therapeutic agents, such as those identified in screens described below. The candidate therapeutic agents are administered to a genetically modified animal having altered PRKC function and phenotypic changes are compared with appropriate control animals such as genetically modified animals that receive placebo treatment, and/or animals with unaltered PRKC expression that receive candidate therapeutic agent.

In addition to the above-described genetically modified animals having altered PRKC function, animal models having defective beta catenin function (and otherwise normal PRKC function), can be used in the methods of the present invention. For example, a beta catenin knockout mouse can be used to assess, in vivo, the activity of a candidate beta catenin modulating agent identified in one of the in vitro assays described below. Preferably, the candidate beta catenin modulating agent when administered to a model system with cells defective in beta catenin function, produces a detectable phenotypic change in the model system indicating that the beta catenin function is restored, i.e., the cells exhibit normal cell cycle progression.

Modulating Agents

The invention provides methods to identify agents that interact with and/or modulate the function of PRKC and/or the beta catenin pathway. Modulating agents identified by the methods are also part of the invention. Such agents are useful in a variety of diagnostic and therapeutic applications associated with the beta catenin pathway, as well as in further analysis of the PRKC protein and its contribution to the beta catenin pathway. Accordingly, the invention also provides methods for modulating the beta catenin pathway comprising the step of specifically modulating PRKC activity by administering a PRKC-interacting or -modulating agent.

As used herein, a “PRKC-modulating agent” is any agent that modulates PRKC function, for example, an agent that interacts with PRKC to inhibit or enhance PRKC activity or otherwise affect normal PRKC function. PRKC function can be affected at any level, including transcription, protein expression, protein localization, and cellular or extra-cellular activity. In a preferred embodiment, the PRKC-modulating agent specifically modulates the function of the PRKC. The phrases “specific modulating agent”, “specifically modulates”, etc., are used herein to refer to modulating agents that directly bind to the PRKC polypeptide or nucleic acid, and preferably inhibit, enhance, or otherwise alter, the function of the PRKC. These phrases also encompass modulating agents that alter the interaction of the PRKC with a binding partner, substrate, or cofactor (e.g. by binding to a binding partner of a PRKC, or to a protein/binding partner complex, and altering PRKC function). In a further preferred embodiment, the PRKC-modulating agent is a modulator of the beta catenin pathway (e.g. it restores and/or upregulates beta catenin function) and thus is also a beta catenin-modulating agent.

Preferred PRKC-modulating agents include small molecule compounds; PRKC-interacting proteins, including antibodies and other biotherapeutics; and nucleic acid modulators such as antisense and RNA inhibitors. The modulating agents may be formulated in pharmaceutical compositions, for example, as compositions that may comprise other active ingredients, as in combination therapy, and/or suitable carriers or excipients. Techniques for formulation and administration of the compounds may be found in “Remington's Pharmaceutical Sciences” Mack Publishing Co., Easton, Pa., 19^{th} edition.

Small Molecule Modulators

Small molecules are often preferred to modulate function of proteins with enzymatic function, and/or containing protein interaction domains. Chemical agents, referred to in the art as “small molecule” compounds are typically organic, non-peptide molecules, having a molecular weight up to 10,000, preferably up to 5,000, more preferably up to 1,000, and most preferably up to 500 daltons. This class of modulators includes chemically synthesized molecules, for instance, compounds from combinatorial chemical libraries. Synthetic compounds may be rationally designed or identified based on known or inferred properties of the PRKC protein or may be identified by screening compound libraries. Alternative appropriate modulators of this class are natural products, particularly secondary metabolites from organisms such as plants or fungi, which can also be identified by screening compound libraries for PRKC-modulating activity. Methods for generating and obtaining compounds are well known in the art (Schreiber SL, Science (2000) 151: 1964-1969; Radmann J and Gunther J, Science (2000) 151:1947-1948).

Small molecule modulators identified from screening assays, as described below, can be used as lead compounds from which candidate clinical compounds may be designed, optimized, and synthesized. Such clinical compounds may have utility in treating pathologies associated with the beta catenin pathway. The activity of candidate small molecule modulating agents may be improved several-fold through iterative secondary functional validation, as further described below, structure determination, and candidate modulator modification and testing. Additionally, candidate clinical compounds are generated with specific regard to clinical and pharmacological properties. For example, the reagents may be derivatized and re-screened using in vitro and in vivo assays to optimize activity and minimize toxicity for pharmaceutical development.

Protein Modulators

Specific PRKC-interacting proteins are useful in a variety of diagnostic and therapeutic applications related to the beta catenin pathway and related disorders, as well as in validation assays for other PRKC-modulating agents. In a preferred embodiment, PRKC-interacting proteins affect normal PRKC function, including transcription, protein expression, protein localization, and cellular or extra-cellular activity. In another embodiment, PRKC-interacting proteins are useful in detecting and providing information about the function of PRKC proteins, as is relevant to beta catenin related disorders, such as cancer (e.g., for diagnostic means).

An PRKC-interacting protein may be endogenous, i.e. one that naturally interacts genetically or biochemically with a PRKC, such as a member of the PRKC pathway that modulates PRKC expression, localization, and/or activity. PRKC-modulators include dominant negative forms of PRKC-interacting proteins and of PRKC proteins themselves. Yeast two-hybrid and variant screens offer preferred methods for identifying endogenous PRKC-interacting proteins (Finley, R. L. et al. (1996) in DNA Cloning-Expression Systems: A Practical Approach, eds. Glover D. & Hames B. D (Oxford University Press, Oxford, England), pp. 169-203; Fashema S F et al., Gene (2000) 250:1-14; Drees B L Curr Opin Chem Biol (1999) 3:64-70; Vidal M and Legrain P Nucleic Acids Res (1999) 27:919-29; and U.S. Pat. No. 5,928,868). Mass spectrometry is an alternative preferred method for the elucidation of protein complexes (reviewed in, e.g., Pandley A and Mann M, Nature (2000) 405:837-846; Yates JR 3^{rd}, Trends Genet (2000) 16:5-8).

An PRKC-interacting protein may be an exogenous protein, such as a PRKC-specific antibody or a T-cell antigen receptor (see, e.g., Harlow and Lane (1988) Antibodies, A Laboratory Manual, Cold Spring Harbor Laboratory; Harlow and Lane (1999) Using antibodies: a laboratory manual. Cold Spring Harbor, N.Y.: Cold Spring Harbor Laboratory Press). PRKC antibodies are further discussed below.

In preferred embodiments, a PRKC-interacting protein specifically binds a PRKC protein. In alternative preferred embodiments, a PRKC-modulating agent binds a PRKC substrate, binding partner, or cofactor.

Antibodies

In another embodiment, the protein modulator is a PRKC specific antibody agonist or antagonist. The antibodies have therapeutic and diagnostic utilities, and can be used in screening assays to identify PRKC modulators. The antibodies can also be used in dissecting the portions of the PRKC pathway responsible for various cellular responses and in the general processing and maturation of the PRKC.

Antibodies that specifically bind PRKC polypeptides can be generated using known methods. Preferably the antibody is specific to a mammalian ortholog of PRKC polypeptide, and more preferably, to human PRKC. Antibodies may be polyclonal, monoclonal (mAbs), humanized or chimeric antibodies, single chain antibodies, Fab fragments, F(ab′).sub.2 fragments, fragments produced by a FAb expression library, anti-idiotypic (anti-Id) antibodies, and epitope-binding fragments of any of the above. Epitopes of PRKC which are particularly antigenic can be selected, for example, by routine screening of PRKC polypeptides for antigenicity or by applying a theoretical method for selecting antigenic regions of a protein (Hopp and Wood (1981), Proc. Nati. Acad. Sci. U.S.A. 78:3824-28; Hopp and Wood, (1983) Mol. Immunol. 20:483-89; Sutcliffe et al., (1983) Science 219:660-66) to the amino acid sequence of a PRKC. Monoclonal antibodies with affinities of 10⁸ M⁻¹ preferably 10⁹ M⁻¹ to 10¹⁰ M⁻¹, or stronger can be made by standard procedures as described (Harlow and Lane, supra; Goding (1986) Monoclonal Antibodies: Principles and Practice (2d ed) Academic Press, New York; and U.S. Pat. Nos. 4,381,292; 4,451,570; and 4,618,577). Antibodies may be generated against crude cell extracts of PRKC or substantially purified fragments thereof. If PRKC fragments are used, they preferably comprise at least 10, and more preferably, at least 20 contiguous amino acids of a PRKC protein. In a particular embodiment, PRKC-specific antigens and/or immunogens are coupled to carrier proteins that stimulate the immune response. For example, the subject polypeptides are covalently coupled to the keyhole limpet hemocyanin (KLH) carrier, and the conjugate is emulsified in Freund's complete adjuvant, which enhances the immune response. An appropriate immune system such as a laboratory rabbit or mouse is immunized according to conventional protocols.

The presence of PRKC-specific antibodies is assayed by an appropriate assay such as a solid phase enzyme-linked immunosorbant assay (ELISA) using immobilized corresponding PRKC polypeptides. Other assays, such as radioimmunoassays or fluorescent assays might also be used.

Chimeric antibodies specific to PRKC polypeptides can be made that contain different portions from different animal species. For instance, a human immunoglobulin constant region may be linked to a variable region of a murine mAb, such that the antibody derives its biological activity from the human antibody, and its binding specificity from the murine fragment. Chimeric antibodies are produced by splicing together genes that encode the appropriate regions from each species (Morrison et al., Proc. Natl. Acad. Sci. (1984) 81:6851-6855; Neuberger et al., Nature (1984) 312:604-608; Takeda et al., Nature (1985) 31:452-454). Humanized antibodies, which are a form of chimeric antibodies, can be generated by grafting complementary-determining regions (CDRs) (Carlos, T. M., J. M. Harlan. 1994. Blood 84:2068-2101) of mouse antibodies into a background of human framework regions and constant regions by recombinant DNA technology (Riechmann L M, et al., 1988 Nature 323: 323-327). Humanized antibodies contain ˜10% murine sequences and ˜90% human sequences, and thus further reduce or eliminate immunogenicity, while retaining the antibody specificities (Co MS, and Queen C. 1991 Nature 351: 501-501; Morrison S L. 1992 Ann. Rev. Immun. 10:239-265). Humanized antibodies and methods of their production are well-known in the art (U.S. Pat. Nos. 5,530,101, 5,585,089, 5,693,762, and 6,180,370).

PRKC-specific single chain antibodies which are recombinant, single chain polypeptides formed by linking the heavy and light chain fragments of the Fv regions via an amino acid bridge, can be produced by methods known in the art (U.S. Pat. No. 4,946,778; Bird, Science (1988) 242:423-426; Huston et al., Proc. Natl. Acad. Sci. USA (1988) 85:5879-5883; and Ward et al., Nature (1989) 334:544-546).

Other suitable techniques for antibody production involve in vitro exposure of lymphocytes to the antigenic polypeptides or alternatively to selection of libraries of antibodies in phage or similar vectors (Huse et al., Science (1989) 246:1275-1281). As used herein, T-cell antigen receptors are included within the scope of antibody modulators (Harlow and Lane, 1988, supra).

The polypeptides and antibodies of the present invention may be used with or without modification. Frequently, antibodies will be labeled by joining, either covalently or non-covalently, a substance that provides for a detectable signal, or that is toxic to cells that express the targeted protein (Menard S, et al., Int J. Biol Markers (1989) 4:131-134). A wide variety of labels and conjugation techniques are known and are reported extensively in both the scientific and patent literature. Suitable labels include radionuclides, enzymes, substrates, cofactors, inhibitors, fluorescent moieties, fluorescent emitting lanthanide metals, chemiluminescent moieties, bioluminescent moieties, magnetic particles, and the like (U.S. Pat. Nos. 3,817,837; 3,850,752; 3,939,350; 3,996,345; 4,277,437; 4,275,149; and 4,366,241). Also, recombinant immunoglobulins may be produced (U.S. Pat. No. 4,816,567). Antibodies to cytoplasmic polypeptides may be delivered and reach their targets by conjugation with membrane-penetrating toxin proteins (U.S. Pat. No. 6,086,900).

When used therapeutically in a patient, the antibodies of the subject invention are typically administered parenterally, when possible at the target site, or intravenously. The therapeutically effective dose and dosage regimen is determined by clinical studies. Typically, the amount of antibody administered is in the range of about 0.1 mg/kg-to about 10 mg/kg of patient weight. For parenteral administration, the antibodies are formulated in a unit dosage injectable form (e.g., solution, suspension, emulsion) in association with a pharmaceutically acceptable vehicle. Such vehicles are inherently nontoxic and non-therapeutic. Examples are water, saline, Ringer's solution, dextrose solution, and 5% human serum albumin. Nonaqueous vehicles such as fixed oils, ethyl oleate, or liposome carriers may also be used. The vehicle may contain minor amounts of additives, such as buffers and preservatives, which enhance isotonicity and chemical stability or otherwise enhance therapeutic potential. The antibodies' concentrations in such vehicles are typically in the range of about 1 mg/ml to about 10 mg/ml. Immunotherapeutic methods are further described in the literature (US Pat. No. 5,859,206; WO0073469).

Nucleic Acid Modulators

Other preferred PRKC-modulating agents comprise nucleic acid molecules, such as antisense oligomers or double stranded RNA (dsRNA), which generally inhibit PRKC activity. Preferred nucleic acid modulators interfere with the function of the PRKC nucleic acid such as DNA replication, transcription, translocation of the PRKC RNA to the site of protein translation, translation of protein from the PRKC RNA, splicing of the PRKC RNA to yield one or more mRNA species, or catalytic activity which may be engaged in or facilitated by the PRKC RNA.

In one embodiment, the antisense oligomer is an oligonucleotide that is sufficiently complementary to a PRKC mRNA to bind to and prevent translation, preferably by binding to the 5′ untranslated region. PRKC-specific antisense oligonucleotides, preferably range from at least 6 to about 200 nucleotides. In some embodiments the oligonucleotide is preferably at least 10, 15, or 20 nucleotides in length. In other embodiments, the oligonucleotide is preferably less than 50, 40, or 30 nucleotides in length. The oligonucleotide can be DNA or RNA or a chimeric mixture or derivatives or modified versions thereof, single-stranded or double-stranded. The oligonucleotide can be modified at the base moiety, sugar moiety, or phosphate backbone. The oligonucleotide may include other appending groups such as peptides, agents that facilitate transport across the cell membrane, hybridization-triggered cleavage agents, and intercalating agents.

In another embodiment, the antisense oligomer is a phosphothioate morpholino oligomer (PMO). PMOs are assembled from four different morpholino subunits, each of which contain one of four genetic bases (A, C, G, or T) linked to a six-membered morpholine ring. Polymers of these subunits are joined by non-ionic phosphodiamidate intersubunit linkages. Details of how to make and use PMOs and other antisense oligomers are well known in the art (e.g. see WO99/18193; Probst J C, Antisense Oligodeoxynucleotide and Ribozyme Design, Methods. (2000) 22(3):271-281; Summerton J, and Weller D. 1997 Antisense Nucleic Acid Drug Dev. :7:187-95; U.S. Pat. No. 5,235,033; and U.S. Pat. No. 5,378,841).

Alternative preferred PRKC nucleic acid modulators are double-stranded RNA species mediating RNA interference (RNAi). RNAi is the process of sequence-specific, post-transcriptional gene silencing in animals and plants, initiated by double-stranded RNA (dsRNA) that is homologous in sequence to the silenced gene. Methods relating to the use of RNAi to silence genes in C. elegans, Drosophila, plants, and humans are known in the art (Fire A, et al., 1998 Nature 391:806-811; Fire, A. Trends Genet. 15, 358-363 (1999); Sharp, P. A. RNA interference 2001. Genes Dev. 15, 485490 (2001); Hammond, S. M., et al., Nature Rev. Genet. 2,110-1119 (2001); Tuschl, T. Chem. Biochem. 2, 239-245 (2001); Hamilton, A. et al., Science 286,950-952 (1999); Hammond, S. M., et al., Nature 404, 293-296 (2000); Zamore, P. D., et al., Cell 101, 25-33 (2000); Bernstein, E., et al., Nature 409, 363-366 (2001); Elbashir, S. M., et al., Genes Dev. 15, 188-200 (2001); W00129058; W09932619; Elbashir S M, et al., 2001 Nature 411:494-498).

Nucleic acid modulators are commonly used as research reagents, diagnostics, and therapeutics. For example, antisense oligonucleotides, which are able to inhibit gene expression with exquisite specificity, are often used to elucidate the function of particular genes (see, for example, U.S. Pat. No. 6,165,790). Nucleic acid modulators are also used, for example, to distinguish between functions of various members of a biological pathway. For example, antisense oligomers have been employed as therapeutic moieties in the treatment of disease states in animals and man and have been demonstrated in numerous clinical trials to be safe and effective (Milligan J F, et al, Current Concepts in Antisense Drug Design, J Med Chem. (1993) 36:1923-1937; Tonkinson J L et al., Antisense Oligodeoxynucleotides as Clinical Therapeutic Agents, Cancer Invest. (1996) 14:54-65). Accordingly, in one aspect of the invention, a PRKC-specific nucleic acid modulator is used in an assay to further elucidate the role of the PRKC in the beta catenin pathway, and/or its relationship to other members of the pathway. In another aspect of the invention, a PRKC-specific antisense oligomer is used as a therapeutic agent for treatment of beta catenin-related disease states.

Assay Systems

The invention provides assay systems and screening methods for identifying specific modulators of PRKC activity. As used herein, an “assay system” encompasses all the components required for performing and analyzing results of an assay that detects and/or measures a particular event. In general, primary assays are used to identify or confirm a modulator's specific biochemical or molecular effect with respect to the PRKC nucleic acid or protein. In general, secondary assays further assess the activity of a PRKC modulating agent identified by a primary assay and may confirm that the modulating agent affects PRKC in a manner relevant to the beta catenin pathway. In some cases, PRKC modulators will be directly tested in a secondary assay.

In a preferred embodiment, the screening method comprises contacting a suitable assay system comprising a PRKC polypeptide or nucleic acid with a candidate agent under conditions whereby, but for the presence of the agent, the system provides a reference activity (e.g. kinase activity), which is based on the particular molecular event the screening method detects. A statistically significant difference between the agent-biased activity and the reference activity indicates that the candidate agent modulates PRKC activity, and hence the beta catenin pathway. The PRKC polypeptide or nucleic acid used in the assay may comprise any of the nucleic acids or polypeptides described above.

Primary Assays

The type of modulator tested generally determines the type of primary assay.

Primary Assays for Small Molecule Modulators

For small molecule modulators, screening assays are used to identify candidate modulators. Screening assays may be cell-based or may use a cell-free system that recreates or retains the relevant biochemical reaction of the target protein (reviewed in Sittampalam G S et al, Curr Opin Chem Biol (1997) 1:384-91 and accompanying references). As used herein the term “cell-based” refers to assays using live cells, dead cells, or a particular cellular fraction, such as a membrane, endoplasmic reticulum, or mitochondrial fraction. The term “cell free” encompasses assays using substantially purified protein (either endogenous or recombinantly produced), partially purified or crude cellular extracts. Screening assays may detect a variety of molecular events, including protein-DNA interactions, protein-protein interactions (e.g., receptor-ligand binding), transcriptional activity (e.g., using a reporter gene), enzymatic activity (e.g., via a property of the substrate), activity of second messengers, immunogenicty and changes in cellular morphology or other cellular characteristics. Appropriate screening assays may use a wide range of detection methods including fluorescent, radioactive, colorimetric, spectrophotometric, and amperometric methods, to provide a read-out for the particular molecular event detected.

Cell-based screening assays usually require systems for recombinant expression of PRKC and any auxiliary proteins demanded by the particular assay. Appropriate methods for generating recombinant proteins produce sufficient quantities of proteins that retain their relevant biological activities and are of sufficient purity to optimize activity and assure assay reproducibility. Yeast two-hybrid and variant screens, and mass spectrometry provide preferred methods for determining protein-protein interactions and elucidation of protein complexes. In certain applications, when PRKC-interacting proteins are used in screens to identify small molecule modulators, the binding specificity of the interacting protein to the PRKC protein may be assayed by various known methods such as substrate processing (e.g. ability of the candidate PRKC-specific binding agents to function as negative effectors in PRKC-expressing cells), binding equilibrium constants (usually at least about 10⁷ M⁻¹, preferably at least about 10⁸ M⁻¹, more preferably at least about 10⁹ M⁻¹), and immunogenicity (e.g. ability to elicit PRKC specific antibody in a heterologous host such as a mouse, rat, goat or rabbit). For enzymes and receptors, binding may be assayed by, respectively, substrate and ligand processing.

The screening assay may measure a candidate agent's ability to specifically bind to or modulate activity of a PRKC polypeptide, a fusion protein thereof, or to cells or membranes bearing the polypeptide or fusion protein. The PRKC polypeptide can be full length or a fragment thereof that retains functional PRKC activity. The PRKC polypeptide may be fused to another polypeptide, such as a peptide tag for detection or anchoring, or to another tag. The PRKC polypeptide is preferably human PRKC, or is an ortholog or derivative thereof as described above. In a preferred embodiment, the screening assay detects candidate agent-based modulation of PRKC interaction with a binding target, such as an endogenous or exogenous protein or other substrate that has PRKC-specific binding activity, and can be used to assess normal PRKC gene function.

Suitable assay formats that may be adapted to screen for PRKC modulators are known in the art. Preferred screening assays are high throughput or ultra high throughput and thus provide automated, cost-effective means of screening compound libraries for lead compounds (Fernandes P B, Curr Opin Chem Biol (1998) 2:597-603; Sundberg S A, Curr Opin Biotechnol 2000, 11:47-53). In one preferred embodiment, screening assays uses fluorescence technologies, including fluorescence polarization, time-resolved fluorescence, and fluorescence resonance energy transfer. These systems offer means to monitor protein-protein or DNA-protein interactions in which the intensity of the signal emitted from dye-labeled molecules depends upon their interactions with partner molecules (e.g., Selvin P R, Nat Struct Biol (2000) 7:730-4; Fernandes P B, supra; Hertzberg R P and Pope A J, Curr Opin Chem Biol (2000) 4:445-451).

A variety of suitable assay systems may be used to identify candidate PRKC and beta catenin pathway modulators (e.g. U.S. Pat. No. 6,165,992 (kinase assays); U.S. Pat. Nos. 5,550,019 and 6,133,437 (apoptosis assays); and U.S. Pat. Nos. 5,976,782, 6,225,118 and 6,444,434 (angiogenesis assays), among others). Specific preferred assays are described in more detail below.

Kinase assays. In some preferred embodiments the screening assay detects the ability of the test agent to modulate the kinase activity of a PRKC polypeptide. In further embodiments, a cell-free kinase assay system is used to identify a candidate beta catenin modulating agent, and a secondary, cell-based assay, such as an apoptosis or hypoxic induction assay (described below), may be used to further characterize the candidate beta catenin modulating agent. Many different assays for kinases have been reported in the literature and are well known to those skilled in the art (e.g. U.S. Pat. No. 6,165,992; Zhu et al., Nature Genetics (2000) 26:283-289; and W00073469). Radioassays, which monitor the transfer of a gamma phosphate are frequently used. For instance, a scintillation assay for p56 (lck) kinase activity monitors the transfer of the gamma phosphate from gamma—³³P ATP to a biotinylated peptide substrate; the substrate is captured on a streptavidin coated bead that transmits the signal (Beveridge M et al., J Biomol Screen (2000) 5:205-212). This assay uses the scintillation proximity assay (SPA), in which only radio-ligand bound to receptors tethered to the surface of an SPA bead are detected by the scintillant immobilized within it, allowing binding to be measured without separation of bound from free ligand.

Other assays for protein kinase activity may use antibodies that specifically recognize phosphorylated substrates. For instance, the kinase receptor activation (KIRA) assay measures receptor tyrosine kinase activity by ligand stimulating the intact receptor in cultured cells, then capturing solubilized receptor with specific antibodies and quantifying phosphorylation via phosphotyrosine ELISA (Sadick M D, Dev Biol Stand (1999) 97:121-133).

Another example of antibody based assays for protein kinase activity is TRF (time-resolved fluorometry). This method utilizes europium chelate-labeled anti-phosphotyrosine antibodies to detect phosphate transfer to a polymeric substrate coated onto microtiter plate wells. The amount of phosphorylation is then detected using time-resolved, dissociation-enhanced fluorescence (Braunwalder A F, et al., Anal Biochem 1996 July 1; 238(2):159-64).

Apoptosis assays. Apoptosis or programmed cell death is a suicide program is activated within the cell, leading to fragmentation of DNA, shrinkage of the cytoplasm, membrane changes and cell death. Apoptosis is mediated by proteolytic enzymes of the caspase family. Assays for apoptosis may be performed by terminal deoxynucleotidyl transferase-mediated digoxigenin-11-dUTP nick end labeling (TUNEL) assay. The TUNEL assay is used to measure nuclear DNA fragmentation characteristic of apoptosis (Lazebnik et al., 1994, Nature 371, 346), by following the incorporation of fluorescein-dUTP (Yonehara et al., 1989, J. Exp. Med. 169, 1747). Apoptosis may further be assayed by acridine orange staining of tissue culture cells (Lucas, R., et al., 1998, Blood 15:4730-41). Other cell-based apoptosis assays include the caspase-3/7 assay and the cell death nucleosome ELISA assay. The caspase 3/7 assay is based on the activation of the caspase cleavage activity as part of a cascade of events that occur during programmed cell death in many apoptotic pathways. In the caspase 3/7 assay (commercially available Apo-ONE™ Homogeneous Caspase-3/7 assay from Promega, cat# 67790), lysis buffer and caspase substrate are mixed and added to cells. The caspase substrate becomes fluorescent when cleaved by active caspase 3/7. The nucleosome ELISA assay is a general cell death assay known to those skilled in the art, and available commercially (Roche, Cat# 1774425). This assay is a quantitative sandwich-enzyme-immunoassay which uses monoclonal antibodies directed against DNA and histones respectively, thus specifically determining amount of mono- and oligonucleosomes in the cytoplasmic fraction of cell lysates. Mono and oligonucleosomes are enriched in the cytoplasm during apoptosis due to the fact that DNA fragmentation occurs several hours before the plasma membrane breaks down, allowing for accumalation in the cytoplasm. Nucleosomes are not present in the cytoplasmic fraction of cells that are not undergoing apoptosis. The Phospho-histone H2B assay is another apoptosis assay, based on phosphorylation of histone H2B as a result of apoptosis. Fluorescent dyes that are associated with phosphohistone H2B may be used to measure the increase of phosphohistone H2B as a result of apoptosis. Apoptosis assays that simultaneously measure multiple parameters associated with apoptosis have also been developed. In such assays, various cellular parameters that can be associated with antibodies or fluorescent dyes, and that mark various stages of apoptosis are labeled, and the results are measured using instruments such as Cellomics™ ArrayScan® HCS System. The measurable parameters and their markers include anti-active caspase-3 antibody which marks intermediate stage apoptosis, anti-PARP-p85 antibody (cleaved PARP) which marks late stage apoptosis, Hoechst labels which label the nucleus and are used to measure nuclear swelling as a measure of early apoptosis and nuclear condensation as a measure of late apoptosis, and TOTO-3 fluorescent dye which labels DNA of dead cells with high cell membrane permeability.

An apoptosis assay system may comprise a cell that expresses a PRKC, and that optionally has defective beta catenin function (e.g. beta catenin is over-expressed or under-expressed relative to wild-type cells). A test agent can be added to the apoptosis assay system and changes in induction of apoptosis relative to controls where no test agent is added, identify candidate beta catenin modulating agents. In some embodiments of the invention, an apoptosis assay may be used as a secondary assay to test a candidate beta catenin modulating agents that is initially identified using a cell-free assay system. An apoptosis assay may also be used to test whether PRKC function plays a direct role in apoptosis. For example, an apoptosis assay may be performed on cells that over- or under-express PRKC relative to wild type cells. Differences in apoptotic response compared to wild type cells suggests that the PRKC plays a direct role in the apoptotic response. Apoptosis assays are described further in U.S. Pat. No. 6,133,437.

Cell proliferation and cell cycle assays. Cell proliferation may be assayed via bromodeoxyuridine (BRDU) incorporation. This assay identifies a cell population undergoing DNA synthesis by incorporation of BRDU into newly-synthesized DNA. Newly-synthesized DNA may then be detected using an anti-BRDU antibody (Hoshino et al., 1986, Int. J. Cancer 38, 369; Campana et al., 1988, J. Immunol. Meth. 107, 79), or by other means.

Cell proliferation is also assayed via phospho-histone H3 staining, which identifies a cell population undergoing mitosis by phosphorylation of histone H3. Phosphorylation of histone H3 at serine 10 is detected using an antibody specific to the phosphorylated form of the serine 10 residue of histone H3. (Chadlee, D. N. 1995, J. Biol. Chem 270:20098-105). Cell Proliferation may also be examined using [³H]-thymidine incorporation (Chen, J., 1996, Oncogene 13:1395-403; Jeoung, J., 1995, J. Biol. Chem. 270:18367-73). This assay allows for quantitative characterization of S-phase DNA syntheses. In this assay, cells synthesizing DNA will incorporate [³H]-thymidine into newly synthesized DNA. Incorporation can then be measured by standard techniques such as by counting of radioisotope in a scintillation counter (e.g., Beckman LS 3800 Liquid Scintillation Counter). Another proliferation assay uses the dye Alamar Blue (available from Biosource International), which fluoresces when reduced in living cells and provides an indirect measurement of cell number (Voytik-Harbin S L et al., 1998, In Vitro Cell Dev Biol Anim 34:239-46). Yet another proliferation assay, the MTS assay, is based on in vitro cytotoxicity assessment of industrial chemicals, and uses the soluble tetrazolium salt, MTS. MTS assays are commercially available, for example, the Promega CellTiter 96® AQueous Non-Radioactive Cell Proliferation Assay (Cat.# G5421).

Cell proliferation may also be assayed by colony formation in soft agar, or clonogenic survival assay (Sambrook et al., Molecular Cloning, Cold Spring Harbor (1989)). For example, cells transformed with PRKC are seeded in soft agar plates, and colonies are measured and counted after two weeks incubation.

Cell proliferation may also be assayed by measuring ATP levels as indicator of metabolically active cells. Such assays are commercially available, for example Cell Titer-Glo™, which is a luminescent homogeneous assay available from Promega.

Involvement of a gene in the cell cycle may be assayed by flow cytometry (Gray J W et al. (1986) Int J Radiat Biol Relat Stud Phys Chem Med 49:237-55). Cells transfected with a PRKC may be stained with propidium iodide and evaluated in a flow cytometer (available from Becton Dickinson), which indicates accumulation of cells in different stages of the cell cycle.

Accordingly, a cell proliferation or cell cycle assay system may comprise a cell that expresses a PRKC, and that optionally has defective beta catenin function (e.g. beta catenin is over-expressed or under-expressed relative to wild-type cells). A test agent can be added to the assay system and changes in cell proliferation or cell cycle relative to controls where no test agent is added, identify candidate beta catenin modulating agents. In some embodiments of the invention, the cell proliferation or cell cycle assay may be used as a secondary assay to test a candidate beta catenin modulating agents that is initially identified using another assay system such as a cell-free assay system. A cell proliferation assay may also be used to test whether PRKC function plays a direct role in cell proliferation or cell cycle. For example, a cell proliferation or cell cycle assay may be performed on cells that over- or under-express PRKC relative to wild type cells. Differences in proliferation or cell cycle compared to wild type cells suggests that the PRKC plays a direct role in cell proliferation or cell cycle.

Angiogenesis. Angiogenesis may be assayed using various human endothelial cell systems, such as umbilical vein, coronary artery, or dermal cells. Suitable assays include Alamar Blue based assays (available from Biosource International) to measure proliferation; migration assays using fluorescent molecules, such as the use of Becton Dickinson Falcon HTS FluoroBlock cell culture inserts to measure migration of cells through membranes in presence or absence of angiogenesis enhancer or suppressors; and tubule formation assays based on the formation of tubular structures by endothelial cells on Matrigel® (Becton Dickinson). Accordingly, an angiogenesis assay system may comprise a cell that expresses a PRKC, and that optionally has defective beta catenin function (e.g. beta catenin is over-expressed or under-expressed relative to wild-type cells). A test agent can be added to the angiogenesis assay system and changes in angiogenesis relative to controls where no test agent is added, identify candidate beta catenin modulating agents. In some embodiments of the invention, the angiogenesis assay may be used as a secondary assay to test a candidate beta catenin modulating agents that is initially identified using another assay system. An angiogenesis assay may also be used to test whether PRKC function plays a direct role in cell proliferation. For example, an angiogenesis assay may be performed on cells that over- or under-express PRKC relative to wild type cells. Differences in angiogenesis compared to wild type cells suggests that the PRKC plays a direct role in angiogenesis. U.S. Pat. Nos. 5,976,782, 6,225,118 and 6,444,434, among others, describe various angiogenesis assays.

Hypoxic induction. The alpha subunit of the transcription factor, hypoxia inducible factor-1 (HIF-1), is upregulated in tumor cells following exposure to hypoxia in vitro. Under hypoxic conditions, HIF-1 stimulates the expression of genes known to be important in tumour cell survival, such as those encoding glyolytic enzymes and VEGF. Induction of such genes by hypoxic conditions may be assayed by growing cells transfected with PRKC in hypoxic conditions (such as with 0.1% O2, 5% CO2, and balance N2, generated in a Napco 7001 incubator (Precision Scientific)) and normoxic conditions, followed by assessment of gene activity or expression by Taqman®. For example, a hypoxic induction assay system may comprise a cell that expresses a PRKC, and that optionally has defective beta catenin function (e.g. beta catenin is over-expressed or under-expressed relative to wild-type cells). A test agent can be added to the hypoxic induction assay system and changes in hypoxic response relative to controls where no test agent is added, identify candidate beta catenin modulating agents. In some embodiments of the invention, the hypoxic induction assay may be used as a secondary assay to test a candidate beta catenin modulating agents that is initially identified using another assay system. A hypoxic induction assay may also be used to test whether PRKC function plays a direct role in the hypoxic response. For example, a hypoxic induction assay may be performed on cells that over- or under-express PRKC relative to wild type cells. Differences in hypoxic response compared to wild type cells suggests that the PRKC plays a direct role in hypoxic induction.

Cell adhesion. Cell adhesion assays measure adhesion of cells to purified adhesion proteins, or adhesion of cells to each other, in presence or absence of candidate modulating agents. Cell-protein adhesion assays measure the ability of agents to modulate the adhesion of cells to purified proteins. For example, recombinant proteins are produced, diluted to 2.5 g/mL in PBS, and used to coat the wells of a microtiter plate. The wells used for negative control are not coated. Coated wells are then washed, blocked with 1% BSA, and washed again. Compounds are diluted to 2× final test concentration and added to the blocked, coated wells. Cells are then added to the wells, and the unbound cells are washed off. Retained cells are labeled directly on the plate by adding a membrane-permeable fluorescent dye, such as calcein-AM, and the signal is quantified in a fluorescent microplate reader.

Cell-cell adhesion assays measure the ability of agents to modulate binding of cell adhesion proteins with their native ligands. These assays use cells that naturally or recombinantly express the adhesion protein of choice. In an exemplary assay, cells expressing the cell adhesion protein are plated in wells of a multiwell plate. Cells expressing the ligand are labeled with a membrane-permeable fluorescent dye, such as BCECF, and allowed to adhere to the monolayers in the presence of candidate agents. Unbound cells are washed off, and bound cells are detected using a fluorescence plate reader.

High-throughput cell adhesion assays have also been described. In one such assay, small molecule ligands and peptides are bound to the surface of microscope slides using a microarray spotter, intact cells are then contacted with the slides, and unbound cells are washed off. In this assay, not only the binding specificity of the peptides and modulators against cell lines are determined, but also the functional cell signaling of attached cells using immunofluorescence techniques in situ on the microchip is measured (Falsey J R et al., Bioconjug Chem. 2001 May-Jun; 12(3):346-53).

Tubulogenesis. Tubulogenesis assays monitor the ability of cultured cells, generally endothelial cells, to form tubular structures on a matrix substrate, which generally simulates the environment of the extracellular matrix. Exemplary substrates include Matrigel™ (Becton Dickinson), an extract of basement membrane proteins containing laminin, collagen IV, and heparin sulfate proteoglycan, which is liquid at 4° C. and forms a solid gel at 37° C. Other suitable matrices comprise extracellular components such as collagen, fibronectin, and/or fibrin. Cells are stimulated with a pro-angiogenic stimulant, and their ability to form tubules is detected by imaging. Tubules can generally be detected after an overnight incubation with stimuli, but longer or shorter time frames may also be used. Tube formation assays are well known in the art (e.g., Jones M K et al., 1999, Nature Medicine 5:1418-1423). These assays have traditionally involved stimulation with serum or with the growth factors FGF or VEGF. Serum represents an undefined source of growth factors. In a preferred embodiment, the assay is performed with cells cultured in serum free medium, in order to control which process or pathway a candidate agent modulates. Moreover, we have found that different target genes respond differently to stimulation with different pro-angiogenic agents, including inflammatory angiogenic factors such as TNF-alpa. Thus, in a further preferred embodiment, a tubulogenesis assay system comprises testing a PRKC's response to a variety of factors, such as FGF, VEGF, phorbol myristate acetate (PMA), TNF-alpha, ephrin, etc.

Cell Migration. An invasion/migration assay (also called a migration assay) tests the ability of cells to overcome a physical barrier and to migrate towards pro-angiogenic signals. Migration assays are known in the art (e.g., Paik J H et al., 2001, J Biol Chem 276:11830-11837). In a typical experimental set-up, cultured endothelial cells are seeded onto a matrix-coated porous lamina, with pore sizes generally smaller than typical cell size. The matrix generally simulates the environment of the extracellular matrix, as described above. The lamina is typically a membrane, such as the transwell polycarbonate membrane (Coming Costar Corporation, Cambridge, Mass.), and is generally part of an upper chamber that is in fluid contact with a lower chamber containing pro-angiogenic stimuli. Migration is generally assayed after an overnight incubation with stimuli, but longer or shorter time frames may also be used. Migration is assessed as the number of cells that crossed the lamina, and may be detected by staining cells with hemotoxylin solution (VWR Scientific, South San Francisco, Calif.), or by any other method for determining cell number. In another exemplary set up, cells are fluorescently labeled and migration is detected using fluorescent readings, for instance using the Falcon HTS FluoroBlok (Becton Dickinson). While some migration is observed in the absence of stimulus, migration is greatly increased in response to pro-angiogenic factors. As described above, a preferred assay system for migration/invasion assays comprises testing a PRKC's response to a variety of pro-angiogenic factors, including tumor angiogenic and inflammatory angiogenic agents, and culturing the cells in serum free medium.

Sprouting assay. A sprouting assay is a three-dimensional in vitro angiogenesis assay that uses a cell-number defined spheroid aggregation of endothelial cells (“spheroid”), embedded in a collagen gel-based matrix. The spheroid can serve as a starting point for the sprouting of capillary-like structures by invasion into the extracellular matrix (termed “cell sprouting”)and the subsequent formation of complex anastomosing networks (Korff and Augustin, 1999, J Cell Sci 112:3249-58). In an exemplary experimental set-up, ,spheroids are prepared by pipetting 400 human umbilical vein endothelial cells into individual wells of a nonadhesive 96-well plates to allow overnight spheroidal aggregation (Korff and Augustin: J Cell Biol 143: 1341-52, 1998). Spheroids are harvested and seeded in 900 μl of methocel-collagen solution and pipetted into individual wells of a 24 well plate to allow collagen gel polymerization. Test agents are added after 30 min by pipetting 100 μl of 10-fold concentrated working dilution of the test substances on top of the gel. Plates are incubated at 37° C. for 24 h. Dishes are fixed at the end of the experimental incubation period by addition of paraformaldehyde. Sprouting intensity of endothelial cells can be quantitated by an automated image analysis system to determine the cumulative sprout length per spheroid.

Primary assays for antibody modulators

For antibody modulators, appropriate primary assays test is a binding assay that tests the antibody's affinity to and specificity for the PRKC protein. Methods for testing antibody affinity and specificity are well known in the art (Harlow and Lane, 1988, 1999, supra). The enzyme-linked inmunosorbant assay (ELISA) is a preferred method for detecting PRKC-specific antibodies; others include FACS assays, radioimmunoassays, and fluorescent assays.

In some cases, screening assays described for small molecule modulators may also be used to test antibody modulators.

Primary Assays for Nucleic Acid Modulators

For nucleic acid modulators, primary assays may test the ability of the nucleic acid modulator to inhibit or enhance PRKC gene expression, preferably mRNA expression. In general, expression analysis comprises comparing PRKC expression in like populations of cells (e.g., two pools of cells that endogenously or recombinantly express PRKC) in the presence and absence of the nucleic acid modulator. Methods for analyzing mRNA and protein expression are well known in the art. For instance, Northern blotting, slot blotting, ribonuclease protection, quantitative RT-PCR (e.g., using the TaqMan®, PE Applied Biosystems), or microarray analysis may be used to confirm that PRKC mRNA expression is reduced in cells treated with the nucleic acid modulator (e.g., Current Protocols in Molecular Biology (1994) Ausubel F M et al., eds., John Wiley & Sons, Inc., chapter 4; Freeman W M et al, Biotechniques (1999) 26:112-125; Kallioniemi O P, Ann Med 2001, 33:142-147; Blohm D H and Guiseppi-Elie, A Curr Opin Biotechnol 2001, 12:41-47). Protein expression may also be monitored. Proteins are most commonly detected with specific antibodies or antisera directed against either the PRKC protein or specific peptides. A variety of means including Western blotting, ELISA, or in situ detection, are available (Harlow E and Lane D, 1988 and 1999, supra).

In some cases, screening assays described for small molecule modulators, particularly in assay systems that involve PRKC mRNA expression, may also be used to test nucleic acid modulators.

Secondary Assays

Secondary assays may be used to further assess the activity of PRKC-modulating agent identified by any of the above methods to confirm that the modulating agent affects PRKC in a manner relevant to the beta catenin pathway. As used herein, PRKC-modulating agents encompass candidate clinical compounds or other agents derived from previously identified modulating agent. Secondary assays can also be used to test the activity of a modulating agent on a particular genetic or biochemical pathway or to test the specificity of the modulating agent's interaction with PRKC.

Secondary assays generally compare like populations of cells or animals (e.g., two pools of cells or animals that endogenously or recombinantly express PRKC) in the presence and absence of the candidate modulator. In general, such assays test whether treatment of cells or animals with a candidate PRKC-modulating agent results in changes in the beta catenin pathway in comparison to untreated (or mock- or placebo-treated) cells or animals. Certain assays use “sensitized genetic backgrounds”, which, as used herein, describe cells or animals engineered for altered expression of genes in the beta catenin or interacting pathways.

Cell-Based Assays

Cell based assays may detect endogenous beta catenin pathway activity or may rely on recombinant expression of beta catenin pathway components. Any of the aforementioned assays may be used in this cell-based format. Candidate modulators are typically added to the cell media but may also be injected into cells or delivered by any other efficacious means.

Animal Assays

A variety of non-human animal models of normal or defective beta catenin pathway may be used to test candidate PRKC modulators. Models for defective beta catenin pathway typically use genetically modified animals that have been engineered to mis-express (e.g., over-express or lack expression in) genes involved in the beta catenin pathway. Assays generally require systemic delivery of the candidate modulators, such as by oral administration, injection, etc.

In a preferred embodiment, beta catenin pathway activity is assessed by monitoring neovascularization and angiogenesis. Animal models with defective and normal beta catenin are used to test the candidate modulator's affect on PRKC in Matrigel® assays. Matrigel® is an extract of basement membrane proteins, and is composed primarily of laminin, collagen IV, and heparin sulfate proteoglycan. It is provided as a sterile liquid at 4° C., but rapidly forms a solid gel at 37° C. liquid Matrigel® is mixed with various angiogenic agents, such as bFGF and VEGF, or with human tumor cells which over-express the PRKC. The mixture is then injected subcutaneously (SC) into female athymic nude mice (Taconic, Germantown, N.Y.) to support an intense vascular response. Mice with Matrigel® pellets may be dosed via oral (PO), intraperitoneal (IP), or intravenous (IV) routes with the candidate modulator. Mice are euthanized 5-12 days post-injection, and the Matrigel® pellet is harvested for hemoglobin analysis (Sigma plasma hemoglobin kit). Hemoglobin content of the gel is found to correlate the degree of neovascularization in the gel.

In another preferred embodiment, the effect of the candidate modulator on PRKC is assessed via tumorigenicity assays. Tumor xenograft assays are known in the art (see, e.g., Ogawa K et al., 2000, Oncogene 19:6043-6052). Xenografts are typically implanted SC into female athymic mice, 6-7 week old, as single cell suspensions either from a pre-existing tumor or from in vitro culture. The tumors which express the PRKC endogenously are injected in the flank, 1×10⁵ to 1×10⁷ cells per mouse in a volume of 100 μL using a 27 gauge needle. Mice are then ear tagged and tumors are measured twice weekly. Candidate modulator treatment is initiated on the day the mean tumor weight reaches 100 mg. Candidate modulator is delivered IV, SC, IP, or PO by bolus administration. Depending upon the pharmacokinetics of each unique candidate modulator, dosing can be performed multiple times per day. The tumor weight is assessed by measuring perpendicular diameters with a caliper and calculated by multiplying the measurements of diameters in two dimensions. At the end of the experiment, the excised tumors maybe utilized for biomarker identification or further analyses. For immunohistochemistry staining, xenograft tumors are fixed in 4% paraformaldehyde, 0.1M phosphate, pH 7.2, for 6 hours at 4° C., immersed in 30% sucrose in PBS, and rapidly frozen in isopentane cooled with liquid nitrogen.

In another preferred embodiment, tumorogenicity is monitored using a hollow fiber assay, which is described in U.S. Pat No. US 5,698,413. Briefly, the method comprises implanting into a laboratory animal a biocompatible, semi-permeable encapsulation device containing target cells, treating the laboratory animal with a candidate modulating agent, and evaluating the target cells for reaction to the candidate modulator. Implanted cells are generally human cells from a pre-existing tumor or a tumor cell line. After an appropriate period of time, generally around six days, the implanted samples are harvested for evaluation of the candidate modulator. Tumorogenicity and modulator efficacy may be evaluated by assaying the quantity of viable cells present in the macrocapsule, which can be determined by tests known in the art, for example, MTT dye conversion assay, neutral red dye uptake, trypan blue staining, viable cell counts, the number of colonies formed in soft agar, the capacity of the cells to recover and replicate in vitro, etc.

In another preferred embodiment, a tumorogenicity assay use a transgenic animal, usually a mouse, carrying a dominant oncogene or tumor suppressor gene knockout under the control of tissue specific regulatory sequences; these assays are generally referred to as transgenic tumor assays. In a preferred application, tumor development in the transgenic model is well characterized or is controlled. In an exemplary model, the “RIP1-Tag2” transgene, comprising the SV40 large T-antigen oncogene under control of the insulin gene regulatory regions is expressed in pancreatic beta cells and results in islet cell carcinomas (Hanahan D, 1985, Nature 315:115-122; Parangi S et al, 1996, Proc Natl Acad Sci USA 93: 2002-2007; Bergers G et al, 1999, Science 284:808-812). An “angiogenic switch,” occurs at approximately five weeks, as normally quiescent capillaries in a subset of hyperproliferative islets become angiogenic. The RIP1-TAG2 mice die by age 14 weeks. Candidate modulators may be administered at a variety of stages, including just prior to the angiogenic switch (e.g., for a model of tumor prevention), during the growth of small tumors (e.g., for a model of intervention), or during the growth of large and/or invasive tumors (e.g., for a model of regression). Tumorogenicity and modulator efficacy can be evaluating life-span extension and/or tumor characteristics, including number of tumors, tumor size, tumor morphology, vessel density, apoptotic index, etc.

Diagnostic and Therapeutic Uses

Specific PRKC-modulating agents are useful in a variety of diagnostic and therapeutic applications where disease or disease prognosis is related to defects in the beta catenin pathway, such as angiogenic, apoptotic, or cell proliferation disorders. Accordingly, the invention also provides methods for modulating the beta catenin pathway in a cell, preferably a cell pre-determined to have defective or impaired beta catenin function (e.g. due to overexpression, underexpression, or misexpression of beta catenin, or due to gene mutations), comprising the step of administering an agent to the cell that specifically modulates PRKC activity. Preferably, the modulating agent produces a detectable phenotypic change in the cell indicating that the beta catenin function is restored. The phrase “function is restored”, and equivalents, as used herein, means that the desired phenotype is achieved, or is brought closer to normal compared to untreated cells. For example, with restored beta catenin function, cell proliferation and/or progression through cell cycle may normalize, or be brought closer to normal relative to untreated cells. The invention also provides methods for treating disorders or disease associated with impaired beta catenin function by administering a therapeutically effective amount of a PRKC -modulating agent that modulates the beta catenin pathway. The invention further provides methods for modulating PRKC function in a cell, preferably a cell pre-determined to have defective or impaired PRKC function, by administering a PRKC-modulating agent. Additionally, the invention provides a method for treating disorders or disease associated with impaired PRKC function by administering a therapeutically effective amount of a PRKC-modulating agent.

The discovery that PRKC is implicated in beta catenin pathway provides for a variety of methods that can be employed for the diagnostic and prognostic evaluation of diseases and disorders involving defects in the beta catenin pathway and for the identification of subjects having a predisposition to such diseases and disorders.

Various expression analysis methods can be used to diagnose whether PRKC expression occurs in a particular sample, including Northern blotting, slot blotting, ribonuclease protection, quantitative RT-PCR, and microarray analysis. (e.g., Current Protocols in Molecular Biology (1994) Ausubel F M et al., eds., John Wiley & Sons, Inc., chapter 4; Freeman W M et aL, Biotechniques (1999) 26:112-125; Kallioniemi O P, Ann Med 2001, 33:142-147; Blohm and Guiseppi-Elie, Curr Opin Biotechnol 2001, 12:4147). Tissues having a disease or disorder implicating defective beta catenin signaling that express a PRKC, are identified as amenable to treatment with a PRKC modulating agent. In a preferred application, the beta catenin defective tissue overexpresses a PRKC relative to normal tissue. For example, a Northern blot analysis of mRNA from tumor and normal cell lines, or from tumor and matching normal tissue samples from the same patient, using full or partial PRKC cDNA sequences as probes, can determine whether particular tumors express or overexpress PRKC. Alternatively, the TaqMan® is used for quantitative RT-PCR analysis of PRKC expression in cell lines, normal tissues and tumor samples (PE Applied Biosystems).

Various other diagnostic methods may be performed, for example, utilizing reagents such as the PRKC oligonucleotides, and antibodies directed against a PRKC, as described above for: (1) the detection of the presence of PRKC gene mutations, or the detection of either over- or under-expression of PRKC mRNA relative to the non-disorder state; (2) the detection of either an over- or an under-abundance of PRKC gene product relative to the non-disorder state; and (3) the detection of perturbations or abnormalities in the signal transduction pathway mediated by PRKC.

Kits for detecting expression of PRKC in various samples, comprising at least one antibody specific to PRKC, all reagents and/or devices suitable for the detection of antibodies, the immobilization of antibodies, and the like, and instructions for using such kits in diagnosis or therapy are also provided.

Thus, in a specific embodiment, the invention is drawn to a method for diagnosing a disease or disorder in a patient that is associated with alterations in PRKC expression, the method comprising: a) obtaining a biological sample from the patient; b) contacting the sample with a probe for PRKC expression; c) comparing results from step (b) with a control; and d) determining whether step (c) indicates a likelihood of the disease or disorder. Preferably, the disease is cancer, most preferably a cancer as shown in TABLE 1. The probe may be either DNA or protein, including an antibody.

### EXAMPLES

The following experimental section and examples are offered by way of illustration and not by way of limitation.

I. Drosophila Beta Catenin Screen

Two dominant loss of function screens were carried out in Drosophila to identify genes that interact with the Wg cell signaling molecule, beta-catenin (Riggleman et al. (1990) Cell 63:549-560; Peifer et al. (1991) Development 111:1029-1043). Late stage activation of the pathway in the developing Drosophila eye leads to apoptosis (Freeman and Bienz (2001) EMBO reports 2: 157-162), whereas early stage activation leads to an overgrowth phenotype. We discovered that ectopic expression of the activated protein in the wing results in changes of cell fate into ectopic bristles and wing veins.

Each transgene was carried in a separate fly stock:

Stocks and genotypes were as follows:

eye overgrowth transgene: isow; P{3.5 eyeless-Ga14}; P{arm(S56F)-pExp-UAS) }/TM6b;

eye apoptosis transgene: y w; P{arm(S56F)-pExp-GMR}I/CyO; and

wing transgene: P{arm(ΔN)-pExp-VgMQ}/FM7c

In the first dominant loss of function screen, females of each of these three transgenes were crossed to a collection of males containing genomic deficiencies. Resulting progeny containing the transgene and the deficiency were then scored for the effect of the deficiency on the eye apoptosis, eye overgrowth, and wing phenotypes, i.e., whether the deficiency enhanced, suppressed, or had no effect on their respective phenotypes. All data was recorded and all modifiers were retested with a repeat of the original cross. Modifying deficiencies of the phenotypes were then prioritized according to how they modified each of the three phenotypes.

Transposons contained within the prioritized deficiencies were then screened as described. Females of each of the three transgenes were crossed to a collection of 4 types of transposons (3 piggyBac-based and 1 P-element-based). The resulting progeny containing the transgene and the transposon were scored for the effect of the transposon on their respective phenotypes. All data was recorded and all modifiers were retested with a repeat of the original cross. Modifiers of the phenotypes were identified as either members of the Wg pathway, components of apoptotic related pathways, components of cell cycle related pathways, or cell adhesion related proteins.

In the second dominant loss of function screen, females of the eye overgrowth transgene were crossed to males from a collection of 3 types of piggyBac-based transposons. The resulting progeny containing the transgene and the transposon were scored for the effect of the transposon on the eye overgrowth phenotype. All data was recorded and all modifiers were retested with a repeat of the original cross. Modifiers of the phenotypes were identified as either members of the Wg pathway, components of cell cycle related pathways, or cell adhesion related proteins.

BLAST analysis (Altschul et al., supra) was employed to identify orthologs of Drosophila modifiers. For example, representative sequences from PRKC, GI# 4506071 (SEQ ID NO:11), and GI#10864650 (SEQ ID NO:12) share 68% and 63% amino acid identity, respectively, with the Drosophila aPKC.

Various domains, signals, and functional subunits in proteins were analyzed using the PSORT (Nakai K., and Horton P., Trends Biochem Sci, 1999, 24:34-6; Kenta Nakai, Protein sorting signals and prediction of subcellular localization, Adv. Protein Chem. 54, 277-344 (2000)), PFAM (3ateman A., et al., Nucleic Acids Res, 1999, 27:260-2), SMART (Ponting CP, et al., SMART: identification and annotation of domains from signaling and extracellular protein sequences. Nucleic Acids Res. 1999 Jan 1 ;27(1):229-32), TM-HMM (Erik L. L. Sonnhammer, Gunnar von Heijne, and Anders Krogh: A hidden Markov model for predicting transmembrane helices in protein sequences. In Proc. of Sixth Int. Conf. on Intelligent Systems for Molecular Biology, p 175-182 Ed J. Glasgow, T. Littlejohn, F. Major, R. Lathrop, D. Sankoff, and C. Sensen Menlo Park, Calif.: AAAI Press, 1998), and clust (Remm M, and Sonnhammer E. Classification of transmembrane protein families in the Caenorhabditis elegans genome and identification of human orthologs. Genome Res. 2000 Nov.; 10(11): 1679-89) programs. For example, the kinase domain (PFAM 00069) of PRKC from GI#s 4506071 and 10864650 (SEQ ID NOs:11 and 12, respectively) is located respectively at approximately amino acid residues 245 to 513 and 252 to 518. Further, the Kinase C terminal domain (PFAM 00433) of the same proteins is located respectively at approximately amino acid residues 514 to 580 and 519 to 585.

II. High-Throughput in Vitro Fluorescence Polarization Assay

Fluorescently-labeled PRKC peptide/substrate are added to each well of a 96-well microtliter plate, along with a test agent in a test buffer (10 mM HEPES, 10 mM NaCl, 6 mM magnesium chloride, pH 7.6). Changes in fluorescence polarization, determined by using a Fluorolite FPM-2 Fluorescence Polarization Microtiter System (Dynatech Laboratories, Inc), relative to control values indicates the test compound is a candidate modifier of PRKC activity.

III. High-Throughput in Vitro Binding Assay

³³P-labeled PRKC peptide is added in an assay buffer (100 mM KCl, 20 mM HEPES pH 7.6, 1 mM MgCl₂, 1% glycerol, 0.5% NP40, 50 mM beta-mercaptoethanol, 1 mg/ml BSA, cocktail of protease inhibitors) along with a test agent to the wells of a Neutralite-avidin coated assay plate and incubated at 25° C. for 1 hour. Biotinylated substrate is then added to each well and incubated for 1 hour. Reactions are stopped by washing with PBS, and counted in a scintillation counter. Test agents that cause a difference in activity relative to control without test agent are identified as candidate beta catenin modulating agents.

IV. Immunoprecipitations and Inmmunoblotting

For coprecipitation of transfected proteins, 3×10⁶ appropriate recombinant cells containing the PRKC proteins are plated on 10-cm dishes and transfected on the following day with expression constructs. The total amount of DNA is kept constant in each transfection by adding empty vector. After 24 h, cells are collected, washed once with phosphate-buffered saline and lysed for 20 min on ice in 1 ml of lysis buffer containing 50 mM Hepes, pH 7.9, 250 mM NaCl, 20 mM -glycerophosphate, 1 mM sodium orthovanadate, 5 mM p-nitrophenyl phosphate, 2 mM dithiothreitol, protease inhibitors (complete, Roche Molecular Biochemicals), and 1% Nonidet P40. Cellular debris is removed by centrifugation twice at 15,000×g for 15 min. The cell lysate is incubated with 25 μl of M2 beads (Sigma) for 2 h at 4° C. with gentle rocking.

After extensive washing with lysis buffer, proteins bound to the beads are solubilized by boiling in SDS sample buffer, fractionated by SDS-polyacrylamide gel electrophoresis, transferred to polyvinylidene difluoride membrane and blotted with the indicated antibodies. The reactive bands are visualized with horseradish peroxidase coupled to the appropriate secondary antibodies and the enhanced chemiluminescence (ECL) Western blotting detection system (Amersham Pharmacia Biotech).

V. Kinase Assay

A purified or partially purified PRKC is diluted in a suitable reaction buffer, e.g., 50 mM Hepes, pH 7.5, containing magnesium chloride or manganese chloride (1-20 mM) and a peptide or polypeptide substrate, such as myelin basic protein or casein (1-10 μg/ml). The final concentration of the kinase is 1-20 nM. The enzyme reaction is conducted in microtiter plates to facilitate optimization of reaction conditions by increasing assay throughput. A 96-well microtiter plate is employed using a final volume 30-100 μl. The reaction is initiated by the addition of ³³P-gamma-ATP (0.5 μCi/ml) and incubated for 0.5 to 3 hours at room temperature. Negative controls are provided by the addition of EDTA, which chelates the divalent cation (Mg2⁺or Mn²⁺) required for enzymatic activity. Following the incubation, the enzyme reaction is quenched using EDTA. Samples of the reaction are transferred to a 96-well glass fiber filter plate (MultiScreen, Millipore). The filters are subsequently washed with phosphate-buffered saline, dilute phosphoric acid (0.5%) or other suitable medium to remove excess radiolabeled ATP. Scintillation cocktail is added to the filter plate and the incorporated radioactivity is quantitated by scintillation counting (Wallac/Perlin Elmer). Activity is defined by the amount of radioactivity detected following subtraction of the negative control reaction value (EDTA quench).

VI. Expression Analysis

All cell lines used in the following experiments are NCI (National Cancer Institute) lines, and are available from ATCC (American Type Culture Collection, Manassas, Va. 20110-2209). Normal and tumor tissues were obtained from Impath, UC Davis, Clontech, Stratagene, Ardais, Genome Collaborative, and Ambion.

TaqMan® analysis was used to assess expression levels of the disclosed genes in various samples.

RNA was extracted from each tissue sample using Qiagen (Valencia, Calif.) RNeasy kits, following manufacturer's protocols, to a final concentration of 50 ng/μl. Single stranded cDNA was then synthesized by reverse transcribing the RNA samples using random hexamers and 500 ng of total RNA per reaction, following protocol 4304965 of Applied Biosystems (Foster City, Calif.).

Primers for expression analysis using TaqMan® assay (Applied Biosystems, Foster City, Calif.) were prepared according to the TaqMan® protocols, and the following criteria: a) primer pairs were designed to span introns to eliminate genomic contamination, and b) each primer pair produced only one product. Expression analysis was performed using a 7900HT instrument.

TaqMan® reactions were carried out following manufacturer's protocols, in 25 μl total volume for 96-well plates and 10 μl total volume for 384-well plates, using 300 nM primer and 250 nM probe, and approximately 25 ng of cDNA. The standard curve for result analysis was prepared using a universal pool of human cDNA samples, which is a mixture of cDNAs from a wide variety of tissues so that the chance that a target will be present in appreciable amounts is good. The raw data were normalized using 18S rRNA (universally expressed in all tissues and cells).

For each expression analysis, tumor tissue samples were compared with matched normal tissues from the same patient. A gene was considered overexpressed in a tumor when the level of expression of the gene was 2 fold or higher in the tumor compared with its matched normal sample. In cases where normal tissue was not available, a universal pool of cDNA samples was used instead. In these cases, a gene was considered overexpressed in a tumor sample when the difference of expression levels between a tumor sample and the average of all normal samples from the same tissue type was greater than 2 times the standard deviation of all normal samples (i.e., Tumor—average(all normal samples)>2 ×STDEV (all normal samples) ).

Results are shown in Table 1. Number of pairs of tumor samples and matched normal tissue from the same patient are shown for each tumor type. Percentage of the samples with at least two-fold overexpression for each tumor type is provided. A modulator identified by an assay described herein can be further validated for therapeutic effect by administration to a tumor in which the gene is overexpressed. A decrease in tumor growth confirms therapeutic utility of the modulator. Prior to treating a patient with the modulator, the likelihood that the patient will respond to treatment can be diagnosed by obtaining a tumor sample from the patient, and assaying for expression of the gene targeted by the modulator. The expression data for the gene(s) can also be used as a diagnostic marker for disease progression. The assay can be performed by expression analysis as described above, by antibody directed to the gene target, or by any other available detection method.

TABLE 1

Seq ID NO

| | 4 | 6 |
|---|---|---|
| Breast | 11% | 39% |
| # of Pairs | 35 | 36 |
| Colon | 8% | 8% |
| # of Pairs | 40 | 40 |
| Head And Neck | 23% | 31% |
| # of Pairs | 13 | 13 |
| Kidney | 10% | 5% |
| # of Pairs | 21 | 21 |
| Liver | 22% | 22% |
| # of Pairs | 9 | 9 |
| Lung | 10% | 8% |
| # of Pairs | 39 | 40 |
| Lymphoma | 0% | 0% |
| # of Pairs | 4 | 4 |
| Ovary | 42% | 63% |
| # of Pairs | 19 | 19 |
| Pancreas | 67% | 67% |
| # of Pairs | 12 | 12 |
| Prostate | 8% | 17% |
| # of Pairs | 24 | 24 |
| Skin | 0% | 0% |
| # of Pairs | 7 | 7 |
| Stomach | 0% | 27% |
| # of Pairs | 11 | 11 |
| Testis | 0% | 12% |
| # of Pairs | 8 | 8 |
| Thyroid Gland | 7% | 0% |
| # of Pairs | 14 | 14 |
| Uterus | 9% | 57% |
| # of Pairs | 23 | 23 |

VII. PRKC Functional Assays

RNAi experiments were carried out to knock down expression of PRKC (SEQ ID NO:4) in various cell lines using small interfering RNAs (siRNA, Elbashir et al, supra). Four siRNAs were used, all of which reduced PRKC mRNA and protein levels.

Effect of PRKC RNAi on cell proliferation and growth. BrdU assay, as described above, was employed to study the effects of decreased PRKC expression on cell proliferation. The results of these experiments indicated that RNAi of PRKC decreases proliferation in SW480 colon cancer and PC3 prostate cancer cells. [³H]-thymidine incorporation assay, as described above, was also employed to study the effects of decreased PRKC expression on cell growth. Results indicated that RNAi of PRKCI decreases proliferation in LOVO and HT20 colon cancer cells, and also in PC3 prostate cancer cells. Standard colony growth assays, as described above, were employed to study the effects of decreased PRKC expression on cell growth. Results indicated that RNAi of PRKC decreases proliferation in SW480 cells.

Effect of PRKC RNAi on apoptosis. Nucleosome ELISA apoptosis assay, as described above, was employed to study the effects of decreased PRKC expression on apoptosis. The results indicated that RNAi of PRKC increased apoptosis in HCT116 colon cancer cells. Apoptosis phospho H2B assay, as described above, was also employed to study the effects of decreased PRKC expression on apoptosis. Results indicated that RNAi of PRKC increases apoptosis in SW480 and PC3 cells. RNAi of PRKC also decreased cell count in SW480 cells. In another apoptosis assay, effects of reduced PRKC expression on DNA fragmentation, PARP activity, caspase activity, and nuclear swelling and condensation were simultaneously measured using the Cellomics™ ArrayScan® HCS System, as described above. Results indicated that RNAi of PRKC has significant effects on caspase activity and nuclear swelling in A549 lung cancer and PC3 prostate cancer cells.

PRKC overexpression analysis. PRKC was overexpressed and tested in colony growth assays as described above. Overexpressed PRKC, in combination with Ras, caused increased growth and formation of foci in NIH3T3 cells. Overexpressed PRKC alone caused increased growth in MDCK cells.

Transcriptional reporter assays. Effects of overexpressed PRKC on expression of various transcription factors was studied. In this assay, rat intestinal epithelial cells (RIEs) cells were co-transfected with reporter constructs containing various transcription factors and luciferase along with PRKC. Luciferase intensity was then measured as the readout for transcriptional activation due to overexpression of the PRKC. Overexpressed PRKC caused an increased expression of SRE (Serum response element) in RIE cells. High Throughput active nuclear beta catenin measurement assay. Beta catenin is a cytoplasmic gene, which when activated, moves into the nucleus. This assay was designed to measure the amount of active beta catenin protein in the nucleus using an anti active beta catenin antibody and a nuclear staining dye. Using this assay, we looked for genes that when knocked out, decrease beta catenin activity, and hence, the amount of active beta catenin in the nucleus. This assay was performed using Cellomics Inc. instrumentation. For this assay, cells were transfected in quadruplicate with siRNAs in 96 well format and stained 72 hours post transfection. The amount of nuclear beta catenin was measured using two different methods. RNAi of PRKC caused a decrease in nuclear beta catenin in SW480 cells.

TOPFLASH beta-catenin reporter assay. Factors of the TCF/LEF HMG domain family (TCFs) exist in vertebrates, Drosophila melanogaster and Caenorhabditis elegans. Upon Wingless/Wnt signaling, Armadillo/beta-catenin associate with nuclear TCFs and contribute a trans-activation domain to the resulting bipartite transcription factor. So, transcriptional activation of TCF target genes by beta-catenin appears to be a central event in development and cellular transformation. Topflash beta-catenin luciferase gene reporter assay is used as a tool to measures activity of various genes in the beta-catenin pathway by transcriptional activation of TCFs (Korinek, V, et al. (1998) Molecular and Cellular Biology 18: 1248-1256). Briefly, cells are co-transfected with TOPFLASH plasmids containing TCF binding sites driving luciferase, and gene of interest. Transfected cells are then analyzed for luciferase activity. RNAi of PRKC caused decreased luciferase activity as compared with normal controls in SW40 and LOVO colon cancer cells, and in LX1 lung cancer cells.

What is claimed is:

1. A method of identifying a candidate beta catenin pathway modulating agent, said method comprising the steps of:
(a) providing an assay system comprising a PRKC polypeptide or nucleic acid;
(b) contacting the assay system with a test agent under conditions whereby, but for the presence of the test agent, the system provides a reference activity; and
(c) detecting a test agent-biased activity of the assay system, wherein a difference between the test agent-biased activity and the reference activity identifies the test agent as a candidate beta catenin pathway modulating agent.

2. The method of claim 1 wherein the assay system comprises cultured cells that express the PRKC polypeptide.

3. The method of claim 2 wherein the cultured cells additionally have defective beta catenin function.

4. The method of claim 1 wherein the assay system includes a screening assay comprising a PRKC polypeptide, and the candidate test agent is a small molecule modulator.

5. The method of claim 4 wherein the assay is a kinase assay.

6. The method of claim 1 wherein the assay system is selected from the group consisting of an apoptosis assay system, a cell proliferation assay system, an angiogenesis assay system, and a hypoxic induction assay system.

7. The method of claim 1 wherein the assay system includes a binding assay comprising a PRKC polypeptide and the candidate test agent is an antibody.

8. The method of claim 1 wherein the assay system includes an expression assay comprising a PRKC nucleic acid and the candidate test agent is a nucleic acid modulator.

9. The method of claim 8 wherein the nucleic acid modulator is an antisense oligomer.

10. The method of claim 8 wherein the nucleic acid modulator is a PMO.

11. The method of claim 1 additionally comprising:
(d) administering the candidate beta catenin pathway modulating agent identified in (c) to a model system comprising cells defective in beta catenin function and, detecting a phenotypic change in the model system that indicates that the beta catenin function is restored.

12. The method of claim 11 wherein the model system is a mouse model with defective beta catenin function.

13. A method for modulating a beta catenin pathway of a cell comprising contacting a cell defective in beta catenin function with a candidate modulator that specifically binds to a PRKC polypeptide, whereby beta catenin function is restored.

14. The method of claim 13 wherein the candidate modulator is administered to a vertebrate animal predetermined to have a disease or disorder resulting from a defect in beta catenin function.

15. The method of claim 13 wherein the candidate modulator is selected from the group consisting of an antibody and a small molecule.

16. The method of claim 1, comprising the additional steps of:
(d) providing a secondary assay system comprising cultured cells or a non-human animal expressing PRKC,
(e) contacting the secondary assay system with the test agent of (b) or an agent derived therefrom under conditions whereby, but for the presence of the test agent or agent derived therefrom, the system provides a reference activity; and
(f) detecting an agent-biased activity of the second assay system,
wherein a difference between the agent-biased activity and the reference activity of the second assay system confirms the test agent or agent derived therefrom as a candidate beta catenin pathway modulating agent,
and wherein the second assay detects an agent-biased change in the beta catenin pathway.

17. The method of claim 16 wherein the secondary assay system comprises cultured cells.

18. The method of claim 16 wherein the secondary assay system comprises a non-human animal.

19. The method of claim 18 wherein the non-human animal mis-expresses a beta catenin pathway gene.

20. A method of modulating beta catenin pathway in a mammalian cell comprising contacting the cell with an agent that specifically binds a PRKC polypeptide or nucleic acid.

21. The method of claim 20 wherein the agent is administered to a mammalian animal predetermined to have a pathology associated with the beta catenin pathway.

22. The method of claim 20 wherein the agent is a small molecule modulator, a nucleic acid modulator, or an antibody.

23. A method for diagnosing a disease in a patient comprising:
obtaining a biological sample from the patient;
contacting the sample with a probe for PRKC expression;
comparing results from step (b) with a control;
determining whether step (c) indicates a likelihood of disease.

24. The method of claim 23 wherein said disease is cancer.

25. The method according to claim 24, wherein said cancer is a cancer as shown in Table 1 as having >25% expression level.


SEQUENCE LISTING (11 sequences)

SEQ ID NO: 1 - DNA, 1796 bp, Homo sapiens
ggtcagctgagttcgccggcccagggcaggcggggcccgagcctagcggtaacccccgggcagggcggggccgctcgcagactccatatgagattcacctcgcaggtggttccctcattcgagtgctccggcgcacagacccgcgccccgccgtctgcgagcctcccgagagccgtcccttcgtccggccctggagcattgcgtttgtcgccggtgtcgcagtgcgaggatggcgccgcgggtgtagcggctctctgcgcaggccgagtgggcccagagaagcgaggaactccgcagctcgtcgacacgtctcgtctcctgtcccaattcagggcttggtgaggtgactcgcggtcgcgggtgactcgccggcaggacactgcctggaacgcctggagcgcctcccactgcagacgtctgtccgcctccagccgctctcctctgacgggtcctgcctcagttggcggaatggcggccacgggagccaatgcagagaaagctgaaagtcacaatgattgccccgtcagacttttaaatccaaacatagcaaaaatgaaagaagatattctctatcatttcaatctcaccactagcagacacaatttcccagccttgtttggagatgtgaagtttgtgtgtgttggtggaagcccctcccggatgaaagccttcatcaggtgcgttggtgcagagctgggccttgactgcccaggtagagactatcccaacatctgtgcgggaactgaccgctatgccatgtataaagtaggaccggtgctgtctgtcagtcatggtatgggcattccttctatctcaatcatgttgcatgagctcataaagctgctgtactatgcccggtgctccaacgtcactatcatccgcattggcacttctggtgggataggtctggagcccggcactgtggtcataacagagcaggcagtggatacctgcttcaaggcagagtttgagcagattgtcctggggaagcgggtcatccggaaaacggaccttaacaagaagctggtgcaggagctgttgctgtgttctgcagagctgagcgagttcaccacagtggtggggaacaccatgtgcaccttggacttctatgaagggcaaggccgtctggatggggctctctgctcctacacggagaaggacaagcaggcgtatctggaggcagcctatgcagccggcgtccgcaatatcgagatggagtcctcggtgtttgccgccatgtgcagcgcctgcggcctccaagcggccgtggtgtgtgtcaccctcctgaaccgcctggaaggggaccagatcagcagccctcgcaatgtgctcagcgagtaccagcagaggccgcagcggctggtgagctacttcatcaagaagaaactgagcaaggcctgagcgctgccctgcacctccgcagacctgctgtgatgacttgccattaaaagcattgtccaaaatcccctgttgtgtggactttgagcacactttacacaagaatctagaaaatcagatcgcgattaagagacagagaatcttggattaaccgcatgggagatgttcttccttttgaagtttcattggagcattttcaatgatgttagcctgatttggggtttcttcaagaacattctaccaaatttttgtactatttctagggaaatttttcagactttaaaattctaatggtagtcagatttcatgtcactaaacaagaaatctgacaatagtgccaggaaactaatttcctgatacattaaaaaaattccatgcaaaaaaaaaaaaaaaaaaaa

SEQ ID NO: 2 - DNA, 1399 bp, Homo sapiens
tgagattcacctcgcaagtggttccctcattcgagtgctccggcgcacagacccgcgccccgccgtccgcgagcctcccgagagccgtcccttcgtccggccctggagcattgcgtttgtcgccggtgtcgcagtgcgaggatggcgccgcgggtgtagcggctctctgcgcaggccgagtgggcccagagaagcgaggaactccgcagctcgtcgacacgtctcgtctcctgtcccaattcagggcttggtgaggtgactcgcggtcgcgggtgactcgccggcaggacactgcctggaacgcctggagcgcctcccactgcagacgtctgtccgtctccagccgctctcctctgacgggtcctgcctcagttggcggaatggcggccacgggagccaatgcagagaaagctgaaagtcacaatgattgccccgtcagacttttaaatccaaacatagcaaaaatgaaagaagatattctctatcatttcaatctcaccactagcagacacaatttcccagccttgtttggagatgtgaagtttgtgtgtgttggtggaagcccctcccggatgaaagccttcatcaggtgcgttggtgcagagctgggccttgactgcccaggtagagactatcccaacatctgtgcgggaactgaccgctatgccatgtataaagtaggaccggtgctgtctgtcagtcatggtatgggcattccttctatctcaatcatgttgcatgagctcataaagctgctgtactatgcccggtgctccaacgtcactatcatccgcattggcacttctggtgggataggtctggagcccggcactgtggtcataacagagcaggcagtggatacctgcttcaaggcagagtttgagcagattgtcctggggaagcgggtcatccggaaaacggaccttaacaagaagctggtgcaggagctgttgctgtgttctgcagagctgagcgagttcaccacagtggtggggaacaccatgtgcaccttggacttctatgaagggcaaggccgtctggatggggctctctgctcctacacggagaaggacaagcaggcgtatctggaggcagcctatgcagccggcgtccgcaatatcgagatggagtcctcggtgtttgccgccatgtgcagcgcctgcggcctccaagcggccgtggtgtgtgtcaccctcctgaaccgcctggaaggggaccagatcagcagccctcgcaatgtgctcagcgagtaccagcagaggccgcagcggctggtgagctacttcatcaagaagaaactgagcaaggcctgagcgctgccctgcacctccgcagacctgctgtgatgacttgccattaaaagcattgtccaaaaaaaaaaaaaaaaaaaaaaaaaaaa

SEQ ID NO: 3 - DNA, 794 bp, Homo sapiens
ggatggcgccgcgggtgtagcggctctctgcgcaggccgagtgggcttggtgaggtgactcgcggtcgcgggtgactcgccggcaggacactgcctggaacgcctggagcgcctcccactgcagacgtctgtccgcctccagccgctctcctctgacgggtcctgcctcagttggcggaatggcggccacgggagccaatgcagagaaagctgaaagtcacaagtctggagcccggcactgtggtcataacagagcaggcagtggatacctgcttcaaggcagagtttgagcagattgtcctggggaagcgggtcatccggaaaacggaccttaacaagaagctggtgcaggagctgttgctgtgttctgcagagctgagcgagttcaccacagtggtggggaacaccatgtgcaccttggacttctatgaagggcaaggccgtctggatggggctctctgctcctacacggagaaggacaagcaggcgtatctggaggcagcctatgcagccggcgtccgcaatatcgagatggagtcctcggtgtttgccgccatgtgcagcgcctgcggcctccaagcggccgtggtgtgtgtcaccctcctgaaccgcctggaaggggaccagatcagcagccctcgcaatgtgctcagcgagtaccagcagaggccgcagcggctggtgagctacttcatcaagaagaaactgagcaaggcctgagcgctgccctgcacctccgcagacctgctgtgatgacttgccattaaaagcattgtccaaaaaaaaaaaaaaaa

SEQ ID NO: 4 - DNA, 1951 bp, Homo sapiens
gcgggaactcccaggatcaggaaggtcccagagaaactccccaggggggaagccgaggccgtccagagagatccagggtacacacagtgacagtgccagctccacattcccgaccaccgggccagggctggttccacgctgtcatttctctccgctggccccagccgcggggccaccgccagacagacagaccggggatcctgcctggcggcgtgaccttgggttagtcctagagccgcttctagcccggtttcctccgccccaggttaggaccacgtggggcggggccgctcgcagactccatatgagattcacctcgcaagtggttccctcattcgagtgctccggcgcacagacccgcgccccgccgtccgcgagcctcccgagagccgtcccttcgtccggccctggagcattgcgtttgtcgccggtgtcgcagtgcgaggatggcgccgcgggtgtagcggctctctgcgcaggccgagtgggcttggtgaggtgactcgcggtcgcgggtgactcgccggcaggacactgcctggaacgcctggagcgcctcccactgcagacgtctgtccgcctccagccgctctcctctgacgggtcctgcctcagttggcggaatggcggccacgggagccaatgcagagaaagctgaaagtcacaatgattgccccgtcagacttttaaatccaaacatagcaaaaatgaaagaagatattctctatcatttcaatctcaccactagcagacacaatttcccagccttgtttggagatgtgaagtttgtgtgtgttggtggaagcccctcccggatgaaagccttcatcaggtgcgttggtgcagagctgggccttgactgcccaggtagagactatcccaacatctgtgcgggaactgaccgctatgccatgtataaagtaggaccggtgctgtctgtcagtcatggtatgggcattccttctatctcaatcatgttgcatgagctcataaagctgctgtactatgcccggtgctccaacgtcactatcatccgcattggcacttctggtgggataggtctggagcccggcactgtggtcataacagagcaggcagtggatacctgcttcaaggcagagtttgagcagattgtcctggggaagcgggtcatccggaaaacggaccttaacaagaagctggtgcaggagctgttgctgtgttctgcagagctgagcgagttcaccacagtggtggggaacaccatgtgcaccttggacttctatgaagggcaaggccgtctggatggggctctctgctcctacacggagaaggacaagcaggcgtatctggaggcagcctatgcagccggcgtccgcaatatcgagatggagtcctcggtgtttgccgccatgtgcagcgcctgcggcctccaagcggccgtggtgtgtgtcaccctcctgaaccgcctggaaggggaccagatcagcagccctcgcaatgtgctcagcgagtaccagcagaggccgcagcggctggtgagctacttcatcaagaagaaactgagcaaggcctgagcgctgccctgcacctccgcagacctgctgtgatgacttgccattaaaagcattgtccaaaatcccctgttgtgtggactttgagcacactttacacaagaatctagaaaatcagatcgcgattaagagacagagaatcttggattaaccgcatgggagatgttcttccttttgaagtttcattggagcattttcaatgatgttagcctgatttggggtttcttcaagaacattctaccaaatttttgtactatttctagggaaatttttcagactttaaaattctaatggtagtcagatttcatgtcactaaacaagaaatctgacaatagtgccaggaaactaatttcctgatacattaaaaaaattccatgcaaaaaaaaaaaaaaaaaaaa

SEQ ID NO: 5 - DNA, 2261 bp, Homo sapiens
gccgcgcggcacgggtgagtgcccagggacttcaccagtgctgccatgctggccccaggctgtgagttggacccagaccaagaagtggtgaggacaaggcctgaagatgtgcctgcttcccattcaacttccaccatgattgtaagtgtcctgaggcctcccagccatgcttcctgtacagcctgtggaactgtgacttttcacatagtagagagaatggcttcagttatacctgcctccaataggtccatgagatctgacaggaatacatatgttggaaaaaggtttgttcacgttaaaaatccttacttggatttgatggatgaagacattctctatcacttggatttgggaacaaaaacacacaacctaccagcaatgtttggagatgtaaagtttgtctgtgtcggtgggagccccaacagaatgaaagcatttgcactgtttatgcacaaggagctcgggtttgaggaagctgaagaagacataaaagacatctgtgctgggacagacagatactgtatgtacaaaaccgggcctgtgctcgccatcagtcacggcatgggcatcccctccatttctattatgcttcatgaactcatcaaattactccaccatgcacggtgctgcgatgtcaccattattagaatcggtacatcagggggaatagggattgcaccagggactgttgtaataacggatatagctgtagactccttctttaagccccggtttgaacaggtcattttggacaacattgtcacccgaagtactgaactggacaaagaactgtctgaagaactgttcaactgtagcaaagaaatccccaacttcccaaccctcgttggacatacaatgtgtacctatgatttttatgaaggccaaggccgactagatggagcactgtgctccttttccagagaaaaaaagttagactacttgaagagagcatttaaagctggtgtcaggaatattgaaatggaatctacagtgtttgcagctatgtgtggactctgtggtctaaaagctgctgtggtctgtgtgacacttctcgacagactcgactgtgatcagatcaacttgcctcatgatgtcctggtggagtaccagcaacggcctcagctcctaatctccaacttcatcagacggcggcttggactttgtgactagacgtcctaactgggcagcccaaccctcccctgcaagtttgtagctcaagttgtaatgtgaaagtcatattttatttgtggcatttttatatagttctcatccacatgctaaatggaaagactttatgaaatccttctctcttaaaaaggaatttattgtaaaagaatactcacactaaattaaattcaaatttcattttagaataagttaactaaatcagtctaataattaaaattttaaaactcctggattatgacatttggagtttcatatgcagcattaattaagctcacatcaaaatagaccagccatttgcacatagttaccagtcactctgtttctgaaaccaatccagaaattcatgttagaacattgtcaggcacttctaggtattggacaagtgacagaaactgatcatccatattcaaaatatttacagaaaaaagtgtcagtgaaatgtaggtctatatggaaaagttaccattaggaaaatatggccatactgcttttttacccctgcactatttcagtgcaccacagaataggattcgagatttacatatgcctgtttgtgattttatctcagaatataacaactctggattacattttcctctcaactgaaaaccctcagtaatgcaaagaaaggccttcctttttcttcagaatcacaagtgactaaattgccttctagccattttttcttgtaaaaaaagtgttttgaggaggctgtattttttattccatttcaatattaaccaaataggctgagaaattcatagcaattgaattaagacttgagttcaagatgttaaaggaatccttttgtatgacttgtccagaccggcctattgccctacccgtttgattctctgggaggcatggtgctcctcctggctgcacatgctctctgtgaactgatcgcatcttgggttcttctcaatttgacatacttgggagtcggaatgactggatgggggatgggtagaggatgactggcacacttgtatagtatgtaatgttgtgtacatattatttatatggttgtggctcaaataaagtttaattccaacacaaaaaaaaaaaaaaaa

SEQ ID NO: 6 - DNA, 2261 bp, Homo sapiens; identical to SEQ ID NO: 5

SEQ ID NO: 7 - DNA, 209613 bp, Homo sapiens
gaattctttttcagataaatcagggatttcttcttggtttgggcccattgctggtgagctagtgtgattttttggcaggtggtaaagagccttgttttgccattttaccagagttggttttctggttctttctcatttatgtaggctctgtcagagggaaggcctagggctgaaggctgttgttcagattcttttttcccacggggtgttgccttgatgtagtactttcccccttttcctatggatgtggcttcctgagagccaagctgtagtgattgttatctgtcttttggatctagccgcctagtgagtctaccaggctcctggctggtactgggggttgtctgctgagtcctatgatgtgaaccacctgggtctctcagccatggataccagcaccttgttatatataaagttttggtgctgcaaaagaaatagcactcgaatataaaattttctttttaattctcagcaaggcaaggtacttctgtagaagcgtgcacccttacagatgaagcaatggtgagtacacacttggacaagggaggggaaggggttcttatccctgacgcgcgtggcccctgctgctgtgtccttcccctcttggctagagttagaccacacaagctaaactaattccgactggctaatttaaagagagtgacgggggtgagtggtttggcgggaaaaatggttatgacacagcaggtaatcagaatgagtcaggatggaacaggtaactgaaatgagtcagggtggagcaggtaatcaaaaaaggttgctttatgagaaagttaagtttaaaagtagacggtaaagaattgaacatactgacatattgattctttgaaaagaaatttagaactcatatctaacaacctgttccagtggaggtggcagaggggtgaaatggactctggggggttcttagcttcggtggtttaatgcaccggttttgcgctggttggcctctggccaggaggtggtgctttccagagagcatcagctgtgataggataggagtaacaggtggtgggcggggcctcagaactcccaagagtataggccttttgtcttcagctacctgagctgagaacttgccccaggctacctgcctcccagctgcaaaagcaactatggctttctttgttcccccacctgtggagtcagcacaccagatagattgacgcgctgccccaagttctggccaggagacttctccctcagttcaaattgttacaaaattcagctggagatttccttctccctgtggccttttcccagcacctttggccatcctcccaaagcgcctctgtgaggccaggcaaaaacagcttgccaggggacccagcgagctcccacggcttttcccactgcttcctctacccctgtatttcactggctttctaaatttactcagctccaggtaaggtcaaaatcttctcccataatctaggccttcagtttctccagtggggaggtgtgtttgggggcagacaatctccctttcctacttccagtttgggcacgcagagtatttgggttgtctcccgggccctgcaggagcaatctgctttcttcagggggtttgtgggtcctccgaagttttctgatttattcctgcagtcgttgtggagcaaaaattcatgatgcgagcctccacacacctctgtctgtcccagtcggagttgcaatctagtcttgcctcgcgtccgccatgatcctcacatgtcttttcacattttttattcttgtgttttagttacttctggtatagctttgatgattttaaagctattatatgacagttgctttctttaaggcccaattctagtcacattcttgggtctaatcctcctgttgactgtgcctcacacaaatattgttacttgcttattctgtgactttctatcatgagcttatcttcaacgaggctggctattttgtgagaatcatgtgtcacctgggtgtgtctgcttccccatgtggttttgcatttattatgctaggagcaccaggggtgtctctgttccatgacccaattttctgtgtatcttttaaaatgagagttcctgaaataaattcaaattccaagccagactcaggcatggagtttgactttcttagggaattgtatttttttcctattgcttgttaaacagagacaaactagattcactagaatctcctgtgtaggtgggaggatttcttcagctgggtattagttccaatatccacctcacctgacatgaggcctcatgtctacatgagctttaaaccttagccttcatgtttagtatcagctgtaagccccggtcacaaccctggctttaagttttctctttttttctggcatgttttgattctcttcccaaaatatttcatgcagccttcgtagatgtttgcaatgccttggggtataatagctctccctcttgcagaacccccagtaaacttcttctcataatatccttccacaactgttctccttgcttactagtctcccttccctctcaatatttcctttcatttttttcttattatttcccttcacttcatgataatttctatctttctctagcaacatattttgaccccacagagtcccatgtatgctatgacaacctgaaaagataaattaacttattagttgttcttccattattgagacaccagagtatttatacccagggtaatgcctattggattgatgcaggaaaattccattttaaatttacagcctttgatcctgtagaaatactccctggtgtgtctaccttcttctcttctctctagtattttagatttaatcattctttctgatagctattgatgtgtcagtcatatcttctcactgcattcctgagtctgataaactgtgctttaaaaataacttcagtcagctatgctgtcttgagcaagaatgcctgaactgccggattcaaagatttaggacttcttggcagatacagtcttatattggagataagaacatggggctacacggtctccctgctggtagccattaccatactcaactcctttgctgagtgactcatcactcaccacagaaccttcttagagccacttaactcctgtagatcagagaagcccacgtagaaatgccaaccagcaggcatgagtatcagcaatgagtaaattaataaaattagcaaattagaatgtgttattataaaaaccacaaaatagacctcactgagcctttacatttttgtgatagtgaaacttacataacatttatcattttaacacttttgcatgtacagttcagtagcattaaatacattcacgttattgtgcaactatcaccattatccatcttcagaactttttcgtcatcccaaactgaaactctgtgctcataaaacaatgaatcccacttcctctttttctcagcccctggtaaccagtatcctactttctttctctgtgaatttgtctactctaagtacctcatataagtgaaatcgtacaatatttatctgtatgtgtgtagtatttcacttagcataatatcctgaggtttatccatgttgtagcatgtatcagaatttcactgagactttactttttaatgaattaaattttatacattttttttggaaaaactaattttttattagccacaaaaccgtaaaaggttttatctgcatagatgctgagatcatcactgacatctctcactcatcagtcatttcaacaccactgtctcccacagcctctattcaggctgtgtcatatttgaactctccttagccttttaacataagaagactttggaagcacatgatctttcacgtgaaaaggcaatttttattctgaaatatgtaagcggcttgactttttaggtaaaaatttgaagccactgaccttcacaagtaagggaactgactaacaagcaaatagtgtctttggaggcctcatcttatctttaatccatctccccctgaccacctaccaccaccttggaaattgtgaaatgtagccgatcaagtattggtcatctacatccaggtgtttcttcttagtgatctttatctgaaaaagaaccagtgcttttacacttttgaacaacttcccattgcctggccaatatggtcccatcttcttaacctgacttttaggaccctacctaatagactcccaaactacatttccagattcagccaaccaggattcttcaccattctagtcatatgtcctgtcttttcctcattttttcttaatgcttttccttttagtgacaacacctccgctagaacctctatcaaaatacccttaagtaaagctcatctcaaatatccaccttagccaagaaaccctttcaaaccctctcagctccatagtatcattttctttgtttaacctctcaaaacacttgtatgtctacctattttttggagtctatcagattatcctttgagttctagtcattcacacatttgtcttagtgccttgaacttactaggttgcaaattgcttaatggagctggctgtatcttgttctctcataacacctagtcttgtgacttacagaaagtagattctttataaatatatttgttctgtcatttgcttgtttacttggagcgagttcatagcattgtagccttaaaccaactttggactggctctttaagaggtgagtcctctggggtcctagtagaaggaagaagatagcactgaaagtaggactgtgaagtgtaggaaacagatctgagtagagggctcgtggtcactgctgatcatttacctagagtaagtggaacctgggtgtcattttatttcatctcatcattggtttttgttggttggaatgctggtaggaaatgtacaaacttgaactcaaatgaaaaaataagttgatttggatatgaaaagcatctagatttttaaagatccatcttcctgttttaacaaggaatgggcaaaatctgatgacccagataatgtctgtaaccttggaacatccacagtgcttcacggtgttaacaattaagctactataggtggaatagcttttccaagaatattagaaatttagaaaaatagacacaaccctaaattactactttttggtaggttaattctagaccagtcctctgcaaaatatgatactggtctaagttgtttgcctaatgaatttctaaactactttatactaataaaaaataatatttaaatcaggcttaatgtttcaaggggaaaaagtggtttaatggctcttgttctcaagtgaaaactgctatgctcagttttttagaaaaacgaatgttaaattgattctagattcaatgacttctgtttgggaggagtgaaaaaacaaatcagaaattgagaaaatcattgagaagtgaataacgaagcaggctgcaaatggtaagtttttttccagttattcattttaggagacagtggtttgcagagtcactggctatagttttaggatcattaaaagacagagaaagtcctggaggattgcgaaagaaaaaaacatgtataattcatttatttttaaaaggaacaaagggagatcttaacaattaaagattagtttacacagctccaaccctgaggtggaggttgttgatcaaatcctcaaaaaaaaaaaaaaaatctatggtatagacatctagaggcacacaggatcctggagaggaacagtgtggctttatgaaactcagtatgtatcagacaattttaatttccttccatgacaggatgacaggacttgtagcttggaggcaaacagtagaggccattaatctgtcttttactcaagttttcaattctatcatacatgtgatgcccagcagccagctaggaaaataagttgagcaccatgtttctcaagctgcaggccctggagagttcacgagaatctctaaataaaagaatacattcttgtacagaagattatatttttaaaagtattttaccagttaaagaattagcttctgttttgcaatttaaaaaatgcataattgaaagatcttcatatagaatgacttcagttccttaagtcagtgattttcaaaatggttttggtccccagatgtattttggaggctgtattactgggtctcagcaggaaccagatggcataattaaacagggaaccgagagaacttactgaagggactgtttacaaggggtgggcaggatttagggagcaacaagggatgatgcagctggaaacaactgttaccagccctgatccgagagggctgaagcagggcaattaccagaacccagggaggacagaagtgtggcaagggccacccacaggagctatggcctttggtagaggacacaggtgtcctgccacaacctgacctaaggaaggtgcacaaacccaggaaattgtatgcagatttgggggcatatgtatgaatgtgtgttttcctgaagatcgggttcatagattttatcaggggtctccaaaatgaatcccaaatgttaaaactgtgtttgtaaaaggaataaataaaattcaaactattatattcctgaagttgatagtttacctcccgtatgaagcctctacaggatgcaacacactaggtaacattctatagctgataaaaataagtcaaaaatgttattttgtgtattttggattacagactgagtttttctaagcaatgattaaacttaactgtttcctgtgctgatgtttgggaatgaaaatggaatacaaaagcaaattatattgactaatatatacatgtcgacctgaagacttgttcaggtgataacatttggataaaattaccctgagagatagtcagctattttttgttatttttgtgattttgttcatttttgtttcatctttcattggggtattttagttttttgtatgtttttgtacataattttatacataccagcaaatatgtgtgtgtacatgtgtatatgtatgaatacataaacacatagtaagattgaatgaattattttattaatagataatatatagtatataattacatcattataattatatataatttttaatatatcacatatgttatataaattataaactgtttcatatattttatatataatataaaaatgtaaaatcctttcagtcttactatgtgccaggcactgtgcaagttgctggaatataaaatgagtaagatactgttgctagcatcgaggagtgagtcaagtaaaagaacttgatctttatataactattccagcggagacaaggcctaaagagttttagaagttcagctggaagagcacatgccagagtcacagactgtccattcttctaaattggtaactctcaacttaggctgcaaaataaaaaatagagcttcccagtcccactcccagagattcttgttgaattggtctgagatggacctctgaacagtgttttcaaaaggcttccctggtgatcctagtatgcacctgtgttgagaacctcagggcggtgtcctggaatgaacacactgacgcccaactggggctcatccccccaactccacacccaggcggtctgagatccacactcatctctgcacactgtggcctcttagaatcaggactagttgttcttctcctgatcattagtttgcatttttattttagagatgctgtaaatttgttctctttacgaggcaaccactagctggccccaagccaattcagagcccacgttctgctgatgcacagactctatggaatgcacagtagcagaagcttcaggcttgtaccagttgatgccccttgcctttgcttttccttttcttgcccagttagttctatcttatactatctcgatgtgctattattcaaccttgtaagctgtctcagattctactggggaagaaggaggaatatatatttataatgaatgaatgaacaaaggaagaaacatacaaaaaatgtacacaaatatacatagaaatgacaagaagagggtttttgcaggccaatctgcttcttgaagggtgttcaaagagggcaagaaagaatgagtagagaagcttgttacaggtgaggtaaaaagccggtgcccagacctagagtgtgcataggtaagcagtcagaagatacctgtagagaacaaaactctatgacacgctaagagtacaaaggaaatgtgagcctcccaaatgaagacagcccaggaacatgaaaaaggataaatgttctcaatacggcatctgcatgagaacaaattcctgtcttcccagtgcaaatctagttctaaaggaaggagggaagaagtgatgacttgagggaaaattgaagttggagacctccccaaaggaggtgaatttggggcttggttttgacagaggggaaagctgtgtgtatgtgagaaaacaagaggttctagacactgccacagatatacactgatggtacagaggactttcctccccacagagtgagatgtagtgggaggaacagccagggagagctgtgctgaccaggactgaataaggaaagtatgagtcttgcaaagattgtgagattatagtgagagtttacaagggaaatgctatgaaaaggattcattcttcacttgacaagtatttattgagtgcctagtatgtgtctggtactgttttaggtgcttgaagcgatagaagcaaataaaatatgtgcagtctttgttctccagaaccttatagtgtagacagtaggcattaattaaataatgaccccaatcatgacatagttgcaagctgcatactttgaagaagtagtcgagggggctctggagggggactacaggtgagcatggaaggaatctggtttgaggattagaaagactttctaagggaagggatctctgagttgagatttaaaccatgagcatgagttaagcgagggcccttcagggagagtagacagcaaaaggctctgaaatgggaagggacattggcactaagagttcccactatcccatataacaccattgtataaatcaggaaaaggcccctctcagtcactgtggctcagggtggactgagattcaccctgtccaggtctcacagctagctggacagggtggatctcagtcccacttgtccccgcagctaagcacccccattggatggatctgttggcagtccttcccaagggattgtgcctttgtgaatttgtgccttgggtaagactgaaggaccaagggtgatgtgcctggagggcagacacaaggggaagaagtgggtgaggtgagctgggctaccaagctgggcccagtcaggaatgcctaagctgcacagagggctttggtcttcattctaagaccaatgtgaaggctttgaagcaggaaaataacccaatcaaacttcaagaatggaggcagggtgatcaggagtgaggcttcctcagtgacctggaggagagatcatggtggcctggactaggatgggtgccaaaaagatggaaggaaatgaacagactggggaaatatttaggaggatcaattaataagacttagtgattgactggctgtggaaattaaggagaaagaatcatcacaggggtttcccaggtttctggcttgtgcaagtgatatggtttactgagatagaaaataccagtgaggaagacacagacataaatgcatgcatgcatgcacctgtgcgcacacactccttgcaggagttgaagagaagattaactgtaattatttcattcttctccctctcatagcctctccttctccgtgtaaaaaaaaaaaaaaaaagattgtccaggagcaggcagataaaagttcaaactgaagcctctctgaggtggttttcactacatggtctaaaaaaatatgaaacaaagtaaaaatcccatcagaaaatgacggcaatgctattctagcttgcttttagctctgatctttgaatttgatggtcaattattgagataccctaaaaatggcacttaaagtgtctctagtgccaaaaagaagttcacaaaccaatgtacctcttgggggattgtttattggtgatgataaatacctcttgtttattgtcattgcttgaaagagaaagacattccccacccaggagcaggccaattcagactcaaccggcctaggacctatttcctgccccaagcctccattcggtcatgtgtgacaatggctctgggatccaaataatctgtactgtgttggaattaaactttatttgagccacaaccatataaataatatgtacacaacattacatactatacaagtgtgccagtcatcctctacccatcccccatccagtcattccgactcccaagtatgtcaaattgagaagaacccaagatgcgatcagttcacagagagcatgtgcagccaggaggagcaccatgcctcccagagaatcaaacgggtagggcaataggccggtctggacaagtcatacaaaaggattcctttaacatcttgaactcaagtcttaattcaattgctatgaatttctcagcctatttggttaatattgaaatggaataaaaaatacagcctcctcaaaacactttttttacaagaaaaaatggctagaaggcaatttagtcacttgtgattctgaagaaaaaggaaggcctttctttgcattactgagggttttcagttgagaggaaaatgtaatccagagttgttatattctgagataaaatcacaaacaggcatatgtaaatctcgaatcctattctgtggtgcactgaaatagtgcaggggtaaaaaagcagtatggccatattttcctaatggtaacttttccatatagacctacatttcactgacacttttttctgtaaatattttgaatatggatgatcagtttctgtcacttgtccaatacctagaagtgcctgacaatgttctaacatgaatttctggattggtttcagaaacagagtgactggtaactatgtgcaaatggctggtctattttgatgtgagcttaattaatgctgcatatgaaactccaaatgtcataatccaggagttttaaaattttaattattagactgatttagttaacttattctaaaatgaaatttgaatttaatttagtgtgagtattcttttacaataaattcctttttaagagagaaggatttcataaagtctttccatttagcatgtggatgagaactatataaaaatgccacaaataaaatatgactttcacattacaacttgagctacaaacttgcaggggagggttgggctgcccagttaggacgtctagtcacaaagtccaagccgccgtctgatgaagttggagattaggagctgaggccgttgctggtactccaccaggacatcatgaggcaagttgatctgatcacagtcgagtctgtcgagaagtgtcacacagaccacagcagctagaagagcaattagcacaactgatggaatgaatgggttcatcttttctatagaagcattagccaaacacaccagcatccctagcacatgtacttttgtgtatttttgttgttgttgttgtaatgtagatgcaacctactgaaccttctggtcttcaatatttttgtggaatcccaccaaacacttcatttaaataagttaactaaaattgttgtgaacactcctccttcctcttttcctccacaattgactagattaaggctcaggaaagcatcatgttcaagataaaggagaaagctttgcaatctgagatatgccatctcctgctttctatcctatttccagttcttagaggagggtcttttacgggaggatataactggagaactagccgtaattcccaaggtgacatttttctccttggtgcaatggttgagtttgcctgctgcattgcagcccttgctgaagaaaaaagtaaatacagaagagatatagaacgctgacctcaggaaataccaataaacgtgagtactggctctcatgaaatttgagaggaaaaaacatgatatgaaatgcttaaatgtaagaagcagcaccatgaaagaggaaatgtattagcatttgatttctaaagaatgagaatgaaatcttataggaaactggaggaaaaaaaacaaaagtcttttttaaaaaatgaaactcttatttttgaaaagttgtcacaagggcaatgtctttggtttcatcagcaagaaaaattgagttccatatgaattctttattgtcagtttcatatcaaatctttgatttcaaggaatgtggttacccacattgaaatcagtgttagccactttgaattgctaacggtgtaataatctccatctgaatctgcatcatttaaaaaaggatttcagtacatatgtgattggattggatgtgtctgtattaacaaacttggaaggcatggagagagttgggcatgagggtagtgtttatttctttgacctctctgctactccagacttctttgtaagatctagatttgggaagaagtagtatactgtaattataatagtacatgctatctttatttttaaatgacaaatagtaattatatataattatgaggtacaatgtgatgttttgatatatgtatacattgtgaaatgattaaattagcctaattcacatatctatcatctcttatacttgcatttttatggtgaaaacaattaaaatctactctcttaaagatttttcactatacgttattacaaactctcatcactatgctatgcaatagatctctcgagcttaatcctccttgtaactgaaattttgcacccttcgaccagtttctctccgttccccatcctgctcccctatactctggtaaccaccattctactctctacttccatgagtttgatttttctagattccacatataagtaaaatcatacagtatttgtctatctgtgcctggcttatttcacttaacaaaatggcctccaaattcatccatgttgttgcaaatgagagaatttccttctttttaaaggctaagtagtatttcattgtgtatatataccacattttaaaagtctattcatctgttgatggacacttggttgattccatatcttggctgttgtgaataatgctgcaacaaacttgggagagcagatgtctgtttacagcacactgataccagttccttcagatatgtaccagaggtgggattgctagcttatatgggaattcttagttttttgaagaaccaccatactgtttttcataattgatgtactaatttacattcccatcaacagtattcaagggttctcttttctccacatcctgccaacactttttatctttcgtcttttttgattatggccattctagcaggtgtgaagcgacacctcgccattgttttaatttgcatttccctgatgactagtgaggttgagtgttttttttaatatatctgttggccttttgtatgtcttcttttagaacctattcagatcctttgcccatttcttaatttgttttcttgctattgagttgtttgagttccttgtatgttctgtgtattacctcttatcagatgtatcagtttagaaatgttttctccatgcgtttctccacatttctccaatccatgtatcttttttgtttgacttagttattgctacatgccaagcatttatgatatcacttaatcctttacagctattccttgagctaggattatttttgtcctctttcgacagatgattaaatgagggcttagaatgatcaaccaaatggcccagtattacacagctggtcagagccaatcaactcctagcttcctaaactgatgagagaagagttctctaccttccatccactcctcccctgagctagcacatggtgaaccattaggcaatcctgccctatcggttatactgacccatcggtcagactgaccctcttgttcagtgacgtgaaaagcaatgaaaatattcaacttgagctgcggcaaatatttcatcacctaggaccctatttattcaaaactcagtcagttgtaaagcaacttggctgtacaggtaaacaagcaaatggaagagctagaaacttcccacagcttttcaagttaatgatcagcaaatctgtgaagcctgacatgggctggcagtcgcacagaaaagttgaaagtccttctctaatccttcactttggcagaaggataaaaggaaaccagtgctctatactttttacagagtgggaaccgactttactaaatgtcttttctggatttcaaggggaatgccaaagatagaaaagaacaggttatagcaatctcagtggtgttttcttgaccctggagaatccactagcgagcagcatataataatcttgtcacttaaaattactatttccatgagcaattaaaagggactaattacccaaaacaatttgctgaaattaagcctgacagctttgctagcaaaatcctcagattaggcagggagggtggctgctggggaggaagagatacttagtgacccagctgccaccaaggagattgtcagtgaactgacctgaggctagaatgttctgggaacacacagacctgaacatggagcttggatgcaccctgagctgaactaggactgcccagtaattgtcaaccctttgggaacatttatggctgctttgaaaatctgatgaaagtcctaattggaacctcttttcacacaaaaatacacatctgccaatacacacaaaatatttgtgtataacttcaggggtcccagggcccctgaaaattcactcatgaatccttaggaatccacgcacccctatggaccaccactttaaccccacctctctgattcagaatgaactttcaacaaggttttcccatgaacagtgggcaaagatagcagggctgggcacaaatgcccattgcacagcagaagtcaaacagggctggtctttggaaaaaggaaaagcagcaaagattagctttggagttcccagcctggcctttcttctacctcttcctggttatgttcataccctcccccattgctcctcaagggtttgagtttattaagcaaacactacctttgctccctatatgcctggtatgtaaagcacaggaaatgttttttaatcaacaacccagagtctttgcacttcctgtatctccgctgaaacattaatctcaggctattggctgcttattaaaaacaaggtggggaaaatttgtattcaaaggggctggtcaatggtaagagacatagaaacaatttgttcttctagagtcatgtttctcaaaatgtgctccactggcctcttgcatcagaatcagagatacttaggctttattccagacatactaaatcacaattggggatgggtgttgggaaagcgcagtgaatctgcatgaaaacatcttcccagttaaattatttttacattaatatttgaaaatgactgcttcaaagactttaaactctatctttgaatcatcattaccacaaatatacatacctggaccccagttctttgggtgcccaaacaatgctgaattagaaccaattcattatacctatgaagaattgtcccataccttttataaaggtgctatcaacgacactgcccattataatatagctgataatttcatttaagtttaacatttggatcaagattttgtgacaaaccgcaaatgtttttactagctaatggtggctgtataatgcacagtggttttttttttttgtttttttttttgagacggattcttgctctgtcacccaggctggagtgcagtggtgcaatctcagctcactgcaagctctgcctcctgggttcacgccattctcctccctcagcctcccgagtagctgggactacagatgcccaccaccatgaccggctaatttttggtttttttcgtatttgtagtagagacgaggtttcaccatgttagccaggatggtctcgatctcctgacttcgtgatttgcccatctcggcctcccaaatgcagtgtttttgtttacagcttggcttccaggaaactcagagctcaatctaagtgtttaaattaagtattgtactcctgggtggcctctttaatactgagaacttctagccagacacagtggctcatgcctgtaattccagcattttgggagggtgaggcacgaggattgcttgaaccctaggagttcaagacagcctgagcaacaaagtgagacctcgactttgcaaaaacaacaacaaccacaacaataaaaacccatacacacacaaattcgtggttgtatgaacctgtggtcccagctactctggaggctgaggtgggaagatggctttagcccaagaggtggaggctgcagtgagctgtggtcataccactgtgttccaatctgagttacagacagagaccttgtctcaaaaaaaaaacaaacaaaaaaaaaccttcctatctatttccttaaataaatgcctcctatttttttttttacctctgtttaaattatattattatgcattcctctttcattgaaagctgccacaaatgcttttttaaatagaggcagtacaggtcatctttcgatagatgaatatattacttttttgcaccttatggaatagtggagagagtggtgatcaggagacaggagatgtctattctaattcatctctgttttagtctgtttcctcaggcaggtcaattacactctctagacctcatttcatcctctgagcaaggagcaggttagatctcaaaggcttcttgcaagtccacatcctcagcttacatgcctcacatgatttggttcgttcaaaaacctgctgtcttctccaggaggtccagatgtaatctcgaggacttgagcttctgccagatgtgggaccgttaaccagagatgctacaggataggtagacgatggagccttgtgtacttactggaggtgggggtgagaggctcaccaggggaatccagatagcctccctggggtcatagcaggggagaggagggccacaaataatgacaaccacagacagggtggaatcaatggcgctaaatgatccctaatgtgtgagttgttggcagtgggaagggaagagtagaatagaggctaggagggtggaattaaaatgaatcaaacaaaactatagacactgaccttgattaaaatatgtgaaaaagcaaaaagacacctttgtaaaaatcttgtccctgtaaaagtaataagtgaattatataccatttatacctgattttccagctacaatgactgattttcttggcaaaatgagggctctgcatactataaaataagtatctgcttaatgttttatttgatcccatcactaagtcaattggtatgtgctcagcccttagggtttgcttgtggcagtggtgcaactatctgctcaccacacttaaaatggaaaaatctgcattgacttttagtggtgcacattttggctcttccgtccagagactatgaacatgttgtgtccacacggaggctgctggcaagaaatggcaggcagggtgcaaagctgcctgagtcagccaagctgggttcaaagtccagctcacctgtttcccagctctgagaaggtgggaaagttatttaacctccctgagctacagtcatgtagaatacatacagaatgaagtttttctcttgtaaaggtgaagagccctgcaattggtctccttgtaccctggcctaaagatttttcaaaatctggatttatctccattttagggttaagaaatactctagctccaaactgattataacctttaatccttgtggtatctttttacctttatcaatgatcctcatgcagttctcagagtccaatatccttcattgttgggaacagcagaagaatataaacatggaattaaagtcaagattctgctccctctcattgaactgtactaaggtaatattataaagactaaatatttccctaagttaccagtgtctgtccaaggaaataacactgaattctttaaagaaaagccactgtgagtgctgcaaacaactagacatagaagaggatttaagcagaacctaagcgtagacaaatattaggcttcatatattgtcaggcaagcctgtgtcaaaaggcagcactgtctttaaaacacgaatacaagttaaacatacttaccacaagttcacagtacaagaggattatcatctttcatcagaatgtttggggcttaattaaagtagattttgctttaagagagacacagatatgtgcatattataaagtgataggagaatgcatttattgagcattgcacagtgcttccattgaacaggtttacattatgctgaaaatttagctttggaataattgggtgaattccactatttcccttcacctgaatctttgttaaaacagcattggattcccaactctcttttctaaaataatgagatcaggatagcaacagtgacttctgggtgtcagtggccttattataggtagtttgtccacctatcaaatgttgacagacccagaggttgtagatctagcaaaataaatatttgtatagaccttagagcaaagggaggcttgactttaaggctttatttccccctttgtggacagttaggagatactcctaagtcctttagacaattctaagccacttatatggctggggaataaaggttaaagtttgcaatggccattttccatagtcaaaaagagtctagtggcccttttttttttcattcactcactcgttcagtaaatattgaaagcccatatggcaggcattattctaagtactggggatacagcaatgacaaacaaggtccctgttcttatggcactaatggagagaaataatcaatatatgcctttatgcactaatatgcatgaaataatatataagttaacatataaacagactggaaatatagtcagtagtggtaaatgtgatgcagataaatgaaatagaatgacaccattgtgagtgagtgagtgtaggtagtcatgcaagacttttttgaggaggtgagatttagggtgagatgtgtaatactaactgcaaagataataagtggtaatccacgtggacccaaggcctccttgtgctaagtagatctagaaagatctgtttctgcaatggtccaaactcagtgagtaattcatctaagttagtacaatggttctctagaagacctttgtttatgtaagtatttggtatttataagccatcaccttagtatgaattctgattggttatccagtcatctggtgtactaaaggaacagaagaggtgtcagaaggctgtctggagagataggatagggtgggccataactaacagtggcagggagggggttagggagacatgcctatgggccctgggctcttggtgccaggtgaaatctttttttccatattgcttaccatcagatgtcctctctaacttcctctgtttcagcagctgtccatgaatgcattctcagcccttccccaagcaagcttttctggctcacacaaagtgcgttataaatgggttaaaatcaaaaggtgccagatgccagcttccaagcttcagtgttacgggaacaggatgttatgaagtagtaatgaaagaagagaatatgatagcaccctatccatggggttttcggtctctatacttggtggctgatcttccaggggacagcataccaggaatccaccttttagaacagtaaatctgagggagataaagagccgctggcaccatgaagacattcagatagaaatactcccaagaactacagagctgcagatgcaacttttagtgtgaatgttggcagcctagaaggggtctactccatgaaaggctgcagcagtggcctgggggagagctatttgcaggcggaaatcagctaccagcaggtccagcagcagcataagaaagaaaatgcagtggagggcagtaactacagaaatgtagagaagaaagcatccaggagggaaacctgcaggtagcagagagagcaaggttacaaagatttgcagcaaggacagaaaggctcttagaataacttcacttaggtttctgcaacatcctaaacagagaaagaaagactcagagtccagggtccacaagcctagaactcttcttggctgccagttggtgcctcttaagaaagaactgctggtcatcacccggttttcatgaaggcaacatgttgataactcggcattttatgtttaactattaataaagtggttaacaaattatgttacacttacacattaggtggagacatgactcaagtgatcacttgtttatgtaataaagaaatgtgaggaggcatttcctggagcagaaccatctgaatccaggtgtccctgcaggctgtgtggggtgctgctttcccaccgtcactaacttgtagagagaaaagcagatgctcttgttctctaaaagaggatactagcaatgaccttcaaaagaatacaaagatgggctgggcgtggtggctcactcctgtaatcctagcactttgggaggccgaggcaagtggatcacttgatgtcaggagttcaagaccagtctggccaacattgaaaccccatctctactgaaaatacaaaagttagccgggcatggtggcacaagcttgtaattccagctatgtgggaggctgaggcaagagaattgcttgaacccaggaggcaaagcaaagattatagtgagccgagattgcgccactacactcagcctgggcaacagagggagactctgtctcaaaaaaaaaaaaaaagagtacaaaaatgcattgcgtgttcttcccacgtttttaccaataatttacacgtacatatattctgacagatgcaagtagttaaggatagtggttcaacaattaatctccttagaactacagcttttttgtaaaatgcattcttagaaatatgaccacatatattctgtttgtaatgtgtagattaaaattcattgactataaggacaagttttgcaagtcaaattccacttataaaaatgaagctttattgtgctgtaattctttacttctttgaagccttcctctttgcatatcaggatgccacacttccctggtgtactgccaacttcatgggccactcctccttggtctctcccatctcaattaacttttaacgtttaagcaaggacctcttgtcatctgtgcctagactcattgtttgttatcttacccattgcatagctttaaatgccatctatatgctttcaacttagaattttatgtctccagtgcaaacgaccaacctaaactccagattcataaattcacctgcttgcttggcctccattttgatgtctactaccctaatgtcttcaccccaatttcctctgtcccagttatggcaacctcatgcttccagtagtttagaccataaacatgggagtcatttttaactcctgcctttctcaccaaatttacatcaagttgtcagcaaatgttgttgggtttgtcttcaaaatgtttctacaatctgactacttctcactacctgcgctgctatcactttgtccatcaactctcactagactactgcgttcgccttctcgttcttctccctgcttctgcccttgaccccattcgggtcttcccagctgagccgccactgtgattctgttaacacacaggtcagatccagccacctctctattcagagctctccagtggcttctcatttcacccagagtgaaagtcattgttctcacagtgtcctacacaatctgtacatcttccctctctcctcccattctccaccactgcctctcccctcctgatctcttccctgttcctccaattctccttgaatgctcctgtctgtggccttggatttgcattgaacagaatgctcttcccccagatatccaagtggcacattcttctcccatccttccctgaccatgctctttaaaattatgattgatggactattttaggaatattttgtttgtatcttgtctcaataaattcaattgtaatagaaactactagccaccaccaggatgaaggaaactgaacttgctgtcaagccctgaacatgtcaccaattagctgttgaaccctgaggtttagtttccttatttgcataataagaataatgtcctggccactcacaagatattatgagataatgtatattatatacaattataagtcatgccttcagcccaaggtaatatatagctggacctctttttcttctcctaaggtgtaaaagtagcttcatgaaagaggagaatttgaccctaagcattcacgaaaagcttaccttttagaccacagagtccacacatagctgcaaacactgtagattccatttcaatattcctgacaccagctttaaatgctctcttcaagtagtctaactttttttctctggaaaaggagcacagtgctccatctagtcggccttggcctgaaagggaggggagaacgtttagtgacttgatgtcccaccccactgacagtggcctcataggaggcttgctgttggctggagcacgctgccgctgtctaactctacagcgtgctccccgtgtaggataaactgagctgtgtgactcttcctaccagaggactgtggagcgatgctgtccctagctgaggagtctcatacgtaggaaggagccttcatggccgaggacctcagttctggggtcagtaactgttgagtggctgcagcagccagcactgtgcccagggaaatcaggaagcggggggttgctgtgagagctgccctttgaggaaaatctcaggttgctccaatgactggggaatggcccagggatgggtgctgggagaaaaaaaaagaggtttcgatgatgctaagctacttgaggatcaggacagtgtttggacattgcgcctctccatatgcctttgcgacattgtccaccttccccctttccgcacacttctcaggctgcacgccaccccttgtgatcacattttcctttccaggctgctcccagtgttcattcatcactgaatttcagctgtatctaccagtgcggacagaagcaaggaaaggaaaccttgatgtcattactccttccgtaccctgtgcacacccagaaatagacctcctaatgcctcctcactcctgagaagacctggattcaagcaagtgggctaatagcactctgttgccatccattattttcacaatcccctttttggtgctctttcctctctttttctccctttcctctatctctttcacgctactttcacccctggcagtctgcttccccttgttccttctcttctcattagagctgcgtgccaccacctctatttaagctgattcaaacacagacatcgcagaagccaagcaggtaatgtcgatgcgagtgtctggtctgaggggagcagcttcacctcaccttcagcaaatgtagccccagatgtgggtcagggcttgtcaaggctgtcatgcatatactggtgtacatttttaaatgctagttatttcaaaaagccagaaatgtgggtgttaatataaaatctccagatattgtaaatgttggcaaaataattcacatttttaaaaatactgtgcaggccaaaggaaacattgactttaacaagcttccatttgcaaattctgctttaacatatcagtgagtgaccctcgagatggactatgagtcagtggccttgaacagagggtcccaaccctattggaccccaacagcccttctattttacaacaaatatgtggtgaaaccacctttactaaccggaagtaaaatgtatagataatatgacctatctccaaacataattttttaaaattcaatatatcttaagtttgagctaaagggaaaataaaagtaatttataatgaaatagtatggattttgaaaagaataaaactactgccagattttcagaatgcccttcaaggcatggtaccaatactgtgtagattctttcttctgggacgagaaagaatcgggggtctgtctttgaaggtttcttccccttggcccttcatccctagtagcaagtgctgctacatagcagtcatcttcaatttgtctctaaacttggtttggggaccccttactcatggctgagagataaaaaccccgctctgagatcatgccatcccgtgggatttgtaccccctccccaagcatttctccccattttctggaccactgtttccgggggacaggatcatttttgaggtacagatgaatggcagaaacatagaatcagcatacttcccttctcttagagctgctaatttaagttaaaggcataaacagagataagattctgtgaacatcactaattgcattttagtgttcaatgtgttcttggaggattaaatccaccctcaatataaattcattctgctgctcattatgtccttgtgtttctatgctttagtaagtaaaaaaggctgtttcatttcctttttaacccacaaatattaattttcttaagtagagttgttaatataaattatgggtttgaataacaaagagttgctggcatttttctgtttccttcacagtttataaattgttctcaccttcataaaaatcataggtacacattgtatgtccaacgagggttgggaagttggggatttctttgctacagttgaacagttcttcagacagttctttgtccagttcagtacttcgggtgacaatgttgtccaaaatgacctgttcaaaccggggcttaaagaaggagtctacagctatatccgttattacaacagtccctggtgcaatccctgcaatgtgggaataaatgattacagaagaatatcgtttagagtttacttttgacacatagtatgtaacttattctagtattaatgctagctgctaatgagtctccctgttttcaactttaaaattattttcttttccaaatgcaattaaataacattcagaagaggcatttacatttgttgtcttttcaaatgcacatttacaatgtgatggggtttagatttcaagacaaacatgactcttggacatgcaaattagtgccagactttgggctcaaatttgaataaattaggtcaggaaaaaactaaataagcctgccagcaagtagctttcataaagtgatgtgacttcttaatatttaatcttctctgcgacttcttgattactgtaatttctttgcatctctatcctctttttcttctgattcttttcacatttgttctctcgccgacttcatgtaaatcagggctcttggctgaccttccatggctacaaagtctgagttgctcaagggacttgggcttacttaagaaactattttctgccaactgttagtctccctgcctgccatgccatctcctcctccccccaccttttcaccacacaccaagtcactcttcaggtgtcagcttaatttacttcctgaaaaaggtctgaatccacagaagggaagcaaacaaatgaaaaaattaaaaggcaacacagataggttacactaaatttccccaggcccatttttgacccagcttttgacccagtctggtttctatttaaagcctgaataagaagatatatcttcataaatgccctagactctctctgtcctaacactttttataattacccatttgtttatctttttctctgatagaacataaactctataaagtcaagaactatgtctgttcactgctctctcctggtctagctctgtgcctgccacattataggtactcaataaaaatttgtcaaatgagtgaatgttttcacaaagatgccatagattatagctccgcaagcctatggttcataaaccaaatcctgtctttgtaaataaagttttattagtcacacagccacacccatttatttacatattgtctatagctgctttcaagctctagaggcagagttgagaagttgcaatagaaatcacacagtcagcaaatcctaaaatatgtactgtcttgtcctttacagaaaaagcttgacaatcctttctatagagtatagacttatccttcagggtggcataaattttgatattgatcaaagtagacttaattacaaaatgctttagaagatccaactagagacccaaggtggccactgataatagagccttgttgggtgaaatatttttttttaatttatttttatttttattttttttttttgagatgaggtcttgctctgtcacccaggctgtaatgtagtggggtgatctcagctcactgcagtctctgcctccagggttcaagcaattctcctgcctcagcctccagaatagctaggactacaggcacatacacccacacctaactaattttttgtatttttagtagagatggggtttcaccacattggccaggctggtctcaaactcctggcctcaagtgatccactgcctcggcctcccaaagtactgggattataaatgtgagccaccatgcctggccagatagtttttaaacaatccaagtaagtggctccttgaattgttcacaatgtcctaagaaagtgattgagagggaagttgaaaatatatacataaaaatgatgttatttatatgccatcttaatatttaaatgtgttagtgtattatgtcatggttaaggtttctcaaaggtcaacatactatttaactgagtagtttatgcttgttaaggtgtgttggctcatgtaggaagggatcatgtgagcgacaatactgtgtacatctttcatctccaaacgcaatactgcttaaaagaaaataagcaaatacatctttttggtaagctaagagttcttaatgaggataaaacttcaaaatgaaggtgtccacaccagtacagtgggttaatttgccaccatccatgggccttcattgtgtgctatatgtatcgtgttgccatgaaataaaataccctaatcaagagctggatgatcattggagcaccttgcccatgctggtgtttgttaatatcaagtggtaactgtagcagtcaggactttacaatgcatgaatgttggctatgaaggcagatccttgtaacccacaacccaagaattatgagcaactttaatagctatgttatctgaaattcatgcccaaatgagcagttccctactactgattatttggggtattgttggataaactacaatctagaaggatgcctgatctgacacttattgctagcactctacttctctctatggatgaacatccacaaaggtcctactttctcccagatgtaagggccttggctggactttattcctggaccacggctgtggaaagccatcatcttttccaacatgtctgatttctagtgaagcatggctcagccatggtggctttgtgatgttgaacacaccacttctccacctacttgtgtgaggatttctgacatccctttttgctccataaatgtctaattctaccaccatttctcttcccaaatctcagccctttttataatgcccattcagttctatcgccctgtcaaccaaacttctccatttgccatgtgcagtctgtatttgcactttggtgacatggtaaaatcataccctgacaagatgtgctccttaagaaaaagctgaaagttttgaggaaacctcaaaacttcatcataacacattctggaagcaaaaccaaggcctctatacagctgactgacaggcctaagaagaagggaggttgggaggctctgtgaaaactataggaagcatcacttaagccttccctgaaagcctggcctggtttctttctagtgtactgatgaggaaaagatacagccagtagaggtgacgtagtaactgatgtgagcaaaaagcaatgtttttctaagagctttgcagtttgctcacttagctgataaaactgggacatttttatgatttttttttttacttgaacacacttatttctgtcactttaaaaaaactcctaagacttgaaatttacccctaccctcaaaacatatcttctaacaatttttctactcttccctttataaacacctaagactatcccgcaataggcagtgagatctctccctgtgggtttccataaagaaaaaaggaataaaagaaaaaggcaatgtgggcagatttccttaagtttcctcaagtccagccttctgacactttttagcccatctgggcttttgctgggctctgaataggatgttatattttggcagctaccatgtatgtaaggatcactcagttctaatagtagacatcaatccgtctcacctattccccctgatgtaccgattctaataatggtgacatcgcagcaccgtgcatggtggagtaatttgatgagttcatgaagcataatagaaatggaggggatgcccatgccgtgctattgagagaaagagaaagtcatcatacatcttgcaaagtgatacatgaacaggacataatttcttccatcattacaagttaacaggcctcagatacatttacataatcattaagactttgccaatgctgaggaaaatgtaagtcctccctaattggcacctacatgacatgctgacagatgtgtccatctgtgaagatcagagggaggcatctcactgccagttctgttgacttcagccctcatcagcaagagcagaaggtggggatggtgtggtatacaggtgtgtcagggcggtgccatttcccatcttcccactgggctcttcatgatgagcctttgaaaaagagctttttgcacgattgacttgattttgaggaaatataattttggttgccaaatcatcagtctttttcaaccatactaaatgaaatagtgaatccatttcatttgctgttaattttgtgtgctctgaaaggtactagagtagaaacgtgggttagattccccacttaacaattttaaacttaaactgaacatggaagtgagtttaaattactatagatgatgaccatagccctgtttttttcagtattcaggacagtggaatcaatgtcaccagaaaagggatcaacacaggctacaggatttagcaactcctggtggctacacaatgtcctaataactctccagaaacatcctccattctgtaatttgcagaaactggaactgatttcaaaactggtagtaacttaattcctgacataacagcagagagtcaaatgtgctcttcaatgtgtcctacttttggacacaaagactttggaaaaccattggccgtgaccatgatgttttcacctgagcttcctccgtctatcaagaatgttgagctatggaaacttcgaacctctgcgttacccctttctggttcccattgcatatgccctgactttcaaatgtttagtaagcatcctggagccaggcatcgaacttgtattgatttgtatatccaagattaacaagtttaggactttggaactggctctgtgactaatgaataaatgctctaagtattattttttcttacctgcaaaataagaagatgaactattatttgatctattagattccttctagttcagacatttagtaagttaatgattgccacttattatgttacgcattgataaaatgacaaatggacttgtgctctctttcacacacacgcaccccattcaaccactactggagaggaagaattttcaaagaaggaatcacatttgtcaatcccctgcctgcctagtaaggcccatttctaattggtaaccttggcatcacacatatttcaggtctttttcccagacaggacaggatcagtctattgctgttttcatttcttactgtgaagttttactgcttatttatggtagtgtcacatttttaagaaacttacttttttgtccttctccatttactatacaaaaaacagttatagtctaaaggtccactaaatgttggtaagaaaaaatttaccaagatcagtggatattcagcttccttctaatgtagatattctgtaatggtttattgaatgcacaagtgaataactgaatggactctcccatttgagcaggtttcaggcgctcttggcccagagaggagagcaatttagctgccttattggtgttgcataattttcccttctttaccgcaggttttcatcaatgattgtccacactgtagggcaatcaatggatgatgcctattccctatggtaggagccacttacagcttagttccttagaattgccaccctgtgcaggagactcttcccctgtcccctggcatgcctgcctgtgcctcttttgagggttgcagtgcccagaaatgtgatggcttcagagttattgcttcagaatacagccctgtgcagagagacaaacatgggaaggcaaatgggatgcctatgcacatacgccctcatcagtgtgtgcccagctgagtccaagggggaaatggtctgcatcttccctaaaactgcaaatcattcccctttcattttgtcttagggcactgtgtgcttccataactgttcacatgttctaaatgcaggagagtaatttacaagagctcagtagaaatgcattagcagcttttcagcgagcttattatggtgttaaacagcagcactttagcagtcctgaaaattagcctctgtgtactaaaagctcaacattcccaaagctgggtaagggatagaagaaggaaaattgccgtggcgggggtgaggggtgttaaccagttacaaagtaaagagaaaggaggggtgctttgagtattttccccctaatttgaatacctaagtggtaactaataaacaaaaatgatgctgcatgctgtgttctgccctgggggctttttccaataatgaactcatacctggacaccttgtttgattgtgtaagaatgcgaagctggagggaaaaagaactcctacatgtaaaagttcccacttttttctctagcctgcaatgactagctgaaatgcaaccatggatacttacactgatggcgagcacaggcccggttttgtacatacagtatctgtctgtcccagcacagatgtcttttatgtcttcttcagcttcctcaaacccgagctccttgtgcataaacagtgcaaatgctttcattctgttggggctcccaccgacacagacaaactgttaataaaaataaacaagggcctgccatagtaactgggataagaaccacgggtcagtttcaactctaatgtttttattttactttttagttaatttacttaagaacacgtgtagatgtcatggtttatgttccatgaattagctaatttgcatcatcaacctgtaaataaagcaacagatgcaaagttttttgcactaaatttgtaaagaaaatcacaaaatcaaaataaatgttgatattattcaaggaggtgtttcctggtgtttttgtttatagtgaacaccaaccagcatcttaaattgacatagacagcctattgttgagcattaccatgacatatgcttaggatgtaagttctgaggccacggcacgtgagtgatgtaagagcaaaaggagaaccaccagttttaggtatgttttctgattccccatcccccatcataaatttgtattgtgaaatggagtatgtgaacttaaaattttaaaggaatcatgtcaagacttttaaatttcttattcagaagaaattctcatggaacaagccttattattgtaaatgtgggatctccctgtggcatttcatcaattccagtgatttatttttgtttttgttttattcacattttaccttctctgaaattaggataggtgttgcagtcaacggtgtctaatagaagatggtaatacaattgtctggcagttactcatacaacagtctgtttaggggtaccctagccccaggtaccaggagtgggagaggtggacgccagggagaggagcagagaagcactacgggtcagcaacaagagggtgagcagtagcagactctcacacccaggaggaagcacaggccacagcaaggtggcaagagtcaagtcctcccggattctgaagtaagtggtgaggaaaatgcagagagagagaagacaacatagtggagggtctgccctttgaggttaggtaaacctggctttgaactccaaccccatcagtgagtaagaaacttgctcagtgtcacacaaataatcgatgtgcctcagaacatctccaaaataggaaaaacacagtacctctcagagagttttgaaagaagaacgtgagattgcatatggcaaaggacattgcacaagtccagacacgttctcagtgatgtgcaaatggtaaccattattttttatatcatgtagcaattttctattcctgcttctgagcttgttacttttcctgtattgcagaatttagtacacaggaagctggtttgagtcctctagtgaggaaaaggcagttggtggtatctggctgatgtaggcagcattggacagaggactgggagggctgacaaggtccatcacagggataaagaatatctgtctaacgaaatacctaactgaggttatagaataggcggccccattcacaaatgatttactctaactattgggtggtctgagtacatgatgcatcttgtttccatttagcaataattcctgagctattctctttctcaattaggggtgtaattaaccctgtatccacgccctctgcttctcttctacccttaatattgtcttttctctgccttcttatgagtttctctgatgaagccttaagctagtaatgaactgcaatagaagtctgccataaagactggtagcaaagaggttaagaccatggtttcaggtgagttttaaactagttattgataattcttagaattttcttctttgttggtatggatagttaagacttggctcacgatctaagtccaaggaaattttttaacacagtacggtagtttgaaaataacctcattgtgtggtgccagtgaatgtaaattacaactattcagattgaccataaaaccaaaacagatgttagaatagcttgtggtttgaattctcctttgaactgtagatattgctataatcgacaggaatttctgaatatttctggagacataaagatcatatgcacagacagaagcagcttggttcttaaagccttccttactcctaaaccagactcactaaacaccacctctggacacaattttcggtctggctgctttaatgagaagtaaaatatggctgaagaccaccaaccaacattgaccttaagtgacacttactgtgtttatgtctcaaacaaaatgatcaattgtcctggttttatcaaagaagttagaaacacactgggaaggagtcagaggcattacaaaaatatgttaaggattttgcctaatgattgccagaaaaaaactcagctttagatatccctagcatcttaaaaggcttaactatgccatgttgtattaatctggtctattgcattttatgcattttctgtgttaacaagggaatataaatacattcaaaatgcattgcatattactaaaatttctagtgagagattagaggataccagttagtgattttataaaagacatatttagatgttgattttgaatagataacttccttttttttttagagttcaatttatttatttttattttattgcttgtgttttggtatcttacataagaaatcatttcctaatccaaggtcacaaagatttatgtctatgttttcttctaacattttatagaattaagccttacatttaggtctttcatccattttgagttaatttttgaatatggtgtgagatagggatctaactttattctttagcatgtggatatctagttgtcccagcaccatttgaagagatgattcttttcacaataaattgtcttagcatgcttgttgaaaatcagttgtacataaaggtgagagtttatttctggtctcttggttctgttctagtgatctttatgtctatccatatgttagtaccacactgtctttatcattataactttgtaataagttttgaagttgggaagtgcgagtcctccaacttttttctcctttgtaaaaactattttggctatcctgcattccaggcattttgacgtggattttattagaagcttgtcaagttctgcaaaataggcaactggaattttaatggggactgcattgaatctgtagaatatttttggtagtatagttatcttaacaatattaaatcttgcaatccataagcataggatgtcttcccatttacttagagctttcaaaatttctttcaataatgcttgttgttttcagagtataagtttgcacttcttttgtttatttgattcctgtatattcctgtaaggtctatatgtcccacttcaagtcgtacaacattgattaaaagtagatttttaaacattaaatatgtttactgtaatccttagagcaaccactaaaacaaataagcagagaaacaaaactgaaacaaaacaaagagatatagtcaaaatataacagataaataaaaatggaatatttaaaaaatgttcaactatcccaaaagaaagctggaaagaggaataaagaaatggaaagcagagcttaatacacagaaatcaaatataaaatggtaaacctaaatccaaacatatcaataattacattaaatgcaaagtgtctaagcacccatttaagacacagagattttcagaatgaataaacaaacatatgatccaattatatgctgtctataatgataatgatacaggcagattaaaagtaaaagaatggaaaaatatagaccatgcaagctttaatctaatgctggaatgactatattaatatcaaaataggcttcagagcaaagaaaattaccagagacagagggatattccataaccataagagtcagtttaccaagaagacataacattcaaagtgtataaaaactaaaaacagagcttaaaaaataaagcaaaaactgtcagaaataaagtattaataaactcacaagcatagtttgagacttcacctctctcagtaatgcatagaacttatggaaagggaatcggcaagacttaaatgttagacctaaaaccataaaaaccctagaagaaaacctaggcaataccatttaggacataggcatgggcaaggacttcatgtctaaaacactaaaagcaatggcaacaaaagccaaaattgacaaatgcgatctaattaaactaaagagcttctgcacagcaaaagaaactaccatcagagtgaacaggcaacctcagaatgggagaaaatttttgcaatctacttatctgacaaagggctaatatccagaatctgcaaagaactccagcaaatttacaagaaaaaaacaaccccatcaagtgggcgaaggatatgaacagacacttctcaaaagaagacatttatgcagccaaaaaacacatgaaaaaatgctcatcatcactggccatcagagaaatgcaaatcaaaaccacaatgagataccatctcacaccagttagaatggcaatcattaaaaagtcaggaaacaacagatgctggagaggatgtggagaaataggaacacttttacactgttggtgggactgtaaactagttcaaccattgtggaagacagtgtggcgagtcctcaaggatctagaactagtcctcaaggatctagaactagaaatatcatttgacccagccatcccattactggctatatactcaaaggattataaatcatgctgttataaagacacatgtacatgtatgttcactgcggcactactcacaatagcaaagacctggaaccaacccaaatgttcatcaatgacagactggattaagcaaatacaccatggaatactatgcagccataaaaaggatgagttcatgtcctttgtaagggacagggatgaagctggaaaccatcattctcagcaaactatcacaaggacaaaaaactaaacaccgcatgttctcactcataggtggaaattgaacaatgagaacacttggacacaggaaggggaacatcacacactcgggcctgtcatggggtgggggcagtggggagggattgcattaggagatatacataatgtaaatgacgagttaatgggtgcagcacaccaacatggcacatgtatacatatgtaacaaacctgcaggttgtgcacatgtaccctagaacttaaagtataataaaaaatatttcttcttttccaacattagcctttaattctataaatttcctctcagcactctacccacattctgtagatataatatgttgtattttcatttttcctcagttcaaactatttcctgccttccccggaaatgtatatcctgctttgtttgagtggagaattttataaatgtccattaaatttgaatagataacttctaaggatcttccaccgtatgtattaattgtctgaatctttctagtgagtatggcagagattgattagctgttcacctaaacagtttgctcttctttctgggcacccagctaaacttaacatctcagcctcctttccagttagacaaggtcatttgtctgggttccagtgactagaatttgagcagaagtaatgggtaccatttccagtcctagcccatagaaatctcccatcctacaaggaggatgtattggtgaccctacatattgtcgctgtcatctgctggacaggtgttgacacactgggaaacagcaatttatgccttgaaggaaacagtaacaattatggaaggagcctgggtcctcgaatcaccagatgtagggaaaccatccatcaactaagaactcccaccttgtacctaatgtgagtgagaagtaaacttaaattgtattaagctactgagattttgcagtagcaagtagcaaaaatttttaaatatggtaagaatatgaaggaaataggagtgattgctggagtctgaagtttcagctttatacacccagagttctcctttcttggaagataatggtattaataatgattatcatctctgtgaagacaagcctgagttttcaggtactgatggattatgtcctcttttcttagccaggactgctgccctgggatttaaggaactcaagaaattgaatgtagaatgttctcattcaaagaggagaacatggctggaggaaggcaagtttctgaggaaaccatctatgagaataaatcagactttacacgctgccttgaaaaaattctacttcagattaaatggccagttctgaacatttggagctaggttacctgctcctcaatttacaaacacacacacacacacacacacacacacacacacacacacacacacgcttttgcctcctattcatcaaaatggctcaatcagtagagttgctgtttgatgggataaaacatgtttgaaacagagcatgtctttttatccaaaagataatttactgaaggttcacaaccaggtgggtggtgaatgtataaggttggatagtggttatgacagacagtgtgtgagaataagtgctgaacaaaatctggacaggcagacgtgaaggtctgtttcctgggattataaccgagatgagacaactatgtgtgtgtgcgtatgtgtgtgtgtgtctgtgtgtgtgcgtgtgtgagagagatacatacatgtgagtccccttatttttatctctaaaacaaagctttccatcccaataatttcccagaaactaatacttggagtactaaaaagtactaaaacatagtgcttttgtttttttgtttttgtttttgtgttattttgttttgtttgaacagagaaagagccattatttttttatcggagaaacctaaggtttaatctcagctatttgacttaccagctgtgttgtgcctattacagacaaacatatatgtgttataacactatgtatatatttgagcctgtgttttctcatctgtaaagtggagataataactatagttgctgtggggactgaatgtaccatttgcatgtgccacacagagtctgatatatagaaagcgcaccataaatggtagaggagacacagacacccaagatatgtagatagcaaatcactttctgtctcaaatatatatctcaagtcttttaaatgtattaaccagctgtgtgccttagttcattaattgtgaaaagtctttcaattaatcagagtcatccctagacaagggtcctgggatattattgagaggcatgaacctcaaatctaacatttgaattctggtacaatctggttattgagtattaaagaccgccttgatcaatgactcttggagcataagtaaaccaaaacagcagcaggggaagaggttacattgagaagacaaagcgcaaaatagaggcaggccacacaaaggcaaggagcacagcctggagaggagtgaagaaagggtgggtcacctcactgccaggacacccacccatgccctcacaccttccttcctaggagagagggagcaggagaaggccactcagcacctactaatcataggcataatagatctatttcttctcattttcccatctttttttctttccttccctccttccttactttcttgttcccttccttttcctctgcctccaacctcttcctcctccccttttctcacctgcctcttgacccactgggtaagtgaaagagctaggattaaataattatggactatcctgagttactgtgactcaaagggtcctcctagtgcaatgagcaagaaccctagagattggacttggaagaacttaatccaggtctcagcactaccactctatggtgtaactgtaaccttcctccacctctctgaacttaatttccttgttttatggagagagttaatatctgctaaacccatccaaaagagctaaatctgcagatgttagtgagatagtagatgtgacgcttttatttttgttctcgtaattgctaaagaaacgaaaaagtcagatgaaaacagcgcgattctccattagtgcaactgtaaatagatacagtcttttggaaaacaaaagttctttaaaaattcagtctctttgaccatttctgtgaatcaccctaagcaaattggcctaaatgtagaaaatctttagttacaaaatattcatttgattcctgtttataatggaaaatattttaacataaaactcagtaatagtctctgtaatgtgtggtacaccacttgacacccataaatgatatgtttcaaaagaatgtaataatatggagaaatgttaatgatgtaaacctaaatgatgaagtgcagaatgcagttatataattacaactattttaaagtctattcattaaaaatgattgaaagaaaaaaatgagttgatcatgtctatgtttaggtggcaggactattgggtgttttttaacaatcatattcttttttgtattccctattttaaataatgtacacaaattgcttatataattaaaatatgtcagataaaataaataatgcagaagtacaattttatggtagtatttaataaataatttgtatgaaatatttcttatgaaacaaaattcttaatatgaaatattagataatggttatttcccagaagtgaaataatattatattgttagttttgttgggttatttttatgagttaataaaacgagaactagtgagattaagctataaatgtagatgctaaatagtgagctgaatctgttaaaacataatgaattctcaacacccagcagtattgtcaataaaattatatttatctccaggggccagagtagccaaataacatgtttgcattagatttagtttgggaacctttatgtcttgttgaggtggaaatgattgttcatgatatacacattccaaattgtttcagagaatgagtgtgacttcagttcctattgggactgtgctagccaaaggtgtatagattattttggcaaggtaagaagaaaaattagtagagaaaactcaatcatttttcctgaaattagaaatgtttttacctttacatctccaaacattgctggtaggttgtgtgtttttgttcccaaatccaagtgatagagaatgtcttcatccatcaaatccaagtaaggatttttaacgtgaacaaacctttttctggaaaaaaaaattatacaaagaagatataaaagaattttgagagaaagctcacaagaaagaaagtctgaacataaaccttcctttagggttaaattatttgttgagaagcaatgtagtataacagtaattatggctaatatttgcccacactatgtgccaagcatagcactgagcatgaaaatggcaagaaaactgcatataacaaatgcattaactcatttgtcccataactgccctcttagctgggtaagattaataatttagtttacaaagaagccaaggcttacagaagttgagaaacttgtgcaagattaacaaccagtaaattacaaaacagccatttctattcagttctgactgtaacggtcatctaatttctttgcatgggaggctgtggaatataaactactgggctctggattcagattgcctgggttccagtacaagttcaaccatatatatcctaattgtgcaaccttgagtaagttatttgtattttattttatcttctcaaagtgtagcatgcataacaatcagccggacacttcatggactcttcttctggaaattctgattcagtgagttgctgatgtgtgtccgaatttgcatttctagcaagctcctcattggtcctgatgttgatggtctatggacaacactttgaatagcgctgccattgaggactccaccaaccatgctgaacacaccactttctttctaccccactggccatggccctggctcaaacccccatcacggcagtgctagctagaacaccaatccttcctcccttttctttgtgaaataccacgaaattcaagacagtgagctgatgacggctataagaatgaccttaattcaatctagttcatatttatgaagcatatcatttgtagctttcggttatttggatagtaatcatatgccactagagttgttagtgataaaagttatctacccccacaccacagtgggtcaagacgtctttgccaagaactgtctgagacttctttgttctttgttcctccatttctctccacatgcctgccccctcccctcccctcccctcccctcccctcccctcccctcccctcccctcccttcccttcccttctcttcccttcccttcccttcccttcccttcccttcccttcccttcccttcccttcccttccctttcagagtttcgctctgtcacccaggctggagtgcagtggcccaatctcagctcactgcaacctccgcctcctggttcaagcgattctcctgcctcagcctgctgagtatccaggattacaggtgcctgccaccatacccagctaatgtttgtatttttagtagagatggggtttcaccatgttggccaggatggtctcaaacctctgacctcaagcgatccacccaccttagcctcccaaagtgctgggattacaggtgtgagccatcatgcctagcctttccacatggctttcattccatacacctcaaaacattttaccaggccttgtgtctttctatatctttatcgattctcacttgggtttgcattaaaattaccagggagctttaaaaacaaaaacaaaaacaaaaaaacactggtgaccaggcactaattaatttagacactctgtattttaaatctaaagattttaagtatttttaaaatctcaaatgatctttggtgaagccaaagttgagaacccctgcttcaggcctttgtccttcaaacatccttccccctttcttccacttttcacctatcaaattccaattttcctttatggtccaattcaaatattacctcttccgtgaaggcttttccaagcatcttctctccttgatcagtattaatttttttctcactgtgttcccaaagcatttgtttcatgctaatgctggtagatagcatttcacattgcaatatagttaagtttccatccctctagaaagactgcgaatcccctgatgcaagctatctcaaatatccttcttgaatccctaagttttaacaaattctattgttgaattaaatttaactgttcagaaattggaacaaaatactattaatttccttctcaaaaacatctagagttatagatttttaattagttactgggtcatacgtacctagtttcaaaaataaagtattccgtggtgttagtacacattactactacagaacaagcttcaaaatctcaaagcaaagggacatttcctttcttctcttgtgaggcaagaatttatccctagtcccttgttatacagtgtgtggtcagtgaaccagcagcatcaggaacatctggggagctcattaggaatgcaaagtcttagattctaccccatacctgctgaatcaaaatctgcattttaacaaactctgtgtgattcacatgcacattaaagtttgacaaacactgccataaactttttcttccaaattctattacactttctgtttcactctgccaagactactttcacgacctggaaatattcctttggattcctaactgaatctatgaatcattactctataaaagagtctcattgctctaccctagtgattccaaactccagctacatatcagaaccaccaggggcactttaaagagtacttatccctagaccctatctctagaaatttggattgacttggtcaaggagacctgggctttggtgtttttaaaatcactcatctatacacacacttcctatcttcaaggagaaattctccaaccagcagagccattaattattttcacttatattaattataatactattaattcattaggattagttttccttgatgccctgtcccaacgtctaatgatttctccgtcagaaaatttgggggactgtctatagaagtttcttgtttcattgggctcatccagaagcacttctaagatgatcactttatcagtaccaaagaaaaaacttgtttgggatttcatgccaaatttctacccaatatgaatgtagtttttggaatatttgatttttccaaaatatataatttttactaaataggtttatatgtgttgttgaatcttcaaactcttcttccttttcctaacacatagaatagtctttttagtaaatcgtgacaaattacactacatgatgagtattaaatattggtaaccaaaagaaagtgctggctgagcagaagtctttccatttgccattaccttcatcttcataatttttccttttctttttctcctgggcttccctgaagaagggaacatgcaggaggctacctctgtttgggatgggcaaatggagtgttttctgaaatgctgacataatggaggctctaagcaagacatttcacacaactctcaggaaatcagagggaaccagcaaaatctccttatgcctcatgatgattaatgctctgttgtgttctgttaacctggggcaggaactggagctaaatttgtagacagcttcagcaattttatatctacccatgagatgaatcacatagacggtatttcagaagtaaattctatgtaaggtccatcatgaagcatatgaaatcactccacaatatcctgcaatacaattaaacctccctcaggcactgggaggcccttttaattatttctcatttgcagcccatggtcttagaattaagggctgaactctatagtgagtcccaggctgaaccttgaatttgtttactactgaaggttggctaaacttttgcatgttttttataatttattttgccccattttcttagatgagccaaaccctaacccccaaaaccaaggagtaaaaatgtcattgaaaatacattttccactgtttttattgctttatgagctgcatccagtgatataatctctacatttaagaaatttgcttaactgtagaggcccattatctaatcatttaatacaaaatccaaggagcaaccatctgatcaaaacaaatttacaaaatcaaaattactcacccaacatatgtattcctgtcagatctcatggacctattggaggcaggtataactgaagccattctctctactatgtgaaaagtcaccttaaattgatgtcaagaaagagagatattgtgacttgttattaaattatagacctagtcataatagttcagttcccagtcagaatgataacctctcttaaaacttaggttttcctgtgaaattgctaagaaaatttttcctttagcattttaaattttcctatcaaataaaagatgtcctccctgacatgacttttataccacagcccagctctcagagcagcacttcctcaagaagataggccccctttgaaggtgtggcatcctagctctttgaccaggttggttagcttgttaactattggtgagaactttcccttacatctgtccttgatcccaacacaggtttgatgttgcaaacaggcttatttgctttgagacaactgtatatggatacgagtcctcttttggtttctataacacacctaccaggtcttctcaaaactgtgtccagtacatacgagaactttcagttctcaaacatttcagttccccatctccatccagcccctcgccaacttgttttcattctttgtcatcacaagttagggaagcatccaaagtctggacctcagaatgtccctttatctgaactttgctcattccagttcacacagaagaactgataattaaacaaaacttatttcccagggcctctttctttagttaaactgtaggccattggtaaaatttattgaaccaggggttaatccacatggaataataatacagcatagtattttcagaaaacggagtctctgccaagaaaggcatgaattcactactttcactaatgcttctcacttttcacttttgggacagaaaaatctagaaaactagacaacttcatcctgatcttttttctcttcactcccatagcaatcagctgaagttctatgctggtgagagcttcaagaacgtagtctcatggctttcaggcaaatcagaacctaaaatgtctataaatgccaaacagcagcaaatacaagacacctcctaatatgcttggatttctttccaagaatgtgcatcacagtcaagaatcaagtgctcatatcctaactgagttctccatgttagaagatgctcctcttcctggtgacagtcattcttggcaaattctttgaatgccccactggcctcctttggtccatcttggtcagacagcaaagtttacagaataggaaggaaaaagaataggggactctatattggggaatccatgtagcctaaatcttgccactgttaccaccaactagacgtttgcaactgtgcccagatagcaaaaaattcctgaggacacttatcctggaaaaggtaatgatgactatgaggcaaaatagtctttaacatgtgtgttgcctttgtaacttagaacagtagttcttgttgatagcttgtatacatctgtctcttacctcctgactcatctcaggctggtctacctgctgctccttcccaggaaccactggcaattttgagactcaacatactcactcactactgctggcaccagcacacctttatttgatattccatgccatatctctcaattttatttttaaggtataatgaggagaaccaccaatttcaatctggcatttacctctggctttgagaggtaaaaatgttagctaattagataagtgttcagtaattaattaccacagtgaattaactaggttgattaacatgtctgtttccggtgcagattcagtcaaccagtgatcttgatcactaacagtgccaggtaatgccatctaagatttatggaccccatgtaacaactcggtcagacatttaggcaagtaattgtcatttggatgaagttcagaataatcttgtggaatctataaaagttgatgaaagtagaatactagttaatgccttctgggcagtcttaaattattctccccacctttcaatcttctcaagactcgatcctgctcttatggtaggttgtttctcagcaatgctttttgattgactgtattgcaaaaggccagtcacctgaagagcaggggaaaaggtttcgtttttaaaaattatgcacacaggagtgcacaaggcaaactcagacctggccactgggtaatttgcaaacaaaggctgtgtgccaggcacagcccatgactcagtccagccttgtgatcaggagggtcttgcatcagatctgccaaatccatgctccatggtgtcttgcctgagacaaaacacttcacttcctaccgtcttcctttatgatgctggcactattacattctcaattccattacagttttctatctggcttaattttgtccctgaacctctaatattagaaaaagaaagcacagtcttgatgtacaactggatagcatttaggcctccctgagaccatgcttttgtgctctttccctgcctgctagactagccctgggaccagagtctcaattcttagatgtctctccatggggtatcagcaaaggttgtattggttcacctgaggctgcagcaggcaccccttcccagaagacactgccccttacacatgcacacacacactagcctcacagcctatgggagtaggggtgtcaacgcacagcaaagtcctcacatctcctccttctttggcgggtctcagagtggggatggggtaaccaagagccctttcctcctgccacatcagattccctggctcttccattcagcccatagcaccagcaatggcttgagagagctccaaaaatgtcctaagctatccattctgaggctaagcagtttatattttcatttattcattcaataaatattaactgagcatacaccatgctccaggaagtctaggaagtgctgaagataaaggcatgaaaaagacagttcttgcttcttttcttccacaaaaatattgacattccatgaataaagaaaacgcaagttaccacactcctcatactaaccttctgtatgttaaaacacatatactaagtgtaaattcagcattctctccttcgtgacaaataactaaaactaattaaagatcttttgatctttccttgagtgtttattttccaatacataggttataggtgcctcaaaacctttgtataaaaggggagctattaagagtgtacaattttttatggtatagatgtgttatagtttaacattattacatatacgtgcaaagaaaatgtacagtattttctattagctatccagtctttctcagtatacgcaccctcctggaagatagagcagaggacagtgtttttaaacttgccaattaacagcatctctgtctttggtttaaatagtctcatttagatgatttattttcttttagatacgcgttcatccaatttcttccttgtgcccactgctcccatctgaggtgacacgtacaaaatggaatggtggggatgagcaaggagcaagagaagggctcctggatccatgtgctcatttccttgtattatcccttggctctatgtgatgatgcagctcaggcattgtccgctgttttctcataagggacctgtggctacttaggtgtagtctatgggtccttgaatgcgctcactgagatgcagctggccccatctggccctaagggagcattatgctggccctccccctcaccatcttggatgcaggcctaagctgaatatggaaattctcctagagaatcacaacctttcttaggctgcctcgaaggatgcaggcctctggctgcagtgttctcaggctggcctatgcctagttggatagggatagcaccctcctcctttattttcacccccaagggtgggtggtcaccgcagaccttggcctgcctcttgcaccatttctcctgccctcttactctcataattaaaagaggagaccctctttgtactttttctctctgtcttatgtgaatttcccttatatcctatcctgagttctctctggtttctagcaatctccgtgttcaagtccagacctaggtttttttgtgttaatgacagtataaacatatctaaaatctttatttttcctcacattgccttattagggactcataattgctattgaaagaattctaatttcctactctaactcccattccccagcagatagacgggagtgtgcactatgaaaaaggccatgtatacagaaaagaaaaaaagagattagtattaggtttcaaagtactatattataattataagcaagaatgatatccctaggacaaatgctacaaccctacggtgtttgttttgctcttttttggagggtgtttccctccacaattctgtcaaaatgcactgctgttctaggcagggccaggcagaggggtggcagctgaatgtttgcacagataccttcccccgagggttggtgtagtggaggctgacccatgctgaggccctggggaggattgcagcatcccctaatccttgcaaaggactgcaagctcctgtttccagtctcaggagatgtggttatgttgtaaaagttttaataattgtgtatgagatttagaaatacaaaacaatgacatattttccaggacccacaaggcattgacatctggaatctttataaagttgaaatttgcctgaggaaaatttcttgattgacgaaatttattttttaccagaattatatacacactctctctatatatatatatatagagagagaaaccaggaatttctctttggacaatgattaaagttaagaaaataacaagtttcaaattgcgtgaacatttatttataggcttttgtaggggtttgagtaaggaggacttgactattgtcagccattccagaaagggtctgtctccaggtaaatggaaacgccacataagaagtgtcatacttatcatgcagtaagtgattattaagcacctactatgtgctaagtttgttaatgctcgaagatgagtaatgacttactttattttatttttttctggttcatattttttatcatgtttttaaaactataaaatatttaatatatattacaaagtattcagaaaaatttaacatgttcttttctaccagacttaatattaatttgaaaacattaaaattaatccatgtacagttgaagatttatccaatctcatttccttccctttctccccagataagtaaacataaccctaaatttgggaagtatacttctggttcaagtgttttataagttgaagtatagatgataaaaaaatagttttggttcttctgcttaaaaattttaccttaatactatcccactctaccttcctttacaactttgttttcaaaaatttaatattatatatgcaatgatatcaaattattgttaggcataggtctaattcatcagattaccatatagtgatctgttatatgacattacagcaatttatccattctcctattgatggccacttattgtttattatatttatttatttatttatttttgagacagagtcttgctctgtcagctaggctggagtgcagtggtgcaatctcagttcaccgcaacctccgcttcccaggctcaagcaattctcctgcctcagcctcctgagtagctgggattacaggcctgtgccaccatgcccagctaatttttgtatttttagtagagacggggtttcaccatgttggccaggctggtctcgaactcctgacctcaggtaatccgcctgccttggcctcccaaagtgctgggattacaggcgtgagccaccacacctggcccactattttattattttttaataatggtctttgttctcagcctgttttttctggcaacatccaaatgaaaagaactttacttacaacttccctggcttttctggtggcctttcttatcacgagcctgactcccacagtgtgaccaaagcgtaaattcactgttttctaatggctgaccttaagccaacaccagagcctcagagtcaacagaaaactctgaacagccagcaaaggtcatcagtgcctctagtggcctggtcaacagagggccttaagactgaaaaacccttgacttctgcatgtacttgacttcaatttttaggatgtttctgttcttaatgatgtttgagaaaatatgaggcaccgcgtgtctaacaaaaattcaagaaaagctatttcattggaaataaagcatattttaaatcaataccagtagggaataaaatgtaaaagcttttgtctaagctcgaaataaccacattcaactcagaatagaaatctgaaggataaggaaaaataatgaaaaaaaaaacaggaagaatgtcagtaacaacagtggctaatagcattactccaggccaggctttggactaaacagttcgactccgtatctcatttcatcctcacaacacgtctgtgagataggtattattatttccccaattttcagatgaggaaatggaagcctagagaggttaaatcaaatgcctgagatcctatagtgagtaaatggagattggagttcagatctagatctgtgcagccacaaaaccatgaaaactctttctactctgcttctggtacatggtggagtccattaaagaagacaaagtccaaggagaatggcctcaacaagagaagggaatttgatctgggtgttgaaggtgaagtaggactgggatgtaaggaacatctctctgccacattttaccctgctagtcatcaggttgttgatttaactcatttataggaaaagagggcacagttaaaccatagcagctgtgtgttctttgaatgctgatgaacaagatggccccatcaaaatgatggcccagtcctaaagctgattttaaagtcatctcattgccaaattttaaattgacccttaaaatgtctctcacacacactttacgattaactcatctcaggctcatcacttccctatgattttacccaaatagcagggcccaactcaccaccctgtctggagcaatttctgtctgcccctgagctgtccctgaaatattagttaaagggtatgagggctgagaggctctgtttctgctggtctcctagcctggcttcctcactgcctgtgactggccattctggtgacttcaagccttggagattaaagcccgtgttagtctttcagcacctgatgaaagcgcttaaccagatcatgaagtacattgggaaatcagcctccacagctggaggaagctctgtgagtttgacaaaccccacactgaagggagagggggtgattttcctgggaactcaaggaaggttcagtcctaacctctaacacacaccacagattattactgctgctgcccaagagggctcagatgggaaaataatgctaatacattgtggaggtggtgaattctaggcccccaaagtcatgaaatttcaaagctgactgattctaatttaggtttagctgagtgaaagagaacagtatgttttatggaactctcttaacattactaagtgcaatatattgagttaaccattaattttttttctaaggaagtagaaaaccttggttgtataatcatggtcacattgacatttctgtaaatgcttcaccttaggcatgtcatgacttttcaaaaatgttattcttttgaacactttacttggttttttatttaaagtaactaaacagaaaatactgttgctgtggtgattgttaggtctttgtgactacttcatgttagttactatagttaaaatttttaattttaaaacatttaaacataaaggtacagatatattagatgcctgtgtagttactacccaaatttaacaattgttacattttgccatatttaacacatttttttttaaaggaaagatttcattaaagccctgtcaccttccttactttagattaccttcactttcttcctgaaggtaatcactgtcctaaaggtgaagagttggtaggtatgcttttggggtatatgttaaaactcgtacttataagtatgtacttaataactacatgagcaggtttaaggtgtgtgcgtgtgtgtatgtatatggtatatgtatatatatggtagatatgtatatataagaatcatatgtatatataatatatggcatatatatataaatgtaacatatactatatatatatttttaaaactggcttttttatgttgcttccaattttttcctgtaaacatccttatgggtatcttcttgtgtatgcacataagagtttctctaatgtgtggtaataatatgtttgcatgtattcagttttactagatagtattacattgccctacctaatagttttaccaagttacacttttgtcaacagtgtataataaaccccatttccccatattgttgccaataaatctgatgttgttagccttataattcttttgttaatgtgatgactgtaaaatggtatctctttgctttatttcatttccctgattactagtgagattaaacgcctttatatatgtttggcagtctttcagctttcttctccattctttgcccgtattttcttttgggtattttatttttgtcttactaatttgtaggagatttttatatatgatgaatactgatcctttgttggttatatatattgtcctttacttccaaactttccatgtcattatgttttaggagtgtatcaggtaaacaatacatcaatggattttcaaaaagttgtcttttatctatgtttaatacatttacaaatactgtgattcctgatatatttttactcctttcttcttattttttttctaattgtgtgtgtgtgtgtgtgtgtgtgtgtgtgtgtgtatgtttctttttaccgccttcctacattctatagaatgattaagttctctgtgctgggcgcaatggctcacgcctgtaatcccagcactttgggaggctgaggtggacagattacttgacgtcaggagtttgagaccagcctagccaatgtggtgaaaccccatttctactaaaaatacaagaattagccagtcatgatggcgtgcacctgtagtccctacttctggggaggctgaggcacgagaatcacttgaacccgggaggcggaggttgcagtgagccgagatcatgccactgcactccagcctgggtggcaaagtgagaaaaaaaaatgattacatgattctttgcagaccccatttccctctgttattttggaagttatatattttatttctattctttcaatagttacccttacatttcaacaagcatacttaatacaattcaaagctaattaatacctatactcttctgcataacacaagcaacttggaacaatttaactctaattactacttcacattttggtataaagtaacttaatctatcttgttttcatcccttcctcccttttagtcttatggttaccactttgttgatagttaataataatttacattatccatgtctgccaatgtctttgatcacaattgcttctcagatctccttccttctgggtttctttctttctaaagtacattatataatagttcttcagcatgggctttgagctgaaaactatctttgttttatcctcatttttgagtgactatttagcatggtctagaattccagattgccagtgattttccctcagcactcatgggatgtttattcattatcttctggtacccattgttgctgatgaaaagtttgttttcatctgcttgtgtttgagtaagtaatctgtctcttctttctggttgcatctagattttctctttatatcctgaagctttattaaaatgtgtttaggtgtgacttttttagaaaataacttattctcatggctcactgtgcttccaaattatgaaaaaacctgtctgttatcaattttaggaaattctcactatttcttcctgaataggttatcttcttctggcacccctcttagatgtatgttaggccacctcagtttgtcctcctaatgtattaacttcccttttatacattcaatttgtctctcggcaataaaatgagaaattttctcagatctttcttcttcacacactctctgggtgtgcttgaccactcttaacctatctgtggttagttgcagtaatgtcctcaattatttactccaccctgtatctacaccctattccatataactatgcaatgccttctcaccataaactgggtaacattgactgctccttgaccatggactcgatcatgccattattttgatcaagaaaatgagtcagaactgacaatgtgccagttgttcatgcctttgcacttccccattgtcatactaacatgcccagggtagtctgctggaggaagagaagcatgtaaaacggagcccaggcacctcgtcaccccagtgagggccagcctagatcatccaacagccagctgagccccagaagtgagcaagcccacgaagatcagcaaagtcactttgccagcccagctgaccccaggcacatgagtaggaaatgcttgctgtgtgtctttcaggacctatggcaatagcattattgtggcaacagggtaacaaatataccatgcattgagatttttcgtttactaaactttttcatttgtaggtaagctaattattactttttcaaattaacctatatttaaaaaccgtatcctattttttctacatatttctgtttcatcttttgtctatgtaatcattttaaacatagatattttataatttctttcaggttgttgatctatctgaagttcttgaagtgctaatcttcccattacagctgatttctctcattagtgtttcctcattttacctataatttcaattttgtagtccatatctattttaccatgggaatccagtacacagaaatatttgaaagcatccccaggcagcagttttgtatttgcttctgctagaggctacaagtgtgtccacagtctaggaccagtttgacattaatgtcttattttaaggattttggtaccatgtaggtagcataaactgtcattctacaaacacaagtgacgcgttacttccgtttctcactggagactgtttttgtacccacgtgccacagagacaaactttctttgcattcaagacagtcattgggatgttagccctctttcactgaaggtacagagtttcccagttttggcttaatgtagaagtttcaggttcaacggtcacctacaaaccaggaccctgtgtatacatcctgcagaatcccagcaccttggcacgggggcctattccaagtttgatcctctctgggcctcagtggcatcagctcatgagcttacattcttcatttctaaaacttgggggttatctcttttattccttaaagctaacccatgctagcctttagacaatactccttttatgtctgttaggaaagttgtccatgtgggtccgtggccgtgctgctggcagatgctttgtttaacaagcaaaaataatattgcaaaaaattagaaatgaaaatttttttaaatttatttttaattgctatgggtaaataatagatgtatatacttagatacatttggtttgtttgtttgtttgttttttgggttttttgggtttttttgagacggagtctcactctgccgcccaggctggagtgcagtggcaccatctcggctcactgcaagctccgcctcccgggttcacgccattctcctgcctcagcctccggagtagctgcgactacaggtgcccaccaccacgcctggctaattttttgtatttttagtagagacagggtttcaccgtgttagccaggatggtctagatccccgacctcgtgatccgcccgcctcggcctcccaaagtgctgggattacaggagtgagccactgtgcccggcctacatttgatgttttaatacaggcacacaatgtgtaataatcacaccagagtaattaaggtatccatcacctcaagaatttatttatttgtgttaggaacattccaattccacccttttagttatttttaaatatataataaatgattgttgactgtagtcactcagttacgctatcaaatactagatcttggggaaaatggaggataggaggcaggactaatttgcagctcccactcggatggacagagcagcatgtagagactaacatcatgaacttttgcttcaagaactactgcagaaacagccaagagagtccacagaccctttgaaggaagtgaactgctgctgcaggccccaggatatacccaaaaaactgtgagtgcccaaaatgtgaaagggggatattctgcccccaaacatatacccttactgggaaacctgaaggtccagatcccagatcacaggagaaggatttgaccttacctggagctgagatgaatttagagagtcaagtgaaatacaggggtagaagcagcagcgagaagagccctgtgggcactttcattctccagggatgccatttctgactttgtctcacagggatcctttgggagggctgccagaggaactgggaaatgaccagagagaaggaaacttccagctgaactttgtaacaatttcaaccgagtgcaaagtttcctggacagaagttggggtcgggggtggttgaatcgggagtgcagacacagcacagaaactgcagcaggcagggaggtgcaaaatctgaaagccctgcttgctttctaaaccaggaggctggtagcctggggcaagttctcagccctgcttacccactgcctggaaacaaactcagtgctgtcaaaggggcacggtgggagtgagcccagtcattcaggctgcagagaagccaggtgaggcctgtaactaacaactttcccccactctcctggcaacctacatgatgcacaaaggcaaccataatccccctgggaacgtaactccattggcctgagaaccacacccctatccccccagcagccactgcaagaccccacaaggagactcagagctcagacacacctaaccctgcccccacctgatggtctttctctacccgctctggtagccaaagacaaaggacataatctcttcagagctctatggccctgcccactccctgatcctccctacactatcacagctaatgctctcttgaaagcaccaccccctggctggaggccaaccaacatgaaactagcacaataagcaaaactacaaccagggaccctcacagagtccacttcactcccctgccacctccaccagagcagatactgttatctacagcagggagacctgaagacggtccacatcacaggactctgtgcggacactcgccagtaccagcccagagtccagtagttgcaatgggtggctagatcccgaagagaaattacaatcactgcagttcagctctaggaagccacatccctagaggaagggggagagcaacacatcaagggagcaccccatgggacaaaagaatctgaacagcagcccttgagcccaagatcttccctctgacatagtctacccatatgagaaggaaccagaaaaaaaattctagtaatatgacaaacaaggttctttaacacccctaaaagatcacactggctcaccagcaatggatccaaaccaagaggaaatctctgaattgccagaaaaagaattcagaaagtcaactattaagctaatcaaggaggtgccagagaaaggtgaagtcaaacttaataaaatttaaaaaatgatatatgaagggaaaaagcttcagtgaaataaatagcataaataaaaaacaatcactacttctggaaataaaggacacacttagagaaatgcaaaatacactggaaagtctcagcaatagaatcaagtaagtagaataaagaacttcagagctcgaagacaaggctttcaaattaactcaatccaacaaagaagaggaagaaagaattttaaaaaatgaataatgcctccaagaagtacgggattgtgttaaatggcagaacctaggaataattggtgttcccgaggaagaagagaaatctaaaagtttggaaaacatatttgagagaataattacagaacacttcccaggccttgtaagcgatctagactccaaaaacaagaacctcaaagaacatttgggaaatttattgcaaaaagactatcacctcagcatagtcatcaggttttctaaagtcaagatgaaggaaagaatcttaagagctgtcaggcaaaaacatcaggtagcctaaaaaggaaactctatcagattaacagcaggtttcttagcagaaatcctacaagctagaagggactggagacctatctccttaaacaaaacaattatcagccaagaattttggatccagagaaactaaacttcataaatgaaggaaagatggtctttttcaggcaaacaaaggctgagagaatttgccactcccaagccagctctataagagctgctaaaaggagctctaaatcttgaaacaaatactcaaaaagcaccaaaatagaacctcattaaagcataaatctcacaggatctatataacaaaagcacaatgaaaaacaaacaaaaaaaggtattcaggcaacaaatagcgtgatgaatagaatagtacctcacatctcaatactaaaattgaatgtaaatggtcaaaatgctccacttaaaagacaaagaatgggagaatggagaattcaccaaccaaggatctgctgtcttcaagagactcacctaacacataaggactcacataaacttaaggtaaaggggtggaagaagatattccatgcaaatgagcatcaaaaatgagcaggagtagctattcttatatcagaccaaacaaactttaaagcaacagtagttaaaaatgccaaagagggacattatataatgataaaaggacttgttaaacagaaaaatatcacaatcctaaacatatatgcacctaacattggagtgcccaaatttataaaacaattactattagacctaagaaataatatagatggcaacatattaatagtgggggactttaatactccactaacagcaatagacacgtcatcatgacagaaagtcaacaaagaatggatttaaactataatttagaacaaagggacttaacagatatttacagaacattatacccaacaactgcagaatatacattctattcctcagcacatggcgcattctttaagatagatcatatgataggccacaaaacaagtctcaataaatttaagaaaaccaaaattatatcaagtactctttcagaccacagtgtaataaaattggatatcaactcaaaaaggaaccttcaaaaccatgcaaatacctgcaaattaaataacctgttcctgaatgatcattgggtcaacaatgaaatcaagatggaaattaaaaaattctttgaactgaaagataatagtgacacaacctatcaaaacctctgggatacagcaaaggtggtgataagaagaaagttcatagccttaaatatctacatcaaaacatctgaaagaccacaaatagacaatctaaggtcacacctcaaggaactatagaaacaagaacaaaccaaacccaaacccagcagaagaaaagaaataacaatgatcagagcagaactaaatgaaattgaaacaaaaaaaatacaaaagataaattaaacaaaaagctggttctttgaaaagataaataaaattgatagatcactagtgagatgaaacaagaaaagaagagagaatatccaaataagttcaattagaaacaaaatgggagatactacaaccaataccacagaaatacaaaagattattcaaggctactatgaacacttttatgtgtataaattagaaaacatacaggagaaactagaaatatataaccctcccagattaaaccaggaataaatagaaactctgaacagaacaataataagcagtgagagataaatggtaattaaaaatgccaagaaaagtaagtccaggatcagatggagtcacacctgaattctatcagacattcaaagaagaattcgtgccaatcctgttgacactattccacaagatagagaaaaagggaatcctccccaaatcattcaatgaagccagtatcaccctaataccaaaaccaggaaaggacataacaaagaaaactgcagaccaatatccctgatgaacgtacaaagatacaaaaatcctcaataaaatactagattaactgaatctaacagcatatcaaaaagataatccagcatgacaagtgggtttcataccagggatgcagggatggtttaacatatgtaagtcaataaatgtgatacaccacataaacagaattaaaaacaaaaatcacatgatcatctcaatagatgcagaaaaagcatttgacaaaatccagcatccctttatgattaaaaccctcagcaaaatcagcatacaagggatatacctcaatgtaataaaagccatctatgacaaacccacagccaacattataccgataggggaaaagctgaaagcattctctctgagaactggcaaaacacaagaatgccctctctcaccacttctattcaacgtagtactagaagtcctagccagagaaattagacaagacaaataaataaagggcatccaaatcagtaaacaggaagtcaaactgtcactgtttgctgatgatatgattgtatacctagaaaaccctaaagactcctccaaaaagcttcctagaactgataaatgaattcagcaaagtttcaggatacaaaattaatgtatgaaaatcagtagctttgctatacaccaacagcaaccaagctgagaatgaaattgaaaactcaatccctgttacaatagttgcaataaaataaaatataatgtaatacttaggaatatacataaccaagaaggtgaaagacctctacaaggaaaactgcaaaacactgctgaaaaaaatcatagacaaaacaaacaaatggaaacatatcccatgctcatgaatgggtagaatcaataatgtaaaaataaccaactgccaaaagcaatcttcaaactcattgcaattcccatcaaaataccaccatcattcttcacagaactagaaaaaaattctaaaattcatatgtaacaaaaaaagagtctgcatagccaaagcaagactaagcaaaaagaacaaatctggaggcatcacattacccaacttcaaactatattataaggccatagtcaccaaaacagcatggtactagtataaaaataggcatacagaccaatggaatagaatagagaacccagaaataaacccaaatacttacaccaaactgatcttcaacaaagcaaacaaaaacataaagtggggaaagtatatcctattcaaaaaattgtgcagggataattggcaagccacatgtaggagaatgaaactggatcctcatctctcaccttattcaaaagtcaactcgaggtgtatcaaatacttaaatctgaaacctgaaactgtaaaaattctagaagataacatcagaaaaacctttgtcgatattggcttaggcaaagacttcttgaccaagaacccgaaagcaaatgcaacataaacaaagataaatagatgggacttaattaaatgaaaaagcttctgcacagcaaaagaaacaatcagcaaacagacaacctacagaatgggagaaaatctttgcaatttatatatccaacaaaggactaatttcctgaatctataaagaactcaaatcagcaagaaaaaaaaatcccatcaaaaagtggtctaaggacatgaatagacaattctattcaagaaggtatacaaatggcaaacatatgaaaaaatgttcaatgtcactaattatcagggaaatagaaatcaaaaccacaacacaatatgagcttactcctgcaagaatggccataattaaaaaataaaaaaataataaatgttggcatggatgtggtgaaaagggagcacttttatactgctagtcagaatgtaaactagtacagccaccatggaaaacagtgtggagattccttaaagaactaaaatagagctactatttgatccagcaatcccactactggatatctactcagaggaaaagaagtcattatatgaaaaagatacctacacatgcacgtttatagcagcaaaattctcaattgcaaaaatatggaaccagcccaaatgccactcaatcaccatagataaagaacttgtgagatatatatatatatataaaaaacatatatatataaacagacataaacatatgtctatatatacgtgtatatatatatatgtatatatatatacaccatggaatactaagccataaaaaggatgaaataatggtattcagagcaacctagatggaattggagacaattatatttttttttaattatactttaagttctgggatacatgcacagaacctacaggtttgtttcataggtatgcacatgccatggtggtttgctgcacccatcaactcatcatctacattaggtatttctcctaatgctacccctcccctaaccccccatcccccaacgggccctggtgtgtgatgttcccctccctgtgtccatgtgttctcactgttcaactcccacttaagagtgaaaacatgcggtgtttggttttctgttcttgtgttagtttgctgagaatgatggtttccagcttcatccatgtccctgcaaaggacatgaaatcatcttttttaatgactgcatagtgttccatggtgtatatatgccaattttctttatccagtctatcattgatggacatttaggttggttccaagtctttgctattgtgaacagtgccgcaataaacatacgtgtgcatgtgtctttatagtagaatgatttataatcctttgggtgtatacccagtaatgggattgctgggtcaaatggtatttctgattctagatccttgaggaatcgccacactgtcttccacaatggttgaactaatttacactcccaccaacagtgtaaaagcattcctgtttctccgcatcctctccagcatctgttgtttcctgactttttaataatcgccattctaactgatgtaagatggcatctcattgtagttttgatttgaatttctctaatgaccagtgatgatgagctttctttcatatgtttgttggctgcatgaatgtcttcttttgagaaatgtctgttcatatccttcacccactttttgatggggttgttttttccctgtaaatttgtttaagttccttgtagattctggatattagccctttgtcagatgagtagattgcaaaaattttctcccactctgtaggttgcctgttcactctggtgatagtttcttttgctgtatagaagctctttagtttaattaaatcccctttgtcaattttggcttttgttgccattgcttttggtgttttagtcatgaaatcttcgcctacgctcatgtcatgaatggtattgcttaggttttcttctagggtttttatggttttaggtcttacgtttaagtctttaatccatcttaagttaatttttgtataaggtgtaaggaaggggtccagtttcagttttctgcatatggtggagaccattattctaagtgaaggcactcaggaatggaaaaccacacattgtatattctcattcataagtggaagctaagctataaggatgcaaaggcctaagaatcatacaatggactttagggacttagggaaagagtgggaggtggtaagcgataaaaggctacacgttgggtacaatgtatactgctctggagataggtgcacccaaatctcacaaatcaccattaaataacttattcatgtaaccagaccaggtataatggctcatgcctgtaatcccagcactttgggaggccaaggcaggcagattacttgaggtcaggagttcgagaccagcctggccaacatggtgaaacccatctgtactacaaatacaaaaattacccgggcatggtaatgggcacctgtagtcccagctactcaagaagaggctgaagcaggagaattgcttgaacccaggaggtggaggttgcagtgagccgagatagagccactgcactccagcttggatgacagagtgagactgtctcaaaaaaaaaaaaaattattcatgtaaccaaacaccacctgttccccaaaaacctattgaaataaaaataaatataaaaaatactagatcttattaattttatctacctatatttttatatccattaatcatctttacttttccccctccccactaccccttttagcctctggtaacatcattctactctccatctccatgagttcaatttttttaatttttagctccccaaaataagtgagaacatgtgaagtttatctttctgtgtctggcttattaatgtgctccagttccatccacgttgttgcaaatgacaggatttcattctttttatggctgaatattattttatggtgtatatgaccacatttcctttatctattcaaccactgatcgacacttagattgcttccaaatcttggctattatgaacagtgctgcaataaacatgggagtgcagctatctctttggtagactgattttctttcttttggatatatacttcacagtgagattgctgcatcatttggtagatctatttttagttttttgaggaactatcatgctattctccataatgactactaatttacattcccttcaatagaaaaaaatgttttaactgacctagtcctcaagcaaaataagaacatagtgcaatacaaaaaatatggtccagggcacaagcttttctttaaagtgaccttaatttttgttaagcattagaaatatcctctatctttgttaagcagtaaaacttgacataagcagtgtttggactaatggtcctgtttttccaaaatcatcatttcatacaattcctctatattctgtgatttaatttatgagactgtatccattctttattttcctggtaacacttgattcttatgtctgtaagtcatttgagatgtaacaacactaaatcaacatttagaagagtacacaggcatgacacacttcattttttaacattagattttatttgaaaatattttaaaataaatgcctttaaatgaatacttttaaaatactgcaacgtggttttctaacaaataactgaggtttctggagatctttcctctcctctctccagagcctgttcaggtgagagaaggtcaggggatcagagagcagagatgttccacactaattctaatcctcccctactttgtcttaatcccttcttcattcccacactaatgagggcacagtctggtggacaaaatacagaggctggctttttggtaaaagtcataattttgtttcaaggtagaaaaaatatttccaacgtttggccagttttgatttgaactggactagctgggaaagataactatggcttcaataagttggaagggagtagtgcatcaagatttataaaaacagactaacaatataccaaatgatgcattgttccgggtataatcagtgtgtggataaatggaaagtaattgtattcagaagaaagccattctcttggctgtggaagcaagaaaaacagtacagcacaattctgtcacaggatatagatgctgttttgtttcaaaatggtagactaacaaattgcacctacagaggcagtgtgatatactgaaaggataattgttttaaagccaaatggacccatgtatcattgggccagcaagctcataatcttagtgttttcttcttgtaaaatggaaataataataattactatgcaggagtattgggaagataaaataagaaaatatatattaaatatctggtttgtaagcacataataaaagataaaattattgttattttaataccatttatcttaacatatactttttaaataaaaatttatgaatatgtaattaaagaagcatgaaatggtgtgtggctcatggaacacaaatctcttaatcaaatgagaaagctctgaaagggaaattccggagagtcaaggggctaagaccagggatatttatgtggctgtgtggatctaagcaagactcttcccccagacagtgagacttcaaacacagatgatgattacgctgaatggcttggttgtctctgtgcaaatagtgcctgacagacaggaaactgactcccatgatacacttcacagaatacagttagctttacatttgttaacactgattatataaaagaacaatgaatgaggcagcatagaacttacctctctctcaattctgactcactcgaactcattcaacattactctgccagaaacacactcattgttggagagcagaaacagtgacaagtcagggagaacatattggaaaaggctttttttcagccatttgaaaaaaatgtatgtaagagaaatgtatgagtggggaaaataattgaatgaagtggaatgggtttagtatgatttaatcgccttttaatttttctaaatttggctaatgatgacaggtgctacctgtctgtgatcttgttttgtcatcacaagccccctgtaacgtagatatttccattttataacaatgttagttcatttgccccagatcttccaggaggtgaatagctcaaggctctccaatcctaaattccacaactctttcattaacccctgatcagccaatccaagaagcatatctaggaccaaattcttataatttggatatcctacagggacatatacaagtaggctttttcctttttttaaaaaaaattatttttaatttttgtgagtacatagtatgtgtatatattatggggtatgtgttagtccgttctcatgctgctatgaagaaatacatgagactgggtaatttataaaggaaagatatttaattgactcacagttccacatggctggggaggcctcaggaaacttacaatcatggcagaaggcacctcttcacatggtgtcaggagagagaatgaatgctgagcgaagggggaagcctcttataaaaccatctgatctcatgaggactcactgtcatgaaaacagtatatgggaaactgcccccatgattcaattatcaccacctggtcccacttttgacatgtggggattattacaattcgaggtgagatttgggtggagatgcagaaccacaccatatcagggtacatgagatactttggtacagacatgcaacatgcaataaccacatcagggtaaacggggtatccatcccctcaaacatttctcctttgtgttacaatccaattctactcttttcattattttaaaatgtgcagtcattatgattatattcaccctgttgtgctgtcaaatactaggtcttctttattctttctaactactttttgtacccattaataatccccacatccccactcccatccccctgctactcttcccagcctctggtaaccatcttctactctctatcttcacaaatacaatcgttttcatttgtatctcccacaaataagtgaagtgtgatgtttgtctttctgtgcctggcttatttcactcaatgtaatgattgccagttccatccatgttgttgcaaatgacaagctcttattctttttatggctaaatagtactccattgtgtctgtgtatcacattcattcatccattcatctgttgatggacacataggttgcttccaaatcttggctgttgtgaacacagctgcaacaaacacaggagtacaaatctttcttcaatatactgatttcttttcttttgagaatatacccagcagtgggattgctggatcatatggtgactctattttactgttttctgaggaaacaccaaacttctccatagtggtcgtactactttacattctcaccaacagtgtacaagggtttccttttctccatatccttcctagcatttgttattagctgtcatttgaataaaagccatttttattggggtgggatgatatctcattgtagttttgatttgcatttctctgatgatcaatgatgttgagcattttttcatatgcctgtttgctatttgtatctcttcttttgcgaaatgtccatttagatcttttgcccatgttttcattggaatactagatttcttcctatagagctgttttgagctctttatgcattctggttattaatcccttgtcagataggtagtttataagtattttctccctcttattctgtgggttgtctcatgcactttgttgattgtttcctttgttgtgcagaagttttaacttgatgtgatcccatttgtccatttttgctttggttgcctgtgcttgtagtttattactcaagaaatttttagccaatccaatgtttcccaatgttttcttgtagcagttttatagttttaggtcttcgatttaagtctttaatccattttgatttgatttttatatatggtgagacataagggtctagttttattcttctgcctatggatatccagttttcccagcaccttaaagagactgtcttgtcatcaatgtacgttcttggcacctttgtcgaaaatgagttcactgtaagtgtatggattagtttctgggttctttattctgttccattggtctatgtgtctgtttttatggcagtaccatgctgttttggttatgatagctctgtagtataatttgaagtcgggtaatgtgattcctccagttttgttctttttgttcaggatagccttggctgttttgagtcttttgtggtttcatataagttctaggattgtttttttctatttatgtgaagaatgtcattggtattttgatagggattgctttagatagtatggacatttaacaatattgattcttccgatctatgaaacggaatatttttccattttttagtgtcctcttaaatttctttcatcaatgtttagttttcattatagagatcttttacttctttggttaattcctaggtatttaattttatgtgtggctgttgtaaataagattacttttttgtttctttttcacattgttcactgttggcatatagaaatgctactgattttcgtatgttgattatgtatcctgcaactttactgaatttgttaatcagttctaatagtttccttgtggagtttttaggtttttccaaatataagatcatatcatctgcaaacaagaataattttacttctttcttttcaatttggatgccctttatatctttctcttctctgatcactatagctaggacttctagtactatgttgaataacagtggtgaaagcaggcatctttgacgtgtttcagatcttagaggaaaggctttcagcttttccacattcaggattatactagctgtgagtctttcacatatggcttttattgtgttgaggtatgtttctcttatacccagttttttgagaatttttatcataaagcaatattgaattctattaaatctgttttcagcatcaattgacataatcatatgattttcatccttcattctgttgatatgatgtacacattgactgatttgcgtatgttgaaccatccttgcatccctatggtaaatcccacttggtcatgatgaacagtcttttaactgtattgttgaatttggtttgctagtattttgttgaagatttttgatcaatagtcatcagaaatattggtctatagttttcttttttagatgtgtctttgtctggttctggtatgagggtaatagtggcctcatggaataagtttggaagtattctctactcttttattttttagaatagtttgaataaagttggtattaattcttctttaaatgtttgataaaactcagcagtgaagccatcgggtcctgggcttttctttattgggataatttgtattacaacttcgatcttgttgttattggtctgttcaggttttggatttcttcatagttcaatcttgttaggtggtatatgtctaggaatttatctatttcttccagattttccaatttattgacatatagttgctcataatagccactagtgatcctttgaattccagtggaatcagttgtaatgtctcctttttcatatctgattttatttatttgtgtctttttttcttagatagtcttgctaaaggtttctcaatttatcttttcaaaaaatcttttcattttgttgatcttttttattattttcttcatttcatttttatttatttctgctctgatctttattatttcttttcttctaataattttgggtttagtttgctcttgattttctaattttttaagatgcatcattaggttgtttatttgaatattttcttcttttctgatgtaagtgcttatagctataaattctcctcttcatactgctttcactgtatcccataggttttggtatgttgtatttctatgatcatttgtttcaagaaatttttcaatttccttcctaatttcttcattgacccaatggtcattcaggagcatattgtttaaattccatgtatttgtgcagttgtcaaaattcaacttgatattgatttctagttctattccatggtggtcagagaagatgcttggtattatttcagtttttttaatgttttaagacttattttgtgacctaacatatggtctatccttatcgatatgctgaggagaaaaatgtgtattctgcagccattggctaaaatgttctcttaaaacattcacatttttattacactaaaaatgttctcttagaacatttacatttatactattccctaatataaatatctcttaggtccatttgttctacagtacagattaaatccaatgtttctttgttaattttctttctgaaagatctgtccaatgctgaaagtggggtgttgaaatcttcagctattactgtattggggtctatctctctccttagctcaaataatatttgctttatatctgggtacttcaattttgggtgcatatatatttataattgttattttctcttgctgaactgaccattttttcattatgtaatgactttccttgtgtcttcttacagtctttttcttaaaatctattttgtctcatataagtattgccactcctgctctttttttggtttccatttgcatcgaatgtctttttccagccctttatttgcagtctatgtatgtgtttataggtgaagtgccttttttgtaggcaacaggtcaattggtcttttaaaaaaaaaaaaaaaattctctcagtcaccctgtcttttcattagagagtctggtccatttgcaatcaatgctattattgataaataaggacttactactgcctctttgttatttttctgtgtgtgtgtgtgtgtgtgtgtgtgtgtgtgtgtgtatgaaggtcttctattccttctttccttccttcctgtctttctttgactaaaggtgattttctctggtgtatgatttaattacttgctttttattttttgtatatctatactatgtttttaaatttgaagttatcatgaagcctgtaaatactgtcatataacccgttgttttaaagtgatgtcaacttaacactgattgcataagtaaacaaataaacaagcaacaaaaaaactaataaaaactctacactttaactttatcccccagctttttaactttttattttcttctctttatgtcttattgaaatgtctatgtcttgaaaaggtgttatagctattatttttcactggttcaccatttagtctttctacttaagaatagtaaacacatcacaattacagttttataatattctgtgcttttctgtgtgcttgctattaccagtgagttttctatcttcagatgatgtattttctttcttttctgtttggattttgtttgtttgttttgagacagggtctcactctgtcaccttggctggaatgcagtggcacaatcttggctcactgaaacctctgcatcccagctcaagtgattttccctcctcagcttcccaagtagctgagactacaagtgcatgccaccacacccagctaatttttaaataaaaatttggagagacaaggtctcactgcattgcccaagctggtctcaagcttccaagcttaagctaccctcccgcactggtctcccaaaatgctggaattacagacgtcagccactatacctggccctcaaaggatttcttcttgtttgttggcatctttcttttcagtttgaagaactccctttagcatttgctgtaggaaagatctggtgttgataaaatccctcagctgttgtttgtctaggaaagtctttatttctctttcatgcttgaaggatattttcattggatatgttctagggtaaaagtttgtttccttcagcactttaaatatgtcatgccactctctcctggcctataaagtttctactgaaaagtctgctgccagatgtattggagctcctttgtatgttgtttcttttctcttgctgtttttaggatcctttctttatccttgatctttgggagtttgattattaaatgccttgaggtagtcttccttgagttaaatctgcttggtgttctatcacctttttgtatttgaatgctgatatctttctctacattttggaagttctttgatattatccctttgaataaactttctactcctatctttttctctgccttctctttaagatcaatactcttagatttgcccttttgaggctgttttctagatcttgtagggatgcttcatttttgtttattcttttttcttttgtctcttttgactatacattttcaaacagcctgtcttcaagctcactaattctttcttctgcttgattaattatactcttaaggaactctgatgcattgttttagcatgtcagttgcatttttcagctccagagtttcagctcaattctttttaattagttcaatctctttgttaaatttatctgatggaattctgaattccttctctttgttaccttgaatttctttgagcttcctcaaaacagctattttgaattctctgtctgaaagatcacatatctctgtttttccaggattggtccatggtaccttatttagtttggtgaagtcaagttttcctggatggtcttgatacttgggggtgttcatcagtgcctgggcattgaaaatttaggtactcattgtagtctacacagtctgggcttgtttgtgcccatctttcttggaaaggctttccaggtatttgaagggacttgtgtcccatgatcaataatgctgtggttcttatagactcattgagttaccaccttggtgttcttggataaaatctagaagaattctctgaattgccaggcagaattcttgttctcttccctgattttctcccaaataaatggagtctgtctgtctgtctgtctgtctgtctgtctgtctctttctctctgtgtgctgagctgcccagagctgaggttggagtgatgccaggagccctgtggccaccaccactgggacggtgctgggttggacctgaagccagcacaccactgggtattgcctaaggcccactgtaaccactatttggctactgcctgtgttcactcaaggccctagggttctacagtctgcagatgggaagatagtcaggtttgtgtcttttctgttagggcagcaagtttccccagacccttggcaggtacagagatgctgtctagaagccagagattggagtaaaaaaacctagaaatctacctgatgttttattctgctgtggttaagctggccctcaaaccacaaaacaaagtctttcctgttcttccctcccttttccactggcagaggaggctctcccagtggccaccaccaccactggcttgcagggagttctgccatgccaccaccaatattcacgtaaagccgaatggctcttcagtcagcttgtggggaatgctgctaggcctgtgactcatacttcagggcagtgggctctcctctggcccagggcaggtccagaaaagctgtccaagaaccaaggcctagactcagggtccccaagagcccacctgatattctactctactgtagccaaatacgtacctaaggtgtaagacaaagttccttttacttttccctctgcttttctcaagcagaaggagtctttcatagtagtcgctgcagctgggaacgtgctgggtctcacctgtaaccagcatatctcacactctcacccaagacccactgcatactaccttagtgtcacttttagttatttggggctgaagggctctttagtcagtagatgatgaatcctgccaggacttggttcttctcaaggcagtgggttcccttctagtccagggtgtatatagaaatgttgtctgggggactcataactctgcccagtgtcctatcttattgtggctgaactggtagtcaagatacaagccaaagtccactttcctcttagctttcttctccttaaatagaaggaagtagtcacttttttttttttttttttttttttttttgagacagaatctcgcttctttgcccaggctggaatgcaataccatgatctaggttcactgcaacctccacctcccgggttcaagcaattctcctgcctcagcctctccagtagctgggattacaggcatgtgccaccatgcccagctaatatttgtatttttagtagagacagggtttcaccatgttggccaggctggtcttgtactcctgaccttaagtgatccgcctcagcctcccaaagtgctaggattacaggcatgagccaccgtgctggaaggagtcacttttgttgctgcgagttgcactgcctggggttgtgggagggatgatgcttgcactcccttagccaccccagctgatgtctccctaggtcacgtgccgccctagtccactggctctaatcctagcccagcattaggacttgcctaggaaatgcagtatttgtgtccctgactgccttttaattttacctagtactccagagcactttagcccttggtgatgaggcttgcagagaaactcaagtcccaaccactgggatggatgatccccctccggctaaggctgatccaaatgatctctctgtgggcaggcaatggctgagcccagctcagctttgctctctgccatggcagaacagcactgagttcaatatcaagtcctacagtcactgtgctctccctccctaaagtgctcagattccctctccatgccatgtagcagctctggggaatagggaagaggtggcatcagtgattcaagactgtgtcctaccctcttcagtgcctctttcaatgatataaagttaaatccaggtactgtgactcctcacctgacttttggttcttatgacagtgcatttttgtttgtagttagttgttaaaattttgtgttcctttgggggcagtcagtggaggcttctgtttagccaccttgctccgccccccataaacagtggcttaattaatatttttagtgaaaaatgccagcatggcagagaggaaagaacattgggttaggagtcagaataccagggttctgatcccagctccataattaactggctatgtgatcataaggaaaccatacagcttttcttgttctcagttatctattaaatgaggttggataattaacactttacttgaatcaagagacctgtacatagatagaaacacgtctctgatttctgggtagggacaggtagagatggcttgtctagaatgggtagagatgtcctatctgaatgacaccaacataaatggaaaagctgctgaatgcttgggggtagctcagtttgggttcattcatgagacagagtccacacggtaatttggacaagggaaagtttaatataaagagttgttaattatagggagagactacagtagcaaaagattggctagtaagaagtgaagagagtgctaaataatataaaaatagcagatacaaggagccactatccctagagctgagctagaacacccaagaaaaagctccctgcactcaaggttgagatccagaccttgtttggtgaggcctttggggggtgattaggtcagagtggagccctcatgattgagattaaagcccttataaaagggaccccagagagttctccatgtgaggatacaagaagaagtcagcagtcttcttgttggaagaaggcctttactaggaactgaccatgctggctctctgatctcaaacttccagcctccagaacttgataaatttctgttgtttataagccatccactctatggtattttgatgtaacagcctgaacaagtcaagacattgttggagggcatggctgtggctcacagagtgactgagagatcactgaggtgccatgccagtggaacttgctgaaaatcctcgctctagggtgtcagggagagctgttcatagggacatgcctcaccagagagattctgctacaaaagaacacaaagggttctaggggaagctgacgaccacggtgcactgtagaagccaggcactaaataagctgtatttactgcaagaactagatgcttaaggagtcacccctactgtaggagctgggcactagagaagctttgcactctgcataagctcgacactggagaagctgcccacactgcaggagcctgccaagagggctcactggaatcagagagcaaacccctttctcctgcaatatctcttctgcacactctactagcatagcttaaccttgtgtcagctgacaactgaaaactaaatggcccagcttcatttcatggagcaagcaaaaaggatgactttggatctcatgggcactaaatcaagaacttgcacaggtggacatcaaaagcagtttcctaaaagataggagttcatgaaatgtgtcttattgtaccttggcaatcatttgttccagaggcgactagcctctggacagcaaaaagccttaaaattttgagtttgatcgtgcctgtaatcctagcactttgggaggccgaggtgggcggatcacgaggtcaggagaccatcctggctaacacagtgaaacctcgtctctactaaaaatacaaaaaattagcctggcgtggtggcgggcacctgtagtcccagctactggggaggctgaggcaggagaatggcatgaacctgggaggtggagcttgcagtgagctgagattgcgccactgcactccagcctgggtgacagagtgagactccgtctcaaaaaaaaaaaaaaaaaaaaaaaaaaaaaaaaatatatatatatatatatatatatatatatatatatatttgaatttgaatgctttaaggaaggccatacctattctatttatcatagtcctcagtaactccataggatcttaagctcagcagcaaacttcatacaaatatatgctgggcccacacaagcatctgagcgtgtggtccttaaccatttaaccacttcattttacaggtgaggaaacagcatgcttaaatggctacttctgcccctgcccccgtctctgtcatagtcaaacagaacagcgggggaggtaatacataaactttcctttagtggtgatttttctcaaacatttgctttgaatttgggctgtttgtattgcacagattttgctcatttacataaaatcaagatcgttggaataaatatgttcaggtaattctctgaggcaagaaaataaccaaatgcttactcagacttgtttctcctcgggttaactagcctgatattaagtatgccttaatgtaaaatcccattcaaaatctcctaaaacaaaaccagtgcttcacaatttgtaattagtactataagaacaatttaattgattctcctattctcccttcttttgcattttcagagccattactttccaacagatgaggttgtagagatttctatcacttcaccagaatgggaaatctatgccattttcattaagtaactaaatgttaggccatgtccaattcttaaataatttcacactcttggcaaggggggaaaaaagattattcaaagttatttcaaagtttttacagaattgctaataataataacagtagcaatgccttattaatgcagaatactttatggtttataaatatgtataatatgtaatataacatgtgacataatgtagataaaatatatataatttcatttgaaataactctttagaaattttacttgctaggcctgttttattgcaacttaaagatgctaaggtgaaaaataaattgaaaacattttagttcattccaaacagaaacataaccctaattattttatttaaaaataaaatgtacctccaatcattgataactactgaaattataggtaaataagtgagtttcaggaattattctggggcacagtatttgtaatgaaataaaaaaatgggcatggtaaaaacttggaatctggcaatctgaacacatcgatttgtaaatcttctctgcccataatgggacaccattcatcttctgaaagttttggttttccaatccctgatgcacgtaggattccatattcctttacaaattttgcttctccctctccccacaaaggcagtgaagaggaattccctatatccttcatctcatttgtctttctttgtcttcactcctgatagggaagaagtttggtttgtttctttttctctctctctctttttttttttttttgatgataaatgcatttcactatttatttagatgtttaagctctcacactaagtttttacaagctgatgaacactgacaaattatttctcccacagaactaaaaccacacatccccagacagtataaatatatggttgaactaaccttaatacacatcaccattttatcaattacataataaaacaaattatcaagtatccaaatattaagttgcacagctcaaaaggcatataaaaattagatgtctgctcaattcattagcagaaacctgcttcttttttttgcaagaatggttaggaagagaaggaaataaaaaatcactttcaaacaaaaataagttggtaaacaacttctgataaatatggaaaaaattacaataattttagaaattttatgattaagaattgttttggctacaataacaaatcatctctacctttaaaaaatattttactaaattaaagggcatatcccacatgctctgcacaagacaaaggcaaaattttgctaaaaataatgaatagccccctcccattcttttctcagtccctgcctgtgaagttgcacctcagattgcaaacattaaatgaactgtaaggcctttctgtctgaagacattaaagacacatgcttctatacaatgtagattttttttaatggaggcttttcataacagcacaaagtaagatttattgaaacacattaaataatcactgtaagacatggtaaaaaaaaaaaaagaatcaatggttaaacaaatctgatatttgctagcatgaatgtgggagatgaaaacatggagtgagcaggcaaatcactatgtaggccctactttcaatgtttgaaatgattgaaggaataaactacataaggtctagagtgttcatagttcctgggattttagttctgtatggtactaaaaatacacaagtattgtgccgggtggcacctaatcttcctaattttcttatatattgataagattctggcaagcaaatagatttaaacaagacataactcaatactgcatgatttcgggaaaggtcagttggtacaaacgacaggcacattttaaatgctaacagcaagaatcttttgccaagtgtccaaataggttgactgtaacccaaacacagaggttgccaaatctgtctagcttgtggcctggtaaaactgcctttcagtactcctatggaagtttcattagacttactcaaagccaacatgttggctaataagtataacaaatcaggtcctatctcaagttccacttccaaggcaatagcatcagggaccccagttttattaactgcaacagttgtatctatgaccactggagtctttgaagtgcagccacatctttaaggatgatacttctccaaatacaaaagttaacttgctgttaaaagctccatactgcttttttcttgcgaattgtactgcatcatcatctttcattttgccttcaattaatgctagggcaacaagtactggagctctcccaaggcctgcaatgcaatgaacagcagtacaacaatgaggttctttatgaaacttaattttcacaagacttaaccagtcatcaatctggttggatggtggtgcaccatcatcaaaaggcctatcgagaacatgggtaccttctttctccagaagagtagtgtcataagttgctttacatactcttatctattgtgaaaactccatacttcttaagttcctctacgaatttgtttaaggtcacattggttgggttgtgtgtagtaagaaatcctcatgttcttgtatgtgacttccacaggagctggacagttcatttgagccatgttaatttagttaaaagacactcaatagggttatgaaagaattttaaaagttgaatacagaaatgatgcaaagaaactgaagtctacttcaatatactccacttgaaattctcagtgctttgagtatgaagttgtaagtaacaataaatgaaatgaacctcctaacaagaagcgacacttcttcaatccagtaatactgaggcaacagaaaggaagcacactgaggtttaccctatctaggtcagaactcttgtaaaatgccctatgggtttcaattcaacacttctgtgtccaggacaatgggtgcttcttggtagagtagtaatgaatttctacagtttccttgtttgcatagaggtcatgctatgcctggcagtgatctccactgcccttcagaaactccataaatatgtgaccaagaacaccacagaactgaggaatgtgtttatgcattgaaaatggtggcctactgcgtcgatctcactgggagctgcagaccggagctgttcctattcggaagcatcacgctacctgacttcaaactatactacaaggctacagtaaccaaaacagcatggtactggtaccaaaacagatatatagaccaatggaatagaacagaggtctcagaaatagcattgcacatctacaaccatctgatctttgacaaacctgacacaaaaaagcaatggggaaaagattcctatttaataaatggtgttgggaaaactggctagccatatgcagaaaactgaaactggaccccttccttacaccttatacaaaaatcaactcaagatgtatcaaagacttaaacgtaagacttaggaccataaaaatcctagaagaaaacctgggcaataccattcaggacataggcatgagcaaagacttcatgactaaaacaccaaaagcaatggcaacaaaagccaaaattgacaaatgggatctaattaaactaaagagcttctgcacagcaaaagaaactatcatcagagtaaacaggcaacccacagaatgggagaaaattttgcaatctatccatctgacaaagggttaatatccacaatctacaaagaacttaaatttacaagaaaaaaaaaaaccatcaaaaagtgggtgaaggatatgaacagacacttctcaaaagaagatattcatgtggccaacaaacatatgaaaaaaatgctcatcatcactggtcattagagaaatgcaaatcaaaaccacaatgagataccatctcacaccagttagaatggcgatcattaaaaagtcaggaaacaacagatgctggagaggatgtggagaaatagaaacacttttacactgttggtgggactgtaaactagttcaaccattgtggaacacagtgtggcaattcttcaaggatctagaactagaaataccatttgacccagcaatcccattactgggtatatactgaaaggattacaaatcattctactataaagacacatgcacacgtatgtttactgcagcactattcacaatagcaaagatttggaaccaacccaaatgtccatcaataatagactggataaagaaaattggcacatatacaccatggaatactatgcagccatacaaaagaatgagtttatgtcctttgcagggacaaagatgaagctggaaaccatcattctcagcaaactatcacaagaacagaaaaccaaacaccatatgttctcactcataagtgggagttgaacaatgagaacacacagacatagagaggggaacatcacacgccaagacctgtcggggggtggaggctaagggagggataacattaggagaaatacccaatgtaggttatgggctgatgggtgcagcaaaccaccatagcacatgtatacctatgtaacaaaactacacattctgcacatgtaccccagaacttaaagtataaaaaaaaaaaagaaaagaaaatggtggcctaatcctacagctggtccccaggcagcagtggcagaggctgcagggcaggaggcagagatggttgttgctgggtggcggtggccatgttgcaagcagccagtgggaacaccagactctaccatgcagtgagcggctcctggtttttcagacatcaaagttatactacaaggtataatcctgattcttttacttattcaaaaatatttgagtacctactttctgtcaggcactgctctgtgtaataagaatacagcaatgaatcaaatagaattcctgctgttgtgtagcctatattctaaagaggcagacagaaaatgagttcattaggtaattgcagagtttgtcaggtgataaagtgctttggggaaaagtaaagcagggaaagaggacaggtgagtgatgtgggcctaaatgaggtgatgaagaatggctctccagaaaaggtaattgttcagaaaaaaaagttaaatcattcaagaaaaggctacacttatattgcaagtattccctctacaactgctgaaatcaggctcaaagggatgattgtgaaactgtgtttgtcagcttggggatgcaggagaataacagaaaagaggggcaggggagcagattgggtacttcctaacagtggattgagtgcttgaaacgtatcagatacccaacaaatgtgcattgaatctgagcatagtcaagtttcacacctctcataaattgaggtaatatagcagtcctatcaagaacagtattttggaatgccagtatgctggatctcccttatagttcagctggtttctatctctgcacctcatccattggatagtcctctgacgggaaaataacaagtgttatctcagaagaagatacagcatcctataagggaagtcctagccacatgaaaaaggagggaattcctgccctcctggacccaagggatagtgtaaaaaggagagtgctacagaattgcctctttgtgtcctatccttgggtcaaacattggggtagggtagatagtgctggacattaaacctttgctagctatggaagaatctggaaagcagggtttaatagcatgttttccccactgtgcctgagaaaagatgcaaacccctggttaaaaaccttgcacgtggatacatgtcccaggagcactaatagagaaatggccagggagaagaccagaaggtagaattcctggctccccaaggcccattcagcatctagaactacactgtccaatatggtagccactagccacacaggctccttaaatttaaagtaaccaagcttaaatgtaatttaaaatgtgttttcttagctgcattagccacatttcaagtgctcaataactatgtatggctcaagactactgcactgtacagcacaaataaaaaatgtttccgtcatcatagaaagttctattagacagcagtagtcagcaaacttttccgtaaagggcttacaagtaaatatgtttggctttgtggacaagaaagtctctgtcacaattattcaatactattgctttagcataaaagcagctgtagtctgtatgtaaataaacaagtatggccgtgttccaataaaactttattttaaaaaacaggctttggtctgagggatgcaacatgcctgtcgctgggatagaggagccaaaagttcccacatgcctcagtaaagggacagaagccactgaaagaatcagtgagctagtggatacctgggctagggccagattacctgatctgtgcacgcaaaggcgatatgggtccaggtgcaactgagacacataggccaagggccagaccacaaacctgcacacagcttcgagtccatcctgggaaggagcaggaagagaggaaagaaattcaaaagaccaaccccaaataatccaaatgatctagaagaaactgtttaaatggaagagacagtgatactgtctgttggcagatgtaggtggccccgccccctgtgccagtcccaccactccccatatccctaaccccctgaaggagtaggagctcaggaggaagtttaaatctattatttaaagaaaataagctacagtttccttgactttctaagttacagcgaaaataaaaagtcaatgctgctacacagtgttttctcaaatgtatgtaaagaccctttagaaaagaaatctttgcactctttctaggagcactaatttgggctaattcatctctacagcccctccagggcctccttgtgactgaagatatacactaaggccttgtgtgaggggaacactggcatggagggagaaaatctgggctccagctgtgtgacttatgctaaacctctaactaccctgggcttcagtgaccttatctgccacatgggaagatagatcaaatgactcctaagttcttatccagttcagagataataattttatgattcatctgaaagaaatctgactggggttgtggcccgttgctataattaacatagttagcctgatcacatgtcaatggattaagccttagctttactattaagataaaactgaattagctaagtttctaccagttcactgaaaagttctgatatttaatacttggatagttgtaaggacatgctatcactcaaactatattagtcatccaggctgccatactaaaatgccatagaccgggtgacttaaagaacaaacatttatttctcacatttccgaaggccggaagtcgaagatcagggtgtcagcataattggtttctggtgagggctctcttcctgacttgtagatggccaccttctcactgtgtcctcacatggcagggagagagtgagctctctggtggctcttctttttaagggtactaatctcattatggggccctaccctcatgacttcatctaaatctaattacctcccaaaggtttcatctccagatactatctcattggaagttagggcttcaacatacgaattttgagggaacacagtttagtccatagcacaaaccttcaaattaaggtaagatgctgacttcttggaattttgcaatctgctcatggacaaaacaaaagatgtgtctctcatcagcaagaaatttttgagaaaggaatatcaagttgtgtcccacctttctgacattcaaagcagaggcaggatgggaggatgactgtctcaattacccactatcataataactaacatttacttgacagcttaccgtgcaccagacactccttgaagtgctttacatgtatcatctcatctcactccacaataacctaaaatgacagcctttattactagacctattgtagagatgaagacacactaatacttggaaagccttaggagctttcatacagtgtcacaagtaatatgagactgagtccagatataaaactcagtagactaactccagagcctgccctcctaatcaccatgatctacaaccttcaggttgaacacaagtcatgggaagagtgcctgtgtgcatgcatgtgtgtacacacatgcatgcacgcacacacacaaatcagatgtggttgaaaatagcgtgcatttaatgtattcataatatgtgttaattaatgtattaatgcctataagttattcaccatctatgatggacttctttaaccaactaagcaaaatgcattctcccaacatcttgatgagattagaatctcagaaaatgggaattttgatgagtggtgaagaccaagaagatcctttgaattggagcccttgaaggatgaaaggtccagatagaacattgaacttagaagccatgaacagctagggtatgagacaaaggctcaagtgctcgatgagaggaaaaggatggaaaaaaggaggaagccatattggggcaagaactatgccaagtgaggtgttcacactccagagctctgtgtgccagacgttgtacaaattctccctgcaataatctgtagtctgtacacagcctcccagagacaaatcacttttacccttttgcccaatatgaccaaagtcaagtcattaagacctctttttgtgccagcaaggtgcttaagtttggtcattcctggctgccaagatatgatgtatgacttatgttaaaatgtgactgactgcatgtggaacaacactcagaggaagctcagaaagttcaatagttgagtttaaagacaagagcccagttcaatgacagaatggatgtgcaaccagtcatcctagccatcaatttaaatggccatataatttgaaaggctgacaggcattagtgttcagtatgaagctgtctgaggcactagcttggttacctgatggtcaagttcattatatggctagagctgggcagcaaggtctgtaagttcggagattccataccatatgttttcccatcatgcttggacttcttgaagatatctatgatctcagactctgaagtcacaatcgactagatagttgttctttggaagtgggtcaggcaatggaacatggatatctttaatcacttcttgaagacctcctcaatggctgagaatcagtagcttagctaataacaaagcccttcgctttgacctcctcagctgactctgggaccttctgtgcttgtcctcttcatatcccttcactcatctctcttcaccttaaccacttgaatctttaaccaggaaatctttgcttgacctgggcaattccctgcctgcctttttcataggcattgtgcctgcaactctcttctccctctcagcttcccaaaccattcactttccttatttaaacccttttcattgcccagatcttggtgtctaatatctttctctaataaaaggaaccagggttcgttccctcaaaacaaatggctgattttaggactgggcaggaaatatacaagcctggagcatcttatagtgccagaaaatatggaagtgcttgaaaaagaaattgatgggatgtattagtctgctagggctgtcttaaaaaaaataccacagactggatggcttaaataatagaattttattttcttatggttcaggaggctggaagtccaagatcaaggtaatgtcagggggttggtttctggagaggcctgtcttcctagcttgtaggagaccaccttctcgttgtgtcctcacacgtcctttcctctgtgcatgcgcgtacacacacacacacacacacacacacacacaggttggggggagagagagagagagagagagagagagagagagagagcatacaagcatgctcccttgctcccttgcgtctcttcttcttcttcttcttttttttttttttttttttttttttttttttgagacggagtctcgctctgtcgcccaggctggcatgcagtggcacaatctcggctcactgcaagctccacctcctgggttcatgccattctcctgcctcagcctactgagtagctgggactacaggcgcacgccacaatacccagctaattttttgtatttttagtagagacggagtttcatcgtgttagctaggatggtctcgatctcctgacctcgtgatccgcccgccttggcctcccaaaagtgctaggattacaggagtgagccactgcgcccaggcccttcttattcttataagaagcacactagtcctattggattaaagcctacccttatcacctcatttaacctttattatctgcttataggccctgtctccaaatacagtcatattggagtttagggcttcaacactgtggatttcaggaggacacaattcagttcataacatgaggttataagccacccacttctacaaaaatttttaatggcagccatagaaaactactacagcatcctggaacaacaaaaaaagaagttaaaaaacaaaaaagaatctgaatcaactgtagactatacttaataataaaaattaggttaaaaaacataaagaatctgaatcaactatagactgtagttaataataatgtatcactattggatcattaattgaaacaaatgtaccatagtgtaatataggatattaataagggaaaacttggtgtggggtagatgagaactctgcagtagtttctcaaaaggacacacatgccaactcaaagagttttcactggcaatgctagaataatctgagcaacaaaatagagtagtaatggattataatccaatgtataaaatatccatgaggtcatgctgatataaatacatgattaaataaattaataaatggggggaaacagacaaatcttccacacagatgaattgcaaataatttatgtagatctttcaccctgaagagcaggagcctaactctccccactccttaggtgcgggctgtgcatagcaacttccttccaaaatgcacaatgtggaaagggagaaaaaagagtaactttacagtagagaaacctgacaaaacactacctcggccaggtgaccaaggtcaacatcaacagtgataaatcttgttgacactgtgtacccttgataagatgtgatgaaaatatcactttacctctgtgaccttcctgccagtaacacataaccctagtgttatcataagaaaagcttcagacacattccaaaaaacgggacatcttacaaaacacctatccagtagtcctcaattttgttgagtcatcagaagataagggaaatctgaaaaactgccacagccaagcaaagcctaaagagacatgacataatgtggtgtcttggatgagatctgttatgggttgaattgcatctcctctcagaaaaaaagatatgttgacattctagctcccagtacttcagaataccaccttaattgaaaataaggtcattgcagtgtaactagttaagttgggatgcagtctagatagggtaggtcctgaatccagtacgactgccgtccttctaagaatctcacggccatgtgaagatacatggagaaggccatttgacggtgaaggcagggattggaaatatgttgctgtaagccctgggacaccaaagattgccagcagccaccagaagctaggaagtgaaaaggaagggctctcctacaggtttcagagggagcatagccttcctggcaccttggttttaggcttctagcctctgtaagacaatacatttctgttatttttaagccaccagtttgtcaaaaatttttacagcggtcataggaaatgaatactggatcctggaacagaaaaaagtaaataaacaaaacaaagacatctgaatcaactatagactgtagtcaataataatgtttccactactggatcattaattgtaacaaatgtaccatggtgtaatataagatattaataatggggaatctcggtgtggggtagatgggagctctgcactcagttctcaattgtttaacaatggaaaactgtttttaaaataaagtatattttaaaagaacctttttcttatttcagcaattgttgaaataaggtctcaatttctcaaagctttgattttgaaaccatctgcctttgtgtagtttatatcaaatgcattctaattttaaagaatcaggaacttctccacagaaagcgatcatcctaaaggcatcatgcctcccagtttagtcttgtctccttcctctcctttcctcacacgtgaatcttttaggtcttgttaaccttccattcattcactgtatgccaggaaatgtgccagggatgtagggtaaaaagtgaccttgccatccagggaaggaggcagacactagaaacatcatcacacatagaaatgacaaatcaccctgtgaccttttaagcttaaatggctggctgtgaggacaacacaatctccgccatgagcagaatgcttagcttagaaagaaaccaacacaggacacagcagagaggccctgggacattgctggaaatgctggagttggttatgcctgaattccctacccttccttgacatcatagttgcacagcctgttaaatacccatttttaacttaaatatcacaactttcaaaaattgtataaaagagaagttcatcatgttatgggaatacttttagagggctatcacttaggagtgtcaagaaagacagctctgaggatgtatatccttgaggacattatgcgaagtgaacaagccagtcacaaaaaaaaatactgtattatttcacttctgtgaggtacctgctatgatttgaatgtcccgtgcaaaattcaggttgaaacttaatccccaatgtaacaatattgagaggcagggcctttaagaggtgattgggtggtgagggctctgcccttatgaatggaataatccattcatggactaatggattaatgtgttatcctgagagggaaactggtggctttataaaagagacccgaaccagcacattagcatgctcagcccctcaccatgtgataccctgcaccgccttgggactcagcagggagagcccccaccagcaataaggctctcaccagatgcgacccttcaatgttggactttccggcctccagaattgtaagaaataaattcctattcttcataagttacccagtttggggtattctgttataagcaacagaaaatgaactaagacagtatctgagtagtcaaactagtagagacaggaagtagaatagtggttgtcaggggctagggagagaggggaatgggaagtttagtctaatgggtacggagtttcagttttgcaagattaaaagaagttgtggagatggatggtggtgataattcacaacactgtgaatttatttaatgccactgaattgtatatacacttaaaatggttaaaatagcaagttttatgttttgtgtatttattgtagtttaaaacatttttaatgggaaaaagatttgttagctcaacatcatgtagaagtcattgactatttatggggatgagaagaaccaattaaaatactagcagcagatgtttctggaaaaggcagtgaaaaagctgcaaaactagtctcccagcaagcccttagtacacaccagattctcaaaagtcactggctttgggatggttttcatgctttttctcagcctcactctttcccatgtcccactacagaaatttcagcacaatctcatattatcagaggaaaatgactttcaatatgcaaagcccatgattctccaagggaacatagagaaatacattaattaatttctctcacttccttctttctctacacattgcaaggatttaaaaaaaaaaaaaaacctcaaaataaccaacaaagcaaaaacatgaaaaagtaaaccaggcatttatcaattatttataagctatatttttccctcttgcactgtttctttctttttatttattcatttatttacttttccttgagacagggtctcactctgtcacccaggctggagtgcagtggcaccatctaagctcattgcaacctcccccaggttcaaaccattctcatgcctcagtagctgaaactacagacgtgtgccactgcgcccagctaatttttgtatttttagtagagacggggtttcatcatgttgggcaggctggtcttgaactcctgacctcaggtaatccacccacctcggtctccttttgcactttttcaaaaatgcagacttttattcaacacagctactaatattttgctgtaagtcaccacaaaattgtgaaaatgcttgtgacagatagttacattgtcccatcaaacctggtaaagaaaaccccttctttggaataacactgcattagctttttcactatttactttttccaattaagaaggcctataatctgtctatgccccattctccacatttgaattgtagaagataaattcctcacacttcgagtaaactatggtaatgtgtgagacatcctgtggctaaaatccttcaaattccctggaaaacctaatctatagcttcataagattatttataaacatgttaacaattctgccaaaggtcaagtagctactgtatggataatgatttacctagaaatcaaagccctatccatccttccatccatccatccatttgcttgttctacgaatggttatagaatggtcctgaatccagccctgtacatggtactgagctaggcctgtacaagatacaagcaatatggtatgggagttaggcattcagacattggagtcagactgcttgaaattgaactctggataaccatgatcacgagtctttccaaatttcagtttccttatctaatatacctgttccttgagaatttggggtgtactaaatgagaaaatgcatataaagtgcttagcacagcagccagcacataacagagcacccagtgtatgttaggtattattatgaaagtatgtaaaacccaacccttgccctccaggttttgttagaaaggctacatctttcttacttattcttctaaatcaggacttcccaaaaggctattgttattcaccacacacagttgacacccaagtcttctaccaccttctttacacttggtgtgggggcttctacacacctcctgctagaaaggtgctccagcttatctaccggttccaggggcacctcctttcttcttttcccatcctttccctgtaatgaggagatttgtgaggtgattgaggagccctgctttaaatcagggattggcatactacagccctcaggccaaatctgacccaatgcctgtttttgtaaataaagttttattggaacacagccatgttcatacttgtatgtattgtttgtcgctgttttcagactataatggcagagttgagtcttgggactgagacattatggcccaacaaacctccaatgtttactatctgggactttacagaaaacatttaccagttcctactctagataatgatgatgaagatgatgatgacgacagtgaagaagacaagccacttctgccatttattgggcaaaaggcaataatattaattctattctacaaataaagacattgacgcccaaagatgttaagtaaccttatcaaagtgacagagctaatatatggcaaccaatgtcactcctaagtttgttctcccaattcagtcattcaatacccatataatgaatggctactgttggtgtccagggcattagccactagggatagaatggtaggccttactccaaggaataataactgactccttcctaggtaagagtaactctcgatagtgtccctgtttattgaaagcttaccccaagtatacttctttccagtttacccacatcttgatatcgccaccatcatactttctactacagcttcatttgatagaataagtcaggtacatcttttgttccttaggaacattcctcactgtttattggaccttagactcattgttgggttccaattccttacctactttttccttcaggtgacatgttcccattattcccttacctagagttaccagataaaatacaggacaccagttgaatttgactgtcagataaacaacaaatttttttagtctaagtatgtcccacataatatttgggacatatgaagggtgttctctatttttatttgctaattctggcaacccctacctaaatatttttgtagctggaagaattatccactgtgggtagcagggtatttactgggaaggcctcatgggttaggacagacagcattggatttcccagcagaggatatgtgtgccaggccagctgggcagcatccctccacagaatgggaatgagagcccctactctcaaggtctggtgaagactcagtgaaatgttgtatatgaatagcattgtgtttggcaaagtgtagttgttttgttttgttgttgttgttgttgttgttgaggtggagtcttgccctgttgcccgggctggagtgcagtggcatgaactcggctcactgcaacctccgcctcctgggttcaagcgattctcctgtctcagcctcctgagtagctgggattacaggtgcccgccaccacacccagctaatttttgtacttttagtagagatggggttttgctatgttggccaggctggtcttgaactcctgacctcgtgatctggccgcctaagcctctcgaagtgctgggattacaggcgtgagccaccgcccctgaccatgtagttgttatttatcttcactccaagtaatatttgttcattcattttagtcatttcctctggtagttcagattctaaaaaacagatattcctcaatataccctcagcacagcactgtcagattgtgtccctctagcctcttctttttccaggatctcactgaaagatttaaatgactatcaaagttaatttaattatgaagtatgcaaaatgctcctcccaatttataaattccatgggataaaaaaaggaagtttgacagactgcgttgttatgaaatgaaagatattatacacacatacacacacacacacacacacacacacacacacacacacacatattcctagagtgaccctcaatgatttttttttaatttttatgggtgcatagtagatatatttatttatataatgtttatgtgtgtgcatatatatatatttatggggtacatgtatatatatttctggggtacctgagatgttttgatacaggcatgctctgtgaattagtcacatcatggagaatggtgtatccatgccctcaagcaatcctttgagttacaaacagtccaattatactcttttagttatttttaaatgtccaattattattgactatagtcactctgttatgctatcaaacagtaggtctttttcatccttcctaactacctcagtgcatttcaatagttcatattcagttacattatctgctttcgagttccaccctgtttcaaattccataatcacatttgacagcagcaaataaaaactcatttttaaaaaattcctggaataatagaatttttattttctttgtgcattcagtaaatgccagatccatcttactcttttttctttttttgagacagggtcttgctatgttatccacactggcctggaacttccaggctcaagggatcttcccgcctcagcctcctaagtagcttggactacaggtgtatatcatcacacctagctagatccttcttattctaaggtctcttttagaaactgaaatttgatggttataattagtgcaatttttcatctcttaccttattagagaataaggataagatagactagtacatttaaaaaaaaaaaactaaataatccatgtaaatacattggagccaaactgtttacttctcagtcttcacttagagtctttgcttctgtgcttttagcaaatagttgctaatgcctctcacactttcttaaaaatgcagaagagaatgatatgttacctgtctccaggagatggtcaatgtgggaatgtgctcatggaaaacctcagttcataaaggtgtctgtttatttctcatgaaactctttagccaaacactctgctaaatttctgtatcaggaattaagcataaaacgctccactttctgttgttgcctatgactcttaatattttgagaactaacaaaaggtaaataaaatgtcaggaatcaagattaaccattagaaataaaaaatgaatcaccacagtgaagaaaaaaagcttaaattcaaggggatttttatttcaaatggtaatggggcctattggttcaagtgaaatcacataagagctattttgctcaagtcttttccaagtgaaaaattgtcaacaggattaaagaaatatattttgaattctaatgtatgatcataagaaactagtgtaaaacatttacaaaattcttctccctaggtaatattccccagttcactttaaaatcccaaatgaaagctgacagactgttctactacagactgagatcttttaaaatggtctcagatcccaaatgcatgagtcaattaaagtgcctggcgctcctcctccccgcagagtgtattagtctgattgctgctgcctgttctccccgactatgatttccatgcacctgttgttcacagtagaggaaaaagctcactaatgtattacccctaaaatgaaaagaggctgaaaaaccattctctcctgctaataaatgctgggctacagtaagcagatgaagaagtttaaaaaggaaaacagggaaagaacatttaacaaaacctgatgtcctctggtttgtactgcagctgaaaatcacttggaacttaaccaatgaggttaaaggaagaggcctgtggcatgaatgggaacttcttttgccacacaggtgctgtggttttgagcatgccactgacagatgcacacacccctaaatctctctccatctgtcacagagcccaccatgatagatgcatgcctttgaaataaactacccatctctcatgctttgcttttgaactctatgtctctatctggagaagttagacatggatccaaagtcaaccattattttagaacccagtgctttagtaagaatcacctagtaaggccctaagggaccaaacaggtaacaattaaattgtgatgaggggtgggcaagcggaatgtctggaaccaccctggggaactctaaattgtcagggctcacacccactgcactgggtgcctggagcaagggagatgccatagtggagaaaggagagatggtcaagacacattgactcacatcacatttttcaaaagcctgctctgctttgcaggcagaagggcaggggtagaaattgggccagagttctcctctatgtctaaacacccctctagccctcatgtggctttctgccttcctccttttcaagccacacaaatggtagatcccagggaagcaagaagatggttaaacatggaatcaggcaccttgtatagctaatttacaattttttaatactctttaaacacttgtttaacttagaagagagactgtgcactacaaagggcaaaagctgagagggaagatatgtagggagtcctcagtttcttttgttgttgttgtggtagtgttgtttgagacatagtctcattctgtcatacaggctggagtgcagtagtgcaatcacagctcactgcagcctccatttcttgggctcgaacaatcctcccaccttagcctcccaagtagctgggaccacaggtgcatggcaccatgcctgggtaattttattttttggtagagatggggtgtcactattttgctcaggctcgtttcaaactcctgggctcaagtgatcctcccaccttgacctcccaaagtgctgggatcacaggcgtgagccaccatgtccaccctcagtttcttttgttttcctgagcaactcagttcttctaactgagcatgggggccccactcaggaaatcaaaactttcatctgcccctgccccaatccaactctcttgtccaagtgttccttccagccacacagaatgcaatccatgaggagagttcttgcaaaaacttgtgtcctttcttttctgtgtctagtactgtttagtgccccttatagattaggtgcttggggaaatgcccagaagcctcaccccttaatccttctctggttgaacatctggctatgtaatctcagatgcccccagccttcattaaccctcactgaactctctgctcaggactgctcactgtacatacttctctcctcagattttgagctgtgacgggagcaaaaatcccacaaacaaacaaatatcctaataaatctgaggtaagagagactccttctagaatttagaaaaaccaattgggaagacttgtaatccagcaatttacaaagagcctggcatatagtatatgttcaccaagtacttgttgaaagaaaattaatgaatgaatgagccactgtggcaataggataaaggatgatatgctgccttctatctgcaactgtaactgaagctacattcctttataatttggctctatcatgatctgttttttaacaattacagatactgataatgaagtgcatttttttgacatatgacactaaggagtggtgctggaaaaaaagacttcctgtgccttctcatttccactggtctcttggtttggccttgggtaaacccatatgatatttctgagtagagaacaatgcccgggtttaaaccggatgccagctacaaaatccttatatcagcatgctcctcttgccactgacacataaactgggaaaaaggcaggtttaaactcagctgatggtagacgttcaagtaaaccagtagccacctctcgttcccaacacccctgggggcatcatgtggctttgccatcagctaatcagcctgtgctcatgctaaagttttcatcacctaaaatgggctttccagttggttccccttaaatgaacctcaatacccccgctgtgtgaacatgtggagttcctgataaagatgtttatgaggtaggaaatgcaccacaggacaccctgaatttgcttttgggtggaatgaaacttagaacaaatgcaacatgagatacacattataagctgacttcgactattaaccaaaactccctcagatgcacatagaaagaacaatgatagctgatttctctatttataacccagagagactgcaaaattctgaactatcttctgaatcttcaagcatggtgtcgttttagttgatctcttttaatatatttaaattctttggaaaatcattctattctataatggcaacatcttattaactataatatctgagtagttataaaaataaaattctgcatgagaaagaatggactgattccctggttccctatataaggaaaagagatgaaacttacagaactccctaagcttatttctggtagtagaaaaactcaatcacaattacataagtggagaggtttgtaggaattacataagtgaagaggtttataagaattacataagtggagaggtttaaagggaagcaacgtgttgtcaagacagaacattttcaatatcttgacaaggcatttttcccagttctcctttggtcataacgttaaccataggcaaactagtgaagaattgactattaatttatctacctttagatgcttccttggaagttcttttgtgtgtagctccgtctccctatttcctaacccaaaattcaaatctcaatccatacaacctggcacagaaaaacaacttggggcaagcagattctctggctgggggagtccttgaaataataaataatgagcaagccagatgcttaagagtaaaaagtgaagttgtaggttatgatttaaagagcaattgagaagttggctgggatgtcatgatggatcatattcaagtgagggggagtggaaactatgagcaaacatacactctggcgatgaggttatacatttatttaaactacaattccactatgcagacaaaatatagcttctttatttgatttagaacagccttaatcttgttaatgagccctacccactggataacaaaaggaacaaaccaaaataacatgctttagtctagttcttagtttgaagcaataattttgacattcttaggcattactcaattttttagaatcttatttcttttccttctctgtcaatgtgatgtctggggctatagggcagattggagcttatatgatcttgtgggctttgaggggaaagaggcccaaatgccatttaatattctttgtctcctctagtctccacagccatgaccttgaacagctgctcatggctggccccactggtacttgcagctacaattggcggctggtaccacttgcgatctagcctctcgtagcttgggtggtggctgatggccttccctggctcatgtggcctcaccgcactttactgatgctaataacccttcctaaagggttgtgcctcccatttgtctgtcacagcatcaaaacacgcttactgagaaatagaactccccaaactctgtttcctctacattgaatctacatcactggagcaccaaaagccaacccaaaaagtaaaacatcaggaagtttatctcaccacatggcacctccaggtggacaacttgatgaagtcgtcccagaggtgcagagggcaacatgggaagtgactgcctcctagagtcccaggcatggtatggagcgaaatgctcatgccacttcagtaccccaaattacctggatgttcctaaagctgcccactgcccttgaagtttggccttgagatggtgttgggagataagaggaagttagtctttccatctctgcctctctcctttttcccaccgtgcttcaggctagaaagggtggggttttccttgtcttcgcctgcatcttaccatcctttcaccatcactagttaagccttgtgactaagccatattagatagaaagggtatcatctctatacctcaggaggaattctatggtctgattcatccaagttagttcctgaaattctaaaagggattttgaaaattcctcctgtcatctaagcctggttttcaagcccattattttctcaagggacaagggggtttcatggcttaacctacaaagaatatgatttggaagggaaaagaaaatacattcaggaaaaaggtggaaaggtggaaaattgcaaagaataggatttgagggggaaagaaaatacattcatttaacattcatttaatatttgcaaagtattcattcattacctatcaagaatggaaaatatatttataaagggtagggaatttgtcagaagtgagaggactgctgttcagacacatggagcggagttaaggctgtgattggtagggactaaaatgaaacagcaaacatcttactagagccgtttaggttacagaggactctctggttaccgtctctctggagcctccttttgttagtttatttggcttttcctgtgttcccacgcttgtgtacaaatctctaattacttgatttaacttaagtgcatttctgttccttaaaaccaaaatacctgatcgaacataagcattaatttaagacttttatattccacaatgtgtatacattgtgcacacgagtgtcactaaatctcatttcctgttccctgtaaatgtcgtaatagtctcagattccggctgccacactcccaggtcaccaccatgtcaccctagctggctgacctgactctctttgcggttatcctgccatccttgcccctttaggctccctgaaactccactgtcactacatcctctcctgccccgagacttacacattgctcttcaccctcctcccagtcattgtaagttggatttgcctcagtgcagttctcgggttctgcccatttaaaataatccaatttcccactatggtcaacttgcctgtactctaggcaggcctacatcaaaactggcctgtaaaggggaacaccctgtctgtgcttgctctctcattttctgcagttcctcaatgatttttgcccccatctaccgctagtctaaatctaacccatttttccaagcttcctccaatccaacttattttctttactctgagctgttagttactctaggccatgctactttagagcttaattatatctcatctttggataataggtatctaaagctaatttttgttgagcaccttttatgggccaagtgccttcccaggatagctcatttgatctgcactccacccctgtgaggtgggcacaatgatgtacatactggaaatgaggaaatggagcctcctggtgactaagtaactttcctaaagtcatacaggtgatgataagtggatggaactaaaattcacaccatgatatcagattccagagactaaacttgtcgttaatgctggcaaaccgcccccagagaatcactgcttccaatttcaagtgtcttgatcttatctccccaaccagatggtcgtaactatgctggcagcaatatgggcaagatcatgaattctgtgttgtctgcatcctcctaagcaagcacatacaagacccttgaatgattatctcccctgactggcctctcccattgaatcaccattatctcccatatagagcaacatggtctttacatttgaggcttattcagattctgttaagtttgagtgctgcacttcctctctttcctcccatgcatactctgtgagcccctggatggctaaggtgctgaccgccaaaggtaggaggcatctggatgctttcccagagactctacttctctctctctgcccatctgccaggcttgacttctgatgtcatgggactctggtcaacccctgatgcccttttgcaatgtcgggattgcacaatgttcctaaatctaccttccagacaaaccaatcccaaaggagttcatgagcaggattatagcctgcagaaggtgttcccaggccagctcccctgaggctgggctttttgtacagtgttgaaaggagacccctaagatttctcattgaaatgctccccagccctgtgatttatttagagggaaaccagctgccccagtgagagcagagaaatgtctagtttgtgaacagattttaaacattagagcttatgccttcaaagggtggtttcccaaggggatctctcttcctgctcctaaatgaaaagctacaagcatttagggctaccttaagatgtggtgaaatgagggtttgagactgtcaacatttggctttgctctttgcggagggtgctggggagcttcacaggcccaggcctggcaggaaaaggctcggcaggggcctctttgttgtgagaggatttggggtggaaatcattcgtttcctccaaaggcctcctccccaccaacagtgtggcgcctcccttcctctccagcatcctcgccggcctctccagtctctctgcttcccctctgccacccagtgtcctccttgggaggaagaagagaacaaagtacattcaaaaaggccttcctctctcttcctataaggtcaccccaaaaaagtctccccaatatcttagagtgcaagtttcttgttataccagggtttggctgcagaaactagaagtaattccatcagtgatgacatgacaagatcatttaaggatccccttaaaagccctgctgtttgtctaaatttctgcccacagctgtctgtttaagaaacctcacagaaacaccatgaaatcctgtttgttctcaacccttaatcagtgctaatccaacaggatgggaaggtggagcttgttttaagatgggggaagggcggcatctgtgatcatgacacacatcagacaacttccccttctagacatgtcaggggatgttatggcttatgggagatgctcaggaaaaaaagcgaggggggttgttcttccctggcctgcttttatctttaattgtcaacactcctggcctgcagaggattgaaaagaaaaaactgtacaggcctaaatcagtcagagtagcagggaaggggtgagatgctccacactcaggatttaagtctcttcctgcaagtggggaacttttttctatgcctggcgcacctgtgcctgcttcaaaatggtcctggacctggcccctggtctgcaaagaatgaagtgaggcagcattacttgacccctaattcactgaagaaatgtatgtatatccaaggaagtgtgattcctggaaatccagatttataaaggtaagattaagtcagggattagataggctttttgatttccttgggaaacatgaaaggatgccttaaaatggtgtctcccaaccaagggcatttgaaatatgatctgattttaagaatttggtccccaatcaaattcttggcaattgaatcatattcagtgtgggggaggtgggagagggaactcatttactcatcaactacatctatgcaattccaacatcggatgggaaagcaaaaattctcaggacagaaaaagattcactaattaattgaccacttatgtaattagttctcatttaatcataagtttccacttatgctgttgtctaccctcacactggcatcaagatctttgttggtcactcccatgaggtcattaatacaatgcaaatcaaatgcaaattgattgcaacaaagtgaaattgcatatcctacaaaagatgtaggctttcatccaacttggatgaggacataatttgagcttcaaaagagagtgatctgaatatcagagaattaaaagagactctcggggaaatgagtgaagtcctttaatatttttgcagaaatgaccctctcatgactgtagcaaaagtcaatgtaaagtaaaggagatcttaaaattttgccagcaaagttaagccaaaaaatcaacatttaattcatttcctgattctaaaattagcaattgattttaactgtaacattctggtaaatataaaataaatactaaatgattttatacctttcatttttaaaaataaaattcaatcagtactttaaaaataccatttattgtaaaaaaaatgcttcttgtttcaagtgatttctctttgaatttttaaagtatcagctatccttggatattgaggaccatctgtggttctttattatttcacatatattatttcatttactgctcacagccaccctgttaggctaagcagctattagcaccatttttacaagtaagaaaattgaggccttgggaggtgatttgaacagtattacaaagttagtaaatattagcttcaattacataggcaatgcaattattcttattttgtaaaggagaaaattctgtctctagcaggttcaatgactaacccttgggcgcatggctagtaatattagttacattccaacccagatcttctgtccctaaatcaaatcttatcctactatagccaccaaatttatttacaaataaaaatagccaagcatgatgtaaccaatgttttgagagctaaagatgtcaaagggaacctctagtctattcagaaagcagggttctgttgcaacctccattgagaggaagggaggggtaatcactattagattaaagtgtatgtttaattcattttagattgagttaaatgagcacttcagtcatgagtacagcaggctatattcttcttcagtgacccatttgccaaaagtaccacagcaattaaacactgagcaaatacatgcataggtaagataaaaatagcactcagatgttgaaaacctaaatttatccagcacatttaagcctagcatagcaaggcatatgtggtatctgtttttattaatggatttttatttctggcttctttttccctttcagctttggtaatgctagatgtatgaacaaaaatctagatatctatccagaaattgtcactttagtactcatcagttgatacaaacaatataactgaggccattataatattaagtgaattttcaacaaatagagttagaaatccaattatctttccatagaaagcttttgtgtggagtcattgttaaataaaggagggaagccagtgggagagttagaacagcaatttctcctccctgcaaaaggacgttggagaagttcatcctgcacatacattaatccattcataatggcctgcccactgcagtcagcccacaaagatcatttttaacttccttttcacttctggttactggaggcttccttttggacacagagcagcaaagcaggagccgacaacttgggcaaaaaactaaaagatgaaacacacagccctgcacacaagcaaatggcttaacactaagttccagatggaaggcacagcacccctcccctggggcctcagtctagggccttcagagcttactggaaatgagagaaaacagaaattccttaccttggttccaatttgactagactttggtcggccttagattagtgcctctcaagctttaataggtgtgtgaaccaagttagtatctgggttaaaatgcagattctgattaacagatttggagtggggtctaagactctacatttccagaaagatggcaggtgattcccatgctattagtccatgaactttgagtagaaagaatcaaaactaacctaagccttagttgggacactgagtcttatgcttgactctcggcaatggccattaaaattatattcaacagggagcttaaagaccctctggcctttgacaattactaactatgtaaccttgagcaagttgcttaacttttctgtaccttgattttcataaaaggttggttataaggattaaatgaaataatgcttactacagtgcctgatacttagcatttgctttgtaaatacaactgatttcactgcttcattttacagatgtacagaaaagtcattctgaggtgaagaatcttgctcaatgtgacacagcttgctagtgccagtggttgcacaggaacacaatctcctgacttttagttgaaagccctttctaccacctgtgggtctgctgcttagttagagcccattactaattaagaaagctgacctagggtacacccaggcccccttcacttccccagcctcagaaacccacagagcaactatggcttacgtcacgcacccaaatatttttgctatagccggtcctgacaacacaagccttctgcctgccattaattatgaagattacccagataattgatttaattgggaacagggagtatctcagtgcaaccagccaaagtggattagatctcctcagtgcggtgtgtagatccctgcagctctcaaggggaaagcatttccctcatggggaaaattatggatgccccgatggaacctattgtctactctctgcataacactagctttggtgacctggtctctggggataaaattaaaattagtgccaggcacatacaaccctttcttcttccctgccatctttatagaaaccctcaatagagttctaatgcccacatttgacaatgatgtcttttgggaggtcaggaagcagagagagtaaaaaaaattctctcccaatgtggttttacaaatgggcattagccacaggctgttccagctcaagggtgaagagtagcacaaactcttttctttctctgcaataaaaagaatgaaattacttctttctatagcagtgctaagatatgaataactgaactggaaaaaaatattagctgtctgtctactgttgctgcataatacaaaatatatatcattccagcatctgcccagtgaaaaattacaagaacactagcaaacatgtgctacaaagtatctttgatttgcttgtcctcattattgatgcaatcaaaatggggttatcaatctggttctttgaaatcaactattttagtatccatttattctaaagacacaacacttttaacaaatcagcctcacatcactttaacctctgtggaatattttcttttctgtttctccatgtatgaaagccatttaaaacatctggtagactgaaagcccaggaaagagaagccacctgaataaaatacaaaaagcagtgttattgatacaaatactaaattatgtgtgctgagattcttaaacttgggaaactgaaaataagatggttctggagactgatgatggaatatagagcctttgtcttttaggcgactgttgacattctaatcagagcagaaatgggaaaacaatgcagcagaaatgcttctacccattgaggatacattctgccttagtttctttctaagggcccatgaacctgccaccataaatcaaaaccatctaagatgttgctttacatgctttggcaacattcactatttgatgggatcagatatttcctcattccctgttatggattgaattgtatctccccccaccccccaaaaaatgcaaaaatattaacccccagtacctcagaattgaacttatttggaaatagggtcgttgcagatgtaattaaattaagatgaggtcatacttgagtagggtggatcctttatgctatgtgactggtgtccttataaaagagatggtcatgtgaagacaggcacacacacagaacaccatggaagatggaccagagattggagtgatgcctctacaagccaaggagtccctgtgactgctggccatgaccagaagctaaagagaggcatggaacagactctccctcggaggcctcagaaggaattcaccctgctgacacttggattttggacttctaggctttagaacaatcagagaatacatttctgtgattttaagctatccagtttgtgctactttattacaatagccctaggaaactaatatatccccccaaaaggatttttttcttttttcttttttttgagacagtctccagcctgttgcccaggctggagtgcaatggcacaatctcggctcactgcaacctccgcctcccaggttcacgcgattctcctgcctcagcctcccgagcacctgggattacagatgcccgccaccacacccagctaatttttttgtatttttagtagagatggggtttcactatgttggccagactggtctcaaactcctgacctcgttattcacccaccttagcctcccaaagtgctgggattacaggtgtgagccaccacacccagccaaggattgttacatttctatcctttatgtcaagtcagagcctggttagccctctgagataggaatgaatctgagattccaaaaaataaatgaacttgaagataggaaactttcccagccccatacagactttctactgagccagtgttttgttggagatgtgtcactttcttttaaggaacatacagaagaccttggggtctccgtgtctgtgtgtctggtacccttcagtgtgaagcaaaacacatttggaaatgtgaaattttgactgtgccccctttcaactgttcaccattctttgatttatttaatgaatatttatcttctgcccacagggacagataactgacatgttgagaacttaaaagtaactggtcttatcttcacctgcctttccataaacatttatattcatagcatcagtgtcacaatctatcattgattgattttgaagttgtgtgtttctcagagccttacttacaccagcttatgaacgaacagataagagctagcaaaatttctgctcagagtctgtacataaaatggcaccactggctatagtttatactggaaaatagctttcatctttcatggcacacaataggcccacactaatggcctggtgatgaatgctgatgctgagagctctgtattgaggtcaattacaagaccacacctggactccacaggaactgtgctgggaggcatcaatgtctccaccaagcgctcgggggtacagtcgtagtgaaggagctgagtatttgcccaccctgcttttcaggttcaagtgaaaagactttgaaattataccttaaaaaaaagaaacttgtgaaacataaaatataactttaataacagaatgtgtgacactatgaatcttggagaatggcttaagaaactcagcaagcatttttttctagcccttctctaatgataatgtatctaattttacagattaaagaagtaaagttgagtattttgttttttaaatcatcactgctactttccaagcttgtgagcttgagttcttttaggatctacattcacgacccttctactgcccaagagacttacatgtcagaggagtgacaacatctccaggagcttggtaaattttggccaactatttgacacaatccttgtcccttggaaaacactaatgtatgtttaacgtgaaaatcaacttcctagaaatcccctattccagtagcttgtaaggatgacagctgtttaacagaggaaaactggtttcaacccttgtgtatttgaacaactgcttacaatgtaaaataagaacatctctggaccagtcaatcccgtccctgcagaattatgtgctggtttttatacagtgctaaacagtttggcagatctaatgtcatcaatgtatgaattagtttcacatctctggctgtaagaaactgataacacccataaagcaattcccaggtagactctgattggccagctcaggttacaagcccatgccgcagtagtagggtatggtgcccaattaataacccatcaagattctgtaaaatggaggaggagttcctttgattgtatccagcacaataggaatgggataatatgctgatcaatgacagactatcagagcccacttcaatattctgatccttggccgggcgcggtggctcatgcctgtaattccagcactttgggaggccaagcaggtggattatgaggtcaggagttcgagaccagcctgaccaacatggtgaaaccccgtatccactaaaaatacaaaaattagctgggcgtggtggtgcatgcctgtaatcccagctactcaggagctgaggcaggagaatcgcttgaacccaggaggcggaggttgcagtgagctgaaatcgcaccactgcactccagcctgggcgacagagcaagactccatctcagaaaaaaaaaaaaaaagattctgatcctcttaaactgaggtaccccatcttatatctctacgtatcctctgtagtttctagcactgtacctgaaaatgcaatcttttaataaattctgataaaaagaagaggatgtagcaaggtaaagagctcttttccacacctctgcttcacatcttcaagaaccccatttgatgccaggccagaaatggggctctaatgcaagctacagtggctctggggcttctgatgtgcactttatttggaggagggccaacttaattcagaaaagagttctccaggctggactgacagtgacctttccacagtgacctcagtttaaggcctcattcaatttgaaggagaatcttaaggctagttcgaacaagctgcccctgagataaagccagaaatggttcatgctctgcagcagaggcatagacataatgaactgctgactggtgccaaacaatctcttccttttcattcactatgcgtaattagtccagatttataggacagacttcctagaatcctcaggcacacacacacgacaaatctctggaaggaaatggacttcaattttcccttcccaagctcccctcttggaagctctgtcactcaaggccctctgtctgtcacatctcctctgagatccttgaaggctgagctggtttggaaaagagcacactccattatcaagccaggctccttacacatcttagaacttactgacacctgctggacggtttccttctctctgaaaagcatctgagcagaagcctttgccctatgtgtgaggtcgaaggagccaggaccttggcttctgcactggaagaggaacagagagctttccctgccctgcggagtgctgtgtctgctcagtgaaaacgtacctcctccccacagagagcagacttgagagtccccaggcagcttcaagaacaagcaccctgatggtaaaaatgcatgcaggaaggaacaccatgaagagcagttcagaggtggaaggttggaattcatgatcatggtcaaactcctagattcttatggcttttcctcctggagagactgaacatacagctagcttgaaaagtatccacttccaagtgtggagccttacactgttctctgggttgccagagccaggagatgctgcttgaccgggtctgtgttgccccagagcaaatcctgaaggaagtaaaggtgacccttggtactgtccactctcaaaacatctggttgtctttggctgccttctccaactcctaatgttttcagggcaatgcactcttctcaacaggttggaataagcaaataaactaatttcattgcatccttgtgaaactgcatgttttaagagaaccttggggtgagatttgtgctaaagtccagtgaatctctcctaatggtagcattttaaacctgaggataggctaataacagaaactacttggaaaagtgggcttgtggaacccctttaattataacgtgttggacagctgcctgcaggtagatcttgcatagaactagttagttattgctgctatcttgccaaggaattgccctacatggagaccaactgctggaccgcccattccccagcctcttctccttcttcccaggaaagaggaggagtactcactggctggctggctggctggctcccctcaagctccctggaagatcccttctggagaagctattcttcagcattccctcctggaaccacccatcccatctgcagtcataagagcacccctgagtctcatctcagttttcctccctaagagcccagctaaatgcagaaagcaggagagtaggtgagtgtgagagcatctgtaagcctttttgccgccagggctctagtgtggcagagagacctccctgggagtggagctatacccttccctatacaggaacacagcctgcccaggccaacctcagaaactaaacacctttatttcaagaaagaaacaccatgctcaaaggtcctccacctttggggcccccctcccatcttcagtatgcttcaggccagaatttaggtagagctctgaagtttgagttctaatagctgggtgttgcctacatttggtgttctaaatgacccctggcctcctaggggtaattatacagcgggatttgagggtttaaggatgggtaggccaatactggtaaataccagaaggaatgtacaatttccccctaaaagcctgttagggccagagcctgcagagatgaaagaggtcaggaaaatgccatgatattacttgcttcctaccttaacatttggcctctctgtgtccctgcacaaagaaacctgtgctgcctgtcagatatgaaaagcattgctatttagcaactcaaaaaccattagaggatttacttcaataacctggaactcatggatttccagaatccagcctttgtagtgctctctaattttatgatctgtatcctcacaaatcctcctctgtcccagacatactgtgttccagcacagtcttttgttcacaatgctccccgtagccccaaatgcagccctgagtcttcctctccagtgatctcagtcccagccatgtttcagcacctcccccatgtcccacctcctaatgaagccctcctctgccactgccctcaccacccacagctcttaatatttcccctgtcctatgtcatacacttccttaggttgttgcttatattttcatgtaggcagcctttgttctggaagaccacaggcccctcagaggtaagctctttgtcatatacctgtttgactccccagttcaaacccagaacttttcaccacaacttagcccatggagggggtcccctacatggcatccctgaaagatgtttccccagtctcgccatgaacatttctggtgacaggatgtgcatgacttcatcctgacccactccaatattgaatagctataagttttattaagttcttatttctactaagccaaaatcagcaatatctgctcccaatgctgcattacaagcagctgctagctctcaatttttgtcaggattctcttctccagagcagagattccccaattcttttgatcttcatctatcatagtttacagaccctgtaccattccggcagctctctcctgggaaatttctggtttccagagtctctcatgaaagttgccacccagaggggaacacaatattcagctgtatgaagatgtgtgctctgaatgatgaacatactcgagggaggtatcttggtgataatggtgatgatcatgatcacgatcaaattctaaccaccttttaaaacacgaggaaattgctaaagaaagaacgctagtattttacccatccaagggattccagttttgaagaaaaccaaaaatgtaggaaatgcagtcacctggattgtggcagacattgaatctactttctcaatgttatttcatagccttctgatggtagagttgtggtttgagtgagttatacaaagaagttcatgtatctgattaaacagctttatgtgttaccaactggaaagataactatggccttccaaaccgacactggctgcctctgcagatccttgttagaatagagctggctcggaacctcagcggtcctggagatgttccaacagctcaggtttcccagatctctgcactcctcacccatccctaagcctcctccagggcctcaaaatctcggaagtcccttcaaagagccaaattaagagaatgaaatcatcaaatgtatgaagaaaaccaccaggaagaaacaatttccttttctccaagtccctcaacataccaagcaatgccttacgtaggtttagggcttgggaggccagattatgctttagttcttgagggccttttatgtagccacaggaccttacgtgagtgaattctccttcctggccctcactttacttcctactccaaaatctatgaacctaaagtctacaactaatgaaggaggaaagcctgataagaaatcaaatgattcccctaatcactccagcccacactgatcttttgttttcttttaattctgaggcatttattttctgtaccatttatttaattttaatattttgctgccttcttatttttattctcataaataaaaggatagtaagctctaggaaaaaaaaaataacacaactcatctaatgcccttggaaagttgagggaactgactggcctaaagttgcaaagctgattagtacaagagtacagataaccatcccagctagatgctaagctcccaagaggtcagaagagaacaggttgacacatccttctttttagatccaaccagcagctcacttttaatagatgtgcctcaggtcaccttaatagcaaactaggttaggtcttgggtctatgactgaaggcttcttagagaagtgggatgaaaaggaactgcccctgggacatttagttatcctggctgtcatctactaagctgtaggatcttctcatattcataatctcttaccttaaatgtggtggtattttgaaagacagtagctgggcttagacatcagcccaagcaaggcatagctcacagtctcaatgccctaccctgtcacctggagtcctgatgaagtttctgcccaaacagaaagttggggcaccctttattctccaagccttaacttcaggagtctgttgccacaaacttacacagaacagaaatctatatggtgattataacatatgctcaaatataagatggccacaaatacaaagtgatttcccataagaacagccaaaggaaaagtttttggccaatttgaatatataaactttcagggatatggatgaaatattcaattgtctactcataataatttgttaattaaattacttgttgatattaaaaatcatgtactacctaaaataatgcttaatttagaatttcttgccaccctcatgtttcagaataattttgtacattctcaaaatatattttccatgttcgttgtctctttcatcataatcgccaattttttatcttttaccaacttgtccagagcacattgtttgacatacagcactttttgaaatcgtgtgagatgatgtctgtaaaatttctttcaaaccataaactttcctaacagtaggggttcccattcattctataagttgacaatacaactacttccctttttgctattttgtgttttcaaaacccaaatttgaaagtttgaaggactaccctaaggaataattacgaaatctgtgtcaaattgccatgcctgctttctaaaacgcatttttgtaagatgttctctataaaactgtagaactagcagtgattgaatcatttcagggaatgtatctttcctaaatagtgttttaaaaaaaaaaaaaaaaagaaacgagatgacctagaatatgagaaacttcaagaagtcttgaataatacctctatttatatctgtgtctatttttatgtctatgtctccctccctttgtgaaataactttgaaggaaaaactttgccttataggtaaggagtgcccagtttatacctgttttctggagtaattattaatagtgcattcttttatgctcaaaagtgtcccagtttggacaatatattcagttgccactctactgatggcaaggaatatgctatatattataaatctgtagccttgtcaaggcattcaccatattggccattcagtaagcttcctataaagctaatatctgcatggcagccagatggaacttgaaggcatggtgcttggaaattaaagcaatgtaactaattatctaaagtgatattacaatgtgtaagatactgaaaatttgactatcagtcaacacagcatctgcttcaaagaaaacatttttttaaagcatctttattttttagaaatctaattatgaggaactcagttacgaagtgctgctttgatcttagaacagtacatttagtttattattagtttaaactagatattttgtttaatatctctaaatatttggagataaattgtccttattaacttactttaccattgaaccattcagacttacaatgatgtcttgtttaaggcaaaatttatttccaaaggactgtcttgaaagtgagtgcattatgtggaatgaggggcgagtgaacagatagactggctgcaaagagctctgggctgacgaagctctgatcagaaatccaagccttgggcaagcctgccagccttcctcactttctcatgtgttgtgggaggggcctggtgggagataatttgaatcataggagtggtttcccctatactgttcttatggtagtgaataagtctcatgaaatctgatgcttttatcagggggtctctgcttttgcatcttcctcattttctcttgctgccgccatgtaagaagtgccttttgcctcctgtcatgattccgaggcctctccagccatgtggaactgtaagtccaattaaacctctttttcttcccagttttgggtatgtctttatcagcagcatgaaaacagactaattcagtaagttggtaccagaaatggggtgttgctgaaaagatacctgaaaatgtggaagcaactttggaactgggtaacatgcagaggttggaacagattggggggctcagaagaagacaggaaaatatgggaaagtttggaacttcctagagacttgttgaatggctttgaccaaaagcctattagtgatatggacaataaggtccaggctgaggtggtctcagatggtgatgaggagcttgttgggaactggagcaaaggtgactcttgttatgttttagcaaagagattggcagcattttgaccctgccctagagatttgtggaactttgaacttgagagagatgatttagggtatctggtggaagaaacttctaagcagcaaagcattcaagagatgacttgagtgttaaaggcattcagttttataagggaagcagagcataaaagtttggaaaatctgcagcctgacaatgtgatagaaaaagaaaaacccattttctaagaagatattcaagcaagctgcagaaatttgcataagtaagaggagccaaatgttaatctccaagaaaatggagaaaatgtctccagggcatgtcagagatcttcacaacagcccctcccaacacaggcccagaggcctaggagaaaatggtttcgtgggcctggcccaggatcccagtgctgtgtgcagcccagggacttggtgccctgcatcccagtcactccagccgtggctgaaagaagccaatgtagagctcaggccatgactttagaggatacaagccccaaaccttgggagcttccacatggtgttgagcctgcaagtgcacagaagtcaagaattggggtttggaaacctccgcctagatttcagatgtatagaaatgcctggatacccaggcagaagtttgctgcaggggtggggcactcatggagaattactgctaggtcagtgcagaagggaaatgtggggtcagatcccccacacagagcccctactggggtaccgtctagtggagctatgaaaagagggccaccatcctccagactccagaatggtagatccaccaacagcttgcaccatttgcctggaaaagccacagacactcaatgccagcccatgaaagcagtcaggagggaggctgtaccctacaaagccataggggtggagctgcccaggatcatagaaacttacctcttgcatcagtgtgacctggatgtgagacctggagtcaaaagagatcattttggcgctttaaaatttgactgccccgctggatttcggacttgcatgggtcctgtaacccctttgttttgtccaattgcttccatttggaatggctgtatttacccaatacctgtacccccattgtatctaggaagtaaccagcttgcttttgattttacaggctcataagtggaagggacttgccttgtctcagatgagactttggactgtggacttttcagttaatgctgaaatgagttaagactttaggggactgttgggaaggcaatgattggttttgaaatgtgaggacatgagatttggaggagccggggtggaatgataaaggtttggctgtgtccccacccaaatctcatcttgaattgtactcccatgattcacaagtgttgtgagagggacccggtgcgagataatttgaatcatgggggcagtttcccccatactgttctcgtagtagcgaataagtctcaagagatctgatagttttatcaggggtttccgcttttgcatcttcctcacttcttcttgctgtcactatgtaagaagtgcctttcccctcccaccatgattctgaggtctctccggccatgtggaaatgtaagtccaattaaacctctttttcttcccagttttgggtatgtctttatcagcggcatgaaaatggactaatacagactgcttgcctgttatattttatattaaacatgacctggtcatctcatggcagtcagcaggtttgaactctttctgtgtggccaactttgggaagccactctcctggagccacaggtaatgtcatggaaaaagagtcttgccaaggcggtgacttatgaagtatcccctctcctcaaccatatatgcatctcctccatctaccaagcagcacagagagggttcccatgagatatagtcctagagagacgctcgttcttacattgggaacatgagtcctgctctctgtatatgctgtcatccaattttgcctgcagtgagctggcctgatcagagaacaaccaaaggacagctgtccctcagaacaatcaggaccttgagacctttacttgggccccacactgtcctactttctgcttgagagctgaaggcatatttctagaaagaaggcagcctaagcaccaagggtaatgctctgggaattttcagcccccgatgctgctctgcgaggataagagaagaggcagaaagagaaacctcttagaatcctctcccacactctccaaatatctaagaaaccaggccgcctcaggtctcttgtcgacaagagctttcccttcccaccatttcctaacctcacaaagagcacgacgggagatacaatgaagaaggctggagaggaaggcctcagctatggaggctgctgtacttcatcaccacctcttatgtcctggtggccttttgcctccctcgattatgtcctctttctttccggggctgcttcttccagacgaagctgaaatgagagggtaggatgtgaggccaactttcccatgtgcccttggaacacgaggatgtgatgtttatccctccagacccccttctactgacctctctacgcacattgttctggaattcagatgcgtcagaggatgtcttccgtgtgaggctctggccaggagttgctctctgaactaattttacacagatgtgtacacatatggcccaaagcccactaagtgaccagcagccttgtccccagcattctctgctggtctgatgccattgctatattttccaacctatccctgtggaatggtgaccgtggcctcaccataaaccccaactactggctgctctgtccttgaggtcacaggctggcagtgaccttgccagtggctcggcctcccggaggcttgaggcagggcttcttggcttgcagttgcactgtggtgagtcctcaaggcctcctgtaccctgctcttagcctgtttggcttccctcccctgtagctgctcctccctgcaggcctgctggcctccacactccaggacagttaggataacatctaaagcacgtggagttgctgggaagaaaggctcaggtctgcatcagccaagccacagctctgctgtcatttttggtccacaatggccctggggttattgcatttatccagagcatttccacacccacacccttccctccaccctgacctttttatattttctgatcttttttttttttttttttgagagggagtcttgctctgtcacccaggctggagtgcaaaggcacgatctcagctcactgcagcctccacctcccgggttcaagcgattctcctgcctcagcctcccaagtagctgggattacaggcaccccgccatcatgcctggctaatttttatatttttgtagagacggggtttcaccatgttgtccaggctggtcttgaactcctgacctcaggtgattcacccgccttggcctcccaaaatgctgggattacaggtgtgagccactgcacctggccctattttctgatcttgattatgcaccaataaaaagaaaaatttaccagatgataatttttttttattttttatgaccagattgcacctgctacttttccaaaaagccaacttttcagaatgctttaaaaaacactccctttaagtgacacatttcattttgcaaagactatggctttttctcaaacagatttttatcttttttctggggtctgatgtggaaacctcactaaattcctccatctagatcatgtcagtatttgattaaaatgtgagtttgaaggctctcctgtgaagccatttcttcttgcatccttttaaaattaacactcacgttaaaaactgatgacatttaacattgaaaataagctaaacctgatggtaactctctgagctcttcaaattgacgccgaaggtggccaccttgtggctgttgtggaaactgccacttagacctgatttttcccaggcgttggggccctactgctaattaaattaaatgactcttaagattatcttttcccatgcagtttacatcccaatcctgctcctctacaggtcaacttgtagtgatatcctggcagtttgggggcaagaaagctattctaaatggcctcagtgggttagttgaaagaaaccttttggtccttaaactaaggagaagagataacctcccaggaagcccaggtctttgtcttcctcaaaccttatatgctctaagtcccatattttaaaaaatatgctccaggggatccctatatccgcattcctgggtaagatctttaaaatgcaggttgctggggtccctcctcaagacctaagaattggaatgtctaagtcctgggatttgcattttaaacaaatttctcaggtgaatttttaatgcacattcaagttctaacaatgctacattttagtctactaaggaaagaaactggagacctaacatatgaaatgcttttgacgcaggcaatctcccttgggccctgggagggaggggatccagagctgaagagagctcaagagaccaatgcaggagttctcaaaagcccctgacaacccaaatgtttgctgcttgaacataggaggctccaccacagtgacctggcaacttcggtcgaaccctctaaacaagaagccatgggccaccagaagagtggctgcttgtgtatggtgtctccctctggcctgcataggaagcagcaaaaggcagttagaaggagaggcacctctgctggctactccatcacccccacccaactcccaccgtgtccaccccaccccaaagctccatgtcccaactcgatgtgaaaggcaagagaccagcttccaggccaggctctcctgctgaccagctttgtgaacttggacaggtcgctggcttcaggtgttattgctgtaaatggaagtagccagataaccatcacttagcttctttccaattctaacattctgtgatctcattttgagctttcagagggagaggaggctctttatgagaagtgatcatgggagtccagattccaagcttagcattaagcctttgctgtttgtagcaattaattattatggagtgtcactgagaattgagatcctctgccaatacaaaaacaataaacttttgcaatgtcaattctctacattgggggtaaagcagcactggactgaaagccttccaaatgcccccccccaactgacagcaaccccatgacctcatcacaccctcccctcctcatgcactattctccaaagactttaacacaccccatcctcacacacgcccttcccacacccacccccacctcccaatcaggcctcctgtctctgaacccacttcctgctctagcattcatcttgttgggtgctgagatacctgtaagaatgtctaggtggagacttcacccagatcatccttcttatcctttcaagatcattgcccccaggtgccactgctctgagactccaccattccctatgcatactcagctcagttcttaacctgtgacattataatcatctgcttttatgcctgtatccctaaatggaagatctgtggctgccacatactgaatattcaataaatgtttccttgaatgaatgacagtaggaatgaaagaatgagtggataaataaggaattaagaagatgcaagctgactatcgtacagaacctctatacctattctttgcacaagtctggatgaggaaagaagaacctggtccagactgtcttgactctggagcattttggggagaagatgggaatggagggctgtgcctgcagtagaagtagcatggtaggttatcaaagcacaggtcagtgaaatcaactattgtgtggtgtcaatttagtgggttcagaccaacatttttaaatgaagtataacataatagaataaattagaaaacatgtctcttagtaagttccagtacagctttatggaactttttttttttttttttttgagacaagtctcactctgttccctgggctggagtgcagtggtgtgatcttgactcactgcaacgtccgcctcccgggttcaagcaattctcctgcctcagcttcctgagtagctgggattacaggcgcccgccactacgcccagctaattttttgtatttttagtagagacagggtttcaccatgttggccaggctggtctcgaactcctgacgtcgtgattcgcccgcctcggcctcccaaagtgctgggattacaggcatgagccaccgcacatggccaagacttttattatatagacatgtgcaaaatatacttctttactacacacagtggtcaaaaaatgtagaagccactcatctaggaaatacttcctaatactttactaaatacttcattgactcaatttgaaaaaaaaaaaaactgctgactttatggtatggcttccatttattggaatgtggggatccatttgtcaggaaatgtatatggattccatgatgtttttgagagagctatatattttctaattacttgaaatgataatgatctgatctcatctaatttcaattatgttcagaataggtatctttcatagagataaataatttaagaaaattttcctaaaatagggaccttccctctgttttgtcttttatgtctccccatgtagagcctgtctatgatgatagtataatactagctctctctctttcctaccaaggtgaaaaagccattttctaagagttagtagaaaagaagtgataaaagcaaattaaaaatcttaaattccaatatcaaactttgataaatctatctaaatatctccatgggaaatcatagtgacaggtgagataataaaattaaaatctcaaaggaaggcaaatatcaactagctttgttaagaacatgaatccaagcacataggatacaatttcttctctgctgtgttttgttctcctccgatgaacagacagccatgtgttaggctttccctggtttggccataacactgtccccagtctcgtctgttgacaccatatgggattgtcacgcttctttgtttatgaagagtctcctggaatcttaaaattaagtttcagtatcagaccagcatccaaaaccaatccagatacatacttagactggcttggtgtttaaaaagacttcatgttaacagttaaaagtcagaacatttcttttaaaaattagggttcttgacttctcttggaaaggttaaaatatttagaagtgcaagacccatattactgcatggagactattgctcttaacctaggtccaggggacccctgtgttttgatacagtttcctctgtaatcctatgtattttattttctttatttaaaagcgtcatcttgagaagggatccatgggcttcactagactgccagaggggttcatggcccaataaaggctgagaccatcctagagtagagccaagcagcagctgtcccctcgatccctgttctcatcattctttactttctctgaagccatgcatctgttttcattttccattgcactaacaattttcctggagtcaagaaatattttcctgtaattatgtctctatcaaaaattggaaaaagaaaaatgaattgagggttttggaattcttacactaagctgctccacttatttatgccagctcactggcctctggtccacagggattgtgtttaacatccttgatcaccagtgaagcacagccaccaaccagtcagaatggttgtcatctgtgcacgaacaatgctggtgcatgccctgtgaacctcagccctgtgcctggaggatttccatccctgcttcagcaggcttctctagctctctgggtgtcattccatgttgcatctgtccttcttcctggaatatcagggagtgtgagcagtgggctatttggagtgtgcttgggagcatcctggggaactaggagaagatcaatttctcctttttggcaaaggagcagagatgccagctccaccctgccgtccagtctctggacttgaggggaaatggagctgatcatcatcatgtccgcacttgtcctgcatagtgcagttaacagagctgtctccctgggccctcagctgagagatcctcataccaaaccttctgaagaaagagctggaactaacgccactttgcaaaggaggaccatggacagtcgaagaacaggagcagtgaagtcagaaggagaacacagacctgcattttagtccactcaaacacttcgaaaacactcgctcctcaagggaagtgacttttggggatttccacagcaagtgcttaataaggctttttttaaaaaaaacaaaaaccaaacaggccttgtttaatgcattaatattttaacagagcaagtctgggcctttcaagcaaatctagtcgctattcaacagtcaccttagcctttatgctaggtataaagagacagattttggattttgtatggaagatgggctttttgtgacagtaaaatgttttattttatttttacaaattataatacaataaaatcgactttttcttttggtgtataaaattttaacccatgtgtaaatttacgtaaccaccttcaggatacagatctatcactctaaaaaactcctgcatgctagttctttgtcttaaaaaatatatatactgatgtacatgtaaaatggaagctacaaggatatttgtaaaataaagtgtacattttatccatatgaaatcaagatagaatttctctctctctctctgtctttcttgtgtgtgtgtgtgtgtgtgtgtgtgtgtgtgtgtgtgtgtgtgtgtgttttaaaggattgtatttccttccactttggaggatataaattcttgagaaataacctatacaggctatcattaaagagaattttcaataattctttaaagcaatatgatggtttgaccattttcagattgtgacggaaggcataagaggaagcaagatgaaccactggggtggtgcctaaacatagcggccccaggagtgaggcttctgaacattttccccaccccaacccacaaggtcttctgcgcctatactactccccgctccgtcctccagcaatggatctgtatatctctccaggtttgtcctctcttctctctcctgcctcctttcatatcccaccaacttgcctcttttttaccttctgtggagacaaaagtgactccatcttggatgctaatccaccatcttgactttccatcagccccagttctaggaaggcctcctgattcctcctttatttactgaccctagtgcaagaacatgtgctcctgattttagatcaaagcaatcttgatgttttcacacaaattataggctatgacacatatagcattcttgcttgttctggatggtttaattgtcttgctagagtatgtataccctttccttatggtggataagacttgggtttgtggagtaaccatgtggagatctacctatcttggggccacgcaagaccatgcttctgtctataagttccctcaataaattacccaatacaaacaaagtggatttgtctaccacctcctttagtttcttggctccttcgacatctgggagctgttaagcatatatggccgttttcacagagctcctaggaaaaggagatataaagatccaagaagagtgggaaagagaaggtgttctgagagacaggaatgtaagagtgtattgacaaaagggaaaagatatcctttttctgtcccactaaaagtttacctagtttggactcttcctgttcacctaacatggcctaaagtaacaggtagctgtcctctctccctacctgctttccaaatagagagattatgcagcccccttcccccactgccacctgggaaacagccatgcacctgccttccagccttgcagcctgctcttctgcacccacttggtcgaccctgcccttgggcccctcccttagaagaaggatgggagctgagagaaaagggaggaggagagagagaagagaagggaggaacaatgggtacaggtaaaaaggaagctaatgctcacttgcattcagtgaattcctaataagaaaaaaagacacagtgccaggttcactgccctctataaatattggaacagagaatattagcataatccaaaccgtcttaataagacttctgaattctgtaaacaagtgcacccttactagatatttttgcaagtagctatttcttgggatatcacaggaagcggagcttttatttagtcactaaaactagtgtcctttggataatgcttactatgacaccacatacgagaggctgtttagtaaatgattttcattggttatgatatcatctagttattctatgtgtcttcagtgggaaagagaagaaatgcctaagtcagtggttttctcatttttaaaacattaattatcattacatccaaatgaggcttcacatacagtgttaaggtaaagaccaaactgtattcattgtgtatgcatatatatatgacatatatttaatagactacaactatacaactattttcaaagtatataagcttaaaaagaagaaacaaagaataatagaaaatgaattgtttgtgcttcattgaaaaatgtacaagagtttgatataatcgaagaattgcaacccagtgaaattttggccataagtaaaaagcacttgaacatcttaattagttttggagtcaaatttggttccccctatatgggaatgaactcatcccacaggcccacatcagcttacagagttgtatgcccaaaaacgaataactacataaaccagatactgagagtatgaacttttcccagtttcaacactcagctatgcaaagatttgactttatgtttctataatacctagctttaacttttgaatccttgtttcaacttggtagcagaaatgttaagtgtgcccatttgaggtaaaacttagttacttctagagttaaaatctgtaagttttgggtaaatagccatttcaagatgcatccctgctcctgcttgctgctgcttccttccaggagagatataagtactaagatgacttctgtattgcccagaaaggtaaatttatggcttaaatatgttgttctattattcacaaggtacctctgggtcacctttgctataaaagtggagactatttttaaaataaagttaggacacactccataaaagttttacaactcacttaaaatcatagaagagctgtatatttcaagatgaagatttaagctctggttatttaacagggtcttaagaggcaggaattgctgtgtcaactaatgttgtgcactatatttcattactgcctttatgtttttttaaatacctcaagaataaagtttatttaaaatgtcacactttgcaaactgcacattatatccatagaatatgctagtaccactcccattccttttatttattacagtagattagttatggaccctctgctatagtatcttggcttattaagatgctgcatgctaaaaagcatagatggccctgtaatgagcagttacaaaatcaagtcaccaattcttccttggctttcttttttgttcagagggaaaaattgaggatttgacctgtgcctgaaacattaaccacctgattctgcttgtggggtggggacagaaaagggccctctcaacacttaattattgagaaatcctccgtatctctccaggattatagaaaaggattaacaatcatcaaggttgcttgaacaaatctcaagttcaaattccagaggaagacgggatggtctagggtgatcaaaacctagacccagggctgggtgcagtggctgacacctgtaatacagcactttagatgccgaggcaggcagatcacttgaggtcaggagttcaaaaccagcctggtcaacatggtgaagccctgtctctactaaaaatacaaaaattagctgggtttggtggcgggtgtctataatcccagccacttgggaggctgaggcaggagaatcgcttgaacccaggaggtggaggttgcagtgagctgagatagcaccattgcactccagcctgggcaacatgagcgaaattctgtctcaaaaaaaaaaatcatctaattcacaagaattttttttgctaatataagtcatccataaacctctatgaaaggtgagccctttatgagacaacttgatccctataaagtattcaataaacacaatgggcactaaaagacagtcttaatgaaatgggacaatttagaaatattttcttaattgacatcaagaaaaagtaatttaaaaaaaaaatctgttagtttttttttttttaaacaagtgttagtgaggatgtggagaaactggagcccttgtgcactgttggtggcattacaaaatggggctactgccatggaaaatagtgtggtggttctccaaaaaattaaaaatagaattaccatatgacccagcaatttcacttctgggtatatgcgttagtcctttttcataccactataaagaactacctgagactgggtaatttatgaacaaaagaggtttaattgactcacagttccacaggctgtacaggaagcatggctgggaggcctcaggacacttacaatcatggtggaagttgaaggggaagcaggcacatcttaccatggtggagcaggagagagagacagagagcaaaggggtaagtgccacacactttcaaacaaccaaatcttgtgagagctcactatcacaagaacagcaagggggaagtctgccccatgattcagtcacctcccgccaggcccctcccctgacacgtggagattacaattcaagatgagatttggtgggaacacagagcaaaaccacatccgtatataactagagtctcaaagagatatttgcacatccatgttcacagcagcactattcacaatagctaggaaatagaagcaatctagatatccaacaacagatgaatagataaacaaaatgtggcatatacatacagtggattattattcagcctaaaaaaggaaggaaattatgtcacatgctgtaacatggttgaaccttgaggatatcatgttaggtgaaataagccagtcacaaaaaaagatcaatactgtatgattccacttatatgaggtatctgaagtagttaaattcatagaaacagaaagtagagtggtggctgccaagagctgggaaagggggagtgggaagttgttgtttaatgggtatagagttttagttgtgcaagataaaaagttctggagatctgtttaacaagaatgtgaatatacttaacactactaaactgtatactttaaaatggttaagatggtaaatttgacattgtgtgtttttgtttaattataattttaaatctgttagtttctctaatgatcataatgaagattaattaaattgtgctcaaaagatgtattatgggataggaatacaggtaattttattctctttattgttgctggaccaagttttctgcttcctacatatcagaatggctaggcattgcaaaataatctatcaccatagaatctaaggattcttttggaatataagcatcattttcaaaagtaatttgtgattcaagagttgcttagaaccaaatttccctaggaaactgaatgatttgggtgttttgttatggggtggagggagaagatgtggtggaagttggcatgatctctatttgggtggggctctttttgtatttttgatagagaatagctgtattaatcagtagtgtattatagaaggcatatgtcttatgtttcattgcttggttttatgttgtgcagcagcaatttgctaaccacagtcctgaagaaatatagccacgcagcaaaagaagcatttattttcagaaacacatttcttttctgacattacaggggagtgggagggaaggtccagattaactgtactttttaataagagatggagtgctattggaaaactattaaggaaattgtggctttagctcagattcaaggaagcccataagaagaatttactaccttccagcttaatactgcagcagggccagttctacagacccactgctccctgaatgattctggagagtatttgcgagctggggttgttctaggagaggcaatcagccgctgatgagctcccctgcttaactttcctaattgattccaccagacctcctagtagactagttgctaaccctgccccatcccaactctggcctggctctttctgatggcagtcttcagagaccatttgccagtttggagttaggccttggagctgcagaacaggcttgtaaagcagatgcgtagtaaatgctgcagccaaatgtacagcacattttcctaagtggaaatcctgtgttacagacagaacattacttaacagtaggtgggcagttcaatcctaaacagctcagttcttccccatcagagaccctgcagaagcaggcttctgccacagctgtgggaggaagaacagttcttgtaactgttaggagcagacagtggctaagagaaacagtctcttgggaagtccctgtctgtgattttcctacgtaaacacaagaaaacaaaacccctgggcttgtcgttcacacaagcaagccgcacaaagctttctttccttcatctgcatgtgctagtgtgctgttcttcctttaggttttactgctgcttaaaggtgtttttgtccagatatgtaaaggtgcctcgcccttctccttctcaggaaaaataactatttattgagtgcctgttgccacgcaggaactctgtatccatcatctcacctcttcctcatcaaacacatctcttccgagtgagttgccaaattacactgctggcttcccaacctgaggttttgcttctgtttacattttaccagagcaagagagaaaaagtcacaaatgatgtgctgtcttctcatcttactaatcccctcttccccaagcctttccacatctagaattaaatacctgggccttggcagccaggccaacccctaacattggtaaagcccaggcaagagcacaaatggatgcccatatgcctaaagctgtaagagttataatgcccctttaatgcatcctcatgtccacgttggatatgtgcattaaaattcatgaaaaagaattattggccaggtgtgatggctcatgcctgtaatgccagcactttgggaggccgaagtcagaggatcacttaaagccaggagtttgagaccagcctgtgcaacatagtgagactccatctccacaaaaaataaaactttttaaaaaaattagccagttttggtggcaatacctgtggttttagctactcgggagactgagatgggaggattgcttgaggccaggagctacgattgcaccactgcactccggcctgggcaacagcaagaatctatttcaaaaaaaaaaataataataacaatagcttaaattcgaaacaatctaaatattcaacaatggggcattgatgaattttattataatatttctccatatagtagggtatgagatcatcacttaaaatttttagtaataagttattttgagtttaagtgaataaagattattaaagtgtctttgtaattattgttttgtttaaagtatatgtacacataaaaacatgatccaacaaaatagttaccatctccttgtagtgacttcataaattatattttcttctatgtattagtattttctaaaaattggtataatgagggaaataaataaatttataataaaaatatttgtatgctttaataattctgtttccaggaatctcttttaaggatcaaagatgtggttaaatatcacaagataatttatcacaatgttattcataattttaaaattatatcagctaggcacggtggctcacacctgtaatcccagcactttggcaggccgaggtgggtggatcacttgaggtcaggagtttgagaccagcctggccaatgtggtgaaaacccgtctctactaaaaatacaaacatcagccaggtgtggtggcacgtgcctgtaatcgcagctactcgggaagctgaggcaggagaatcacttgaatccggtaggcagaggttgcagcgagccgagatcgtttcactgtactccagcctgggcaactggagaccccctctcaaaaaaaaaaaaaaaaaaaaaaaaaggtagaaacaattcagaaagattattgaacctttgaaacttcctcatgcttattctgttcttaaaattttctcttttcctcagagtgattagcaatcaaactcttttttaaaaggcttacatgtactggtctatgcgggcagagagtacagggaggctcagagagtgagcatgcaaacactaaaggcaggaccgcagaggagaagatacccaaaggataaaaaagatgctcactgagtcaagtagatgaaaaccagcattcttggtgtaagctctaagcaagaggggagaccatatacggccatagccagggagtggaaaaacccagaattttagaaggaaaggaaccttgtggtttcttcagctccaggaaatagcaaacacacccctatccccaccccactaccatttacacctaccctaggatcttctattcaattagaaaatctttcatttatcgagcaaatatttgttgagtgatgtgccaggcatagacaggagaggaaaggaggaagtctgtttgtttcaattagccaagttggtttattcatctcttccttctttaaacaaatggctccttccctccctccctccctccagacaagtgttctgtgtgaaaacttgatcttttcacatgaccctacatcctcctccacagtgattggttcccttcagtggtggacaacttacccaagctgagcctagctcccttgaccattgcaattgatcaaagggtggtcatgtggcccaaaacaagccaatcagacttcttccctagctttttttcaaaactttatgggaaaggagggctccttctctccagttagagagccatgagcccagatgtgagcctggaaactgtaaagagccagaaaagaagcagaggggaaaaagacaatgagcaaaacactcagagacgcaggagggagaaagagtcctgctggcattcagaaccctgtgtcaggtctttctgaatcacccatttcctctgtccttctggctgtctggttacctgagtcattaccttcgcattcttctgaagctatttcctattgtatttctgccgtgtgaaaccaaagggtgttccccagcacaagggtttggaattgcagcgctctgtatgagctctggtaagcctccttggcctagtgaagcagacaggtgccagatcagaagctttattaagataatgagggaggaggccgggcactgtggctcacacctgtaatcccaacactttgggaggtcgaggtgggtggatcatttgagtcaggagttcgagaccagcctggccaacatggagaaaccccgtctctactaaaaatacaaaaatcagctgggcatggaggcgtgcacctgtagtcccagctactagggaggctaaggcaccagaatcacttgaacccaggaggcagagattgcagtgagcccagattgtgccactgcactccagcctgggtgacagagcgagactctgtctcaaaaaaaaaaaaaaaaaaaagaaaaagagggagctaatgatgctctcattggagggaactcattgtattaccttttacctattaatataactgtgccttcacttaacattaatgcaaacaatccttgttgaatacctgccctgtatttccctcaccagtttttggccttttaaattgaattcatgcatacccgttcttctttcctgcttaatttgattcagcctactaggaaaggtaaaagggaggattccacttatgcccgtaactgtgaaaatgcaacatatctttagtggaaaatttacaaacaaccacaagatggggctgttatgcaaggaagtaaaagttaatggttggcgggaagaggatgcaacagtttccagcatccaggataggatatttaaatctttcctcactagaaaataaggaaaggaaaatattttaagaacatatattcataaaatcaaagcactacttataacaaattcagggattactctttttaacaatctagtgttcctttttgggaaagtgatacataattattatagtagatattttccgcctatatcaaggttgattgcttgtctttcttccgggttttcttcatcctcattcttctgtcgctttcgatgaaaagccataacataaaaccaaaatacatgcattaatttattaatttatttacagacttctatgtcctctgtattcaatttcaaagacaaagaaatagcatttgatatatttgattaacatactcgtttcctggtgtcttccacctcaatctaaattaaatcactttgacctcatttttccccccttgggtttgcagtttgaagatctggaaaattcttgattaatagcaataatgtaaggtagccacgtttttagaaaatccaaaagaagcaagaggaaagtaaccctgcatagccctttgccttacgagagactacagcatttgctggggcaagatgaccacagaaggcagcccgcaggagaatgcccattcaccctggagctgtgcaaagcaagaagtctgcaggtagatgcaggggaagccacaaagaggaagcattcctgttccttctagggcacagtccagtctgcgttgcgctaactcaaaactgggagccttctgttgagaaatgggaaacctaaaaatagacctgcagaaagtactttgaccttggcttgaaagtcagcccagtgctatgacaagaaaagccttttccatgaagattatgttcttaaaagccatatttcatctgcacacagactcccagtaaaaccacactcagaaaatgtgcgtcttggctatccttcaactgtaaaatcattatgtttggctctggttattctggacattagatgtctatgtaagggtttctttctgaaattccttgtacaaaatgttaggggttataaactggattaatagcgacatttaagcaaatctgctattactgtgtgtatccaataccttatgatttttggaaagaattcaaggacaaagcagaaaaactgttgtattcaagtaattcagaatgtacatgctttagaattaactacaataaactaataacttttcctgaaaaaatctagccagaaagccaagagaatatatttcattaaagctttagtaattaaatccaaaattaaagggtactgtcctataatttcttgcatgtgttagaattataatatttaaaatttgcaatatcaaagcatttcaggagaccatcatatgtagtctgcatgtatttcaaggataacccttaaagggtgataaaaagctattaaagaggggagaaaaagcacttttatatacttaggtaactttctaaggaacagctgccaccaattaaacaacaaataccttttaaagactcttgggggttcaatgttctttcagtggactgaaaaatctgggaaaatggacttcattagcaactacaaaattattgaaaggatatttgaaatggcatttaatacctgcaaagactgaaacaatcagtagtttaaaatttcgccataaagattggtaaataacactgattacaggtcttattcaaacagaaaataatgaatatgtaatgaaaagagaataatgatccaaattgcttttaatcttggagttatgcctattgattggcctggaaataatagtgctaatcttacagtaggaaagtcaagtgctttagaaagtcattagctttagggaaacgggatacccatttcattgtgccagtacagtattttattgagtaaagcctcaaacaactgtgtgtttaagaaaaattccaaggttagcctaagtcaaataacattcctctccactctgctctgtaactggtaaaagcaatttaggtactctttctaattcatctacctaaacacctgctatattcctgcaacacaaaataatcaaatacagtaaataattcagcattccaaaaggtaattattgatctcacactggcttatttctaataagctgaagaagtaataaccttctcaaaatagtaaatttacattttagactggcattcctgagacactctggcaaagtttcatgaacgaatatatttataagcaaatatacattaaaaacacacaaagtctttttaaaagtcataatttctcatctctaggaaaagcctcaaaggattctgaatttgaaatatctttacttcccattaactttccatgtatccaattacaagttaaatttacattcttttcaatccatttttgagaccttaaatgtattcttagatcctgagcttgaaaattgattgcttttattttaatttaaatgttaacacagaagcatctggttaatgaaaatagagatacatgaaaagccaggcacggtgactcactcctgtaatcccagcactttgggaggcctgtaatcccagcactttgggagtccaaggcaggcggatcacgaggtcaggagttcaagaccagctgaccaacatgttgaaaccctgtctctagccagatgtggtggtgcgtgcctgtaatcccagctactcaggaggctgaggcaggagaattgcttgaacctgggaagtggaggttgcagtgatcactccactgcactccagtctgagcgacagagcaagactccgtcttaaaaaaaaaaaaaaaagagagagagagaaatacgtgaaaaacccacgctcctcttagcaaatgtgcatggaacaaggaactggccggtgttaaaagttgccaggcagggcagcagagagcacttcacaatgacatggttagatgaggccctcaagacaagctggttgccagggaacactcagggcacctgagaagtgggcaagatcaatttgttatggggaagtcagctgaatgcagagctgaaaagagaaagtgcaagccagagggaacaagaaaagactgaagtcagagtggaaagagcttgggcttggaagtcacgaaaacctgacttctgacccttatcaaatgtgtcacctctgggaaaggaaacacctttgtctctccgacttggtgtttttcacttgtcaactgagtataatgatagcaccgcctcgcagaattattatagggttataagatgatgcatatcaagcacttagcacactgcctatgagtacacagaaagggctgagcaaaggatatgagtattactactgagtgctgctgccagagaggctggcaaagggaaggtgaatgccagtctcagagaaggatacaaatgggtacctcacagagtcaaagtcagggaggcaaaggaaacaaggcaaagggaactaggatggtgtggaatctcctcactaggctttgcattgaaccaagggccttctggtacacaaaatcttgcacactgaaatatgccccttggaatgttcacctttcctgaccctgtcttaaagataccttgctctaaatccagtcatttgtaacagctgctctggaaagcaaatctggtgtcatttcagatctgacaagtgcctttgagtaagagcaggaaggagaagctcattccattgaaaggcttcctcgagtatgcctaggaagacgcccactggagatggtttgttagcttgtttgccaaaacaagtgagtcttggaagttgcttttatttttttaattcaagtgaatggtccctttattttttaaaatgtgaggtaagtatctctcccttgtttctggagaagatggtgaagtttccatgcatctgagtgcagacactgcagtctggaagatgttgagtgttctgcttcatattgggaattatggactttttttttttttttttttttttgagacggagtctcgctctttcacccaggctggagtgcagcggtacgatctcggctcattgcaagagccgaggctccgcctcccttgttcacgccattctcctgcctcagcctccagagtagctgggactacaggcgcccaccaccacgcccggctaattttttatattcttagtaaagactaaataacacggggtttcaccgtgttagccaggatggtcttgatctcctgacctcgtgatctgcccgcctcgtcctcccaaagtgctgggattacaggcgtgagccaccgtgcccagccattatggacattcttaaccacattgtgctcttgaagaattaaataactttattagccataaaatggtgtttgtaggctgaactctcctctacttactgccctttaggactaacagcccagacttgggtaaaggggaatgcgctttggggtgttttgctgatcactgtagctgcgcagtgtaatgagaccattccctaggtgagtgtgaggaggggatgaacacgcctcaggatagagaaggagagaagcttatttgcttcaaggggactgatggttgggcacaggactgaggcttgcggatgcctagaagtcctgtgtgtggagtgtgagctgctcctggccatggagaggccttcgggggctgggccaggaccctgaagtttgtttagggtcagctgtaagccggaagagcccccactggtccctgcagtgtcaagcactcaacttgattccattggcacttagtaaagtttttattaaatatcatgttacttttcatttttggtaaatggcagagccatataaattcaccaaaatgttcaaactgaaaacttccccgttccttcccattcttccactgaaaccatcaacaagtactatcaattctgcctccaaagagccttaagctcaagctcgtgctttgtttgcctctcctgtttcccaccctgattctgattctgttgttttccacctgggtgacaacaggatcctcttcctctcctggctttctcttttcatcttccatcccattctttaccagcaacctgagaatcccctttacaatataaatgaagtcaagtcgttctcctgcttaaacatttcagtttcccagtattctcaggataaagatccaaatgcttagcatggcttgcaaggtcaagtctgggcttggtgagctttggagaactcagtgagagccctggcctctggaaaaagctgcctggaaaacacaattatagtcagtgcaaagtgatcactcaaagtgtgggctgcagggtaggtgacaagagaccataacagatttcttgagattccttctagaagggaacccagtagaagaacaggctcctcattcctgttggtgagaggttgtgacagggcagtaaaggaaacatgactagaggtgtaggcactggttgataagatttagggatatatcagtgacacacccaacctagggtactgtaactgtaaatcaaaacaatcttcttttagtcttaaaaacaagaaccaaaaattgtacaatttaacatgtaataatggcttctatttatcaagcatctgcaaaggggctggctttatgccaggcattttacatatttgattacaaaaaccttataagataattattatctccatttttgatatatgggaaccaaggctcaaaaggttactaaaagtcaaatgaataataaggcacaaactttaaaaaaagttaccaaattgcatttctttatgaaggaaagggacagatctaatgttaatttgttattaactgaggaagcacagaaagttgggcgtttacttgtttcttatccatctcccccgactaggaggagagagcttcatgaggggagagcctgttttgttcacagctgtctcccgagcacactgaatagtgactggtcgcagcagacctgtaggggtttgttcacagactgaagcaatgaagtttgacagctgctttcattactcagtgattttgattcagagctttacaaaagtaggaaaaattagtgtgaatatccatttttccatagattaggtgttggatttttttttttcttattctctccactacgtactgctattaaaatacaactaagccttattcaccgggaggtttccattagcaacaagaattaattaaattatgagtcatcagcagatgacatactaaggcagatcatcccccgacgatgattgttttttatttttctaattataaaagtaatatagtaaagaacactagcaagatttactaattttaaaaagtatgtacagttatgaaaatatttctaagatatatttttagtgaaaagaataagatgtagaatagtgttcattgtaagcttttctcccatgggataataataatagttattgagagcttactgggtgccaggcattatattatatccactgcatgaattatctcatttaatccttcaataacaccatgttgttggcactataacattattagtactatactccatttgaaatagtactattgctctcatattatttttcttgtttttattttgaaacaatccagcacttagagaaatgttgcaacatgcaaaatacacatacacacacacaagcatatacatgtgtacattttacatctgtatttacttctatatacccatatattaaaagccatgaggttgtactaatatctacaattccattcctaggccacagagttgtagttttctctcttttttttttttttttattttttgagaaacagtctcactctgttgcccaggctggagtgcagtggtgtgatctcagctcattacaacctctgcctcccaagttcaagtgattctcctgcctcagcctcccacatagctgggattacaagcacacaccaccacacctggctaatttttgtatttttagtaaagatggggtttcaccattttggccaggctggtctcgaactcctgacctcaggtgatccacccaccttggcctcctaaagtgtttggattacaggcatgagccaccctgcccagctggctctctttctatatgtatatttattctcccaaataatgtgaaggctggctccaattgtcttcaatttatttattcatcgaatcactctcctgttgcctccactgtccctatccctggcaggaatgttcctcctcccactggtctccagcactctgcatcggaccctctcaccccctacaacctccaagaccttgtgccccatcatgctctgacacctccaccaacacaaaagtccttctttctctgtgtcaggacctccctgagccccctaagttgtgacaccccacctacccatccctgacaaagacagctcccttgcccatcagggatagccatgaggaggtggctccatgggttctagtattttgagactgcatcaaacttaatatgttagcatctttggtagcccattcttaacctggaccagcagttagacctaaagcatagatggccatttagctagtcatttgcatgagaattaaacatgagagatgaaagaaaagaaaaaggaaaaaaaaaaattgtaccaggaaggaagatatttcatgagctgaatcccaagcagcaaagtcaccactaatgtcacaacggtgttgccagctaataatggggctgcttcttaactgtcaggaaatgcaaacagcacaacattaaaatggttctatttttttccatccttacaatagcaacacagcaatggagtgctgatctctgaaagacaaaggctgtgaaaataaacatgcacttaagccagggggcacttgggttgtgatttttctttcaagatatttggtaacatatcccaaatcctcaaaccaccccagggatcttcccttcaatacggtctgaatcacctacctgtctcaaatggcccagcaaagctcttgctctgtgcaggcaggcctgccagatagctgcctttttcttttcctcttaccttggctaagtggcttccaaggtgtttcggaaaggaaaatgtaggattcaaaataaagagcagagagttgggccaggcctgcattgtcctctaactgtgataatgggtagctgctcggcccctggttcatcttccacagcaaaaactggaccaaggcctgctttttctctctaatggtgggattgcctctggcttcgagttgcacgtttatctttggtactcatcactttgattttgttattacttcaccctcccctttgtcttcacttttatctgtctgtatatttcctaacaacctgtttttacctcatctccctcttttattattaccatttttattcccctgccctgcatccttggcttcccctgtaagcactttgaaaaatgaggtgaattttatattggtaaaagatgctgaaaggtgaatctgtgaagcccccgcccccgtagcctccttccatatgagtaccttctttccatgtaaggtaacagctctctctcttctttttttttttttttttttttttttggtgctcattctccccaggcaaccagaaaagctgtccgcttcaaggaatggcttgatctaagatccaatcatgggatacaactaaagaacaaaacctcctgttaccaggttgcaaatgtccaggtatttgcaagcagtgttttgctttcttaaataccagactatggctagcaatcttccccaacttcaggttatagaaattcagagctccctatctcaagaccaggaaaatccagagcagacacatgttttgtgaatatgaccaggaataagttatttcatcttgggaaaaaaataagaccaagatctagggcgtgcttgatgtttcccagttgttcttccagtccaccctattcccttcatcactcttctctgtgccccaagtaggaggctgacctattgggctgtataaacaggctcttttgcactatagcttcttattggattcaccccatggggagcattggagatcagaaagagggtagaatagtgagagcagaaggccagggtatttattcaccagctccctctccttgggccactacaggctggctgaatatccctgctcctgtcaggtgaccctctctctccagcaccaacatcattctgtcctttgtcccatcaggccttctactagcccagggtactgcactactccttgtgatttccctataacctgctaatacctttataaataatctccttaataaactctactcaaattacccaatttgagtggaccatttgtttcctgctgctggtttgatgcttacaaaagatctctgcttaggcaaggcagaaacaggtgcatttgagaaatggcaatggcaaatgatcgggaacccagttacctggaaactgaggcccatggtaggaattcaagcaactcttggcacagagaaacaaaggagaaacttctttactgaggattagcaatacagagaagtgaatgcagttactgacaactggaggtactatgctcagatttaataataacacatttaccatgcacttatgataagccaagtacctaataaatacccggtcatattttagaattttttatccactttactgatgaggaaactgggtgaattgatcattgctgttttgtagacccaaattccctccttcagcatcttctagtaataaaactactctctcctgcaaggagctcatttcacaggttttaactagtgctaacccaggccttgcctgtcagaacacacctcctcccatgcacaatgcttggctcagtaatggacatgtgactctaacaggccaataaatttctccgagatttgatacttggacctagggaaagactagaaggatgatgatagcagccaccattgcttcaggccacctttccctctgtttggaggaagccatctccagcaagaaatcatttagcatcagcaaagagaacaggagagagagaaaagggatagatggaggaagagcaacagtgagcacaaaagagatgacctcatttgctactgcagccagtctcttgagtgagttttatccttcagttttccagtattatgagacaatgaatactttatttttacttaatataagttggatctctcatttatatttccaaaaaaccctgtaagtagattaaggctgccttgcagagattcttcagctgggcagtgatcagcccatgcgtgtgagaaaactaccaaaagcaggaaaaagagccactcagaaggagcaggtagagcaatctccagagcttacagagatccagaggcaggagaatcacttgaacccaggagaatcacttgaacccaggagacagaggttgcagtgagccgagatcgcgccattgcactctagcctgggggacaagaccaagactctgtctcaaaaaaaaaaaaatttgaaaggcacaaaatgaagagaaaaacatattatcttttctaccattcttacattaatacttattttgaaaagttttaataacttttgacatttctccctctctctctccttccctccttccctttttatcctaatccctctagtgcaatctccatctcttttgatgtttttagtgtacttctcttattctctctcacactcactgattctttttgggtaagtgtgtgtgtgatttgaatagcatcccaaaataaaaatagatgtaaaatttttcttaacttttattccatttactttttagacttttttgttaaaaactaagacaaaaacatgcaggcctacatagagtcaggattatcaagatgtcagtaggcaatagaaatttgtcagctttattaaaattttatggaactactgtcatatatgtaactcatcattggccaaaacgttattatgtggtgaatgactatagcagtcatggttattaaaacaggtttataactatagtctatatgtttaagaaagtagaggaaaccataagtgtgttaagaaaagacatagatgatgacataaaagagacccaaataggctgggtgaggtgtctcacacttgtaatcccagcactttgggaagccaaggcgagtggatcacttgaggtcaggagttcaggaccagcctggtcaacatagtgaaaccctgtctctactaaaatacaaaaattagctgggtgtggtggcaggcacctgtaatcccagctacttgggaggctgaggcagaagaatcgcttgagcccaggatgtggaggttgcggtgagctaagatcacaccattgcactccagcctgggcaacagagtgagactccatctcaacaacaataacaacaacaacaacaacaacaaaaagacccaaatcaaacttctagagttgaaaaatacaatatgtgagatgaaaaataaactagatgggattgaaagtagattagacactacaaatgaggacatgtcaatagaaactactcaaaatgaaacacacagataaaaaatgactgaaaaagatcaacagaatttcagtgagcagtgggacaacctcgtgcagcctactctgaatgtaaatgtggtctccaagatgagagagaagagaggacaggaacaaagtttgaagaaatatggctgaaatttccccagatttgttgaagaacataaacatatagatccaagaaactcaataaactttatttatttttattttattttattttatttttgcgacagagtcttgctctgttacccaggcagtgcagcggtgcaatctcggctcactgcaacctccacctcctgggttcaagtgattctcctgcctaggcttccctgagtagctgggattacaggcatgcgccaccacacccagctaatttttgtatttttagtagagatgggatttcaccatgttgtccaagctgatatcgaactcctaatctcaagtgatccagccacctgggcctctcaaaatgctgggattacaggcatgagccgctgtgcctggcctcaataaatgttaatgaaaagaaagatgaagacacacatcaaggcacatcataatcaaatcagggagcccatctaagtgggcaacatcccccacagtcatttctgaccttcgaagtagagtaatcacagagcatttcaaggattcagacgtgccctgcactaaagtatttctgaatatcttagtgaagggaaagtcttcgaggtttctggtggagcattaggtgggaggatgggcaccaggttgcacatgataaatccagttcaaccttatgtcctaagcctgtggattcccttctccatagcatgccaaggaagctctggcatggcatccccattaaatgtagactaccattgagtccaagttttagtcaatccaccaagaaagatgttaaaaccacctccagttgcatgagctaatacattaaatccagaatctctactaagtacacccatatccatgatttaacccaatctaatattgtatcttcttatgaaaaagacccttagaacatactctcacacatgttccccaggtttctgttgctatatatttattagcaaagtcttgaaatgtttggggcatgcaagttaatttttcccaggtcattgtgtgtacttatccacctacaacatactgagatttggacctgtccaaacgtttagtggtaagagggggtgattgtagtgcatcttaaggaaaatgaacatcccctttcaaagcatcttccccagatgaaggttttcctagcaatgatctaaatgcctcatcacacacccaatctctcagaaatggctgggctatttcaaagctggaatagttcactgaagactctgttatggtgccaactgggagacaacggcttaaaaggatgatattctgtcttaacaggatgtggtacatgctttgaattagagaccatggtggcatcttccccataatcaaaatatatgtgtctgggaattaaggcatggaagtgaaggagcaatctgtcctcaaaagtaaattcaaaattttacctctataaacagaagaatttgctaacaatttaaaaatagctatttccagtttctagtcccactttctccttattactaccaggcaacaagtttccaaaaaaatattaacatccaattttagctacctaaaaatgtgaagacctttacagatgttcaatggtatattataaaatggtaaacttataataaagaaatgagcaaaggttcccaaaatgagcagtagaatgactggcagtaaaataaaaataaaaaaaaataggaggtccaaacacaaaatattatgtagtgtcctaactatattttactgattttttgataagctccctagttcatcctcttatttgatctaataatcagacaagcagttccctgatgttacaggcaaatgaattaccctcagagtttccaaagaaacgtttgagaaaacaatagggaagaactgcagcaatctgctactgcagtgatgtgaagacacactgttgaggttaaaagtcaaaggtcaagttggtgttattctcttggtccttctgcagttgattaaacaatattacttgagatgaaagcaatcagtgaaaagaattatgattactttattagcattactgaataaatgtcttttcagaatccaagttatcactcaaaaacatgtcatgttgccctaaaattacttattcaatttctggccagagaggatttctttataagaaagactttcctattaaacttctcattgaagtttaatcaatatgcttcatcgatatgtgtcccctaaagcaatcctaggatttagaaaaggatctaagtatgtgggcttctatgagtaatatctgagtctgagcttactcaaaaggttggttttcaggctgtttcttctctaattctagaaagctctagaaggccatagtaaatcctcacagaatacaactaaagcataaaaataactttcaaagatttataattagccggaataattttctttgggatctaattcttatttacaagtcattttagagaccccaaattaggcaacaacattgcaacatgactaacactgacttcagattggaaaaaagccatacatttattcaatagggagtaaacaagttctagggaaatatgtgagagaataaattaataatatcatttagcaagaaaaaagagctcaataaatattatagcctagcttaaaaaagaaaaaaaagtatgtttaggcaatagttgtatctcctcactaaaggtatggtaagctaagatatagccaaagctatagtaaaccaagaaaagctcttctctccagtggagagaggcaatgttgcctctacaatcatgtggtcacagtgtgtatggtagagtggcaaggcatcttctgttccccttagtccaaagtatgcaccttggttgataagcagctagaaggagaaaaccaaatagatttagatctgaacttttaaaaacaggtttatgcaaacaggcattcccacattgagctgggactatatactggtaaaaccaatcttgaagacaatttagcaatatatatcaagggtcttatgagtgtatatgtcctttgaataggatatttcactttagcttaagaaaataatcagatttttttacaacaatggacttcatagaaaaaaaaacacaggaaatcatttaaatgaccaaaaaaactattatagctttttttcaaaaccattaatgctcagtataagtaggaaaaaatccagattacaaaaaattatgcccaccattttctattttgtaagtctgtgtatgtgtgtgtatgtgcctgtgaaaaaaatgcagaaagacttagaataatttttctggggtctttctttttgtattttcaatttttttctatttttttttaaaaattatgtattaaagggatcgttagagtggtcaactaccttaaagagtattatgaattcatggtttaaaaaggtgattgaaaataaggcaccagtgtagggctgttcacatagcatgtttttctaaaataacctcttccatcgtctgtagtagctacctgaatttcagtgggcctagctctaaaaaagttgagaaattgtaccaatatgtgttgcatcatcccttaattatttgtgccagttgatggaagctgaaatgtggaaaacatcaaccacataattcaaaattaatttgtactgagacttggaatatgaccagacacatgtgcttagacatgaatgtatttctcctcttccctcactaaccttcaggccttgtcctcaccacttcttggtctgggtccaactcacagcctggggccagcatggcagcactggtgaagtccctaggaataaaaaaaatgtttgagaaaagagtggtgacaatgactgggctctgaattgtgaacatttacacgtgtaaatgcagaaacacccatatacgatcatccacatattaattttccacctagactcattacttttccataactctgatgaaaaggggagttttatttgtgtagaatctttccgatttggtgatagtggtgactggaataatagtaagaaaagatatcttggcaggactggctacatcatttaagggacccaatgcaaaatgaaaatgcaggctccttgttctaaaatgattaagaattttaatactgagacagcagagcatgaaaccaagctcagagcccttctaaattcaggtctctgtgtgactgtactggtcacgggcccatgaagccagtcctgagttttagagtcaggtagatatgggatcaaattctcgattttttcatttataagctgagtatcttggctaaattatttaaactttcaaagtttcagttatgcaatcttaaaatgggagtgatgtgagtgtggcattattctcaagattgctgtgaggattaaaagagagagcattctattaggtgcataccatagtgcctggctcagggagactgttcatcttctctatgttgtatactgcagttagagtttgatatagtttgaatatttgtcccctccaaatctcatactgaaatttgatccccaatgttggaggtggggcctagagagaggtgttggggtcatgaggatgaatccctcacaaatggcttggtgccattcttgcgggattgagtgagttctcactcttagttcctgttagatctgttaaagagtctgacacctccctccccacccctcttcctctctctctcattctctctctctgttctttccctttaccatgtgatgcctgctctcctctgccttccaccatgagtggaagctccatgaagccctcaccagaagcagacgctggtgtcatggtcatacagcctgcagagccatgagccagataaacctcttacttttacaaattacccagactcaggtattcttttatagcaacatgaaacagtctaagaaagagttcatattctaaagtgcaaacttgaaagtttgatatggtttggctctgtgtccccatccaaatctcatgttgaattgtgaccctatgtgttggaggtagggcctggtgggaggtgattggatcataggggtggtttctgatggtttggcaccatccccctagtactgtctcatgatagagttctcaaaagatctggttgtttgaaagtgtgtagcacttccccctttgctctctctccatctcctgctccgccatgatgaatatatgcttggttctgcttcatcttccgccataattttaagtttcctgaggccccgcagccatgctgcctatacagcctgcagaactatgagtatattaaatctcttttcttcataaattagccagtctcaggtagttctttataacagtgtaagaacagacttaatagagggtttctccctgatttaagattatttttggttatccattatctacgaaacaaaatgaaaatgcacattatgacatataaggaccttaactattcaggttttatcaatctctctaaaacttgccacctaccactctcacatatgttcaaagtataatttgtatttattttcacttttccttaagaaccagtaaatataataggcatagatgacaaataataaattagcatgtgccaggcactgtgctaatcattctgtgggagggaatactcattctccactcctgctgtttcctcccatgagagagagctgcatgagctgtaacattcctggatacttccacctcctcatttatcctcccctggcaaggataagttgagagaatctgggtatttgtattccagcctctttctcttttggtagcaactctcacagggagcatgattttaggcaagctgtaagcatagtggcaaagaccttttcccggtggaacgacccattgtccaggactacctgataccagttggtgagcctgtctatttctggtattcagcgtcaggaagcctaagttctccagtgtcagctttatcaatgataatggtctcctgcctgcgttactcttgtctgtttctaaattggttcagaacttgggtggggaaaatgggtattatttactgaggatacgtaagtatcccaacaaagtctacattcattgtctcctttatacctcagagctaccatatgaggtaagcatatctatcatccaatttttatagataaggaaagtaaggattagcaatcaagtaagtggcataatcaagatctaaacagttctatctgatgccacagattatgcccttacattttcccattttgtttttgcatgaaggtttaataataaactgataagaacattcaaaatttgagttcatgttgaaaaaaaaaaagctaacacagtattatatgcagtgtacttatattctatctggcagaaaagttagccaagctaaacttaaattcagcacatgctgaattttattttagggagaacaagctgcaatcataataaagaactgtatttacagctaacagcaatacccacgtttctctataggtcttacactacaatctgaaattgcatttcatgatttacaaggtctgagacataaacagtaaaaggtatgtttctgggaattttctcattaacagccacaaaacaagcaattttatgtggaattgctttgttcaccttttctgaaatatgtgcaagatctctctttgatgttgaatccattctgatttgaacatctcaatatttttgatgttccatgaactcctccacaccccacctatctccgggaagctcagacctccctacctacttggtgaaaagaaggaaatttagaacatttgagctcgcaggctgcctacttaagactacccgaagaatgagctctgcttacccctgttccttcgtaggtacatgaatgagtcgtcaaaaattcactaatagttgattaatgcagttgtgacagtatgaaaaacaacaaaaggagtgcagtgctgaagaaattctagatcttgcagatttaatgcatccacttatatccataactcaatcccttttcatatgacactagttattgttgttactgtcttccataaccattcctaccacgactccagtccaggccatacccccttcttcttgtcacaagaacatcaacactctgcagtcttttcttctttatcatgtcttcatccctccatactttgctcaccaagatgtgtttttccatctgtcatatttgtctaatcctgttgatttaatgcactatatttctactaagaaaacagtacatacaaaatactcccaagcatctaactacttttaaaaatatagatctttctaaattacccatatgtattgcctgacctggcatctctctctattttcatacagtgctcagcactcctgattaattgtctctttaatattttatccaaaattgcactgggaaaccaagaaaacaggacaaaggaatgtcatttgtactttttgtaatcttcattatttaccaaatgtatttttcttataaccacctttgtagtacttcaagatatgcctaacaggcactgttagctcttttacaggcctgtggataatggaaagtatggagaatcctggcttggaaaggcaataaataaggttattttgacactactctctattggaggaattgtgattgtttttgattttgtttctgttttgagagaagaagcttgcctaatggcaatccataagacattctggagaacagttatagattcaaaatgtgagttttagtctacttgaatgtagcaccttgttaacaaaaaggcttcacttttattgcctcactgataattcacataacctgctgacatagtgctatggtctgaatgtgtgtatacccccaaaatttgtatactgaaacttagttctcaaggtgatggtgttaagaggtggggcctttgggaggtgattaaatcatgagggctctgccctcaagaatgatgttaggtaccctcttaaaaggccttgagtaagtgaagtttggctctcttgttcttccacctcccaccccgtgatattgcagatttcacaacaacaaggcaccatcttgggagcagagagcagctctccaaacattaatgctagcaccttgatcttgggcttctcagcttccataattgtaagaaaccaatttcttttctttataagttacccagcctaaggcattttgttatggcagcacaaacagactaagacatatagagttcatggtggccactttttaaaaaaatgtacaaagaacttgaggcactgagaggcaaatgactggctcaagaacatgcaaagcatgatcaagtaattttgaggtatacatttttaattcaaactcccttggtaatctgatggtgaaaagaattttcaattttcaaatttcctagactacatgcaggagagaactcaggagcatggcaccaacttattttgtcttatgtagaaggatatctgacttgaggttgagaattccacatcagaatctataggccttttcccctgcccagtagagtttagttgcctaccttcaatctctgccaacagcaatcctctacccctctggtttaagcaggctgttctaaatagttgccggtggttaccgtggtaacagacgcttaggtttcagcttcgaatgccccttttaatgaggcaatttcaagaatatctgtgataaattaccagaccacagacttcactgcagggagttttgctttggaaaatctaataaaaagacagcagaaagaatgagcagtaaatgcaacagagaaaatggggccagatttggaggggcaaatggaagagatgccagatctgactaccaaaagccaaatcagaatcagttcaaaccagagctttaaattataaacggtccctgaagggcttcctgacatgctgagccaagattccttgggaggaggaatggtaaagtgagaggtggtgagggcaagagttgagtatgtcctgcagagggctgtgtattttaaagcttccattttctattttatcattaattgtagtggctaaatcagaaacggtctccttttgtttctccatttctccatgtatgagaccaagagacattgccacctggagaggacataatgaactaaaatgtggactttcgcctaagtgaatttcacagcaacctaggttatccacatttcccaaagacatacctgacttgataatggtccaggattctgttattaatctcttatccattgtacccattaaaaatttcacagaagggtaaacgtacttaaacacattgctacactattgtgtggaaagaaacatctcctaattacccaaaggaagacatgtcattcatccttgttatgattgaatataaaaactatgcctccactatataaaatagcttcagtttatacttggttgaaggcaagagggtagcctaaaagagccttcacttacactgtaagtgccttgccaggctccctaggtcccttcaggaacatcttactctcgctgctcacggaaagcaccagggaacagatgcattacagtttgtgacaggagaacagcctgtggattccgcctactccctccagtccatctgggaggtgggtgagacagggagcccacagagaggggggctctcctgggaatccctcagggtaccatggctattggtgctttattttcagtagatggcactgtgtctattgagataagcaaagggagattctagatgggaaatttaattgtttctagagaaagcacaaggcatgtgagaatgcacggtattttatttttgccttagttacatgataacgcaaatactattcagtattcaggttacattctctttggagcccatggaattttatgatttattattctggctccatcttaggcctgtggtgaagcccagggccatcctatacagtatatacttggaataagaataaatcatgctgaatgttcacctctgttacccaacacatacagcagcacctatactccatctgtgccctggatccccacctgtgcactggctctctccctttttcccagacaggaaactctgcctggaacccaggcagttcctgcctggaacccaggagtttttacaccccaagaatgagacaaccagaacagtttcttgcacactggtggtcccatggagcagacattctcctttgtccgtccatcctttttcttttctaatagacatccagttatttttttcctctcttctgagtatttccaggagagtaaactctagcaccagaagatgaatcaatttttttttaattatgctttaagtctgggatacatgtgcagaacgtgcaggtttgttacataggtatacacgtgccatggtggtttgctgcactcatcaacccatcatctacattaggtatttctcctaatgctatccctcccctagccccccacccccaacaggccctggtatgtgatgttcccctccctgtgtccctgtgttctcatgaatcaattttattttaagccatatgtagtcttttaaatgggaggagagaacctcaagtatgtttcatagaaaatgaactgtagacacaggatggaagatggaggacatatgggccaaacactaggtgatgcacttaatataaaactcttttattccttatgataatcttgagatagatattttgctacctgtcttacagatgagcaaatggaagttcagtaagtttaaataatctacctaaaatcatcctcaatattgactgggcatataatgagattctaaacttttgagtagtttgctagaatacttctggaaagggacttgcatggagaaacttacttctgtccattgacattatcttctgaatatgacccctggaactgcagcagtcatcttgagacaattatggaaggacacctaagagaacaagataaaagtccaaagacaacagagcagaatgatagaaagaacctggaaccttgatgataccttttgagctgctgatttaatcaaccttcaaacttcagatgtatgttttagccactttgagatatgtcttctattactcatagcctgaaataccctaaacaatactacctaaatactctagccatagatgactagaaaatgtattagtgcctgagccataggcagccatccataggatgactgctgaggaggtaggatatgaaagttcactcatccaggcatccaaattggataatatctaatgaaaccaatcagatcctttctaggggcatctgaataagacagcattttccctgtctcctcaggaaccttgctccatcaattaacccatctctcctgtgtcttcaatgttttttccttaaactaacactttttttttcttttcttgagacagagtctcactttgtcacccaggctggagtgcaatggtgcagtcatggctcactgcagcctcgacctcctcttgggctcacatgatcctcccaccttaatctcctaaatagctggaactacaggcatgtaccaccatgcctggctaagtttttgacttttttttgtacagatgagttctcactgtgtttcctaggctggtcttgaactcctggcctcaagctttcctcctgcctcagcctctcaaagtgctgggattataagcatgaggcaccacctaactaaccccttcttaaataccgtttaagtttttcctattaaaaacaaacaaaacatcctttgtcaaatacagaacttcttccaataccactcaaaatagctcctctctttcacagtccacttcttaaaatagatgtctccagtttctatccttcaagtcactcttcaacccattgcaatttacacctgcagttccccaacaggacagctgctcttatggactttacaaaatagtaagaaaataggttgtcaattgctgagtaacacactacctcaaaacttagtcatttaaaacaacagacaccactgattatctgccatgattctgtgggttgacaaggctcagttagacaattcctctgttctgcacagaactgcagtcacctggaggcttgactgagctggaacatccactatggctcattgacatggctggcagatggtgtttgcttttggttgggaactcagctagaagttagtccttctcctcctggcctctctacatgtgggcttctctcaacatgatggctgaatttcaagaggaacaatttcaaccttgtgaaaatagaagctgctcagcctcagaaattaaagaacctcacttctgccacattggtcaatacaagtccaatccagcccagattcaacagaaagcaaatagacttcacttcttgatagagagaacagcaaataatttacagctatctttaatgtaccacagttattaatgaatttctttattgttaaatccaatgaaaacattgtagtccttattttctgtggcaccttgattctggaaatactttcttccttcaactttcatgacaacacactctcctgcttgtccaggcacttctagggcttctccttctcttttttgggaagaatcttttcctcaaagtcaaatattactgttccttggaattccatccttcttcttcttctattatcactatattcactcttactgggcattctaataaacttactaggcttccattactatctacatactaatcactcaaatctatactatactcacagttcatattgctcactttagtttcaaatctagatatcgcactctttattgatcatctctagtctccatttgaatatccaacaagtatcacaaattcaaaaggtctaaaactgagcttgttgcattttctcccaaagctcctatttcaatgttatctgtttctatgaatagcaccatatcccttcagttacatgatgcagaaagctaaacatttttcctcttctattttcttcaaccctacattcagtcaccatagctagttaatattatatctttaacatttttcaaatttgtcgtcagcctctgttctctaacacttcagtattgaactccagtttagactactatcatcttttatctgtttactccagcatcttgtatcagtcttcttacctccacactggcctcctctttaatccatttttcatgctgtcagagtgaagtttttaatggtaaaatcggattacattgtgctcaattcagcaacacatataccaaaattagaacaattcagagaagactagtatggcccttgtgcaaggatgacttaagatttgcaaagcatttcatattttttctgtatccaagggcacacaatgtactggagatgatcagtaaagagtaggatatctaaatttggaattatgtattataatttgcatgacaataacaacaaaaaatgaggggaaaggggatgggctatatgggaacaaagttgtgtatattattgtaattaagttggcattaacttgaaatagattgttataaattatgatgttgtaattctaacagcaacctgtgatgattaattttatatgtcaccttgactggcctaaggaatgggaatgcccagatagctggtaaagaattatttatgggtgtgtctgtgaaggtgtttcttgacaaggttagtatttgaatcaacagactgagcaaagaagacccaccctcatccatgtgggtggacataatctaatctgttgaaggcatgaatcaaataaaaaggcagaggaagggttaattttctctctcttcttgaattgggacattcggcttctcctgccctcagatattgaaactactggttcttgggcctttggacttcaggacttccaatgcctccacaactcaagttctcaggaaatagagttccaccagtgtacaccctacaccaggattaaagttttcaggactgggacttatgtcatcaggtagtcttgttctcaggcctttgaacttaaattataccactgactttcctggtttcccaggaaatatgcagacagtatatcattggacttcttggcctccatagttccatgagtcaatccctataataaatctcctcctacatatctctatatagcccattgtttctgtttctctggagaaccctggctaacatacacccactaagaaaacaattcaaaaatagcacaacagaacactaggtagagttctaaggttttttactctagttcttggtgcctggatggccccccgacccacacaagacctgggggaatttgctgccctgaagagaaggacataggcctggctggttttgccagctaatgattgtagagccctagagcattgagtgaacataagtggtagccagggagtggttacagcaggccttgggccagacccagtgctgttctcgctttaggtctgacccagcacagtcctaagggtggtagccacaggggtgcttgtgtcactccatccccagctccaggtggctcagaacagagaaagagagactctctttgtttgggagaacgtaaaggaagagaagatgagtctctgcctagtaatacagagaattcttccagatcttgtccaagaacatcaaggtagtacctctatgagtctgcaagacccagagcattgctgcctcctaaagcagatacagcttagatcacaaaaccctagtcctttcaaatagctggaaagcctccccaagaaagatgggtatgaacaagcccagactgtgaagactacagtaaatacctaactcttcaatacccagactcagaagatctacaagtattaagatgatccaggaaaacatgatctcaccaaaagaagcgaataaggcacgtgggaccaatcctggagaaacagagatatgtgactgttcagacagaattccaaatagctataatgaagaaactcagagaaattcaagataacacagagaaggaattcagaattctatcagataaatctaacaaagagattgaaataattaaaaagaataaatagaaataatggaatgaaaaatgcaattgacatactgaagaatgcatcagagtcttttaatacagaattgatcaagcagaataaagaattagtgaggctgaaggtaggctatttgaaaatacatagtcagaagagacaaaagaaaaaagaataaaaaacaatgaagcatacctacggaatctagaaaatagcttcaaaagggaaaagctgagagttattggtctgaaagaggaggtaaagaaagagatagggatagaaaatttagtcaaagggataataacagaactttccaaacctagaaagatatcaatatccaagtataagaaagttatagaacaccaagcagatttaatccaaagaagactacctcaaggcatttaataatcaaactcccaaaggtcaaagatgaagaaaggatcctaaaagcagcaagagaaaagaaacaaataacgtacaatgaagctctagtacatctgacaggagacttttcagtgtaaaaccttacaggctagaagagagtggcatgacatatttaaagtgctgaagagaacaaattcttaccctagaatagtatatcccacaaaattgcccttcaaacatgaaggagaaataaagactttcccagacaaagaaaagccaagtgatttcatcaacaccagacctgtcctacaagaaatgctaaagggagtacttcattcagaaagaaaaggatgttaatgagcaagaagaaatcatctaaagtacaaaactcactggtgatagtcagtacacagaaaaacacagattattataacactgtaactgtggtgtgtaaactacttttatcttaagtagaaagataaacagtgaaccaatcaaaaataataactacaacgttttaagacatagtacagtaagatataaatagcaacaataaaaagttaaaaagcagggggacgaagttaagacatagtgtattagttttcttttggcttgtttgtttatgcaaacagtgtcaagttgttatcagtttaaaataatgggttataagatagtatttgcaagcctcatggtaacctcaaattgaaaaacatacagtaagtacacaaaaaataaaaagcaagaaatgaaatcatatcaccagagaaaataatcttcactaaaaggaagacaagaaagaaaggaggaagagaagaccacaaaacaacctgaaaacagataacaaaatggcaggaataagtccttacttatcaataataacactgaatgtaaatgaattaaactctccaattaaaagaaatagagtaaatgaatggattaaaaataaataaataaaactaggcccattaatctgttgcctacaagaaatgcacttcacctacaaacacaaacattgaatgcaaataaagggctggaaaaaatatatttcacgccaatggaaaccaaataagagcaaaagtagctatacttatatcagacaaaatagatttcaagacaaaaactataagagacaaagaaggtcaagatataatgataaaggcatgaaatcagcaagagaatataataatttgaaacatatgtgcacccaacactggagcacacagaaatataaagcaaatattatcagagctagagagagagagagaatccaatacaatagtaactggagatttcaccaccccactttcagcactgaacagatcttccagagagaaaatcaacaaagaaacattgggccatggcccaaagaaacattgtactatataccaaatggatctagtagacatttacagaacatttcatccaatggctgcagaatacacattcttttcctcagtacatggattattctcagagataaactatgtgttaggtcactatatgttaggtcacaaaacaagtcttaaaatattcaaaataattgaaataatatcaagcatcctctctgaccacaacagaatcatactagaaatcaataaaaagggcggggcgcggtggctcacgcctgtaatcccagcactttgggaggccaacgcaggtggatcacgaggtcaggagatcaagaccatcctggctaacacagtgaaaccccgtctctactaaaaataaaaaaaattagccaggcgtcgtggcgggcgcctgtagtcccagctactctggaggctgaggcagtagaatggcaagaacccgggaggcggagcttgcagtgagcggagatcacgccactgcactccagcctgggcaacagagcaagactccatctcaaaagtaataataataataataataagaagaagaattttggaaactatacaaatacttgtttagtatttgtatgtgcattagtccattttcatgctgctgataaagacagacccaagactggtcgatttacaaaagaaagaaatttaatgggccaggcacagtggctcatgcctgtaatcccagcactttgggaggccaaggtgggcggttcacaagttcaggagatcaagaccatcctggtcaacatggtgaaactctgtctctactaaaaatacaaaaattagttgggtgtggcagcgcgtgcctgtaatcccagctactcgggaggctgagaaaggagaatctcttgaacccaggaggcggaagttgcagtgagctgagatcatgccactgcactccagcctggtgacagagctagactccatctcaaaaaaaaaaacaaagaaaagggtttaatggacttacagttccacatggctagggaggccttgcaatcatagtggagggcaaggaggagcaagtcatgtcttacatggatggcagcaggcaaaaagagagaagttgtgtacagaaactcccccttataaaaccatcggatctcatgagacttattcactatcatgagaacagcatgggaaataactgccccatgattcaattacctcccatggggtccctctcacaacacatgggaattcaaggtaagatttgggtggcagcacagccaaaccatatcattccaccccagcccctcccaaatctcatgtcctcacatttcaaaaccaatcatgccttccctatagtcccccaaagtcttaactcatttcagcattaactcaaaagtccatagtctaaagcctcatctgagacaaggcaaggtcttccacctatgagcctgtaaaatcaaaacaagctagttacttcctagatacagtagggtacaggcattgggtaaatacacctattccaaatggaagaaattggccaacactaaggggctacaggccccatgcaagtctgaaatccagcagggcagtcaaatcttaaagtttcaaaatgatctcctttgaatccatgtctctcatccaggtcatgctgatgcaagaggtgggtttccatggtcttgggcagctctgctcctgtggctttgcagggtacagcatctctcccagctgctttcatgggctggtgttgattgtctgtgggttttccaggtatatggtgcaagctgtcagtggatctaccattctggagtctggaggatggtggcccttttctcacagttccactaggtggtgcccaggtagggactctgtgtgggagctctgagcccacatttcccttctgcactgccctagcagaggttctccatgagggcctcaccccttcagcaaacttcttcaccccttcagcaaacttcttctaggtttcctctgaaacctagatggaggttcccaaaacccaattcttgacttctgtgcactcatagactcaataccatgtggaagctgccaaggcttgaggcttgcaccctctgaaaccacggcccaatctctatttcagccatggctggagtggctcggatgcagggcaccaagtccctagaccgcacacagcatgggaccctgggcttggcccacaaaaccatttcttcctcctaggcctccaggcctgtaatggaaggggctgctgcaaaagtctctgagatgccctggagacattttccccattgtcttggggattaacattgggatccttgttacttacgcaaatttctgcaggcatcttgaatttctccttagaaaatgagtttttcttttctattgcattgtcaggctgcaaattttctgaacttttatgctctgtttcccatttaaaactgaatgcctttgacagcacccaagtcacctcttgaatgctttgctgcttagaaatttcttctgccagataccttaaatcatctctctcaagttcaaagatccacaaatctctagggcaggggcaaaatgccaccagtctctttgctaaaacataacaagagtcacctttgctccagttccaaacaagtttctcatctccatctaagaccacctcagcctagatttcattgttcatatcattatctgaattttggacaaagccattcaacaagtctctagggagttccaaacttttccacattttcctgttttcttctgagccctccaaatttttccaacctctgtctgttacccagttccaaagttgcttccccatttttgggtatcatttcagcagcaccttactctactggtaccaatttaatgtgttagttcgttttcatgctgctgataaagacatacccaagactggccaatttacaaaagaaagagatttattggacttacagttccacgtggattgggaggcttcacaatcatcatggaaggcaaggaggagcaagtcccatcttacacggatggcagcaggcaaacagagaaagcttgtgcagggaaactcccccttataaaaccatcagatctcatgatacttattcactatcatgagaacagcacaagaaagacctgcccccatgattcaattacttcccatggggtccctcccacaacatgtgggaattcaagatgagatttgggtggggacacagccaaacaagatcaacgtggaaattaaacaatatgcttctgaatgaccagtaggtcaatgaaataattacaaaggaaatttaaaaatttcttgcaacacatgctaatggaaacacaacataccaaaacctatgggatacagcaaaagcagcactaaaaaggaaagtttatagctataaatgcccacattaaaaaaagaaaaacatcaaataaacaacctaacaaagcatcttaaagaactagaaaatcaagagcaaaccaaatccaaaattaacagaagaaataaaaaagatcagagcagaaataaataaaattacaatgaagaaaacaatacaaaagatcaacgaaacaaaaagctggttttttttaagttaaacgaaatggacaagcctttagccagattagctcagaaaaaaaaagacacaaataaataaaatcagagatgaaaaaggagacacaacaactgattctgcagctattcaaaggatcataagtcactactgtgaacaacaataagccaataaattggaaaatctagaagaaatgggcaaatttctagacacatacaacctacaaagattaaaccatgaagaaatccaaaacatgaacagatcaataacaatgagatggaagccataatcaaaagtctttcagtaaagtaaagctcggaacctgatggcttcaccactgaattctaccaaacatttaaggaagaactaataccaatcttactcaaactatctgaaaaatcaaggagaagtgactacttccaaactcattctacaaggctattattgccccaataccaacaccaaagatacctcaagaaaagaaaactacaggtccatatctctgatgaatactgatgcacaaatcttcaactaaatacatatttaacaatacattaaaaagatgattcatcatgaccaagtgggatttatcccagggatgcaatcatggttccaacatatgcaaaataatcaatgtgatacattatatcaacagtatgaaggacaaaaaccatacaatcatttccatcaatgctgaaaacccatttgacaaaattcagcatctcttcatgaaagaaaaccctcaaaaaactgggtacagaaggaacatacgtcaacctaataaaagccatatacaaaagatccatagcagatgtcatactaactggggggtgagggggtactgaaagcttttcctctaatatctggaacatgacaaggatgcccactatcaccactgttattcaacgtaatactggaagtcctagatagagcagctagacaagagaaaaagaaaagggcatccaaattggaaaggaagaaatcaaattatccttgttggcagattatgtgatcttatatttggaaaaacctaaaggctccaccaaaaaactgttagaacgtataatcaaattcagtaaagttgcagggtgcaatatcaacatacaaatatcaatagcatttttatatgccaacagtgaacaaatcgaaaaagaaattttaaaaagtaatctcatttactttagctgcaaataaaattaaatgcctagaaattaatcaaagaagtgaaagatctttataataaaaactataaaacactgatgaaaaaaattgaagaggacaccacaaattggaaagatatttcatgttcatggattggaagaatcaatattgttaaaatgtccatactacccaaagcaatctatagattcaatgcaatccctatcaaaatgccaatgacattattcacagaaatagaaaaaaaaatcctaaaatttatatggaaccacaaaagacccagaatagccaaagctatcctaaacaaaaagaacaaaactggaggaagcacattatctggcatcaaattatacttcacgactatagtaaccaaaacatcatggtactagaataaaaacagacacacagaccaatgggatagaatagagaacccagaaacaaatccacacacctacagtgagctcatgttcgacaaaggtaccaagaagatacactagggaaaagacagtctcttccataaatgatgctgggaaaactggacattcatatgcagaagaaagagactagacccctatctctcatcatatacaaaaatcaaatccaaatggattaaagacttaaagacctcaaactatgaaactactacaagaaaacattggggaaacttccaggacattggactgcactaagatttcttgagtaataccccacaagcacaggcaaccaaagcaaaaaagcataaatgggatcacatcaagttaaaaagcttttgtatagcaaaggaaacaacccaaaaaggaaagaggcaacccacagaatgggaaaaattattcacaaactacctatctaacaagggattaataacaagtatatataagtagctcaaccaactctgtcaaaaaaaaaaaaaatctggccaggcatggtgggacacacctgcaatcccagcactttgggaggccaaggcgggtggatcaccagaggtcaggagttagagaccagcctggccaacatggtgaaaccccatctctactaaaaatacaaaaattagccgggcgtgatagcacatgtctgtaatcccagctactcgggaggctgaggcaggagaatcgcttgaacccaggaggtggaggtcgcagtgagccgagatcgcaccattgcactccagcctgggtgacaaaaaaaaaaaaaaaaagtaaaaaaaactaataatccaattttaaatgggcaaaagatttgaaaagacatttctcaaaagaagagatacaaatggcaaacggacctatgaaagatgctcaatgtagtgatcatcagaggaagggacaaatcaaaactacaatgagatgtcatctcaccccacttaaaatggcctcaaaaacaggcaataacaaatgctggctaggatgtggagaaaagggaacctttgtacactgttgatggaatgtaaattagtacagccactgtggagaacagtttggaggtttctcaaaaaatgaaaaatagagttaccatatgattcagcaatcccaccactgagtatatacccaaaagaaaggaaatgagtgtgtcgaagggatatctacactcccatgtttgttgcagctctgttaacaatagtcaagatttgaaagcatttaagtgtctatcaacagataatggataaagaaaatgtgattctcatacacagtgtagtactattcaaccataaaaaagaataagatccctgtgcaacaacatggatggaaccagaattcattatgttaagtgaaataagccaggcacagaaatataaacatcacgtggtctcatttatttgtgggacctaaaaataaagacaattgaacccattgagatagagagtagaaggatggttaccagaggttgggaaggttagtgggggataggaggaggtgagatccttaataggtatcaaaaagttaaaatgaataaataaggcctagtatttgactgcacaacagggtggctaccattaatataatttaattgtgcattttctattaagaatagcaaagacatgaacacaacctaaatgcccatcaacgatagactggataaagaaaatatggtacatatacaccatgtaatactatgcagccacaaagaaaaggatgagatcatgtcctttgcagggacatggatggagttgaaagccattatcctcagcaaactgatgcaggaacagaaaaccaaacaccacatgttctcacttataagtaggagctaaatgatgagaacacagggacatattggggggaaaacacacattggggcctgttggagcaggggtgaaggcaggcagagcctcaggaaaaacagctaatgcatgttgggcttaatacctaggtgatgggatgatccatgcagcaaaccaccacagcacacatttacctatgtgaaaaaaccctgaacatctggcacatgtacctctgaacttaaaatgaaagttgaagaaaaaaattaaataatttaattgtgcattttaaaataactaaaagaatacaactggattgtttgtaacacaaaggataaatgcttgagagtgtgaatactccattttccatgatgtgattattacacatagcatgactctatcaaaacatctcatgtaacccatgaatatatacagctgctgtgtacccacagaaattgaaaattaaaaattattttaaaaaagaaaataaaaataaatacagtaaaagaaacaatgaaggattaaaacaatacattagaaaatatatttataacacaaaagtaggtattaataaaggaaggattgaaaaagacatgaaacatctaaaaaacaggtagaaaaatggcaaatgagaacattacattattagcaatttcattaaatgtaaactggttgaacattcaaattaaaaataagagattcagccaggtgcagtggctcacacctgtaatcccagcactttgggaggttgagatgagtggatcacctgaggtcaggagttcaagaccagtctggccaacatggtgaaattccgtctctattaaaaatacaaaaaaaaaaaaaaaaattagccaggtgtggtggcaggtgcctgtagtcccaactacttgggaggctgaggcaggagaattgcttgaacccaggaggcagaggttgcagtgagccgagattgtgccattgcactccagactgggtgacaagagtgaaactatgtctcaaaaaaagaaaaaaaaggggttagcagatttaaaagcaagattcaactatatgctgtctatgagagactttagatttaaagacacaaagaggttgaaaataaaagtatgaaaaaagatgtaccagtcaaactgtaactaagagagacctagaatgactatattattaaagtagactttaagacaaaaaattgttactaaagactaagaaggacatcttacaatgataaaagagttaatcagccaggcatggtgtctcatgcctgtaatcccagcactttgggaggccaagccaggtggatcacttgaggtcaggagttcgagaccagcctggccaacatggagaaaccctgtctctactaaaaataccaaaattagccaggcatggtggcatgcacctgtaatcccagctactaaagaggctgaggcaagagaatcacttgaacccgggaggtggaggttgcagtgagctgagatcgtaccactgtactatatagcctgaacaacaaagtgagactctgtctcaaaaaaaaaaagagttaattgatcaaaaacatgtaagaattataaacatatatgcaccaaacaaccccaaattctaacagaattgaaactgacagaattgaaggagaaaatagtcaattcaacagtagttggagattaattaacccactttcaatggtggttagaatagctagacagaatgtcttccagaaaatggaagacttgaacaaggtatagtccagtttccagagatctaaagaacctccatttattaataaaataatacacattcttctcaagcacacaagtaacatttgccaggataaaacatatgttacaccataaagcaagtcttaataaagtataaaagactgtaatggtacaaattatactatgtgaccacagtgaaatgaaattagaaattaataacatgaaaatttggaaatttcacaagaaaattgagcacagttgtaaataattaaaggtcaaaaagtaaattacaaaagaaatttgaaaatgccttgagataaatgaaaataaaaacacagtacaccagatcttaggagatgcagtaaaaatagtgcctagagggaaatgtataattagaaatgcctatttttttttaaaaaaagatagatctcaaatcaaaaccaaaactttatcttaaagagctaaaaaaaaaaagcaaatccgaagcaaacagaagaaaagaaataataaatattacagagaaaataaaggaaatagaaaatagaaaaatcatagagaaaatctgtaaaaccaaaagttggttctttgaaaagaccaacaaaattgacaaaactttagctaatctcaccaggcaaggaaaaaaaaaaacaaacctcagccgggcgtggtggctcatgtctgaaatcccagtactttggaaggctgaggcaggtggactacctgaggtcaggagttcgagaccagcctggccaacatggtgaaacctcatctctactaaaaatacaaaaattagccaggtgtggtggcacgcacctgtaatcctagctacttgggaggctgaggcaggagaatcacttgaacccaggaagcaaggttgcagtgagctgagattgcagcactgcactccagcctgggtgacagagctgacagagcaagactctgtatccaaaaaaaaaaaaaagaaagaaagaaaagaaaaaacagactaaaattactaacatcagaaatgaaaaatgggacactattaataactttacaaaacaaaaaatattataaggtgtttttgactgaatgttccccctcactgtctctaaatccacatgctgaagccctagctctcaatatgatgatatttaaagatggggttagggtaaggtaaatagggttaggttaggtcatgagcttgggccctcatcatgggattagtggacttagaaaaagaaggagagatgagatcatggcagacaggcaggactagattgaagctcccacttggacagacacaacagcctgtggaggcttgtactgtgaacttttgctccagtatgactgtagaaatatattaggaaagccaagagaacccatatatcttccgaaagaagtggattgttcctgcaagactctggagacaccccaaatactgtgagtgcccaaactctgcaagtgggaaaggaagattgttcatccatgaacatacatccccactggggaacctgaaggtctagattatgggagaagattctgaccttacctggagctgagtcaatttagagagctgagtgaaatacagaggtagaggaagcagaaggaaaatctctgtgggcttgctgggtcccttagcaagacatttctgcctcacctcacaggcatccttgaggagagctaccagagtcactgggaaaaggccacagggagaagaaaagcaccagctgaactttgtaacaatttgaaccaatttagaagcctcctggccagaaactgggggagggtgtgaatctggtgtgcatactccacaggtgggagaggaaggaaagccctagttattttcacagctgggaggtgggtagcctggggcaagttctcagtcctgctggcccactgcctggaaacagacttgttgctgttgggggtgggagcatggtggaagtgagatcagccctttgggttgggtgggagctgggtgaggcctgtgactgacagctttcccccacttccctgacaacctgcatgacacagcagaggcagccataatcctcctggaaacaaaactccattgacctaggaacctgaacctcacccccacccccaacaggagccgcagcaagacttacccaagtagagtctgagctcagacacacctagtcttgcccccacctaatggttcttccctacccaccctggtaactgaagacaaaggacataaactcatgggagttctaaggcctctcccaccacctgttcctccacatactaccacagctgatgctctcttgaaagcaccacctcccagcaggaggccaaccaacacaaaaatagtgcaataatcaaccaaagctaaggaccctcacagggtccatttgatacccctgccacctccaccagagcaggtgccagtatccatggctgagaaacccacagattgttcacatcataggactctgtgcagacaacccccagaacctgtctactagagcaagtctactagcccagagcctggtaggcttgctggatggctagaagagagataacaattactatagctcagctctcaggaagccacatccctaggaaaagggggacagtactacaacaagaaaacaccccgtgggacaaaaggacctgaacaacagccttgagcctgagatgttcctgctgacaaagcccaaatgagaaggaacccaaaaaccaactcttgtaatatgacaaaaaaaggttttttaacaccccccaaaaatcatactagctctccagcaattgatccaaaccaagaaaaaatccctgatctacctgaaaaagaattcagaaggaggttagttattaagctaatcagggaggcaccagagaaaggtgaagcccaatgtaaggaaatccaaaaaatgatacaagtgaagggagaaatattcaatgaaaaagatagcataaataaaaatcaatcaaaacttcaggaaacaatggacacacttatgcaaatgcaaaatgctctgaaaagtctcagcaatagaattgaacaagcagaataaagaacttcagagcttaaagacaaggttttcaaattaacccaatctagcaaagacaaagaaaaaagaataagaaaatatgaacaaagcttccaagaagtctgggattatattaaataaccaaatctaagcatcttcagcgttcctgaggaagaagataaatctaaaggcttggaaagtgtatttgggggaaaaatcaaggaaaacttttctggccttgctagagaactagacattatacaagaagctcaaagaacatctgggaaactcatcacaaaaagatcattgcccaggcacattgtcatcaacttatctgaagttaagatgaaggaaaaaatattaaaagctgtgaggcaaaaacaccaggtaacctataaaggaaaacctatcagattaatagcagatttctcagagaaagtctacaagctagaagggattggggccctatcttcagccttcacaaacaaacaattatcagccaagaattctgtacccagtgaaactaaactttgtagattaaggaaaaatacagtctttttcagacaaacaaatggtagaaatcactactaccaagccagcactacaagaactgctaaaaggagctctaaattttgaaacaaatcctggcaacacatcaaaacagaacctctttaaagcataaatctcacaagacctacaagacaaaaatacaatttaaaaaaaaaaaaacaacccaagatatacaggaaacaaatagcataatgaatggaatgatagctcacatctcagtcaaacgttcaatgtaaatggcctaaatgctccactgaaaagatacagaattgcagaatggataagaattcaccaaccatctgctgccttcaagagacacacctaacacaaaaggactcacgtaaacttaaggaaaggaatggaaaaagacattgcatgcaaatggacaccaaaagtaagcaggggtagctgttattatatcagacaaaacaaacattaacgcaacagcagttgaaaaaacaaagagggtcattatataatgataaaaggtcttgtccaacaggaaaatatcacaatcctaaatatatatgcacctaacactggagttcccaaatttgtaaaagaattattaatagaagtaagaaataagatagacagaaacacaataatagtgggggcctttaatactccactgacagcactagacaggtcatcaagacagaaagtcaacaaaaaacaataaatttaaacaataccctggaacaaagggacttaactgatatacacagaacattctacccaacaaccacagaatatacattctattcaacagtgcatggaactttctccaaaagaccatatgataggctgcaaaacgagcctcaataaaggtaagacaattgaaattgaattcaagtattctctcaggccacagtggaatacaactggaaatcaactccaaaaggaaccttcaaaaccatgcaaatacatggaaattaaccttctcctgaatgatcactgagtcaaaaatgaaatcaagatgaaaatttaaaaattcttctaactgaatgacaatagtgacacaaccaatcaaaacctctggaatacagcaaaggcagtgctaacaggaaagttcatagccctaaatgcctacatcaaaaaggttggaagagcacagacaatctaaagtcatacctcaaggaactagagaagcaagaacaaactaaacccaaacccagcagaagaaaggaaatcaccaagatcagagcagaactaaatgaaattgaaacaaacaaaaaaatacaaaagataaacaaaacaaaaagctgcttctttgaaaagataaataaacttgataaaccattagaaagattaaccaagaggagagaaaatccaaataagcacaattagaaacgaaatgggagctattacaactgacaccacagaaatacaaaagatcatcaaggctactatgaacacctttatgtgcataaattagaaaacctagaagagatggataaagtcctggaatgatacaaccttcctagctttaatcagatagaattaggcactctgaacagaccagtaacaagtagagagactgaaatggtaatagcaacaattaccaacaacaaaaaaagtcaaggaccaaacgaattcacagcagaattctaagagacgttcaaagaagaattggtaccaatcctattcatgctattccacaagacagagaaagaggggaccctcccctcaaatcattctatgaagccagcataaccctaataccaaaaccaggaaaagacataacagaaaaagaaaatgagaggtgacagtgtgctggcagtgctcacagccctcacttgctctgggcgcctcctctgcctgggctcccactttggcggcacttgaggagcccttcagcctcccgctgcactgtgggagcccctttctgggctggccaaggctggagctggctccctcaacttgcggggaggtgtggagggagaggcgcgtgtgggaactggggctgcgcccggtgcttgtgggccagcgcgagttccaggtgggcatggactcagcggaccccgcactcggagccgccggccggccccaccggcccccagcagtgaggggcttagcacctggccagcagctgctgtgctcaatttctggccaggccttagctgccttcccacgggacagggctcaggacctgcagcccgacatgcctgagcctcccctcccactccgtgggctcctgtgccgcctgagcctccccgaggagtgctgccccctgctccaagagcccagtcccatccaccacccaagggctgaggagtgtgggcgcatggctcgggactggcaggtagctccacctgcaggctccgtgggggatccactgggtgaagccatctgggctcctgagtctggtggggacgtggagaacctttatgtctagctaagggattgtaaatacaccagtcggcactctgtatctagctactctggtggggtcttgcagaacctttgtgtccacactctgtatctggtggggacatgaagaacctttgtgtctaactcagggattgtaaatgcaccaatcagtgccctgtcaaaacagaccactcggctctaccaatcagcaggatgtgggtggggccagataagagaataaaagcaggctgcccgagccagcagtggcaacccgctcgggtccccttccacagagtggcaacttgcttgggtcctcttccacacagtggaagctttgttctttcgctctttgcaataaatcttgctactgctaactctttgggtccacactgcctttatgagctgtaacactcacggcgaaggtctgcggcttaactcctaaagccagcgagcccgcgagcccacagggaggaaggaacaactccagacgcccgccttaagagctgtaacactcaccgggagggtctgcagcttcactcctgagctagcgagaccacgaacccaccagaaggaagaaactcggaacacatccgaacatcagaaggaacaaactccagacatgccgccttaaagaactgtaacactcactgcaagggtacttggcttcattcttgaagtcagtgagaccaagatcccaccaattccggacacaaaaactacagaccaatatctctgatgaacatagatgttaaaatacttaacaaagtactacctaaccaagtcaaacaacatatcaaaaagataatccaccatgatcaagtgggtttcataccagggatgcggcagtggtttaacatatgcaagtcaataaatgtgatacaccacttaaacagaatcaaaaacaaaaatcacacgatcatctcaatagatgcagaaaaagcattcaacgcaatccagcatccctttatgattaaaactctcagcaaaatcagcatacaagggacatacctcaatgtaataaaagctgtctatgacaagcccacagccaaaataatactgaataaaaaaaagtggaaagcattccctctgagaactggaataagacaaggatgcccactctcaccactccccttcaacataggactggaaatcctagccagagcaatcagacaaaagagagaaataaaggacatacaaattggtaaaaaggaagtcaaactgttgctgtttgctgatgatatgatcctttacctagaaaaccataaagactcctcaagaaagctctagaagtgataaaagaattcagcaaagtttctggatacaaaatcaatgtatgcaaatcaatagctcttctatacaccaacagcaaccaagctgaaaattaaattaagaactcgaccacggccaggcgtggtggctcacacctgtaatcccagcactttgggaggccgaggtgggcagatcacgaaatcaggagttcaagaccagcctggccaacatagtgaaacctcgtctctactaaaaatacaaaaaaattagctgggcatggtggcatgtgcatgtagtcccagctactcaggaggctgaggcaggagaatcacttgaacctgggaggggggggttgcagtgagccgagattgtgccactgtactccagcttgggcaatagagtgagatttcgtctcaaaacaacaacaacaacaacaacaaaactcaaccccttttacaatagctgcaaaaaaataaaataaaatgcttaggaatatacctaaccaaggaggtgaaagacctctacaagcaaaatggcaaaacactgctgaaatatatcatagatgacacaaacaaatggaaacacatttcatgctcatggataggtagaatcaatattgtgaaatggccaggtgcagtggctcaggcctgtaatccccgaactttgagaggctgagatgggtggctaacttgaggccaggagttcaagaccagcctggccaacatggcaaaccccctctttactaaaaatataaaaattagtcaggagtggtagtgcacacctgtaattccagctactgaggaggctgaggcatgagaatcacttgaacccagggttcaagtgttgcagtgagccaagatcatgtgactgcactccagcccagatgacagagtgagactgtgtctcaaaaaaaaaaaaaattgtgaaaatgaccatgctgccaaaatcaatctacaaattcaatgcaattcccataaaaataccatcgttcttcacagaattagaaaaaacaattataaaattcgtatgcaaccaaataagagcctccatagccaaagcaagactaagcaaaaagaacatatctagaggcatcacattacctgatttcaaactatagtataagaccatagtcaccaaaacagcgtggtactggcataaaaataggaacatagaccaatggaacagaatagagaacccagaaatgaagccaactgatctttgacaaagcaaacaaaaatataaagtggggaaaggacacaccctattcaacaaatggtgctgggaagattggcaagccacacgtaggagaatgaaactggctccttatctctcaccttatacaaaaatcaactcaagatggatcaaggacttaaatctaagacataaaactataaaaattctagaagataactttggaaaaacccttttagacattggcttaggcaaggatttcatgaccaaaaacccaaaagcaaatgtaacaaaacaaagacatatagctgggacttaataaactaaagagtttttgcatggcaaaaggaacagtcagcagagtaaacagacaacccacagagtgggagaaaatctctgcaatctgtacatatgacaaaagactaatacccaggatctacaacaaattagcaaggaaaaaaacaaacaatcccatcaaaaattgggctattctcaaaagaaaatatacaaaaggtcaacaaacatatgaaaaaatgctcaacgtcactaatgatcagggaaatggaaatcaaaaccacaatgtgataccataatggtattacattaatggccataatcaaaaaatcaaaaaataatagatgttggcatggatgcagtgaacagggaacacttctacactgctggtgggaatgtaaactagtacaaccactatggaaaacagtgtggagatcccttaaagaactaaaagtagaactaccatttgatccagcaatctcactattgggtatctacccagagaaaaagaagtcatcatatgaaaaatatacttgcacccccgtgtttttagttgcataatttgcaagtaaaatcatggaaccaacccaaatgcccatcaattaacatgtggataaataaactggtataaggctgggcatagtggctcacgcctgaaatcccagcactttgggggtgccgaggcagatggatcatttgaggtcaggagttcaagaccagcttgccgaacatggtgaaaccctgtttctactaaaaataaatacaaaaaaatgagccaggcatggtggtgcacatatgtaatcgcagctactcaggaggctgaggcatgagaatcacttgaacccaggaggtggaggttacagtgagatgtgattgtgccacttcactccagcctgggtgacagaatgagaccctgtctaaaaaaaaaaaaaaaaaagatgtgaaactggtgtgtgtgtgtgaatatatatatatatgatggaatactactcagccataaaaaggaatgaattaatggcactcacagtgacctggatgagattggagactattattctaagtgaggtaactcaggagtgggaaactaaacatcgtatgttctcactcatatgtgggagttaagctgtgaggatgccaagacataagaaggacaaaatggattttggatactcagggggaaaaggtgggaaggggatgagggataaaagaccacaaattgtgtacagtgtatactgctcaggtgatgagtgcaccaaaatctcaaaaattaccaccaaagaacttactcatgtaacgaaacatcagctgttccccaataacctacggaaataaaaaataaaataaaaaagagggagagatctcatctctctcttctttctagtgctctctctttacatgcatgcaccaaagaaaagctttgtgaggacatatcaagatagtggctgtctgtaagtcaggaagcaagccctcaagaggaagtaaatcagctaggaccttgatcttggacttcccaaagtccagaactgtaagaaatgaatttctgttgtttaagtcacacagtctgtgatattttgttacggcaacccaagccaactaagacattaggcatactatgaacaattataagccaacaaagtagataacttagatgaaatagacaaattcctagaaacatacaaacaaccaaatctaattgaaaaaggaatagaacatctgaaaagatcaataacaagagtctgaattagtaataaaaaaacttttgaaaaggcccaggactatacagatttactggtgaattctaccaaacgtttgaagaagaattactactgattcttcataagcttttgcaaataataaaagaggagggaacagtttctaactcaatatttgatgtcagtacttccctgatataaaagcaagacaaacacatgacaagaaaagaaaactacagagcgatatctttataaatatggaagcaacattgctcaaccaaatactggcaaactgaattcaaaaaaacatataaaaacattttagaccactcagcatccccttcccttcactgtctctttcccttgttccctctcttccccctcccagagcaccttccccgtgaggctgcagtagtggcaatggggttcaactgggtcccggtgaacaggagcgggggctggcagacatggcggccaacatgtaccaggtaggagattttgtctactttgagaattcctccagcaatccataaataatcaggatagaaaatctcgacaagactgcaagtggcaacatggaagcaaaagtagtatgcttttacagattacatgatatttttaacacactgataatgcttgtggagaagcatgctaaagaaattgaggaagaatctgaaacaatagttgacccttacttgaccaatactcagaaacatcaatcaaaacataagttactttctttgttaccccaatatgaatctttgccttcaacacatattaggggaaaatgcagtgttggcttatgaatgagacagaatcagtattgtcatatcttcataaaaaggatactttcttctacttattgatcatgacccctcatcgaaaacactattagctgatgaaggtgaattcagaatgggacctagatatcaagtagatgtcccagaaatgctcatagaaggagaatcagatgagagggagcagtgaaaatggaaagttaaagtttggggatccaaatagcccacttaaagattgatcaattaaccagtttttaattgtagcaagtgctattgggacactccctagacctctcgattgcagcagttctttgaggcagcctagtttgcatatgagtgctgctgcagcttcccaagacatcaccttctttcatgctatcaatacattgcgtagacatagctatgatttgagcagtgcaattagtgtctcagtaccacttgcagaagctgttttatgcagagataaattggaagaatggtcagcctcttaagctagcttattcgaagaggtactggaaaaaatatggcaaagacttcaacgacatatgccaaaactttttcccttggaaatcattgactagcatcactgaatgttatttcttgtggagaactactgacagatatgtgcaacagaaatggttaaaagcagcagaaactgagagtaagctgaaacaagtatatatcccaatttacaggaaaccaaatcccaaccaaatatctaccagcaatggcaagcctggtgctatgagtgaaaccatgaggttgacattcctgcctcagaatccccttgtagtgcaagcctgggagggctgctgtgctacacagtctaaccagtagtattcttggggcccacctaatatgcagtgcaatttgttggctttattgagaaaaatatggagacctgaaaatgcccacccagtcagaagaaaagttatctcctagcccaattacagaggactctcatgttagaaatgctgccaggccattcaagggatgccactccaaaacactgtcagtacaaagtctgcagtgaaaactcaccaagctttcttccttcatactgtgtttttcacaaaatttgctctccatttattcaaaagtatcctctggctgaagcaggcagcaagatggccacttgtagctattaatttatgctgccattagggcagaatgtaagcttttttttttttttttttttagacagggcttcactgtatcacccaggctggagtgcagtggcatgatcatggatcactacaaacttcctctcccaggttcaagtgatttttctgcctcagcctcctgagtagctggaattacaggtggctgctgccacgcctggctaatttttgtatttttagtagagacagggtttcaccatgttggccaggctggtcttgaactcctgacctcaggtcatcctcccacctcggcctcccaaagtgctgagattacaagtgtgagccattatgcccagccactgctttaaaattcttaatcttatatgttgtgcttctgaccatttcctctcgctcctttttttgtttgtttgtaataaatgtaagttattgtgtacaaccttttatttggtttattttttaacattatttttgtctgttgctatttgtatcatgccaatctgaaaaaaatcaaagcattaaaaacttctgttatcttatcagggaatactgctcagcaaaagattggtgggaggtgatcctatcccatggggttttatgatggaattgcctacaaagcccataagcacttttacctactaggtagtgcaacaagataacccctaaggatggttgatatagtatggctgtgtcccacccaaatctcatcttgaattgtaattctgataatccctatgtgtcatgggagggaccaggtgggaggtaattgaatcatgggggtggttacctccatcctgttctcatgatagtgagtgagttctcatgagatctgatagttttataagggttttttctccctttcactctgcacttctccttgctgccaccatgtgaagaagggtggatttgcttccccttccaccatgattgtaagtttcgtgaggcctccctagtcctgctgaactgtgactcaactaaacctctttccagccaggtgcagtggctcacgcctgtaatccaagcactttgtggggctgaggcgcgtggatcacctcaggtcggaagtttgagaccagcctgaccaacatggagaaacctcatctctactaaaaatacaaaataattagccgaacgtggtggggcacgcctgtaatcccagcaactcaggaggctgaggcaggagtattgtttgaacccaggaggcagaggttgtggtgagccgagattgcaccattgcactccagtctgggcaacaagagcgaaactgacattaaaaaaacaaaacaaaacaaaacaaaaccctctttacttcataaattacccagtctcaggtgtgtctttatcagcagtgtgagaacagactaatacagtaaattgataccaggaatggagctgttcgggtaaacataccccaaaatgtggaagtgactttggaactggataacaggcagaggttggaagagtttggaagagttagaagacaacagaaagatatgggaaagtttggaacttcctagaaacttgttgaatggctttgaacaaaatgctgatagtgatatggacaataaagtccaggctgaggtggtctcagatgacgatgaggaacttgttgggaactgaagtaaaggtcacttgctatgcaaagagactgatgacattttgcccctgccctagagatctgtgaaactttgaacttgagagaaatgatttagtatatctggcagaagaaatttttaagtgccaaagcattcaagagagagtagaacataaaagtttggaaaatttgcagcctgatgatgcgatagtaaagaaaaacccattttctagggagaaattcaagcccactgcagaaatttgcataagtaacaaggagccaaatgttaatcaccaagacgatggagaaaatgcctacagggcatgtcagagaccttcagaacatcccctttcatcacaggcccagaggcctaggagggaaaaatgacttcatgggctgggcccatggccaccctgctctatgcagcctctggacatggtgcccagcatcccggctgcttcagctccagccatggctaaaagggaccaatgtacaactcaggctgttgcttcagaaggtgcaagcccttggtggctcccacaaggtgttgggcctgtaggtgcacagaagtcaagaactgaaatttgggaaccttcacctagatttcagaggatatatggaaacatctggatgtccagagagaagtttgctgcaggggtggaaccctcatggagaacctctgttagggcagtgtggaagggaaatgtgggttcagagccccaacacagagctcccactggggcactgcctcgtagagctgtgagaagagggccactgtcttaccccagaatggtagatccaccaacagcttaccccatgtacctggaaaagtcacagacacccaatgccagcccatgaaagcagctaggagggaggctgtaccctgcaaagccacaaaggtggcactgctcaaggccataggagcccacctcttgtatcagcatgacctggatgtaagacatgaagtcaaaggagatcattttgtaactttaaggtttaatgattgccctgctgggtttcagacttggatggggcctataacccctgtgttttggccaattttttccccattggaatgagtgtatttgctcaatgcctgtacccccagtgtaactagaaagtaactaacttgcttttgattttacaggctcataggcagaagggtcttgccttgtctcagataaaactttgaacctggacttttgagttaatgctggaatgaattaagattttgggggactgttgggaaggcaggattatgttttgaaatgtgagaacatgggatttggaaggggccagggcataatgctgtggtttggctgtgtccccacccaaatctcattttgaattgtagttaccataatccccatgtgtcataggagggacccagttggaggtaattgaatgttgttcacatgacagtgagtgagttctcatggatctgatggttttaaatggggctttcccccactttgctctgcacctctccttgctgctgccacgtgaagaaggacatgtttgtttccctttccatcatgactgtaagtttccttaggcctccctagccctgaagaactgtgagtcaattaaacctctttcctttataaattacccagtctcaagtatgtctttattagcagtgtgagaatggaataatacgatgatgttataatgaaactccataattgagacagtgaggtccattcatatgtagattaattaggtctaaataaaatttgcccagcctggcaaacacagtggaaccccacctctaccaaaaaatacaaaaattagccaagtgtggtggcacatgcctatagtctcagctccttgggaggctgagacatgagaattgcttgagcccaagaggtggaggttgcggtgggccaagattacactactgcactcctgggcaacagagcgagaccctctcataaataaataaataaataaaatttgccaatttacacttaaaaaaggattttataccataaccaaacgagatttatcacaggaatgcaaggttggctcaacataaagaaatcaatcaataggctataccatattaataaagaaaaatcccatatgatcatctcaattaatgcaaaaaatgcatttgacaaaatgcaacaccatgtcatgatttaaaaaaaaaaaaaacacttggcaaatggggagtaaaagggaacttaacctgataaagagaatctatgaaaaccccagaactcatataatatttaatgatgaaagactgacagttttactcctaaaatcagaaaaaagacaatggtcttccagtctgatattactttatgattataaagtcatgtgtgttgtaatttaaaatactttctataaagtctagatgtggtggcacatgcctgtaatcccagcactttgggagattgaagtgggaggattgcttgaggccaagagtttgagaccagcctgggtaacatagtgagaccctgtctctacaaaaaattaaaatattagctgggtgttgtggcatgtacctgtagtcctacctacttaagaggctgaggcaggaggattgagaggatcaattgagctcaggagtttgaggttgcagtgagctatgattgcaccactacatccagcctaggtaataaagtgaaatattgtatctaaacaacaacaacaacacttcttaaaggatatgcatggtgtaatccaattttcattctttaacaagtatatgtgatgcacagaaatgagtttagaaggctgtataccaagatattggcaatggtaattgggatggtggcagtgttgatgatgtatatagcttatatatattttttaacaacaactttattgagatgtaactcagatgccatacaatccacccatatttaaagtatacaatccagtggtttttggcacagttatgcaagcataaccactatctaattccataatattttatcaccctaaaaagaaaccttctactcattagaagtcactccccacttccttctcttccagcccccaacaaccactaaccatctttctgtttgtatgagtttacccattctggatacatgctatgtaaatggtatcatataatatgtatttttttgcatgtctggcttctttcatgtgtcatcatgttttcaaggtccatccatatcgtagcatgtatcagtactttattcctttttacagctaaacaatattccattgtatggatatactatattttgtgtatgcttttatcagttgatggacatttgggttgtttctacttcttgactattattattaacggtgctatgatcattcatgtacaagcttttgtgtagacatatattttcacttatcttggatatatagtaggtatgtgtttaacatcttaagatatatagtaggtatgtgtttaacatgttaagaaattgcaaagcagtttccaaaatgattgcgtattttatatttctatcagcaatatatgaaatgtatgaaagtcctagttgctctatatcctctccaatatttggtttgattagtcttttaaattttaacccttctaatggatgtgtcatagtatctcattgtgcttttaatttgcatttgcctgatgattaatgatgttgagtgtcttttcatggccttattggctattagcgtatcttcttttttaaagtgtcttgccaagacttttgttcctggttaaaattgggttttctttttgatacctatttgtagaagtttttacatattctgaacacacatcttcatgaatattttctatagtttgtggcttgccatttcatattccaactttgagtcaaggtctactattagactccaaacactgggcagacctcaagctttgtcttctgttctccaccccacttcctgtgaaaccattataaatgcaatgtcaagatcactacagtttagcatatgttcttaaggcaaaagccttagcacttttctcacctcttaggaggttccatgttcactctatttttagtttctgggagcctttactttcttggcatttcagtaatatgtttgaaagtttatttgtttatatatattttttttttctttttttttttttttttttgagatggagtctcactctgtcacctaggctggaatgcagtggcgcaatctcagctcactgcaacctccacttcccaggttcaagcgattctcctgcctcagcctcctgagtagctggggttacagccacacaccaccacgcctgactaatttttgtatttttagtagagacggggtttcactatgttggtcaggctcgtctcgaactcccgacctcgtgattcacctgcctcagactcccaaagtgctgggattacaggcatgagccacggtgcctggcttgtttatattttatatggcagcagtattactttgagggagaggatgatagcttcccttgcagctagcactgatcatgtgacatagttctggccaataagagaagaggaagtctgtgggcaatgatcctttcctcttccttccacttttctgcttcgaatatagatgtgagaaccagagatgaagcacccatctttaattcattaggaagtaacacatttgctaagaatggctaaatagaaataaaacagaaaccataaacaatgatgacatcatgagctgtgggaccaatcctgggttgcctaactccaaacttcttagtgaaaagttatatactctaatttgtctaaactactgttggttgggttttctgatacttacagtcagaaattaaagttatttttgagatttaatatttatataatcattcatccatctaatcaataagcatgtattaaatacatatcaagtgccagacattgtttataatagagtttctcattaggggtggtattactattagttagcattttaaacatttgtgagaaagtttttgtagtgttacaactggaagcaactaacatttagtggattggtactgggaatacaatatttctttttttacatcttattttttttttttttttgagacagagttttgctcttgttgcccaggctgaagtgcgatggtgcaatcctggctcactgtagccactgcctcccgggttcaagcgattctcctgccccagcctctcggatagctgagattacaggcatgcaccaccacacccggctaattttgtatttttagtagagacagggtttctccatgttggtcaggctggtcttgaactcccgacctcaggtgatctgcccacctcagcctcccaaagtgctaggattacaggcataagccactgcgcccagccaatacagaatttcttaaaatgcataagactgtcccatgcagtaagaattgccctacatctcgcatacttttcaaagtccaccaaagacatatctctagctgaaaaaggtgtttttaatgatctaaaattagaacttaattcattttgcaaataaataccaagtagtttcttcatgtgtttaatagatttaaattttccagaaattttttttaaagagacagggccttgctctattgcccaggctggagtgcagtggcatgatcatagctcactgcatcctcaaactcctagattcaagcagtcctccaacctcaacctcagtctcctgagtagctaggactacaggggtagtcctaccatgtagcatggtagtagtaactaccatgcccagcttttgctttttttttttttttttttttagagatgggggtctcccaatgttgcccaggctggtcttgaactttggcatcaagtgatcctccttccttggcctcctaaagtgctggtattacagacatgagccaccaagcccagcttccaggaatattttgttagaaatataccataaggtattcaacatttaggaaaatcacataatcactcgcaatgctgcatatgattttaaagtttccaatataacacacctatatcagactacatgtatagctgtctcatttacatttattctatatattagtgtaagtaagcatctaagttattttgtctagaagtcaaaactagatatttaccttttgaaatacatactatattataaacaactttgcttttattactcctttacataatcaggtgttatgttgcttttttaaattatgttcacttgtaagttatatagtctatttagttaatgacagaattgtaagcaggtcttataaaatatctataataggaagggagttgcgcctggatggagttaagaatcattggttttaatggtaattccttatctttaaaaaattgttggccaggcgcagtagctcatgcctataatcccagcactttggggggccaaggtgggaggatcacttgagcccgagttcaagacaagcctggacaacatggcaaaactccatctctactaaagatacaaaaaattagccaggcatgttggcgtacccctgtagtcccagctacctgggaggatgaggagagaggatcacctgagcctgggaggttgaggccgcagtgagctgtgattataacactgtactccagtctgggtgacagagtgagaccttgtctcaaagaaaaaaaaattgttacatagtatttaatacttttcattcaagatgacaagaaaatgcctaaaagcaaagaaaatccctggttgcttggaaaacctgacccccagctagaattccctgcctatagatttttaatttctgagtacactttatggctctttgactcagattgcattttactccggtgggcagcagaagttactgccactctgccagctcctggcgactactggggagggttgtgaagtcacctcattccagcagcaaatccatatggctctgcagcaatttgattcttgcctcctcagaggaaaaaatttggctaaggggcataaggcagagtgagagacaggggcaagttttagagcaagagtgaaagtttatttaaagttttagtacaggaatgaaaggaagaaaagtacacttggaagagggcccagcagaccacttgagagatccaagtgccccgtttgtcccttgacttggggttttctacattggcacggttctggggttggcatttcttcttccttgatttttcccttggagtgggcagtccatatgtgtgatggcccgccagcacttgggaggggctgcatgtgcagtgtgtttactgaagttgtgtgcatgctcacctggggcattttttccttaccagttgagcgttcctagaggaaggtcatataccagttaaactctgccattttgcctcttagtgtgcatgtttgagcccatttgcccaactcctgaaattctatcggaaagctactgatcaccagcttcgggtattttctgtctattgggaggctgtcttttcgtggcgccagctgcaaccaattattttggagagacagtttaacaacaacctgaccataacctgatggtcacctgacattcctggtgggggccctctcctgccctgctcgtgtctgcctaactaccactctaaaaacctaagaacttggctgtatttctcttgaagtttgacaccattcctctgaagaggaaaacacaattaaggaggacttactgacttacagatggagaatgaatgaaaggagaaatgcaacttgtttaagatcatgcaaaaagcagtgagtgttcagaggtaggaaccaaatctaattttccaattcctgaagctaaattaggcttaacccattagtgtatgttttatatttatgtatttatttacttatttttgagacagagtctcactctatcacccaggctggagtgcagtgtcacaatctcggctcactgcaacctctgactcctgggttcaagcaattttcctgcttcagcctttctgtgtattttttatatttttgtattattagtagagacggggtttcatatgttggccaggctggttggaactcctgacctcaggtgatccacatgccttggcctcccaaagtgctgagattacaagtatgagccaccatgcccagcctgttttctattaaaaaaaaaaaaaaaaagacatgagaatatctgtgtatggtcagaaagacctctctcctcttgaagtttaataacttaaaaaaagtggcttactacaggcaacaataggaaaaatagataaatggataaatgggattacatcaaaccaaaaagcttctgtataacaaagaaacaatcaaaagggtgaagaaacaacctacagaatgggagaaaatatttgcaaatcatacaaagaggttaatgtccaaaatatataaagaacttgtacacctcaatagtaagaaaacaaataacccaattttattttattttatttttattttttgctgttccttgcagagctaattcttaggcagtgggcccagggtcagccccaattttaaaatggaccaaaaacctgaataaacatttttgcaaaaaagacatacaaatggccaacaggtatatgaaaaaatgcttaatatcactaatcattagaaaaatgcaaattaaaatcacaatgatctatcacctcatgcctgttagaatggctataatagaaaacacaagtgttggtgaggatgtggagaaaagagaacgcttgtacactgttggtggaaatgtaaattagtgcagccattatagaaaagaatatggaggttcctcaaaacaattaaaaatagaactaccatatgatcccgcaatcccacttctgggtatatatccaaaggatatgaaattaatatgtcaaaaagatatctgcttgcccatgtttattgcagcattgttcacaataaccaagatatagaatcaacctaagtgtccatcaatggatgaataaagaaaatgtgtatctatacaatggaatgctattcagtcttaaaaaagaagggaatcttgtcatgtgacaacatggataaacgtggaggacattatgtcaagtgaaatatgccaggcacggaaagacaatctgaaaaagtcaaactcatagaagcagacagtagaattagaatggtggttattaggggctcatggttccccgttggtcgggaaggtcatgacataaaatagcaggattggagatatagatgctagtcaagaaaagtcaagaaatataaattttgagttaactatcagaaataccttcaggagatgtatgttgcggtgactattgttaataacatattgaatatttgaaagtagctaagagagttggttttaagtgttctcagcacaaaaaaataagtgaagtaatgcatatgctaattagcttgatttagccattccacaatgtatgcatatatcaaaacatcatgttgtacaccatacatatatacaatttttagtcatcagttaaaaaagggaaagaaaaggtggctcacatttctcccattgaatcttctttttttctaagtgcagtcatcccttcgtatcctcaggggatttgttccaggacccccgaggataccaaaatccatggaagctcaagtccctgatataaagtggtgtagtatttgcttattatctgagcacatcctcctatacactttaaatcatctctagattacttataatgccaacacaatgtgaatgctatgtgaagcattgttgtattgcattcttttgaattggcattcttttttatcattgtcttgttatgtttaattatttttttccaaatattttctaactgagactggttaaatccatggatggggaaccacagacacaacaaaggaccaactataatttcacttgtataagttccttatatgtgaggagagtaaaggaaagaaaatttcaaaggtcacagaattattaattgacaaggtagaagctcagcttccttaagttctagccaagcttctttgatatagtaaagtatttacaaaggagtcaatgttcaactcttattaatattatgggtttaaaatgttttcattatctaattatacttggtctcctctgattttgttaccactccccactcatgcaacgtcatgctctcttactggatacctgatggtttcttcccttttatactgggatctttccatttggctctaccagacaaatttcatttaagcaaattattagaatcagacgagaaattttaaaggcaatgtgacacgttttgcctaagacttgggcaaatgtctgaaactctctatactccagttcattttagcttcatatctgtgattgtttgctctttataaatactcacaacagggttataatatcccaggtattaactgagcctgtaaaactgcctgtgattttaggtggaggataaagggaaagagacagagctgattctaccggggatccaacagagctttgatgatatggactgtttcctgttttgcctgtgaagattgtccagagctaaagtaagaaaacatgtggagaatatggagataaaatatgagacaaaaggtagaaaatggacaaaaggaaaataatatatgtattatacattttataattaagagtgtaaaggattagctggaggtagtggtgtgggccggtagcctcagctacttggcaggctgaggtgggagaatgatttataagcccaagagatggagactgcagtgagctgtgattatgccactgcactttagcctgggcagcagagtaggaccttgtctctaaattaaattaaagaatgtaaaggggagccaggcacagtaacacacatctgtaatctcaactacttgggaggctgaggtgggaggattacttgagaccagcagtttgaggtcagcctggacaacatagtgagaccccatctctacataaaagaaaaagaaagaatgtgaaggggaatgctcaggtggtattgctatgaactacactgtcactatatcaaacagatcaattttagctgtaaatgacagagaccttaaaaaatggtgacttaaccattaggaaagttcttcctcaacagacagcaatctggaggtaggtgatccccagttgggtggggagctcatgacatagcaagggcccacacttttcccacctgcttagaggaccatcctctgcatgtcgaccaagtttcccattttgatcataaggtggctgctggagctcccagcacaaaattctcacacatcagctcctatcaatacctaagcaggaaggaagtggatatgggtaaagagagacttttctttttctatgcctttctcttaactgagaggacagtctttttctaaactactcaacagaacttagtagcatgcccctagaccaacacaaagagaaatgggatggcagtgttgtaaaccaatcctaattcatgtcttaggattagacatactactataagagagaaggtagactggccaatgggtaggcaagcatcagaatcagtcacacaaatacacacacacacacaaacacagaatcaatcaactatcagaattaataagggatttgggcgaggttgacagacgcaagactatactaaaaaacggattgcctttagcctcaataacaaaataagagtattttgtaaattatgccattcatttttgcaacaaaaataaagtatcttaaattaacccagatattttgttaacattagccaattgaccaagaatacacaagacatctatggaggaaattttaagacttttataaagggcacagatatgaatttagtacatgaaaagactttatagtctcaaacataatattttaaatatctaattacactaacgtaaaaatcaattaatatatgtaaagcagttgattaaaattccagttagatttcgtgaggaaatttatactctagaatttatatgccaactaatttcctagactaagttgccattttaaatgtagactcctactttgtaatattaaaacataaacagtagtaataaggagcaggatggtgtttatttgaataaggaattgaaaagggaagcagaatttctattcatagaataatggatttactatagtgttaagctttaggcaattatgggagctggcagaagagtctatataagcttttgcatctgcatctagtgttgacctcaagcgtttataggtcagacagactagcagatggaaaggaaagctagctgtggctgagaagatgggcaaactggaaccagtgaagacaaactggagctggaactggaacccacaagacagagcagaaccagtgtgtgtctcttgccatttccaacatcagtgctatgagtgatcagtagaaggtgggacctttcaccatggaactgtccacatacttggggcagaaattggagaatctggtggagatttgacaggagctggaggagctggatgtctcagttgattcaataagataagccagcagatcagtaacaatgcatgcaagatgttgctatacatggcactttctgaggaccttcagagcagatagatgatagatagatagatagatagatagatagatagatagatagatagatagatagacagatagacagatagatagacagatagctgccccttcacttaccacttttcaagtatcacacaaaatgcctcctgtggccaaaacaaactgaggaccataaggaaaaaataattacaggaaatgtaattccagtaaaactaatctgacacagttcaaagctatcacaatattgtattgttggtttggttcaagaacaaacagacaaaaaacagaagtgagtgccagggacatgtttatacagaaacttaatagacactaaaggaaaactagctatatgcacacaaagactggatctttatacaacataataaaaaatggactccagatagatttaagaataaatatgctgtaatcccagcactttgggaggccgaggtgggtagatcacaaggtcaggagttcaagatcagcctggccaagatggtgagaccccatctctactaaaaatacaaaaaattagctgggcatggtggcgggcgcctgtagtcccagctactcgggaggctgagggagagaattgcttgaatccgggaagcggaggttgcagtgagccaagatcgcaccactgcactccagcctgggtgacagagcgaaactccgtctcaaataaataaataaatgaatgaataaataaataaaagaataaatatgaatgttataactataaatttataaaataaaatatagaacatttttgtgacttaggagtagggaagggttacttatacaaaacttcagaagcacaagctataacaaaaagaaaaaaagtaaagtaatttgattacacctaaattgaggctttctgttgaacagagaatactttggacaagattgtgtagttaggcctgggtgtggtggttgacacctataatcccagcactttggaatgctgaggcaggaggatcacttgaggccaggaatttgagaccagtttgggcaacatagtgagactccatctctattaaattgaaaaataaatattttttaaaataaaaatttttgaatgtacaaataaaatcacagttgagcaaaggaatacaaaaaaacactcaagtgtattagctggatgcacacatatttattcccgcccccattgtgtaaaagcagccctactgcctcctaaagaacagggtccgtgatccctgccaagatgataacttctcttcgtgtctgccagtccctggacacaagaagcatgaaatgcctggtggtggttgttgtctacatttgctggatctcctagcaaaaccgtgcctcctttggtaaccaggaccctaacccttcagagcctagggttgtggggagagaaagttcaagtctccactgagtctttaggagtgatggtaagtggggtcactcctgcttctattccttagttcttggacccctgatttttcttactggagatacagtgtcatgtaaaaatgttttgattcaatcttctgggagatggaagccaatctatcctgatgcccagtagctggagctctttctggagaatgaaacccctttttggtaaagtattgtctctaagctagcatttcaactgtgtcttcggcatgacatttcaatctcccagactgccagcttctgagtggtgtggtatgtgatatgaccagtggatccaattgtcataggctcactcctatacctcctttgcagtaaagtgaactccctgctctaacagtgtcaggaaaggatcctttcctaaggagaaaacatttcacaaattcttggatggtgatggtagctaaggctgtgcatgcaggaaaagtaaacccacttccaaaacgtgtgtttattcttattagaacaaacctctggcttttccaagttctaaggggcttgtagtagttaacttaccactaaattgtctccttgagaaactatatatcagatgctcagcatcagtgtctgcccctggcaagttgaacatttgatgccagcaacagctaaattcaccttggtaagtgggaactcatactatctggcccatacataacatctgtctctcctaccatgcccatttcatcatcaacccattgtgccagcactggggtagctgatgacagaaactggtgtagtaatgctgtctacttggctgtttagatcaatctccatggttaacgctctttggtggatatgtgatacaaagactttctcacttcattcctactcccatttgttcaaccacctccttatctcattccttcaatcttactctttcatgggctcctgcccatgagtctggatttattgttacatcaagccgctactccttttacacaaagtggatcaacaggtgaatcattccaagctacctattagaaagattgtccttgtccctcactatagtcttttaaaacctcttcggaatgagactgcagttcaattgtttccatttttaagaaacacatactaagctcttccatttctaaatcaagctttggattttggttcttccattagctggtcatgagagattcccaatatgatcatagtgaggtgaaggagaggtcttagtatgcagtggtgaaattcatgggggtctgggctacatgcttatgctgcttgttttttgctagtccttcttatgcttggagctcatgcttatctttagataactgctgtggcgcttaagcaaccttatgatttggagattctgtgtgaatcaagctcatgttgggcatttcttggaggcatgaccattggtgtctcctggtcaggcatttttatgaattctatgaatgccgggcagtatcccaggagttttgttttggttttttcaagtatataattctctttgctccagaatctcagaagctggcactgttattctcctgctggggcttactgtagccccattgccactttgtatcttttcttgctactggtatttctaataccacagtatctgcagagtcttgtcgtccaagtagtggagctgctagtcctccaccttggatttggtacagaaatcttttctgcactaggaccttcttaaagatgccaatatttctttctttttcttttttttttttttttttttgacagggtcttgctctgttgcctaggctagagtgcaatggtgggatcacagctcagtgcagcctcaactgcccaggctcaagtgattcttctacctcagcctcctgagtagctgggactacaggctcatgccaccacaccagctgattttttttattttttgtacagatgaggttttgcaaggttgctcaggctaaagatggcaatatttcttatcagttggtgcatgggctgaagtattccctagtgcggaataagctatctccagaattgagggcaacgtattatgcatagaggtttcttccttgtagtgaaaggtgcaagatgtaatggtttgtcttttactttgaaatatgtgccctgatatattaatacttttgactacaggactcctaaaattttgcttacatgtcagcccctggttctctataggctttatctcatatcctatggagcacatgtatcttactaagtcctctaatgcgctagccattttttattcattagctccaattaacataatatcattgatgtagtaggtcattgtgaagttttataggatgcccagatggtccatatctctctggactgtattattacagaaagcagaagaattagcatagctttgggacaaaaccataaatatgtctttttacccataccatgagaatgcaaactctttctgatcagaatataaaagaatgtttttgccaagtcagtggctatgtaccatgtacctgaaacccttttcatctcctctagcaaaggtattaattatatctagtcctgtggctgaaattagggaaacaacttatttaagtttgcagtaaactgacactttccaggagcaatctggtttcttgtggagccaaattggcaaattaaatggaaatatgagagcatctattatctctgcatcctcaaaagtagtactaatttctgccatctaccaacctggggtggttatattgtttttggtcctttgatgatatcttagttcatttgtgctgctacaacaaaatgccacatattgggtaatatttaaacaacagaaatttattttcttgcagctccagaggctaggaagtctaggattaaggtgacggaaagtttgatatctgtcaagggcctgttccctgtttccaggctagcactttgttgctgcgtcctcatatggtggaagagatggaagggaataaacctgctccctcaaagccttttataagtcctaatcctatccaggatggctctgccctcataatttaatcacctccaaaaggccctccatctggcagatcacttgaggttaggagttcaagaccagcctggacaacatgtcgaaaactcatctctactaaaactacaaaaattagccaggcatggtggcacatgcctgtaatcccaactacttgggaggctgaggcaggagaattgcttgaacccaggaggtggaggttgtagtgagctgagatcacaccacagcactccagcctgggcaacagagcaatttgctgtctcaagaaacaaaacaaaacaaaacacctccctcttaatactatcacattaaccattaagtttcaacacatgaattttgggggacacatttagaccgtatcagatgggattgagtagaagatggggtatttttagaggcttccacttggcatttcccactatgagatcccttactctacaggccaaagacccaaagttagggttgtgccaagtactacatagggcaatcccaattatacttttgtgaaccagaggaatgacagctagttgggaaaacaaacccaataggcctgctataagcaagactttggtcataacactgtaaggcctcattctaacagggagaccatgatgttactttgggtctctggattttgatgtcacctcagacctttgcctcccacaatgtccaaaatatttgggatacctgagtaagtgggcatttgtccctttggagaaagactagcaggtattattaacacgtatatttggctgggcacagtagctcacacctgtaatcccagcactttgggaggccgaggtgggtggatcacctgaggtcagaagttcgagaccagcctgaccaacatgaataaaccccatctctactaaaaatacaaaattagctggccgtggtggctcatgcctctaatcccagctactcaggaggctgaggcaggagaatcgcttgaacctgggaggcggaggtgagccgagagatgtcgccactgcactccagcctgggcaacaagagcaaagctccatctcaaaaaccaaagaaacaaacacacaaaaaaatgtatatttggcatgaggttagggtctatccttctggaagcctggcccttttttcagtcaatgggttctgtagctgaaaacgaatttagatccaggagctaaattgctctcaatttagttgtcacccatattcaatttcttctgatagtacaggccgagttatactcttactgtcaatatattttgcccctatggatactgtattccaaccatctcctttactctctgacggtcaggcccttttggttataaccctatgagtctgctactcatttagataattatgtcaatgtggcttctggcagttcctaccataacacaccctctattgttttggaatcctaacatccccactgcttttattgagtctactctgtaatggcctctcctaatattaaccctgtctaataaagaaatcccagcactaaacattttagtgatgctggtacccctctcacctgtgcattactatggccttgctaatggtgtgcacaatagtccctctcatggtacataatcatctggtgagtctttcagcattacatagatatttaatgcctatttcagcatgcccatttccttaagcttttaatcctttcctccactgcctaccatagcaattctggcatttcaactttagtatggtttgttattttctgcatgcttctgggatctatactagtagtgagttgctaccgtctcctcagatccttgacagagtattgaatcttgtatttttggagagtgccccaaataaataaagtctctcctatccaatatattgttccagattgcttaatggtgcaccctcagaatccagtgccaataatgccaaccaatacattctgtttaggtcttgcaattcctttgggtagagcttcttttcatccttatcaagcccagcaaatctgaagctgtattacgttggaacttaaccctggttatagatctagtggccaggaagggaaatgggagcaaatcaggaggcaaatcatgtgcctacaggaaaagccctctccattgtcttcaagcaagaaggatgtgctaactcttggagagggatggtcacttctgcagaccaggagaattcaaagaagcctaggggttcaaaatcttaaatgacattcacccaaatctccccagctcaagggacagtgtcctgtttttcctaatcttggccctaaccttagcataacagaattgctttaattctgcatttcctttcttgccattattattactaattaagaaaaaagccctctctgtctgtatgtgtacaagtgtacacacatgcatgtacatccaatgaagtgaatatgaatttaaagaaatctctagaagaaaatatcaaacttttaatactggttaaatacatgaaggagagtacaatgaaaagctttggagaaagaaatttgcacaattttctatacattcttccatgtctaaatattttttgcaaatatttgtataacattttaaagtattaggaaaagtaaaaggatcaatcaacgtaggggaagagaactggaagagggaagaaaactttagccatgtcctttattctctcctaagaagtttgacctcacttcacccaacccaagaggagagaatgaatgggtgatgggcattgagagcttgtgcgatgagtcatttccttgcccctggagaaggtgggaatgaggagaagggggcttactgtccgtagtcagccatactctttcattcctccttcagaaagggcccaagggcttggggattgcagctctgatccagggaccatgggaaccacatgagaggcatatgaacctgggtacattgcagaaggatgattcaactagggacagtcatgagtgacaccacaggaggaggtgtatctgtgcagaaacagctcttcacagctttttggaacatcggcatggtaggaaagggcaaaataatctatataaataaagttaagtttctgtctgttaaaacaattgattagacagattttcaatacatttgacaaaggtatttagaatcacatattggttttctgtagctactatagtaacaaattaccacagacctggtggtttcaaacaacaggaatttattctctcacagttctggaggtcagaattccaaaattggattcataaggcagaaatcagggtgttggcaggaacatgctccccctaaaggctctaggggccttgactcttccagtttctggtggctggcagtattccttgacttgtggccatgtcaccccaattctcctatgacttcacatcatctctctgtgtagataaaacttccctctccctccctcctataaaggcccttgagattccatttaaggtccaccgtaaatgtaatttggataatctccccatcccaagatccttaacttaatcaaatctatagaaactctttatccttatcaattaacacttacaggtcccagaggttgcaacttgacatgcttgggagccattcagcctactgcagatggactgaattaaaacatgcaacacacatgaagttaatgttgggaattctggtttttcaaagagcagtctgcttctagagaacccaaaattgagaaacaatagcagtttcgtgtatctgtcaatcagaagagagctatggtatgtattaagacaccaattagagaaaataattggtggcttcaaagtcgagctctgcctccccaactcaacctattgagtagaatttctgcatttgattcttaccctgactaaaggcataagagtcgacactgatgtgtaggcttcagttcacaggaagtctgcttctcacatgagcaactccctatagctgacttctcagtaataagtagggatttatttgtgtaaccatgtagaatccaagtaggaagtcaaattgtagtaataatactagctaacgtttcttgagcatttattatatcccttgtaatgtgctaattgcctctcatgtattgtcctatttaatttcaaagcaaacttttgaggtagcttgactcactaaggctctttgattatatccaccacaagcaaacaagattaattggaaggcctctgggctggctcacaaatcaggagagctgcaaaaacagggttggtaaggacagaaataacagcagttcaagtgtgaagaactcagttcttggtgcttttctcagttatcattggataactatctagatgacctttcacctctgtgggtttccactcagagtttgaattccaagcagacttaatctcaccctggtgggacagagccacaatcattggctttgcctgggacaaatgcccaccctgggccaggggagcaggttcagacatgaaaaaagagtgaggagtcgggtgcagtggctcaaacctgtagtcctaacactttgggaggccgaggcgggtggatcacctgaggtcaggggtttgagaccagcctggccaacatgccgaaaccccgtctttactaaaaatacaaaaattagctgggtgtggtggcacatgcctgtaatcccagctacttggcaggctgagacaggagaatcgctggaacctggaaggcagaggttgcagtcagcctagatcatgccactgcactccagtctgggtgacagagcgagactctgactcaaaaaaaaaaaaaaaagtatgtgtctcaaacactattttgcaagcgtgaaagaaggaagagatagaattgtgacgttaagttccccaagtcactaagcaagctgatagtgaaactataatatatctttcagtctgtgacctttggaactctctgataagatgcagactactttagccatgtagtagacagctgatgtttttgctaggccagtactgtggattaaattgtgtcctcccaaaattcacatgttgaaaccctaaccccccaatgtgatggtatatggagataaggcccttgggagataattcggtttagataaggtcatcagagaagagtcctcttgatgggattaatgcctttataaaaagaagaaaaagttaccagagcgctctctctgccacatgaggacacagcaagatgacagctttctgcaagccaagagtgtgtcctcaccagaactcatccatgccagcaccctgatcttggtcttccaacctccagaactgtgaaaagggtctctgttatttaaatcaccagtctatggtatttgttatagttggtgcccaagccaactaagacaaccatgccatccatgccagcaccctgatcttggtcttccaacctccagaactgtgaaaagggtctctgttatttaaatcaccagtctatggtgtttgttatagttggtgcccaagccaactaagacaaccatgccatccatgccagcaccctgatcttggtcttccaacctccagaactgtgaaaagggtctctgttatttaaatcaccagtctatggtgtttgttatagttggtgcccaagccaactaagacaaccagcatccgtaccccttattttggaagcagctttccaattctaggaagtatttttttttttttagacagagtctcactttgtcacccaggctggagtgcacagtggcatgatcttggctcattggaaccttcgcctcccaggttcaagtgattctcctgcctcagcctctccagtagctgagattacaggtgcctgccaccgtgcccagctaatttttgtattcttagtaaagacggggtttcaccattttggccaggctggtgtgaaactcctgatctcaggtgatccacccatctaggcagtcttaataggaaagtcaaggcccggtgcggtggctcacacctgtaatcccagcactttgggaggccgaggcaggcagatcacgaggtcaggaattagagaccaacttggccaatatagtgaaaccctatctctactaaaaatacaaaaattagccgggcatgatggtgcgcgcctgtaatcccagctactcaggtggccgaggcaggagaatggcgtgaacccgggaggcggagcttgcagcgagccaaggtcgcgccactgcactccagcctgggtgacagagtgagactccgtctcaaaaaaaaaaaaaaagtcaagcacagggccctctaccaaaccaagccaatcagactctccagggagaatgacagagagactctaggtgggtgaaatggggttattctcatggcatgacctaaggaaagaaggtatgtatatttctgactaagattcggtagcttcctgctcctctttcttcccaagacctggctcatcagtgtttcctttaattctgtaagctataaagtgtccttccaataaattcctttttccatctaggtgagtcagagcttctttcagttttttgcagaactgattcagcaatggtagctgggagtgagcactgcaagcaacagaactgctgtggaaatagcagaattaggtgcaaggcaatgagaattc

SEQ ID NO: 8 - DNA, 2236 bp, Homo sapiens
gggacttcaccagtgctgccatgctggccccaggctgtgagttggacccagaccaagaagtggtgaggacaaggcctgaagatgtgcctgcttcccattcaacttccaccatgattgtaagtgtcctgaggcctcccagccatgcttcctgtacagcctgtggaactgtgacttttcacatagtagagagaatggcttcagttatacctgcctccaataggtccatgagatctgacaggaatacatatgttggaaaaaggtttgttcacgttaaaaatccttacttggatttgatggatgaagacattctctatcacttggatttgggaacaaaaacacacaacctaccagcaatgtttggagatgtaaagtttgtctgtgtcggtgggagccccaacagaatgaaagcatttgcactgtttatgcacaaggagctcgggtttgaggaagctgaagaagacataaaagacatctgtgctgggacagacagatactgtatgtacaaaaccgggcctgtgctcgccatcagtcacggcatgggcatcccctccatttctattatgcttcatgaactcatcaaattactccaccatgcacggtgctgcgatgtcaccattattagaatcggtacatcagggggaatagggattgcaccagggactgttgtaataacggatatagctgtagactccttctttaagccccggtttgaacaggtcattttggacaacattgtcacccgaagtactgaactggacaaagaactgtctgaagaactgttcaactgtagcaaagaaatccccaacttcccaaccctcgttggacatacaatgtgtacctatgatttttatgaaggccaaggccgactagatggagcactgtgctccttttccagagaaaaaaagttagactacttgaagagagcatttaaagctggtgtcaggaatattgaaatggaatctacagtgtttgcagctatgtgtggactctgtggtctaaaagctgctgtggtctgtgtgacacttctcgacagactcgactgtgatcagatcaacttgcctcatgatgtcctggtggagtaccagcaacggcctcagctcctaatctccaacttcatcagacggcggcttggactttgtgactagacgtcctaactgggcagcccaaccctcccctgcaagtttgtagctcaagttgtaatgtgaaagtcatattttatttgtggcatttttatatagttctcatccacatgctaaatggaaagactttatgaaatccttctctcttaaaaaggaatttattgtaaaagaatactcacactaaattaaattcaaatttcattttagaataagttaactaaatcagtctaataattaaaattttaaaactcctggattatgacatttggagtttcatatgcagcattaattaagctcacatcaaaatagaccagccatttgcacatagttaccagtcactctgtttctgaaaccaatccagaaattcatgttagaacattgtcaggcacttctaggtattggacaagtgacagaaactgatcatccatattcaaaatatttacagaaaaaagtgtcagtgaaatgtaggtctatatggaaaagttaccattaggaaaatatggccatactgcttttttacccctgcactatttcagtgcaccacagaataggattcgagatttacatatgcctgtttgtgattttatctcagaatataacaactctggattacattttcctctcaactgaaaaccctcagtaatgcaaagaaaggccttcctttttcttcagaatcacaagtgactaaattgccttctagccattttttcttgtaaaaaaagtgttttgaggaggctgtattttttattccatttcaatattaaccaaataggctgagaaattcatagcaattgaattaagacttgagttcaagatgttaaaggaatccttttgtatgacttgtccagaccggcctattgccctacccgtttgattctctgggaggcatggtgctcctcctggctgcacatgctctctgtgaactgatcgcatcttgggttcttctcaatttgacatacttgggagtcggaatgactggatgggggatgggtagaggatgactggcacacttgtatagtatgtaatgttgtgtacatattatttatatggttgtggctcaaataaagtttaattccaacacaaaaaaaaaaaaaaaa

SEQ ID NO: 9 - DNA, 2236 bp, Homo sapiens; identical to SEQ ID NO: 8

SEQ ID NO: 10 - protein, 310 aa, Homo sapiens
MAATGANAEKAESHNDCPVRLLNPNIAKMKEDILYHFNLTTSRHNFPALFGDVKFVCVGGSPSRMKAFIRCVGAELGLDCPGRDYPNICAGTDRYAMYKVGPVLSVSHGMGIPSISIMLHELIKLLYYARCSNVTIIRIGTSGGIGLEPGTVVITEQAVDTCFKAEFEQIVLGKRVIRKTDLNKKLVQELLLCSAELSEFTTVVGNTMCTLDFYEGQGRLDGALCSYTEKDKQAYLEAAYAAGVRNIEMESSVFAAMCSACGLQAAVVCVTLLNRLEGDQISSPRNVLSEYQQRPQRLVSYFIKKKLSKA

SEQ ID NO: 11 - protein, 317 aa, Homo sapiens
MASVIPASNRSMRSDRNTYVGKRFVHVKNPYLDLMDEDILYHLDLGTKTHNLPAMFGDVKFVCVGGSPNRMKAFALFMHKELGFEEAEEDIKDICAGTDRYCMYKTGPVLAISHGMGIPSISIMLHELIKLLHHARCCDVTIIRIGTSGGIGIAPGTVVITDIAVDSFFKPRFEQVILDNIVTRSTELDKELSEELFNCSKEIPNFPTLVGHTMCTYDFYEGQGRLDGALCSFSREKKLDYLKRAFKAGVRNIEMESTVFAAMCGLCGLKAAVVCVTLLDRLDCDQINLPHDVLVEYQQRPQLLISNFIRRRLGLCD